(19) United States
(12) Patent Application Publication
(10) Pub. No.: US 2020/0216417 A1
(43) Pub. Date: Jul. 9, 2020

(54) INHIBITORS OF FIBROBLAST ACTIVATION PROTEIN

(71) Applicant: Praxis Biotech LLC, San Francisco, CA (US)

(72) Inventors: Sebastian BERNALES, Piedmont, CA (US); Brahmam PUJALA, Greater Noida (IN); Dayanand PANPATIL, Noida (IN); Gonzalo Andrés URETA DÍAZ, Santiago (CL); Sebastian BELMAR, Santiago (CL)

(21) Appl. No.: 16/734,214

(22) Filed: Jan. 3, 2020

Related U.S. Application Data
(60) Provisional application No. 62/863,853, filed on Jun. 19, 2019.
(60) Provisional application No. 62/788,722, filed on Jan. 4, 2019.

(51) Int. Cl. C07D 401/14; C07D 401/12; C07D 405/14; C07D 417/14; C07D 413/14; C07D 471/04; A61P 35/00

(52) U.S. Cl. CPC C07D 401/14; C07D 401/12; C07D 405/14; A61P 35/00; C07D 413/14; C07D 471/04; C07D 417/14

(57) ABSTRACT

Compounds and compositions for modulating fibroblast activation protein (FAP) are described. The compounds and compositions may find use as therapeutic agents for the treatment of diseases, including hyperproliferative diseases.

### CROSS REFERENCE TO RELATED APPLICATIONS

This application claims priority to U.S. Provisional Application Ser. No. 62/788,722, filed Jan. 4, 2019, and U.S. Provisional Application Ser. No. 62/863,853, filed Jun. 19, 2019, both of which are hereby incorporated herein by reference in their entirety.

### FIELD OF THE INVENTION

The present disclosure relates generally to therapeutic agents that may be useful in modulating fibroblast activation protein.

### BACKGROUND

Fibroblast activation protein (FAP), also referred to as FAPα, Seprase or α2-antiplasmin converting enzyme, is a type II integral membrane serine protease that belongs to the prolyl oligopeptidase family S9, which also includes DPPII, DPPIV, DPP8, DPP9, and PREP enzymes. This family is characterized for having an exo-dipeptidyl peptidase (DPP) activity. FAP is the only member that also has an endopeptidase activity (Aertgeerts, K., et al. J Biol Chem, 2005. 280(20): p. 19441-4). FAP has a high degree of homology with DPPIV. It is mainly found as a cell surface homodimer but it has also been reported to form heterodimers with DPPIV in vivo (O'Brien, P., et al. Biochim Biophys Acta, 2008. 1784(9): p. 1130-45). Purported physiological substrates of FAP endopeptidase activity include α2-antiplasmin, type I collagen, gelatin, and Fibroblast growth factor 21 (FGF21) (Lee, K. N., et al., Biochemistry, 2009. 48(23): p. 5149-58), and for the exopeptidase activity include Neuropeptide Y, B-type natriuretic peptide, substance P and peptide YY (Brokopp, C. E., et al., Eur Heart J, 2011. 32(21): p. 2713-22; Coppage, A. L., et al., PLoS One, 2016. 11(3): p. e0151269; Dunshee, D. R., et al., J Biol Chem, 2016. 291(11): p. 5986-96; Lee, K. N., et al., J Thromb Haemost, 2011. 9(5): p. 987-96).

FAP has been implicated in diseases involving proliferation, tissue remodeling, chronic inflammation and/or fibrosis, including but not limited to fibrotic disease, wound healing, keloid formation, osteoarthritis, rheumatoid arthritis and related disorders involving cartilage degradation, atherosclerotic disease, and Crohn's disease.

FAP expression is related to poor prognosis in several types of cancer including gastric cancer, pancreatic adenocarcinoma and hepatocellular carcinoma, (Wen, X., et al., Oncol Res, 2016; Cohen, S. J., et al., Pancreas, 2008. 37(2): p. 154-8; Ju, M. J., et al., Am J Clin Pathol, 2009. 131(4): p. 498-510) and in colon cancer, increased FAP expression has been associated with a more aggressive disease (Henry, L. R., et al., Clin Cancer Res, 2007. 13(6): p. 1736-41). Purportedly, FAPα on CAFs has critical roles in regulating antitumor immune response by inducing tumor-promoting inflammation (Chen, L., et al., Biochem Biophys Res Commun, 2017; Wen, X., et al., Oncol Res, 2016; Hugo, W., et al., Cell, 2016. 165(1): p. 35-44).

Val-boroPro (Talabostat, PT-100) is the only FAP inhibitor that reached clinical stages. This compound was originally developed as a DPPIV inhibitor and subsequently evaluated as a FAP inhibitor regardless of its lack of selectivity (Cunningham, C. C., Expert Opin Investig Drugs, 2007. 16(9): p. 1459-65). This agent was tested in Phase II in a variety of cancers in combination with standard cytotoxic chemotherapy, however endpoints for efficacy were not met (Eager, R. M., et al., BMC Cancer, 2009. 9: p. 263; Narra, K., et al., Cancer Biol Ther, 2007. 6(11): p. 1691-9; Eager, R. M., et al., Clin Oncol R Coll Radiol, 2009. 21(6): p. 464-72). Two Phase III trials were early terminated, apparently because of both safety and efficacy concerns (Jansen, K., et al., J Med Chem, 2014. 57(7): p. 3053-74). Since Val-boroPro rapidly loses protease inhibitory activity due to cyclization upon standing in pH 7.8, effective concentrations were difficult to achieve in patients given the clinical toxicities seen with this agent at higher doses (Narra, K., et al., Cancer Biol Ther, 2007. 6(11): p. 1691-9).

There is scope to improve FAP inhibitor selectivity and the properties of the inhibitors to improve safety and efficacy in vivo.

### BRIEF SUMMARY

Provided herein are compounds, salts thereof, pharmaceutical compositions of the foregoing and methods of making and using the same. In one aspect, provided a compound of formula (I):

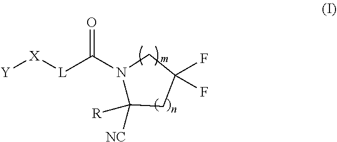

a pharmaceutically acceptable salt, stereoisomer or tautomer thereof, wherein R, m, n, X, Y and L are as detailed herein.

In one aspect, provided is a compound of formula (I):

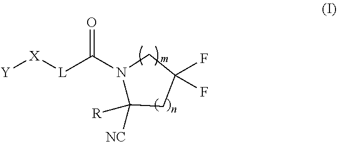

a pharmaceutically acceptable salt, stereoisomer or tautomer thereof, wherein:

R is hydrogen, C₁-C₆ alkyl, C₃-C₈ cycloalkyl, 3- to 12-membered heterocyclyl, 5- to 10-membered heteroaryl, or C₆-C₁₄ aryl, wherein the C₁-C₆ alkyl, C₃-C₈ cycloalkyl, 3- to 12-membered heterocyclyl, 5- to 10-membered heteroaryl, and C₆-C₁₄ aryl of R are independently optionally substituted by R^{d};

m is 0, 1, 2, 3, or 4;

n is 0, 1, 2, 3, or 4,
wherein m+n is 1, 2, 3, or 4;

X is —C(═O)—, —O—, —CH(OH)—, —S—, —S(═O)—, or —S(═O)₂—;

L is

(a)

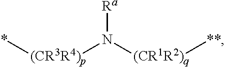

wherein
* represents the point of attachment to the Y—X— moiety,

** represents the point of attachment to the remainder of the molecule,

R^{a} is hydrogen, C₁-C₆ alkyl, C₃-C₈ cycloalkyl, 3- to 12-membered heterocyclyl, 5- to 10-membered heteroaryl, or C₆-C₁₄ aryl, wherein the C₁-C₆ alkyl, C₃-C₈ cycloalkyl, 3- to 12-membered heterocyclyl, 5- to 10-membered heteroaryl, and C₆-C₁₄ aryl of R^{a} are independently optionally substituted by R^{e},

R¹ and R², independently of each other and independently at each occurrence, are hydrogen, C₁-C₂ alkyl, C₃-C₈ cycloalkyl, 3- to 12-membered heterocyclyl, 5- to 10-membered heteroaryl, or C₆-C₁₄ aryl, wherein the C₃-C₈ cycloalkyl, 3- to 12-membered heterocyclyl, 5- to 10-membered heteroaryl, and C₆-C₁₄ aryl of R¹ and R² are independently optionally substituted by R^{f},

or R¹ and R² are taken together with the carbon atom or atoms to which they are attached to form a 3- to 8-membered cycloalkylene optionally substituted by R^{f},

q is 1, 2, or 3,

R³ and R⁴, independently of each other and independently at each occurrence, are hydrogen, C₃-C₈ cycloalkyl, 3- to 12-membered heterocyclyl, 5- to 10-membered heteroaryl, or C₆-C₁₄ aryl, wherein the C₃-C₈ cycloalkyl, 3- to 12-membered heterocyclyl, 5- to 10-membered heteroaryl, and C₆-C₁₄ aryl of R³ and R⁴ are independently optionally substituted by R^{g},

or R³ and R⁴ are taken together with the carbon atom to which they are attached to form a 3- to 8-membered cycloalkylene optionally substituted by R^{g}, and

p is 0, 1, or 2;

(b)

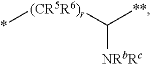

wherein
* represents the point of attachment to the Y—X— moiety,

** represents the point of attachment to the remainder of the molecule,

R⁵ and R⁶, independently of each other and independently at each occurrence, are H, C₁-C₆ alkyl, C₃-C₈ cycloalkyl, 3- to 12-membered heterocyclyl, 5- to 10-membered heteroaryl, or C₆-C₁₄ aryl, wherein the C₁-C₆ alkyl, C₃-C₈ cycloalkyl, 3- to 12-membered heterocyclyl, 5- to 10-membered heteroaryl, and C₆-C₁₄ aryl of R⁵ and R⁶ are independently optionally substituted by R^{h},

R^{b} and R^{c} are independently H, C₁-C₆ alkyl, C₂-C₆ alkenyl, C₂-C₆ alkynyl, C₃-C₈ cycloalkyl, 3- to 12-membered heterocyclyl, 5- to 10-membered heteroaryl, C₆-C₁₄ aryl, or —C(═O)OR¹⁷, wherein the C₁-C₆ alkyl, C₃-C₈ cycloalkyl, 3- to 12-membered heterocyclyl, 5- to 10-membered heteroaryl, and C₆-C₁₄ aryl of R^{b} and R^{c} are independently optionally substituted by R¹, and

r is 1, 2, or 3; or

(c)

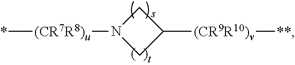

wherein
* represents the point of attachment to the Y—X— moiety,

** represents the point of attachment to the remainder of the molecule, R⁷ and R⁸, independently of each other and independently at each occurrence, are hydrogen, C₃-C₈ cycloalkyl, 3- to 12-membered heterocyclyl, 5- to 10-membered heteroaryl, or C₆-C₁₄ aryl, wherein the C₃-C₈ cycloalkyl, 3- to 12-membered heterocyclyl, 5- to 10-membered heteroaryl, and C₆-C₁₄ aryl of R⁷ and R⁸ are independently optionally substituted by R^{j},

or R⁷ and R⁸ are taken together with the carbon atom to which they are attached to form a 3- to 8-membered cycloalkylene optionally substituted by R^{j},

R⁹ and R¹⁰, independently of each other and independently at each occurrence, are H, C₁-C₆ alkyl, C₃-C₈ cycloalkyl, 3- to 12-membered heterocyclyl, 5- to 10-membered heteroaryl, or C₆-C₁₄ aryl, wherein the C₁-C₆ alkyl, C₃-C₈ cycloalkyl, 3- to 12-membered heterocyclyl, 5- to 10-membered heteroaryl, and C₆-C₁₄ aryl of R⁹ and R¹⁰ are independently optionally substituted by R^{k},

s is 1, 2, or 3,

t is 1, 2, or 3,

wherein s+t is 2, 3, or 4,

u is 0 or 1, and

v is 0 or 1;

Y is C₆-C₉ aryl substituted by R¹¹, 6- to 10-membered heteroaryl substituted by R¹², or 3- to 12-membered heterocyclyl substituted by R¹³, wherein

each R¹¹, R¹², and R¹³, are independently C₁-C₆ alkyl, C₂-C₆ alkenyl, C₂-C₆ alkynyl, C₃-C₅ cycloalkyl, C₄-C₈ cycloalkenyl, 3- to 12-membered heterocyclyl, 5- to 10-membered heteroaryl, C₆-C₁₄ aryl, —OR¹⁴, —NR¹⁵R¹⁶, —SR¹⁴, —NO₂, —C═NH(OR¹⁴), —C(O)R¹⁴, —OC(O)R¹⁴, —C(O)OR¹⁴, —C(O)NR¹⁵R¹⁶, —NR¹⁴C(O)R¹⁵, —NR¹⁴C(O)OR¹⁵, —NR¹⁴C(O)NR¹⁵R¹⁶, —S(O)R¹⁴, —S(O)₂R¹⁴, —NR¹⁴S(O)R¹⁵, —NR¹⁴S(O)₂R¹⁵, —S(O)NR¹⁵R¹⁶, —S(O)₂NR¹⁵R¹⁶, or —P(O)(OR¹⁵)(OR¹⁶), wherein the C₁-C₆ alkyl, C₂-C₆ alkenyl, C₂-C₆ alkynyl, C₃-C₈ cycloalkyl, C₄-C₈ cycloalkenyl, 3- to 12-membered heterocyclyl, 5- to 10-membered heteroaryl, and C₆-C₁₄ aryl of R¹¹, R¹², and R¹³ are substituted by R^{L};

R¹⁴, R¹⁵ and R¹⁶, independently of each other and independently at each occurrence, are hydrogen, C₁-C₆ alkyl, C₂-C₆ alkenyl, C₂-C₆ alkynyl, C₃-C₈ cycloalkyl, C₆-C₁₄ aryl, 5- to 10-membered heteroaryl, or 3- to 12-membered heterocyclyl, wherein the C₁-C₆ alkyl, C₂-C₆ alkenyl, C₂-C₆ alkynyl, C₃-C₈ cycloalkyl, C₆-C₁₄ aryl, 5- to 10-membered heteroaryl, and 3- to 12-membered heterocyclyl of R¹⁴, R¹⁵ and R¹⁶ are independently substituted by C₁-C₆ perhaloalkyl, C₁-C₆ alkoxy, C₁-C₆ perhaloalkoxy, C₆-C₁₄ aryl or C₆-C₁₄ aryloxy wherein the C₆-C₁₄ aryl or C₆-C₁₄ aryloxy is further optionally substituted by halogen, —OH, cyano, C₁-C₆ alkyl, C₁-C₆ perhaloalkyl, C₁-C₆ alkoxy, or C₁-C₆ perhaloalkoxy; and, wherein at least one of R¹⁴, R¹⁵ and R¹⁶, when present, is not hydrogen;

R^{L} is C₁-C₆ alkyl, C₂-C₆ alkenyl, C₂-C₆ alkynyl, C₃-C₈ cycloalkyl, C₆-C₁₄ aryl, 5- to 10-membered heteroaryl, or 3- to 12-membered heterocyclyl, wherein the C₁-C₆ alkyl, C₂-C₆ alkenyl, C₂-C₆ alkynyl, C₃-C₈ cycloalkyl, C₆-C₁₄ aryl, 5- to 10-membered heteroaryl, or 3- to 12-membered heterocyclyl of R^{L} is substituted by halogen, —OH, cyano, oxo, —NH₂, —NH-(3- to 12-membered heterocyclyl), —O-(3- to 12-membered heterocyclyl), C₁-C₆ alkyl, C₁-C₆ perhaloalkyl, C₁-C₆ alkoxy, C₁-C₆ perhaloalkoxy or C₆-C₁₄ aryl, wherein
the C₁-C₆ alkyl is further optionally substituted by 3- to 12-membered heterocyclyl, wherein the 3- to 12-membered heterocyclyl is further optionally substituted by C₁-C₆ alkyl,
the 3- to 12-membered heterocyclyl of the —NH-(3- to 12-membered heterocyclyl) and the —O-(3- to 12-membered heterocyclyl) is further optionally substituted by C₁-C₆ alkyl, and
the C₆-C₁₄ aryl is further optionally substituted by halogen, —OH, cyano, C₁-C₆ alkyl, C₁-C₆ perhaloalkyl, C₁-C₆ alkoxy, or C₁-C₆ perhaloalkoxy; and
   R^{d}, R^{e}, R^{f}, R^{g}, R^{h}, Rⁱ, R^{j}, and R^{k}, independently of each other and independently at each occurrence, are halogen, C₁-C₆ alkyl, C₂-C₆ alkenyl, C₂-C₆ alkynyl, C₃-C₈ cycloalkyl, C₆-C₁₄ aryl, 5- to 10-membered heteroaryl, 3- to 12-membered heterocyclyl, —OR¹⁴, —NR¹⁵R¹⁶, cyano, or nitro.

In one aspect, provided is a compound of formula (I):

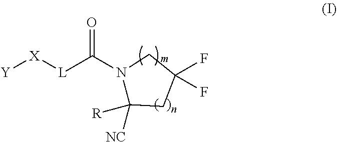

a pharmaceutically acceptable salt, stereoisomer or tautomer thereof, wherein:

R is hydrogen, C₁-C₆ alkyl, C₃-C₈ cycloalkyl, 3- to 12-membered heterocyclyl, 5- to 10-membered heteroaryl, or C₆-C₁₄ aryl, wherein the C₁-C₆ alkyl, C₃-C₈ cycloalkyl, 3- to 12-membered heterocyclyl, 5- to 10-membered heteroaryl, and C₆-C₁₄ aryl of R are independently optionally substituted by R^{d};

m is 0, 1, 2, 3, or 4;

n is 0, 1, 2, 3, or 4,

wherein m+n is 1, 2, 3, or 4;

X is —C(═O)—, —O—, —CH(OH)—, —S—, —S(═O)—, or —S(═O)₂—;

L is

(a)

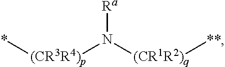

(wherein

* represents the point of attachment to the Y—X— moiety,

** represents the point of attachment to the remainder of the molecule,

R^{a} is hydrogen, C₁-C₆ alkyl, C₃-C₈ cycloalkyl, 3- to 12-membered heterocyclyl, 5- to 10-membered heteroaryl, or C₆-C₁₄ aryl, wherein the C₁-C₆ alkyl, C₃-C₈ cycloalkyl, 3- to 12-membered heterocyclyl, 5- to 10-membered heteroaryl, and C₆-C₁₄ aryl of R^{a} are independently optionally substituted by R^{e},

R¹ and R², independently of each other and independently at each occurrence, are hydrogen, C₁-C₂ alkyl, C₃-C₈ cycloalkyl, 3- to 12-membered heterocyclyl, 5- to 10-membered heteroaryl, or C₆-C₁₄ aryl, wherein the C₃-C₈ cycloalkyl, 3- to 12-membered heterocyclyl, 5- to 10-membered heteroaryl, and C₆-C₁₄ aryl of R¹ and R² are independently optionally substituted by R^{f}, or R¹ and R² are taken together with the carbon atom or atoms to which they are attached to form a 3- to 8-membered cycloalkylene optionally substituted by R^{f},

q is 1, 2, or 3,

R³ and R⁴, independently of each other and independently at each occurrence, are hydrogen, C₃-C₈ cycloalkyl, 3- to 12-membered heterocyclyl, 5- to 10-membered heteroaryl, or C₆-C₁₄ aryl, wherein the C₃-C₈ cycloalkyl, 3- to 12-membered heterocyclyl, 5- to 10-membered heteroaryl, and C₆-C₁₄ aryl of R³ and R⁴ are independently optionally substituted by R^{g}, or R³ and R⁴ are taken together with the carbon atom to which they are attached to form a 3- to 8-membered cycloalkylene optionally substituted by R^{g}, and

p is 0, 1, or 2;

(b)

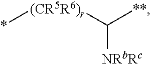

wherein

* represents the point of attachment to the Y—X— moiety,

** represents the point of attachment to the remainder of the molecule, R⁵ and R⁶, independently of each other and independently at each occurrence, are H, C₁-C₆ alkyl, C₃-C₈ cycloalkyl, 3- to 12-membered heterocyclyl, 5- to 10-membered heteroaryl, or C₆-C₁₄ aryl, wherein the C₁-C₆ alkyl, C₃-C₈ cycloalkyl, 3- to 12-membered heterocyclyl, 5- to 10-membered heteroaryl, and C₆-C₁₄ aryl of R⁵ and R⁶ are independently optionally substituted by R^{h},

R^{b} and R^{c} are independently H, C₁-C₆ alkyl, C₂-C₆ alkenyl, C₂-C₆ alkynyl, C₃-C₈ cycloalkyl, 3- to 12-membered heterocyclyl, 5- to 10-membered heteroaryl, C₆-C₁₄ aryl, or —C(═O)OR¹⁷, wherein the C₁-C₆ alkyl, C₃-C₈ cycloalkyl, 3- to 12-membered heterocyclyl, 5- to 10-membered heteroaryl, and C₆-C₁₄ aryl of R^{b} and R^{c} are independently optionally substituted by R¹, and

r is 1, 2, or 3; or

(c)

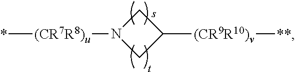

wherein

* represents the point of attachment to the Y—X— moiety,

** represents the point of attachment to the remainder of the molecule,

R⁷ and R⁸, independently of each other and independently at each occurrence, are hydrogen, C₃-C₈ cycloalkyl, 3- to 12-membered heterocyclyl, 5- to 10-membered heteroaryl, or C₆-C₁₄ aryl, wherein the C₃-C₈ cycloalkyl, 3- to 12-membered heterocyclyl, 5- to 10-membered heteroaryl, and C₆-C₁₄ aryl of R⁷ and R⁸ are independently optionally substituted by R^{j}, or R⁷ and R⁸ are taken together with the carbon atom to which they are attached to form a 3- to 8-membered cycloalkylene optionally substituted by R^{j},

R⁹ and R¹⁰, independently of each other and independently at each occurrence, are H, C₁-C₆ alkyl, C₃-C₈ cycloalkyl, 3- to 12-membered heterocyclyl, 5- to 10-membered heteroaryl, or C₆-C₁₄ aryl, wherein the C₁-C₆ alkyl, C₃-C₈ cycloalkyl, 3- to 12-membered heterocyclyl, 5- to 10-membered heteroaryl, and C₆-C₁₄ aryl of R⁹ and R¹⁰ are independently optionally substituted by R^{k},

s is 1, 2, or 3,

t is 1, 2, or 3,

wherein s+t is 2, 3, or 4,

u is 0 or 1, and

v is 0 or 1;

Y is C₆-C₉ aryl substituted by R¹¹, 6- to 10-membered heteroaryl substituted by R¹², or 3- to 12-membered heterocyclyl substituted by R¹³, wherein

each R¹¹, R¹², and R¹³, are independently C₁-C₆ alkyl, C₂-C₆ alkenyl, C₂-C₆ alkynyl, C₃-C₈ cycloalkyl, C₄-C₈ cycloalkenyl, 3- to 12-membered heterocyclyl, 5- to 10-membered heteroaryl, C₆-C₁₄ aryl, —OR¹⁴, —NR¹⁵R¹⁶, —SR¹⁴, —NO₂, —C═NH(OR¹⁴), —C(O)R¹⁴, —OC(O)R¹⁴, —C(O)OR¹⁴, —C(O)NR¹⁵R¹⁶, —NR¹⁴C(O)R¹⁵, —NR¹⁴C(O)OR¹⁵, —NR¹⁴C(O)NR¹⁵R¹⁶, —S(O)R¹⁴, —S(O)₂R¹⁴, —NR¹⁴S(O)R¹⁵, —NR¹⁴S(O)₂R¹⁵, —S(O)NR¹⁵R¹⁶, —S(O)₂NR¹⁵R¹⁶, or —P(O)(OR¹⁵)(OR¹⁶), wherein the C₁-C₆ alkyl, C₂-C₆ alkenyl, C₂-C₆ alkynyl, C₃-C₈ cycloalkyl, C₄-C₈ cycloalkenyl, 3- to 12-membered heterocyclyl, 5- to 10-membered heteroaryl, and C₆-C₁₄ aryl of R¹¹, R¹², and R¹³ are substituted by R^{L};

R¹⁴, R¹⁵ and R¹⁶, independently of each other and independently at each occurrence, are hydrogen, C₁-C₆ alkyl, C₂-C₆ alkenyl, C₂-C₆ alkynyl, C₃-C₈ cycloalkyl, C₆-C₁₄ aryl, 5- to 10-membered heteroaryl, or 3- to 12-membered heterocyclyl, wherein the C₁-C₆ alkyl, C₂-C₆ alkenyl, C₂-C₆ alkynyl, C₃-C₈ cycloalkyl, C₆-C₁₄ aryl, 5- to 10-membered heteroaryl, and 3- to 12-membered heterocyclyl of R¹⁴, R¹⁵ and R¹⁶ are independently substituted by C₁-C₆ perhaloalkyl, C₁-C₆ alkoxy, C₁-C₆ perhaloalkoxy, C₆-C₁₄ aryl or C₆-C₁₄ aryloxy wherein the C₆-C₁₄ aryl or C₆-C₁₄ aryloxy is further optionally substituted by halogen, —OH, cyano, C₁-C₆ alkyl, C₁-C₆ perhaloalkyl, C₁-C₆ alkoxy, or C₁-C₆ perhaloalkoxy; and, wherein at least one of R¹⁴, R¹⁵ and R¹⁶, when present, is not hydrogen;

R^{L} is C₁-C₆ alkyl, C₂-C₆ alkenyl, C₂-C₆ alkynyl, C₃-C₈ cycloalkyl, C₆-C₁₄ aryl, 5- to 10-membered heteroaryl, or 3- to 12-membered heterocyclyl, wherein the C₁-C₆ alkyl, C₂-C₆ alkenyl, C₂-C₆ alkynyl, C₃-C₈ cycloalkyl, C₆-C₁₄ aryl, 5- to 10-membered heteroaryl, or 3- to 12-membered heterocyclyl of R^{L} is substituted by halogen, —OH, cyano, C₁-C₆ alkyl, C₁-C₆ perhaloalkyl, C₁-C₆ alkoxy, C₁-C₆ perhaloalkoxy or C₆-C₁₄ aryl, wherein the C₆-C₁₄ aryl is further optionally substituted by halogen, —OH, cyano, C₁-C₆ alkyl, C₁-C₆ perhaloalkyl, C₁-C₆ alkoxy, or C₁-C₆ perhaloalkoxy; and

R^{d}, R^{e}, R^{f}, R^{g}, R^{h}, Rⁱ, R^{j}, and R^{k}, independently of each other and independently at each occurrence, are halogen, C₁-C₆ alkyl, C₂-C₆ alkenyl, C₂-C₆ alkynyl, C₃-C₈ cycloalkyl, C₆-C₁₄ aryl, 5- to 10-membered heteroaryl, 3- to 12-membered heterocyclyl, —OR¹⁴, —NR¹⁵R¹⁶, cyano, or nitro.

In one aspect, provided is a compound of formula (I) or a pharmaceutically acceptable salt thereof, wherein the compound has any one or more of the following features:
(i) X is —C(═O)—, —O— or —CH(OH)—;
(ii) L is

(a) —NH—CR¹R²—, such as —NH—CH₂— or —NH—CH(CH₃)— or wherein R¹ and R² are taken together with the carbon atom to which they are attached to form a 3- to 8-membered cycloalkylene such as a cyclopropylene;

(b) —CR⁵R⁶—CH(NR^{b}R^{c})—, including but not limited to, when R⁶, R^{b}, and R^{c} are each H, and R⁵ is H or C₁-C₆ alkyl; or

(c)

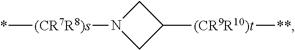

wherein * represents the point of attachment to the Y—X— moiety, ** represents the point of attachment to the remainder of the molecule, such as

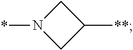

(iii) Y is:

(a) C₆-C₉ aryl substituted by R¹¹, such as 2,3-dihydro-1H-inden-2-yl, phenyl and naphthyl, which are substituted by at least one R¹¹;

(b) 6- to 10-membered heteroaryl substituted by R¹², such as a pyridinyl, pyrimidinyl, pyridin-2(1H)-onyl, and quinolin-6-yl, which are substituted by at least one R¹²; or (c) 3- to 12-membered heterocyclyl substituted by R¹³, such as 2H-pyran-2-only, isoindolinyl, piperidin-2-only and piperidinyl, which are substituted by at least one R¹³.

In another aspect is provided a compound of formula (I), a pharmaceutically acceptable salt, stereoisomer or tautomer thereof, wherein the —X-L- moiety is selected from the group consisting of

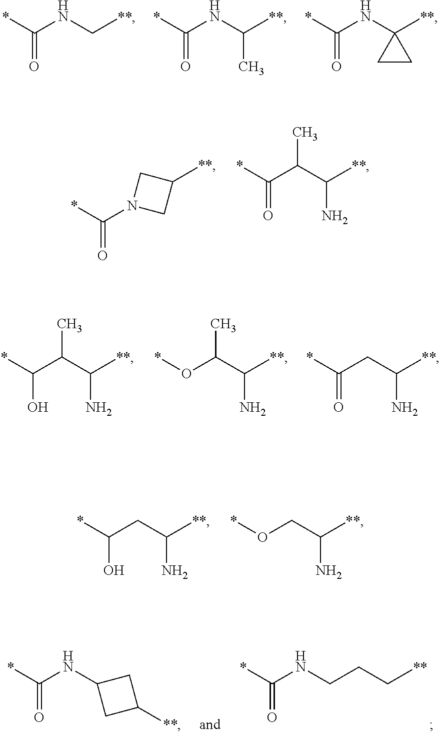

wherein * represents the point of attachment to the Y moiety, and ** represents the point of attachment to the remainder of the molecule.

Also provided is a pharmaceutical composition comprising a compound of any formula herein, including formula (I), or a pharmaceutically acceptable salt thereof, and a pharmaceutically acceptable carrier.

Also provided is a method of treating a disease or disorder mediated by fibroblast activation protein (FAP) in an individual in need thereof comprising administering to the individual a therapeutically effective amount of a compound as detailed herein, including but not limited to a compound of formula (I), or a pharmaceutically acceptable salt thereof, or a pharmaceutical composition comprising such compound or salt. Such disease or disorder in one aspect is characterized by proliferation, tissue remodeling, chronic inflammation, obesity, glucose intolerance, or insulin insensitivity. In one aspect, the disease or disorder is breast cancer, colorectal cancer, ovarian cancer, prostate cancer, pancreatic cancer, kidney cancer, lung cancer, melanoma, fibrosarcoma, bone sarcoma, connective tissue sarcoma, renal cell carcinoma, giant cell carcinoma, squamous cell carcinoma, leukemia, skin cancer, soft tissue cancer, liver cancer, gastrointestinal carcinoma, or adenocarcinoma. In a particular aspect, the disease or disorder is metastatic kidney cancer, chronic lymphocytary leukemia, pancreatic adenocarcinoma, or non-small cell lung cancer.

In a further aspect, the disease or disorder is a fibrotic disease, wound healing, keloid formation, osteoarthritis, rheumatoid arthritis and related disorders involving cartilage degradation, atherosclerotic disease, Crohn's disease, or Type II diabetes. In another particular aspect is provided a method of reducing tumor growth, tumor proliferation, or tumorigenicity in an individual in need thereof, comprising administering to the individual a compound as detailed herein, such as a compound of formula (I), or a pharmaceutically acceptable salt thereof, or a pharmaceutical composition of the foregoing.

### BRIEF DESCRIPTION OF THE DRAWINGS

FIG. 1A shows PRXS-AMC degradation over time by rhFAP. FIG. 1B shows Z-Gly-Pro-AMC degradation over time by rhFAP.

FIG. 2A shows PRXS-AMC degradation over time by rhPREP. FIG. 2B shows Z-Gly-Pro-AMC degradation over time by rhPREP.

FIG. 3A shows PRXS-AMC degradation over time by rhDPPIV. FIG. 3B shows PRXS-AMC degradation over time by rhDPP9.

### DETAILED DESCRIPTION

Described herein are compounds according to formula (I):

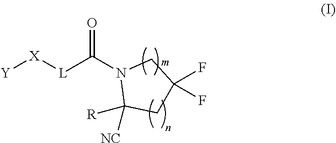

and pharmaceutically acceptable salts, stereoisomers or tautomers thereof. The compounds can be useful for inhibiting fibroblast activation protein (FAPc). In certain embodiments, the compound is used to treat a disease or a disorder mediated by FAPc in an individual. Such diseases or disorders can include or be characterized by proliferation, tissue remodeling, chronic inflammation, obesity, glucose intolerance, and/or insulin insensitivity. In some embodiments, the compound is used to treat cancer.

### Definitions

For use herein, unless clearly indicated otherwise, use of the terms “a”, “an” and the like refers to one or more.

Reference to “about” a value or parameter herein includes (and describes) embodiments that are directed to that value or parameter per se. For example, description referring to “about X” includes description of “X”.

“Alkyl” as used herein refers to and includes, unless otherwise stated, a saturated linear (i.e., unbranched) or branched univalent hydrocarbon chain or combination thereof, having the number of carbon atoms designated (i.e., C₁-C₁₀ means one to ten carbon atoms). Particular alkyl groups are those having 1 to 20 carbon atoms (a “C₁-C₂₀ alkyl”), having 1 to 10 carbon atoms (a “C₁-C₁₀ alkyl”), having 6 to 10 carbon atoms (a “C₆-C₁₀ alkyl”), having 1 to 6 carbon atoms (a “C₁-C₆ alkyl”), having 2 to 6 carbon atoms (a “C₂-C₆ alkyl”), or having 1 to 4 carbon atoms (a “C₁-C₄ alkyl”). Examples of alkyl groups include, but are not limited to, groups such as methyl, ethyl, n-propyl, isopropyl, n-butyl, t-butyl, isobutyl, sec-butyl, n-pentyl, n-hexyl, n-heptyl, n-octyl, n-nonyl, n-decyl, and the like.

“Alkylene” as used herein refers to the same residues as alkyl, but having bivalency. Particular alkylene groups are those having 1 to 20 carbon atoms (a “C₁-C₂₀ alkylene”), having 1 to 10 carbon atoms (a “C₁-C₁₀ alkylene”), having 6 to 10 carbon atoms (a “C₆-C₁₀ alkylene”), having 1 to 6 carbon atoms (a “C₁-C₆ alkylene”), 1 to 5 carbon atoms (a “C₁-C₅ alkylene”), 1 to 4 carbon atoms (a “C₁-C₄ alkylene”) or 1 to 3 carbon atoms (a “C₁-C₃ alkylene”). Examples of alkylene include, but are not limited to, groups such as methylene (—CH₂—), ethylene (—CH₂CH₂—), propylene (—CH₂CH₂CH₂—), isopropylene (—CH₂CH(CH₃)—), butylene (—CH₂(CH₂)₂CH₂—), isobutylene (—CH₂CH(CH₃)CH₂—), pentylene (—CH₂(CH₂)₃CH₂—), hexylene (—CH₂(CH₂)₄CH₂—), heptylene (—CH₂(CH₂)₅CH₂—), octylene (—CH₂(CH₂)₆CH₂—), and the like.

“Alkenyl” as used herein refers to and includes, unless otherwise stated, an unsaturated linear (i.e., unbranched) or branched univalent hydrocarbon chain or combination thereof, having at least one site of olefinic unsaturation (i.e., having at least one moiety of the formula C═C) and having the number of carbon atoms designated (i.e., C₂-C₁₀ means two to ten carbon atoms). An alkenyl group may have “cis” or “trans” configurations, or alternatively have “E” or “Z” configurations. Particular alkenyl groups are those having 2 to 20 carbon atoms (a “C₂-C₂₀ alkenyl”), having 6 to 10 carbon atoms (a “C₆-C₁₀ alkenyl”), having 2 to 8 carbon atoms (a “C₂-C₈ alkenyl”), having 2 to 6 carbon atoms (a “C₂-C₆ alkenyl”), or having 2 to 4 carbon atoms (a “C₂-C₄ alkenyl”). Examples of alkenyl group include, but are not limited to, groups such as ethenyl (or vinyl), prop-1-enyl, prop-2-enyl (or allyl), 2-methylprop-1-enyl, but-1-enyl, but-2-enyl, but-3-enyl, buta-1,3-dienyl, 2-methylbuta-1,3-dienyl, pent-1-enyl, pent-2-enyl, hex-1-enyl, hex-2-enyl, hex-3-enyl, and the like.

“Alkynyl” as used herein refers to and includes, unless otherwise stated, an unsaturated linear (i.e., unbranched) or branched univalent hydrocarbon chain or combination thereof, having at least one site of acetylenic unsaturation (i.e., having at least one moiety of the formula C═C) and having the number of carbon atoms designated (i.e., C₂-C₁₀ means two to ten carbon atoms).

Particular alkynyl groups are those having 2 to 20 carbon atoms (a “C₂-C₂₀ alkynyl”), having 6 to 10 carbon atoms (a “C₆-C₁₀ alkynyl”), having 2 to 8 carbon atoms (a “C₂-C₈ alkynyl”), having 2 to 6 carbon atoms (a “C₂-C₆ alkynyl”), or having 2 to 4 carbon atoms (a “C₂-C₄ alkynyl”). Examples of alkynyl group include, but are not limited to, groups such as ethynyl (or acetylenyl), prop-1-ynyl, prop-2-ynyl (or propargyl), but-1-ynyl, but-2-ynyl, but-3-ynyl, and the like.

“Alkoxy” refers to the group R—O—, where R is alkyl; and includes, by way of example, methoxy, ethoxy, n-propoxy, iso-propoxy, n-butoxy, tert-butoxy, sec-butoxy, n-pentoxy, n-hexyloxy, 1,2-dimethylbutoxy, and the like. Similarly, “cycloalkoxy” refers to the group “cycloalkyl-O—” and “aryloxy” refers to the group “aryl-O—”. “Substituted alkoxy” refers to the group “substituted alkyl-O—”. “Substituted cycloalkoxy” refers to the group “substituted cycloalkyl-O—”. “Substituted aryloxy” refers to the group “substituted aryl-O—”.

“Aryl” or “Ar” as used herein refers to an unsaturated aromatic carbocyclic group having a single ring (e.g., phenyl) or multiple condensed rings (e.g., naphthyl or anthryl) which condensed rings may or may not be aromatic. Particular aryl groups are those having from 6 to 14 annular carbon atoms (a “C₆-C₁₄ aryl”). An aryl group having more than one ring where at least one ring is non-aromatic may be connected to the parent structure at either an aromatic ring position or at a non-aromatic ring position. In one variation, an aryl group having more than one ring where at least one ring is non-aromatic is connected to the parent structure at an aromatic ring position.

“Arylene” as used herein refers to the same residues as aryl, but having bivalency. Particular arylene groups are those having from 6 to 14 annular carbon atoms (a “C₆-C₁₄ arylene”).

“Cycloalkyl” as used herein refers to and includes, unless otherwise stated, saturated cyclic univalent hydrocarbon structures, having the number of carbon atoms designated (i.e., C₃-C₁₀ means three to ten carbon atoms). Cycloalkyl can consist of one ring, such as cyclohexyl, or multiple rings, such as adamantyl. A cycloalkyl comprising more than one ring may be fused, spiro or bridged, or combinations thereof. Particular cycloalkyl groups are those having from 3 to 12 annular carbon atoms. A preferred cycloalkyl is a cyclic hydrocarbon having from 3 to 8 annular carbon atoms (a “C₃-C₈ cycloalkyl”), having 3 to 6 carbon atoms (a “C₃-C₆ cycloalkyl”), or having from 3 to 4 annular carbon atoms (a “C₃-C₄ cycloalkyl”). Examples of cycloalkyl include, but are not limited to, cyclopropyl, cyclobutyl, cyclopentyl, cyclohexyl, cycloheptyl, norbornyl, and the like.

“Cycloalkylene” as used herein refers to the same residues as cycloalkyl, but having bivalency. Cycloalkylene can consist of one ring or multiple rings which may be fused, spiro or bridged, or combinations thereof. Particular cycloalkylene groups are those having from 3 to 12 annular carbon atoms. A preferred cycloalkylene is a cyclic hydrocarbon having from 3 to 8 annular carbon atoms (a “C₃-C₈ cycloalkylene”), having 3 to 6 carbon atoms (a “C₃-C₆ cycloalkylene”), or having from 3 to 4 annular carbon atoms (a “C₃-C₄ cycloalkylene”). Examples of cycloalkylene include, but are not limited to, cyclopropylene, cyclobutylene, cyclopentylene, cyclohexylene, cycloheptylene, norbornylene, and the like. A cycloalkylene may attach to the remaining structures via the same ring carbon atom or different ring carbon atoms. When a cycloalkylene attaches to the remaining structures via two different ring carbon atoms, the connecting bonds may be cis- or trans- to each other. For example, cyclopropylene may include 1,1-cyclopropylene and 1,2-cyclopropylene (e.g., cis-1,2-cyclopropylene or trans-1,2-cyclopropylene), or a mixture thereof.

“Cycloalkenyl” refers to and includes, unless otherwise stated, an unsaturated cyclic non-aromatic univalent hydrocarbon structure, having at least one site of olefinic unsaturation (i.e., having at least one moiety of the formula C═C) and having the number of carbon atoms designated (i.e., C₃-C₁₀ means three to ten carbon atoms). Cycloalkenyl can consist of one ring, such as cyclohexenyl, or multiple rings, such as norbornenyl. A preferred cycloalkenyl is an unsaturated cyclic hydrocarbon having from 3 to 8 annular carbon atoms (a “C₃-C₈ cycloalkenyl”). Examples of cycloalkenyl groups include, but are not limited to, cyclopropenyl, cyclobutenyl, cyclopentenyl, cyclohexenyl, norbornenyl, and the like.

“Cycloalkenylene” as used herein refers to the same residues as cycloalkenyl, but having bivalency.

“Heteroaryl” as used herein refers to an unsaturated aromatic cyclic group having from 1 to 14 annular carbon atoms and at least one annular heteroatom, including but not limited to heteroatoms such as nitrogen, oxygen and sulfur. A heteroaryl group may have a single ring (e.g., pyridyl, furyl) or multiple condensed rings (e.g., indolizinyl, benzothienyl) which condensed rings may or may not be aromatic. Particular heteroaryl groups are 5 to 14-membered rings having 1 to 12 annular carbon atoms and 1 to 6 annular heteroatoms independently selected from nitrogen, oxygen and sulfur, 5 to 10-membered rings having 1 to 8 annular carbon atoms and 1 to 4 annular heteroatoms independently selected from nitrogen, oxygen and sulfur, or 5, 6 or 7-membered rings having 1 to 5 annular carbon atoms and 1 to 4 annular heteroatoms independently selected from nitrogen, oxygen and sulfur. In one variation, particular heteroaryl groups are monocyclic aromatic 5-, 6- or 7-membered rings having from 1 to 6 annular carbon atoms and 1 to 4 annular heteroatoms independently selected from nitrogen, oxygen and sulfur. In another variation, particular heteroaryl groups are polycyclic aromatic rings having from 1 to 12 annular carbon atoms and 1 to 6 annular heteroatoms independently selected from nitrogen, oxygen and sulfur. A heteroaryl group having more than one ring where at least one ring is non-aromatic may be connected to the parent structure at either an aromatic ring position or at a non-aromatic ring position. In one variation, a heteroaryl group having more than one ring where at least one ring is non-aromatic is connected to the parent structure at an aromatic ring position. A heteroaryl group may be connected to the parent structure at a ring carbon atom or a ring heteroatom.

Where applicable, a heteroaryl group may be depicted in a tautomeric form. Such compounds would be considered to be heteroaryl even if certain tautomeric forms are, for example, heterocyclyl. For example, the heteroaryl group

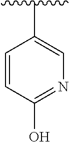 [SMILES: *c1ccc(O)nc1]

may be depicted in the heterocyclic tautomeric form

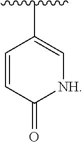 [SMILES: *c1ccc(=O)[nH]c1]

Regardless of which tautomer is shown, the group is considered to be heteroaryl.

“Heterocycle”, “heterocyclic”, or “heterocyclyl” as used herein refers to a saturated or an unsaturated non-aromatic cyclic group having a single ring or multiple condensed rings, and having from 1 to 14 annular carbon atoms and from 1 to 6 annular heteroatoms, such as nitrogen, sulfur or oxygen, and the like. A heterocycle comprising more than one ring may be fused, bridged or spiro, or any combination thereof, but excludes heteroaryl groups. Thus, it is to be understood that, in fused-ring “heterocycles”, one or more of the fused rings may be cycloalkyl, cycloalkenyl, or aryl, but not heteroaryl. The heterocyclyl group may be optionally substituted independently with one or more substituents described herein. Particular heterocyclyl groups are 3 to 14-membered rings having 1 to 13 annular carbon atoms and 1 to 6 annular heteroatoms independently selected from nitrogen, oxygen and sulfur, 3 to 12-membered rings having 1 to 11 annular carbon atoms and 1 to 6 annular heteroatoms independently selected from nitrogen, oxygen and sulfur, 3 to 10-membered rings having 1 to 9 annular carbon atoms and 1 to 4 annular heteroatoms independently selected from nitrogen, oxygen and sulfur, 3 to 8-membered rings having 1 to 7 annular carbon atoms and 1 to 4 annular heteroatoms independently selected from nitrogen, oxygen and sulfur, or 3 to 6-membered rings having 1 to 5 annular carbon atoms and 1 to 4 annular heteroatoms independently selected from nitrogen, oxygen and sulfur. In one variation, heterocyclyl includes monocyclic 3-, 4-, 5-, 6- or 7-membered rings having from 1 to 2, 1 to 3, 1 to 4, 1 to 5, or 1 to 6 annular carbon atoms and 1 to 2, 1 to 3, or 1 to 4 annular heteroatoms independently selected from nitrogen, oxygen and sulfur. In another variation, heterocyclyl includes polycyclic non-aromatic rings having from 1 to 12 annular carbon atoms and 1 to 6 annular heteroatoms independently selected from nitrogen, oxygen and sulfur.

“Halo” or “halogen” refers to elements of the Group 17 series having atomic number 9 to 85. Preferred halo groups include the radicals of fluorine, chlorine, bromine and iodine. Where a residue is substituted with more than one halogen, it may be referred to by using a prefix corresponding to the number of halogen moieties attached, e.g., dihaloaryl, dihaloalkyl, trihaloaryl etc. refer to aryl and alkyl substituted with two (“di”) or three (“tri”) halo groups, which may be but are not necessarily the same halogen; thus 4-chloro-3-fluorophenyl is within the scope of dihaloaryl. An alkyl group in which each hydrogen is replaced with a halo group is referred to as a “perhaloalkyl.” A preferred perhaloalkyl group is trifluoromethyl (—CF₃). Similarly, “perhaloalkoxy” refers to an alkoxy group in which a halogen takes the place of each H in the hydrocarbon making up the alkyl moiety of the alkoxy group. An example of a perhaloalkoxy group is trifluoromethoxy (—OCF₃).

“Carbonyl” refers to the group C═O.

“Oxo” refers to the moiety ═O.

“Optionally substituted” unless otherwise specified means that a group may be unsubstituted or substituted by one or more (e.g., 1, 2, 3, 4 or 5) of the substituents listed for that group in which the substituents may be the same of different. In one embodiment, an optionally substituted group has one substituent. In another embodiment, an optionally substituted group has two substituents. In another embodiment, an optionally substituted group has three substituents. In another embodiment, an optionally substituted group has four substituents. In some embodiments, an optionally substituted group has 1 to 2, 1 to 3, 1 to 4, 1 to 5, 2 to 3, 2 to 4, or 2 to 5 substituents. In one embodiment, an optionally substituted group is unsubstituted.

Unless clearly indicated otherwise, “an individual” as used herein intends a mammal, including but not limited to a primate, human, bovine, horse, feline, canine, or rodent. In one variation, the individual is a human.

As used herein, “treatment” or “treating” is an approach for obtaining beneficial or desired results including clinical results. Beneficial or desired results include, but are not limited to, one or more of the following: decreasing one more symptoms resulting from the disease, diminishing the extent of the disease, stabilizing the disease (e.g., preventing or delaying the worsening of the disease), preventing or delaying the spread of the disease, delaying the occurrence or recurrence of the disease, delay or slowing the progression of the disease, ameliorating the disease state, providing a remission (whether partial or total) of the disease, decreasing the dose of one or more other medications required to treat the disease, enhancing effect of another medication, delaying the progression of the disease, increasing the quality of life, and/or prolonging survival. The methods described herein contemplate any one or more of these aspects of treatment.

As used herein, the term “effective amount” intends such amount of a compound of the invention which should be effective in a given therapeutic form. As is understood in the art, an effective amount may be in one or more doses, i.e., a single dose or multiple doses may be required to achieve the desired treatment. An effective amount may be considered in the context of administering one or more therapeutic agents, and a single agent may be considered to be given in an effective amount if, in conjunction with one or more other agents, a desirable or beneficial result may be or is achieved. Suitable doses of any of the co-administered compounds may optionally be lowered due to the combined action (e.g., additive or synergistic effects) of the compounds.

A “therapeutically effective amount” refers to an amount of a compound or salt thereof sufficient to produce a desired therapeutic outcome.

As used herein, “unit dosage form” refers to physically discrete units, suitable as unit dosages, each unit containing a predetermined quantity of active ingredient calculated to produce the desired therapeutic effect in association with the required pharmaceutical carrier. Unit dosage forms may contain a single or a combination therapy.

As used herein, by “pharmaceutically acceptable” or “pharmacologically acceptable” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

“Pharmaceutically acceptable salts” are those salts which retain at least some of the biological activity of the free (non-salt) compound and which can be administered as drugs or pharmaceuticals to an individual. Such salts, for example, include: (1) acid addition salts, formed with inorganic acids such as hydrochloric acid, hydrobromic acid, sulfuric acid, nitric acid, phosphoric acid, and the like; or formed with organic acids such as acetic acid, oxalic acid, propionic acid, succinic acid, maleic acid, tartaric acid and the like; (2) salts formed when an acidic proton present in the parent compound either is replaced by a metal ion, e.g., an alkali metal ion, an alkaline earth ion, or an aluminum ion; or coordinates with an organic base. Acceptable organic bases include ethanolamine, diethanolamine, triethanolamine and the like. Acceptable inorganic bases include aluminum hydroxide, calcium hydroxide, potassium hydroxide, sodium carbonate, sodium hydroxide, and the like. Pharmaceutically acceptable salts can be prepared in situ in the manufacturing process, or by separately reacting a purified compound of the invention in its free acid or base form with a suitable organic or inorganic base or acid, respectively, and isolating the salt thus formed during subsequent purification.

The term “excipient” as used herein means an inert or inactive substance that may be used in the production of a drug or pharmaceutical, such as a tablet containing a compound of the invention as an active ingredient. Various substances may be embraced by the term excipient, including without limitation any substance used as a binder, disintegrant, coating, compression/encapsulation aid, cream or lotion, lubricant, solutions for parenteral administration, materials for chewable tablets, sweetener or flavoring, suspending/gelling agent, or wet granulation agent. Binders include, e.g., carbomers, povidone, xanthan gum, etc.; coatings include, e.g., cellulose acetate phthalate, ethylcellulose, gellan gum, maltodextrin, enteric coatings, etc.; compression/encapsulation aids include, e.g., calcium carbonate, dextrose, fructose dc (dc=“directly compressible”), honey dc, lactose (anhydrate or monohydrate; optionally in combination with aspartame, cellulose, or microcrystalline cellulose), starch dc, sucrose, etc.; disintegrants include, e.g., croscarmellose sodium, gellan gum, sodium starch glycolate, etc.; creams or lotions include, e.g., maltodextrin, carrageenans, etc.; lubricants include, e.g., magnesium stearate, stearic acid, sodium stearyl fumarate, etc.; materials for chewable tablets include, e.g., dextrose, fructose dc, lactose (monohydrate, optionally in combination with aspartame or cellulose), etc.; suspending/gelling agents include, e.g., carrageenan, sodium starch glycolate, xanthan gum, etc.; sweeteners include, e.g., aspartame, dextrose, fructose dc, sorbitol, sucrose dc, etc.; and wet granulation agents include, e.g., calcium carbonate, maltodextrin, microcrystalline cellulose, etc.

It is understood that aspects and embodiments described herein as “comprising” include “consisting of” and “consisting essentially of” embodiments.

When a composition is described as “consisting essentially of” the listed components, the composition contains the components expressly listed, and may contain other components which do not substantially affect the disease or condition being treated such as trace impurities. However, the composition either does not contain any other components which do substantially affect the disease or condition being treated other than those components expressly listed; or, if the composition does contain extra components other than those listed which substantially affect the disease or condition being treated, the composition does not contain a sufficient concentration or amount of those extra components to substantially affect the disease or condition being treated. When a method is described as “consisting essentially of” the listed steps, the method contains the steps listed, and may contain other steps that do not substantially affect the disease or condition being treated, but the method does not contain any other steps which substantially affect the disease or condition being treated other than those steps expressly listed.

When a moiety is indicated as substituted by “at least one” substituent, this also encompasses the disclosure of exactly one substituent.

### Compounds

In some embodiments, provided is a compound of formula (I):

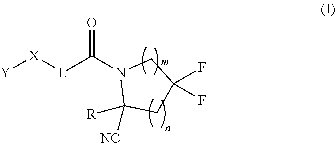

a pharmaceutically acceptable salt, stereoisomer or tautomer thereof, wherein:
R is hydrogen, C₁-C₆ alkyl, C₃-C₈ cycloalkyl, 3- to 12-membered heterocyclyl, 5- to 10-membered heteroaryl, or C₆-C₁₄ aryl, wherein the C₁-C₆ alkyl, C₃-C₈ cycloalkyl, 3- to 12-membered heterocyclyl, 5- to 10-membered heteroaryl, and C₆-C₁₄ aryl of R are independently optionally substituted by R^{d};
m is 0, 1, 2, 3, or 4;
n is 0, 1, 2, 3, or 4,

wherein m+n is 1, 2, 3, or 4;

### X is —C(═O)—, —O—, —CH(OH)—, —S—, —S(═O)—, or —S(═O)₂—;

### L is

(a)

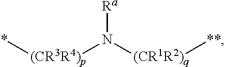

wherein

* represents the point of attachment to the Y—X— moiety,

** represents the point of attachment to the remainder of the molecule,

R^{a} is hydrogen, C₁-C₆ alkyl, C₃-C₈ cycloalkyl, 3- to 12-membered heterocyclyl, 5- to 10-membered heteroaryl, or C₆-C₁₄ aryl, wherein the C₁-C₆ alkyl, C₃-C₈ cycloalkyl, 3- to 12-membered heterocyclyl, 5- to 10-membered heteroaryl, and C₆-C₁₄ aryl of R^{a} are independently optionally substituted by R^{e},

R¹ and R², independently of each other and independently at each occurrence, are hydrogen, C₁-C₂ alkyl, C₃-C₈ cycloalkyl, 3- to 12-membered heterocyclyl, 5- to 10-membered heteroaryl, or C₆-C₁₄ aryl, wherein the C₃-C₈ cycloalkyl, 3- to 12-membered heterocyclyl, 5- to 10-membered heteroaryl, and C₆-C₁₄ aryl of R¹ and R² are independently optionally substituted by R^{f},

or R¹ and R² are taken together with the carbon atom or atoms to which they are attached to form a 3- to 8-membered cycloalkylene optionally substituted by R^{f},

q is 1, 2, or 3,
R³ and R⁴, independently of each other and independently at each occurrence, are hydrogen, C₃-C₈ cycloalkyl, 3- to 12-membered heterocyclyl, 5- to 10-membered heteroaryl, or C₆-C₁₄ aryl, wherein the C₃-C₈ cycloalkyl, 3- to 12-membered heterocyclyl, 5- to 10-membered heteroaryl, and C₆-C₁₄ aryl of R³ and R⁴ are independently optionally substituted by R^{g},

or R³ and R⁴ are taken together with the carbon atom to which they are attached to form a 3- to 8-membered cycloalkylene optionally substituted by R^{g}, and

p is 0, 1, or 2;
(b)

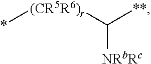

wherein

* represents the point of attachment to the Y—X— moiety,

** represents the point of attachment to the remainder of the molecule,

R⁵ and R⁶, independently of each other and independently at each occurrence, are H, C₁-C₆ alkyl, C₃-C₈ cycloalkyl, 3- to 12-membered heterocyclyl, 5- to 10-membered heteroaryl, or C₆-C₁₄ aryl, wherein the C₁-C₆ alkyl, C₃-C₈ cycloalkyl, 3- to 12-membered heterocyclyl, 5- to 10-membered heteroaryl, and C₆-C₁₄ aryl of R⁵ and R⁶ are independently optionally substituted by R^{h},
R^{b} and R^{c} are independently H, C₁-C₆ alkyl, C₂-C₆ alkenyl, C₂-C₆ alkynyl, C₃-C₈ cycloalkyl, 3- to 12-membered heterocyclyl, 5- to 10-membered heteroaryl, C₆-C₁₄ aryl, or —C(═O)OR¹⁷, wherein the C₁-C₆ alkyl, C₃-C₈ cycloalkyl, 3- to 12-membered heterocyclyl, 5- to 10-membered heteroaryl, and C₆-C₁₄ aryl of R^{b} and R^{c} are independently optionally substituted by R¹, and
r is 1, 2, or 3; or
(c)

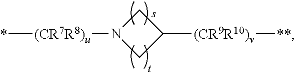

wherein

* represents the point of attachment to the Y—X— moiety,

** represents the point of attachment to the remainder of the molecule,

R⁷ and R⁸, independently of each other and independently at each occurrence, are hydrogen, C₃-C₈ cycloalkyl, 3- to 12-membered heterocyclyl, 5- to 10-membered heteroaryl, or C₆-C₁₄ aryl, wherein the C₃-C₈ cycloalkyl, 3- to 12-membered heterocyclyl, 5- to 10-membered heteroaryl, and C₆-C₁₄ aryl of R⁷ and R⁸ are independently optionally substituted by R^{j}, or R⁷ and R⁸ are taken together with the carbon atom to which they are attached to form a 3- to 8-membered cycloalkylene optionally substituted by R^{j},
R⁹ and R¹⁰, independently of each other and independently at each occurrence, are H, C₁-C₆ alkyl, C₃-C₈ cycloalkyl, 3- to 12-membered heterocyclyl, 5- to 10-membered heteroaryl, or C₆-C₁₄ aryl, wherein the C₁-C₆ alkyl, C₃-C₈ cycloalkyl, 3- to 12-membered heterocyclyl, 5- to 10-membered heteroaryl, and C₆-C₁₄ aryl of R⁹ and R¹⁰ are independently optionally substituted by R^{k},

s is 1, 2, or 3,

t is 1, 2, or 3,

wherein s+t is 2, 3, or 4,

u is 0 or 1, and

v is 0 or 1;

Y is C₆-C₉ aryl substituted by R¹¹, 6- to 10-membered heteroaryl substituted by R¹², or 3- to 12-membered heterocyclyl substituted by R¹³, wherein

each R¹, R¹², and R¹³, are independently C₁-C₆ alkyl, C₂-C₆ alkenyl, C₂-C₆ alkynyl, C₃-C₈ cycloalkyl, C₄-C₈ cycloalkenyl, 3- to 12-membered heterocyclyl, 5- to 10-membered heteroaryl, C₆-C₁₄ aryl, —OR¹⁴, —NR¹⁵R¹⁶, —SR¹⁴, —NO₂, —C═NH(OR¹⁴), —C(O)R¹⁴, —OC(O)R¹⁴, —C(O)OR¹⁴, —C(O)NR¹⁵R¹⁶, —NR¹⁴C(O)R¹⁵, —NR¹⁴C(O)OR¹⁵, —NR¹⁴C(O)NR¹⁵R¹⁶, —S(O)R¹⁴, —S(O)₂R¹⁴, —NR¹⁴S(O)R¹⁵, —NR¹⁴S(O)₂R¹⁵, —S(O)NR¹⁵R¹⁶, —S(O)₂NR¹⁵R¹⁶, or —P(O)(OR¹⁵)(OR¹⁶), wherein the C₁-C₆ alkyl, C₂-C₆ alkenyl, C₂-C₆ alkynyl, C₃-C₈ cycloalkyl, C₄-C₈ cycloalkenyl, 3- to 12-membered heterocyclyl, 5- to 10-membered heteroaryl, and C₆-C₁₄ aryl of R¹¹, R¹², and R¹³ are substituted by R^{L};

R¹⁴, R¹⁵ and R¹⁶, independently of each other and independently at each occurrence, are hydrogen, C₁-C₆ alkyl, C₂-C₆ alkenyl, C₂-C₆ alkynyl, C₃-C₈ cycloalkyl, C₆-C₁₄ aryl, 5- to 10-membered heteroaryl, or 3- to 12-membered heterocyclyl, wherein the C₁-C₆ alkyl, C₂-C₆ alkenyl, C₂-C₆ alkynyl, C₃-C₈ cycloalkyl, C₆-C₁₄ aryl, 5- to 10-membered heteroaryl, and 3- to 12-membered heterocyclyl of R¹⁴, R¹⁵ and R¹⁶ are independently substituted by C₁-C₆ perhaloalkyl, C₁-C₆ alkoxy, C₁-C₆ perhaloalkoxy, C₆-C₁₄ aryl or C₆-C₁₄ aryloxy wherein the C₆-C₁₄ aryl or C₆-C₁₄ aryloxy is further optionally substituted by halogen, —OH, cyano, C₁-C₆ alkyl, C₁-C₆ perhaloalkyl, C₁-C₆ alkoxy, or C₁-C₆ perhaloalkoxy; and, wherein at least one of R¹⁴, R¹⁵ and R¹⁶, when present, is not hydrogen;

R^{L} is C₁-C₆ alkyl, C₂-C₆ alkenyl, C₂-C₆ alkynyl, C₃-C₈ cycloalkyl, C₆-C₁₄ aryl, 5- to 10-membered heteroaryl, or 3- to 12-membered heterocyclyl, wherein the C₁-C₆ alkyl, C₂-C₆ alkenyl, C₂-C₆ alkynyl, C₃-C₈ cycloalkyl, C₆-C₁₄ aryl, 5- to 10-membered heteroaryl, or 3- to 12-membered heterocyclyl of R^{L} is substituted by halogen, —OH, cyano, C₁-C₆ alkyl, C₁-C₆ perhaloalkyl, C₁-C₆ alkoxy, C₁-C₆ perhaloalkoxy or C₆-C₁₄ aryl, wherein the C₆-C₁₄ aryl is further optionally substituted by halogen, —OH, cyano, C₁-C₆ alkyl, C₁-C₆ perhaloalkyl, C₁-C₆ alkoxy, or C₁-C₆ perhaloalkoxy; and

R^{d}, R^{e}, R^{f}, R^{g}, R^{h}, Rⁱ, R^{j}, and R^{k}, independently of each other and independently at each occurrence, are halogen, C₁-C₆ alkyl, C₂-C₆ alkenyl, C₂-C₆ alkynyl, C₃-C₈ cycloalkyl, C₆-C₁₄ aryl, 5- to 10-membered heteroaryl, 3- to 12-membered heterocyclyl, —OR¹⁴, —NR¹⁵R¹⁶, cyano, or nitro.

In the descriptions herein, it is understood that every description, variation, embodiment or aspect of a moiety may be combined with every description, variation, embodiment or aspect of other moieties the same as if each and every combination of descriptions is specifically and individually listed. For example, every description, variation, embodiment or aspect provided herein with respect to R of formula (I) may be combined with every description, variation, embodiment or aspect of Y, X, L, m, and/or n the same as if each and every combination were specifically and individually listed. It is also understood that all descriptions, variations, embodiments or aspects of formula (I), where applicable, apply equally to other formulae detailed herein, and are equally described, the same as if each and every description, variation, embodiment or aspect were separately and individually listed for all formulae. For example, all descriptions, variations, embodiments or aspects of formula (I), where applicable, apply equally to any of formulae tailed herein, and are equally described, the same as if each and every description, variation, embodiment or aspect were separately and individually listed for all formulae. The same applies to any other formula provided herein.

In some embodiments, the compound of formula (I) is of the formula (Ia):

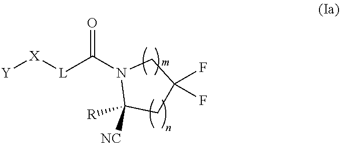

a pharmaceutically acceptable salt, stereoisomer or tautomer thereof, wherein Y, X, L, R, m, and n are as defined for formula (I).

In some embodiments, the compound of formula (I) is of the formula (Ib):

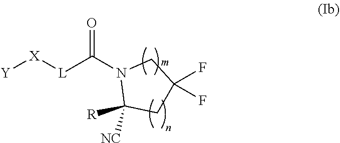

a pharmaceutically acceptable salt, stereoisomer or tautomer thereof, wherein Y, X, L, R, m, and n are as defined for formula (I).

In some embodiments of the compound of formula (I), where m is 1 and n is 1, the compound is of the formula (II):

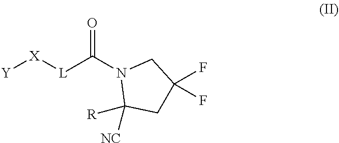

a pharmaceutically acceptable salt, stereoisomer or tautomer thereof, wherein Y, X, L, and R are as defined for formula (I).

In some embodiments, the compound of formula (II) is of the formula (IIa):

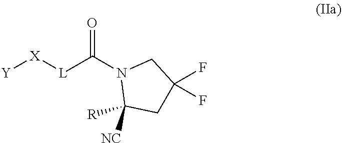

a pharmaceutically acceptable salt, stereoisomer or tautomer thereof, wherein Y, X, L, and R are as defined for formula (I).

In some embodiments, the compound of formula (II) is of the formula (IIb):

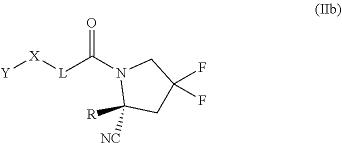

a pharmaceutically acceptable salt, stereoisomer or tautomer thereof, wherein Y, X, L, and R are as defined for formula (I).

In some embodiments of the compound of formula (II), where L is —NH—CH₂—, the compound is of the formula (III):

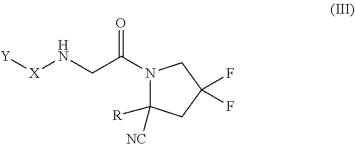

a pharmaceutically acceptable salt, stereoisomer or tautomer thereof, wherein Y, X, and R are as defined for formula (I).

In some embodiments, the compound of formula (III) is of the formula (IIIa):

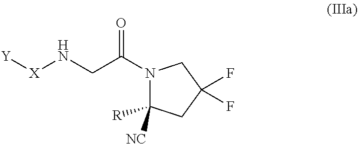

a pharmaceutically acceptable salt, stereoisomer or tautomer thereof, wherein Y, X, and R are as defined for formula (I). In some such embodiments, X is —C(═O)—. In some such embodiments, X is —C(═O)— and R is hydrogen. In some such embodiments, X is —C(═O)— and Y is

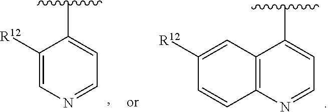

In some embodiments, the compound of formula (III) is of the formula (IIIb):

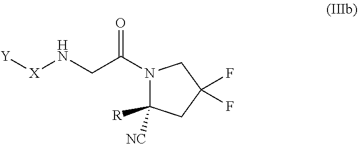

a pharmaceutically acceptable salt, stereoisomer or tautomer thereof, wherein Y, X, and R are as defined for formula (I). In some such embodiments, X is —C(═O)—. In some such embodiments, X is —C(═O)— and R is hydrogen. In some such embodiments, X is —C(═O)— and Y is

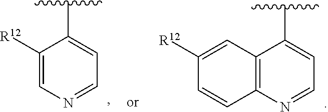

In some embodiments, the compound of formula (III) is of the formula (III-1):

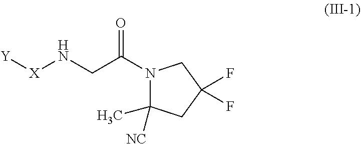

a pharmaceutically acceptable salt, stereoisomer or tautomer thereof, wherein Y and X are as defined for formula (I).

In some embodiments of the compound of formula (II), where L is —NH—CH(CH₃)—, the compound is of the formula (IV):

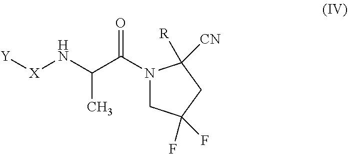

a pharmaceutically acceptable salt, stereoisomer or tautomer thereof, wherein Y, X, and R are as defined for formula (I). In one aspect of a compound of formula (IV), the carbon bearing the methyl group of L is in the S configuration. In one aspect of a compound of formula (IV), the carbon bearing the methyl group of L is in the R configuration.

In some embodiments, the compound of formula (IV) is of the formula (IVa):

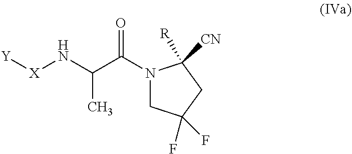

a pharmaceutically acceptable salt, stereoisomer or tautomer thereof, wherein Y, X, and R are as defined for formula (I). In one aspect of a compound of formula (IVa), the carbon bearing the methyl group of L is in the S configuration. In one aspect of a compound of formula (IVa), the carbon bearing the methyl group of L is in the R configuration.

In some embodiments, the compound of formula (IV) is of the formula (IVb):

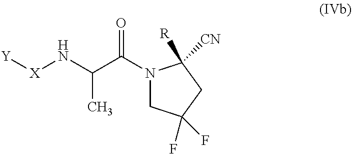

a pharmaceutically acceptable salt, stereoisomer or tautomer thereof, wherein Y, X, and R are as defined for formula (I). In one aspect of a compound of formula (IVb), the carbon bearing the methyl group of L is in the S configuration. In one aspect of a compound of formula (IVb), the carbon bearing the methyl group of L is in the R configuration.

In some embodiments of the compound of formula (II), the compound is of the formula (V):

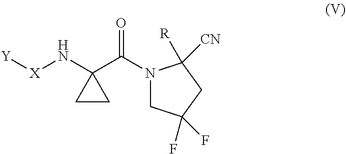

a pharmaceutically acceptable salt, stereoisomer or tautomer thereof, wherein Y, X, and R are as defined for formula (I).

In some embodiments, the compound of formula (V) is of the formula (Va):

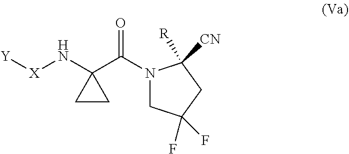

a pharmaceutically acceptable salt, stereoisomer or tautomer thereof, wherein Y, X, and R are as defined for formula (I).

In some embodiments, the compound of formula (V) is of the formula (Vb):

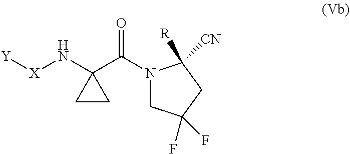

a pharmaceutically acceptable salt, stereoisomer or tautomer thereof, wherein Y, X, and R are as defined for formula (I).

In some embodiments of the compound of formula (II), the compound is of the formula (VI):

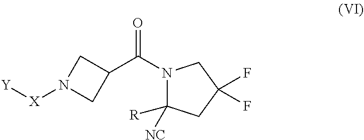

a pharmaceutically acceptable salt, stereoisomer or tautomer thereof, wherein Y, X, and R are as defined for formula (I).

In some embodiments, the compound of formula (VI) is of the formula (VIa):

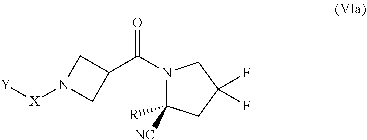

a pharmaceutically acceptable salt, stereoisomer or tautomer thereof, wherein Y, X, and R are as defined for formula (I).

In some embodiments, the compound of formula (VI) is of the formula (VIb):

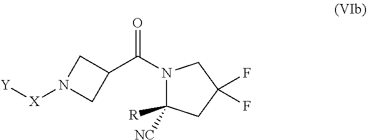

a pharmaceutically acceptable salt, stereoisomer or tautomer thereof, wherein Y, X, and R are as defined for formula (I).

In some embodiments of the compound of formula (II), where L is —CH₂—CH(NH₂)—, the compound is of the formula (VII):

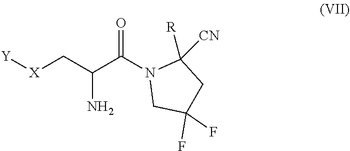

a pharmaceutically acceptable salt, stereoisomer or tautomer thereof, wherein Y, X, and R are as defined for formula (I). In one aspect of a compound of formula (VII), the carbon bearing the —NH₂ group of L is in the S configuration. In one aspect of a compound of formula (VII), the carbon bearing the —NH₂ group of L is in the R configuration.

In some embodiments, the compound of formula (VII) is of the formula (VIIa):

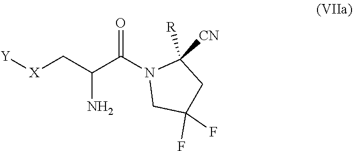

a pharmaceutically acceptable salt, stereoisomer or tautomer thereof, wherein Y, X, and R are as defined for formula (I). In one aspect of a compound of formula (VIIa), the carbon bearing the —NH₂ group of L is in the S configuration. In one aspect of a compound of formula (VIIa), the carbon bearing the —NH₂ group of L is in the R configuration.

In some embodiments, the compound of formula (VII) is of the formula (VIIb):

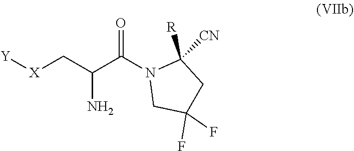

a pharmaceutically acceptable salt, stereoisomer or tautomer thereof, wherein Y, X, and R are as defined for formula (I). In one aspect of a compound of formula (VIIb), the carbon bearing the —NH₂ group of L is in the S configuration. In one aspect of a compound of formula (VIIb), the carbon bearing the —NH₂ group of L is in the R configuration.

In some embodiments of the compound of formula (II), where L is —CH(CH₃)—CH(NH₂)—, the compound is of the formula (VIII):

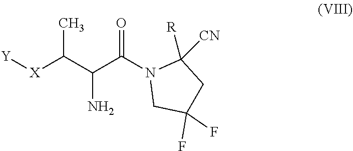

a pharmaceutically acceptable salt, stereoisomer or tautomer thereof, wherein Y, X, and R are as defined for formula (I). In one aspect of a compound of formula (VIII), the carbon bearing the —NH₂ group of L is in the S configuration. In one aspect of a compound of formula (VIII), the carbon bearing the —NH₂ group of L is in the R configuration. In one aspect of a compound of formula (VIII), the carbon bearing the methyl group of L is in the S configuration. In one aspect of a compound of formula (VIII), the carbon bearing the methyl group of L is in the R configuration.

In some embodiments, the compound of formula (VIII) is of the formula (VIIIa):

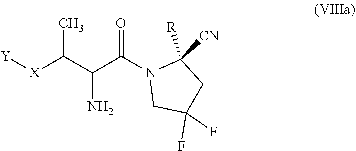

a pharmaceutically acceptable salt, stereoisomer or tautomer thereof, wherein Y, X, and R are as defined for formula (I). In one aspect of a compound of formula (VIIIa), the carbon bearing the —NH₂ group of L is in the S configuration. In one aspect of a compound of formula (VIIIa), the carbon bearing the —NH₂ group of L is in the R configuration. In one aspect of a compound of formula (VIIIa), the carbon bearing the methyl group of L is in the S configuration. In one aspect of a compound of formula (VIIIa), the carbon bearing the methyl group of L is in the R configuration.

In some embodiments, the compound of formula (VIII) is of the formula (VIIIb):

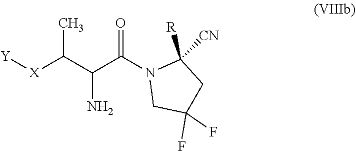

a pharmaceutically acceptable salt, stereoisomer or tautomer thereof, wherein Y, X, and R are as defined for formula (I). In one aspect of a compound of formula (VIIIb), the carbon bearing the —NH₂ group of L is in the S configuration. In one aspect of a compound of formula (VIIIb), the carbon bearing the —NH₂ group of L is in the R configuration. In one aspect of a compound of formula (VIIIb), the carbon bearing the methyl group of L is in the S configuration. In one aspect of a compound of formula (VIIIb), the carbon bearing the methyl group of L is in the R configuration. In some embodiments of the compound of formula (II), the compound is of the formula (IX):

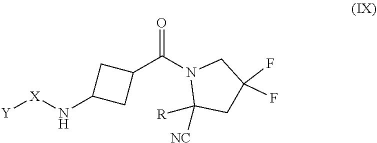

a pharmaceutically acceptable salt, stereoisomer or tautomer thereof, wherein Y, X, and R are as defined for formula (I). In one aspect of a compound of formula (IX), the 1,3-cyclobutylene is the cis isomer. In one aspect of a compound of formula (IX), the 1,3-cyclobutylene is the tran isomer.

In some embodiments, the compound of formula (IX) is of the formula (IXa):

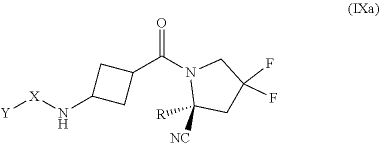

a pharmaceutically acceptable salt, stereoisomer or tautomer thereof, wherein Y, X, and R are as defined for formula (I). In one aspect of a compound of formula (IXa), the 1,3-cyclobutylene is the cis isomer. In one aspect of a compound of formula (IXa), the 1,3-cyclobutylene is the tran isomer.

In some embodiments, the compound of formula (IX) is of the formula (IXb):

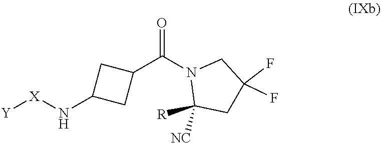

a pharmaceutically acceptable salt, stereoisomer or tautomer thereof, wherein Y, X, and R are as defined for formula (I). In one aspect of a compound of formula (IXb), the 1,3-cyclobutylene is the cis isomer. In one aspect of a compound of formula (IXb), the 1,3-cyclobutylene is the tran isomer.

In some embodiments of the compound of formula (II), where L is —NH—CH₂—, the compound is of the formula (X):

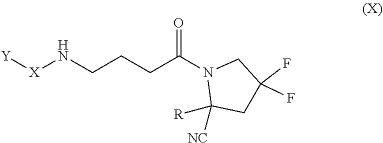

a pharmaceutically acceptable salt, stereoisomer or tautomer thereof, wherein Y, X, and R are as defined for formula (I).

In some embodiments, the compound of formula (X) is of the formula (Xa):

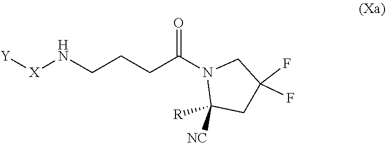

a pharmaceutically acceptable salt, stereoisomer or tautomer thereof, wherein Y, X, and R are as defined for formula (I).

In some embodiments, the compound of formula (X) is of the formula (Xb):

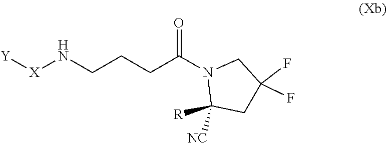

a pharmaceutically acceptable salt, stereoisomer or tautomer thereof, wherein Y, X, and R are as defined for formula (I).

In one variation a compound of formula (I), a pharmaceutically acceptable salt, stereoisomer or tautomer thereof, is provided wherein X is —C(═O)—, —O— or —CH(OH)—. In another variation a compound of formula (I), a pharmaceutically acceptable salt, stereoisomer or tautomer thereof, is provided wherein X is —S—, —S(═O)—, or —S(═O)₂—. In some embodiments of the compound of formula (I), a pharmaceutically acceptable salt, stereoisomer or tautomer thereof, X is —C(═O)—.

In other embodiments of the compound of formula (I), a pharmaceutically acceptable salt, stereoisomer or tautomer thereof, X is —O—. All variations of X apply equally to any applicable formulae herein, such as formulae Ia, Ib, II, IIa, IIb, III, IIIa, IIIb, IV, IVa, IVb, V, Va, Vb, VI, VIa, VIb, VII, VIIa, VIIb, VIII, VIIIa and VIIIb.

In some embodiments of the compound of formula (I), a pharmaceutically acceptable salt,

stereoisomer or tautomer thereof, L is

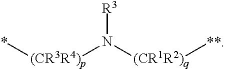

In a particular such embodiment, R^{a} is H, and R¹, R², R³ and R⁴, if present, are each H. In one embodiment, L is

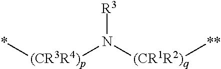

and X is —C═O. In another embodiment, L is

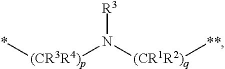

### X is —C═O and p is 0.

In some embodiments, L is

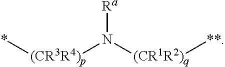

In one particular variation, R¹ and R² are attached to the same carbon atom. In another particular variation, R¹ and R² are attached to different carbon atoms.

In some embodiments, L is —N(R^{a})—CR¹R²— (i. e., p is 0). In one particular variation, L is —NH—CR¹R²—. In another particular variation, L is —NH—CH₂—. In another particular variation, L is —NH—CH(CH₃)—. In another particular variation, L is —NH—CR¹R²—, wherein R¹ and R² are taken together with the carbon atom or atoms to which they are attached to form a 3- to 8-membered cycloalkylene (e.g., cyclopropylene).

In some embodiments, L is —N(R^{a})—(CR¹R²)₃— (i. e., p is 0). In one particular variation, L is —NH—(CR¹R²)₃—. In another particular variation, L is —NH—(CH₂)₃—. In another particular variation, L is —NH—(CR¹R²)₃—, wherein R¹ and R² from two non-adjacent carbons are taken together with the carbon atoms to which they are attached and interstitial carbon to form a 3- to 8-membered cycloalkylene (e.g., 1,3-cyclobutylene).

In other embodiments of the compound of formula (I), a pharmaceutically acceptable salt, stereoisomer or tautomer thereof, L is

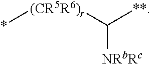

In one such embodiment, X is —C═O.

In another such embodiment, X is —O—. In a further such embodiment, X is —CH(OH)—. In yet another such embodiment, X is —S—. In still another such embodiment, X is —S(═O)—. In still another such embodiment, X is —S(═O)₂. In one aspect of such embodiments, r is 1. In another aspect of such embodiments, r is 2. In still another aspect of such embodiments, r is 3. In any embodiment provided where L is

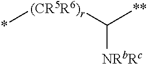

in one variation, R^{b} and R^{c} are both H. In any embodiment provided where L is

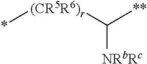

in another variation, R^{b} and R^{c} are both H, r is 1, R⁵ is H and R⁶ is a C₁-C₆ alkyl such as methyl.

In some of these embodiments, L is —CR⁵R⁶—CH(NR^{b}R^{c})— (i.e., r is 1). In one particular variation, L is —CH(R⁵)—CH(NH₂)—, including but not limited to aspects wherein R⁵ is hydrogen or C₁-C₆ alkyl. In one particular variation, L is —CH₂—CH(NH₂)—. In another particular variation, L is —CH(CH₃)—CH(NH₂)—.

In some embodiments of the compound of formula (I), a pharmaceutically acceptable salt, stereoisomer or tautomer thereof, L is

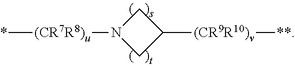

In one such embodiment, X is —C═O. In another such embodiment, X is —O—. In a further such embodiment, X is —S—. In still another such embodiment, X is —S(═O)—. In still another such embodiment, X is —S(═O)₂. In a particular variation, L is

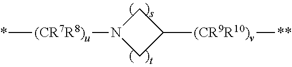

and u is 0. In another variation, L is

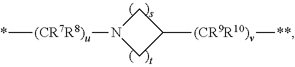

u is 0 and X is selected from the group consisting of —C═O, —O—, —S—, —S(═O)— and —S(═O)₂. In any embodiment or variation where L is

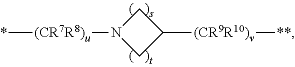

in one aspect, s is 1 and t is 1.

In one variation, L is

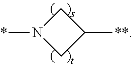

In another particular variation, L is

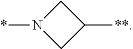

In one variation, L is

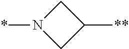

and X is selected from the group consisting of —C═O, —O—, —S—, —S(═O)— and —S(═O)₂.

In one variation, provided herein is a compound of formula (I), a pharmaceutically acceptable salt, stereoisomer or tautomer thereof, wherein the —X-L- moiety is selected from the group consisting of:

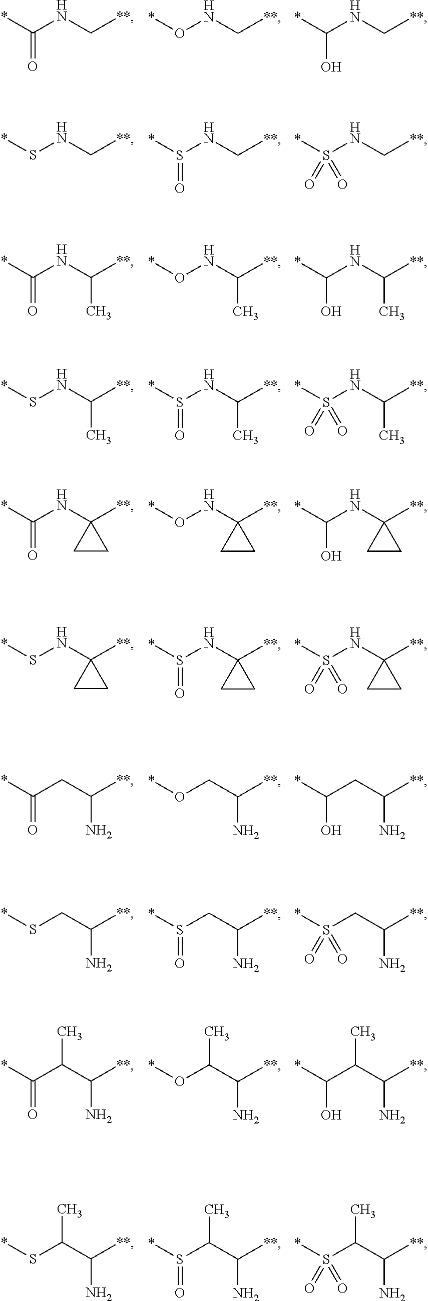

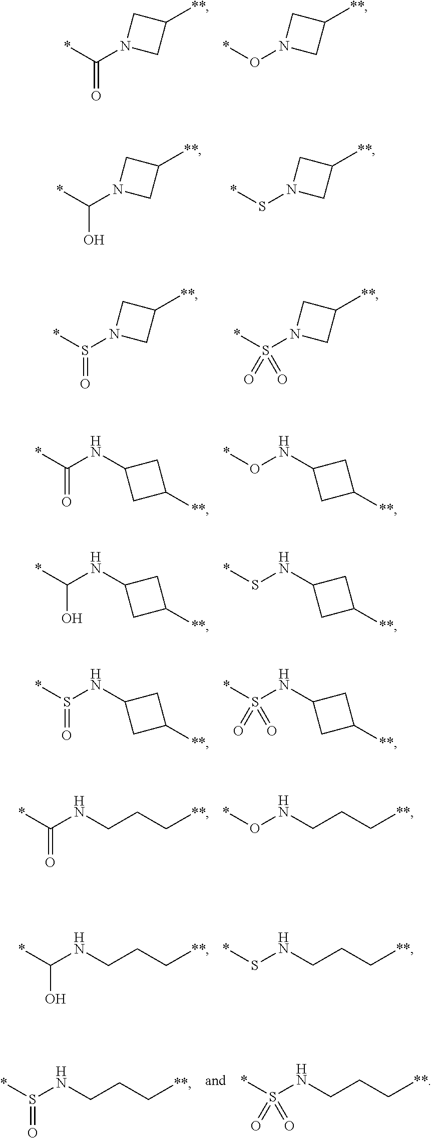

wherein * represents the point of attachment to the Y moiety and ** represents the point of attachment to the remainder of the molecule.

In another variation is provided a compound of formula (I), a pharmaceutically acceptable salt, stereoisomer or tautomer thereof, wherein the —X-L- moiety is selected from the group consisting of:

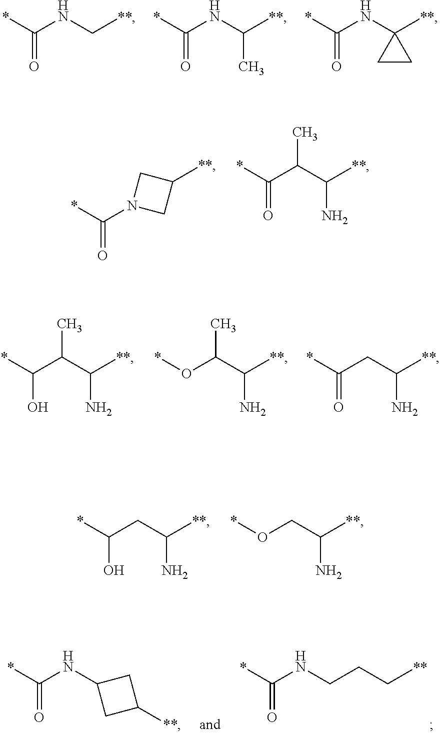

wherein * represents the point of attachment to the Y moiety and ** represents the point of attachment to the remainder of the molecule.

In one aspect, provided is a compound of formula (I) or a pharmaceutically acceptable salt thereof, wherein the compound has any one or more of the following features:
(i) X is —C(═O)—, —O— or —CH(OH)—;
(ii) L is:
   (a) —NH—(CR¹R²)_{q}—, wherein R¹ and R², independently of each other and independently at each occurrence, are hydrogen or C₁-C₂ alkyl, or R¹ and R² groups attached to the same carbon atom are taken together with the carbon atom to which they are attached to form a 3- to 5-membered cycloalkylene, or R¹ and R² groups attached to two different carbon atoms are taken together with the carbon atoms to which they are attached to form a 3- to 5-membered cycloalkylene (examples of such —NH—(CR¹R²)_{q}— moieties include

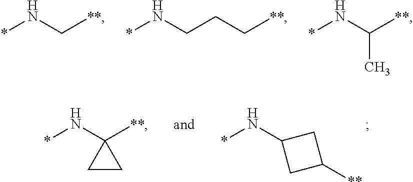

(b) —CR⁵R⁶—CH(NH₂)—, wherein R⁵ and R⁶, independently of each other and independently at each occurrence, are hydrogen or C₁-C₂ alkyl, (examples of such —CR⁵R⁶—CH(NH₂)— moieties include

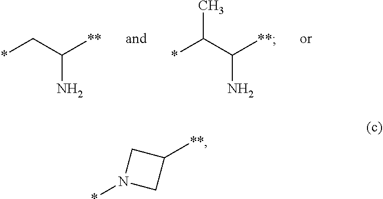

wherein * represents the point of attachment to the Y—X— moiety and ** represents the point of attachment to the remainder of the molecule; and
(iii) Y is:
   (a) C₆-C₉ aryl substituted by R¹¹, such as 2,3-dihydro-1H-inden-2-yl, phenyl and naphthyl, which are substituted by at least one R¹¹;
   (b) 6- to 10-membered heteroaryl substituted by R¹², such as a pyridinyl, pyrimidinyl, pyridin-2(1H)-onyl, and quinolin-6-yl, which are substituted by at least one R¹²; or
   (c) 3- to 12-membered heterocyclyl substituted by R¹³, such as 2H-pyran-2-only, isoindolinyl, piperidin-2-only and piperidinyl, which are substituted by at least one R¹³.

In one aspect of this variation, (i), (ii)(a), and (iii)(a) apply. In another variation, (i), (ii)(a), and (iii)(b) apply apply. In another variation, (i), (ii)(a), and (iii)(c) apply. In another variation, (i), (ii)(b), and (iii)(a) apply. In another variation, (i), (ii)(b), and (iii)(b) apply. In another variation, (i), (ii)(b), and (iii)(c) apply. In another variation, (i), (ii)(c), and (iii)(a) apply. In another variation, (i), (ii)(c), and (iii)(b) apply. In another variation, (i), (ii)(c), and (iii)(c) apply.

All variations of L, or combinations of X and L, apply equally to any applicable formulae herein, such as formulae Ia, Ib, II, IIa, IIb, III, IIIa, IIIb, IV, IVa, IVb, V, Va, Vb, VI, VIa, VIb, VII, VIIa, VIIb, VIII, VIIIa and VIIIb.

In some embodiments, Y is C₆-C₉ aryl substituted by one or more R¹¹, 6- to 10-membered heteroaryl substituted by one or more R¹², or 3- to 12-membered heterocyclyl substituted by one or more R¹³. In one variation, Y is substituted with 1 to 3 R¹¹, R¹² or R¹³ moieties which may be the same or different.

In some embodiments, Y is C₆-C₉ aryl substituted by R¹¹. In one aspect, Y is phenyl substituted by R¹¹. In one variation, Y is substituted with 1 to 3 R¹¹ moieties which may be the same or different.

In some embodiments, Y is 6- to 10-membered heteroaryl substituted by R¹². In one variation, when Y is a 6-membered heteroaryl substituted by R¹². In some embodiments, Y is pyridine substituted by R¹². In some embodiments, Y is pyridin-4-yl substituted by R¹². In some embodiments, Y is pyridin-4-yl substituted by R¹² in the 3-position. In some embodiments, Y is quinolinyl substituted by R¹². In some embodiments, Y is quinolin-4-yl substituted by R¹². In some embodiments, Y is quinolin-4-yl substituted by R¹² in the 7-position. In some embodiments, Y is

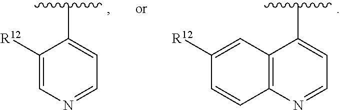

In one variation, R¹² is C₁-C₆ alkyl substituted by R^{L}. In another variation, R¹² is C₂-C₆ alkenyl substituted by R^{L}. In another variation, R¹² is C₂-C₆ alkynyl substituted by R^{L}. In yet another variation, R¹² is 3- to 12-membered heterocyclyl substituted by R^{L}.

In some embodiments, R^{L} is C₆-C₁₄ aryl substituted by halogen, —OH, cyano, C₁-C₆ alkyl, C₁-C₆ perhaloalkyl, C₁-C₆ alkoxy, C₁-C₆ perhaloalkoxy or C₆-C₁₄ aryl. In some embodiments, R^{L} is 5- to 10-membered heteroaryl substituted by halogen, —OH, cyano, oxo, —NH₂, C₁-C₆ alkyl, C₁-C₆ perhaloalkyl, C₁-C₆ alkoxy, or C₁-C₆ perhaloalkoxy. In some embodiments, R^{L} is 3- to 12-membered heterocyclyl substituted by halogen, —OH, cyano, oxo, —NH₂, C₁-C₆ alkyl, C₁-C₆ perhaloalkyl, C₁-C₆ alkoxy, or C₁-C₆ perhaloalkoxy.

In one variation, R¹² is —NR¹⁴C(O)R¹⁵. In some embodiments, at least one of R¹⁴ and R¹⁵ is C₁-C₆ alkyl, or C₆-C₁₄ aryl, wherein the C₁-C₆ alkyl, or C₆-C₁₄ aryl of R¹⁴ and R¹⁵ are independently substituted by C₁-C₆ perhaloalkyl, C₁-C₆ alkoxy, C₁-C₆ perhaloalkoxy, C₆-C₁₄ aryl or C₆-C₁₄ aryloxy, wherein the C₆-C₁₄ aryl or C₆-C₁₄ aryloxy is further optionally substituted by halogen, —OH, cyano, C₁-C₆ alkyl, C₁-C₆ perhaloalkyl, C₁-C₆ alkoxy, or C₁-C₆ perhaloalkoxy. In some embodiments, at least one of R¹⁴ and R¹⁵ is C₁-C₆ alkyl, or C₆-C₁₄ aryl, wherein the C₁-C₆ alkyl, or C₆-C₁₄ aryl of R¹⁴ and R¹⁵ are independently substituted by C₁-C₆ alkoxy, C₁-C₆ perhaloalkoxy, C₆-C₁₄ aryl or C₆-C₁₄ aryloxy, wherein the C₆-C₁₄ aryl or C₆-C₁₄ aryloxy is further optionally substituted by halogen, —OH, cyano, C₁-C₆ alkyl, C₁-C₆ perhaloalkyl, C₁-C₆ alkoxy, or C₁-C₆ perhaloalkoxy.

In one variation, R¹² is —NR¹⁴R¹⁵. In some embodiments, one of R¹⁴ and R¹⁵ is hydrogen and the other is C₁-C₆ alkyl, wherein the C₁-C₆ alkyl is substituted by C₁-C₆ perhaloalkyl, C₁-C₆ alkoxy, C₁-C₆ perhaloalkoxy, C₆-C₁₄ aryl or C₆-C₁₄ aryloxy, wherein the C₆-C₁₄ aryl or C₆-C₁₄ aryloxy is further optionally substituted by halogen, —OH, cyano, C₁-C₆ alkyl, C₁-C₆ perhaloalkyl, C₁-C₆ alkoxy, or C₁-C₆ perhaloalkoxy.

In some embodiments, Y is B′ or a tautomer thereof. Thus, it is understood that in some embodiments, Y is

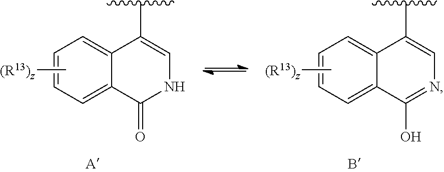

wherein z is 0, 1, 2, 3, 4, or 5; [symbol] indicates tautomerism between A′ and B′; and R¹² and R¹³ are identical for any pair of tautomers. In some embodiments, Y is D′ or a tautomer thereof. Thus, it is understood that in some embodiments, Y is

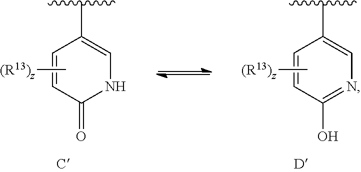

wherein z is 0, 1, 2, 3, 4, or 5; [symbol] indicates tautomerism between C′ and D′; and R¹² and R¹³ are identical for any pair of tautomers.

In some embodiments, Y is 3- to 12-membered heterocyclyl substituted by R¹³. In some embodiments, Y is substituted isoindolin-2-yl. In some embodiments, Y is piperidin-2-on-5-yl substituted by R¹³.

Also provided are salts of compounds referred to herein, such as pharmaceutically acceptable salts. The invention also includes any or all of the stereochemical forms, including any enantiomeric or diastereomeric forms, and any tautomers or other forms of the compounds described.

Some of the compounds described herein exist in equilibrium with a tautomeric form. For example, amide A is a tautomeric form of B and imidic acid B is a tautomeric form of A. Similarly, amide C is a tautomeric form of D and imidic acid D is a tautomeric form of C. Amide A exists in equilibrium with a tautomeric form of imidic acid B, and amide C exists in equilibrium with a tautomeric form of imidic acid D. Regardless of which tautomeric form is depicted, the compounds are understood by one of ordinary skill in the art to comprise both the amide and the imidic acid tautomers.

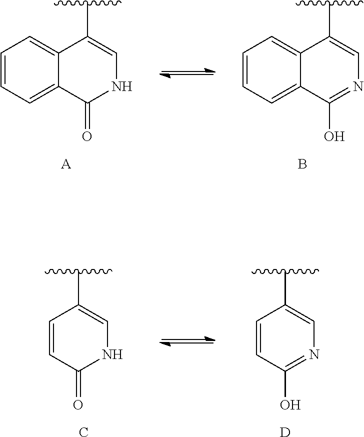

A compound as detailed herein may in one aspect be in a purified form and compositions comprising a compound in purified forms are detailed herein. Compositions comprising a compound as detailed herein, or a pharmaceutically acceptable salt, stereoisomer or tautomer thereof are provided, such as compositions of substantially pure compounds. In some embodiments, a composition containing a compound as detailed herein, or a pharmaceutically acceptable salt, stereoisomer or tautomer thereof is in substantially pure form. Unless otherwise stated, “substantially pure” intends a composition that contains no more than 35% impurity, wherein the impurity denotes a compound other than the compound comprising the majority of the composition or a pharmaceutically acceptable salt, stereoisomer or tautomer thereof. In some embodiments, a composition of substantially pure compound, or a pharmaceutically acceptable salt, stereoisomer or tautomer thereof is provided wherein the composition contains no more than 25%, 20%, 15%, 10%, or 5% impurity. In some embodiments, a composition of substantially pure compound, or a pharmaceutically acceptable salt, stereoisomer or tautomer thereof is provided wherein the composition contains or no more than 3%, 2%, 1% or 0.5% impurity.

Representative compounds are listed in Table 1.

TABLE 1

| Compound | |
|---|---|
| No. | Structure |
| 1 | 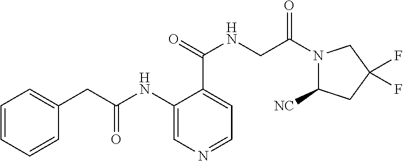 [SMILES: N#C[C@@H]1CC(F)(F)CN1C(=O)CNC(=O)c1ccncc1NC(=O)Cc1ccccc1] |
| 2 | 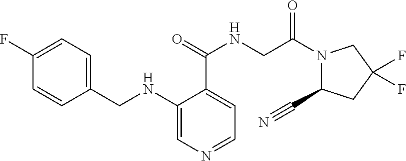 [SMILES: N#C[C@@H]1CC(F)(F)CN1C(=O)CNC(=O)c1ccncc1NCc1ccc(F)cc1] |
| 3 | 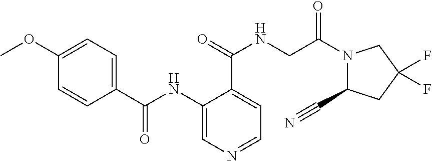 [SMILES: COc1ccc(C(=O)Nc2cnccc2C(=O)NCC(=O)N2CC(F)(F)C[C@H]2C#N)cc1] |
| 4 | 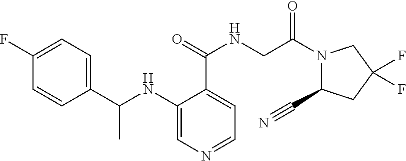 [SMILES: CC(Nc1cnccc1C(=O)NCC(=O)N1CC(F)(F)C[C@H]1C#N)c1ccc(F)cc1] |
| 5 | 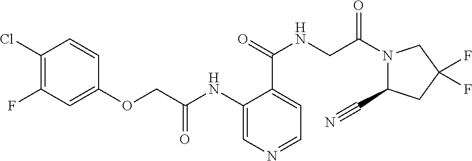 [SMILES: N#C[C@@H]1CC(F)(F)CN1C(=O)CNC(=O)c1ccncc1NC(=O)COc1ccc(Cl)c(F)c1] |
| 6 | 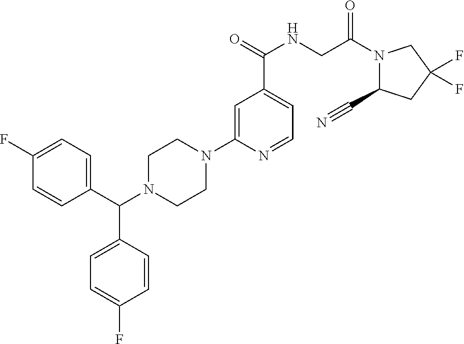 [SMILES: N#C[C@@H]1CC(F)(F)CN1C(=O)CNC(=O)c1ccnc(N2CCN(C(c3ccc(F)cc3)c3ccc(F)cc3)CC2)c1] |
| 7 | 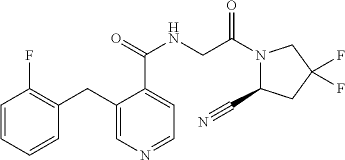 [SMILES: N#C[C@@H]1CC(F)(F)CN1C(=O)CNC(=O)c1ccncc1Cc1ccccc1F] |
| 8 | 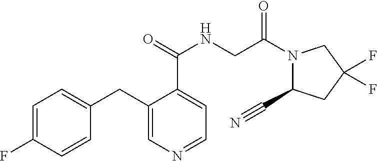 [SMILES: N#C[C@@H]1CC(F)(F)CN1C(=O)CNC(=O)c1ccncc1Cc1ccc(F)cc1] |
| 9 | 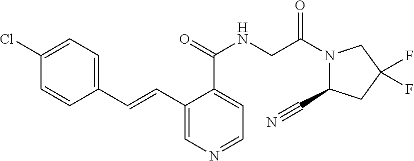 [SMILES: N#C[C@@H]1CC(F)(F)CN1C(=O)CNC(=O)c1ccncc1/C=C/c1ccc(Cl)cc1] |
| 10 | 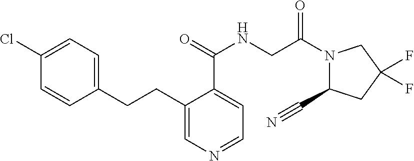 [SMILES: N#C[C@@H]1CC(F)(F)CN1C(=O)CNC(=O)c1ccncc1CCc1ccc(Cl)cc1] |
| 11 | 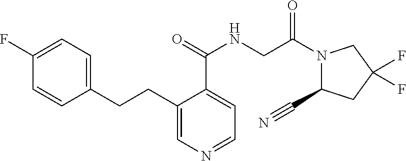 [SMILES: N#C[C@@H]1CC(F)(F)CN1C(=O)CNC(=O)c1ccncc1CCc1ccc(F)cc1] |
| 12 | 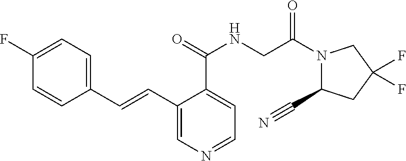 [SMILES: N#C[C@@H]1CC(F)(F)CN1C(=O)CNC(=O)c1ccncc1/C=C/c1ccc(F)cc1] |
| 13 | 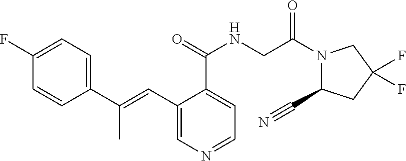 [SMILES: C/C(=C\c1cnccc1C(=O)NCC(=O)N1CC(F)(F)C[C@H]1C#N)c1ccc(F)cc1] |
| 14 | 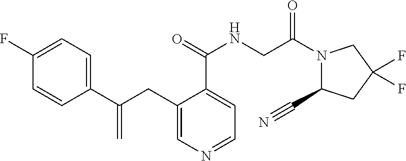 [SMILES: C=C(Cc1cnccc1C(=O)NCC(=O)N1CC(F)(F)C[C@H]1C#N)c1ccc(F)cc1] |
| 15 | 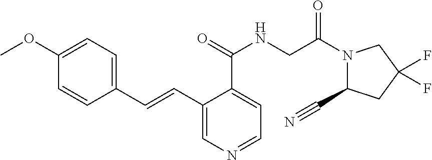 [SMILES: COc1ccc(/C=C/c2cnccc2C(=O)NCC(=O)N2CC(F)(F)C[C@H]2C#N)cc1] |
| 16 | 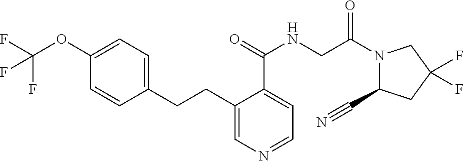 [SMILES: N#C[C@@H]1CC(F)(F)CN1C(=O)CNC(=O)c1ccncc1CCc1ccc(OC(F)(F)F)cc1] |
| 17 | 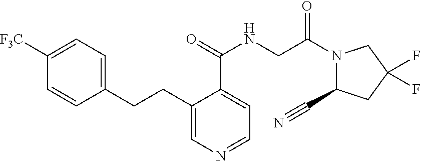 [SMILES: N#C[C@@H]1CC(F)(F)CN1C(=O)CNC(=O)c1ccncc1CCc1ccc(C(F)(F)F)cc1] |
| 18 | 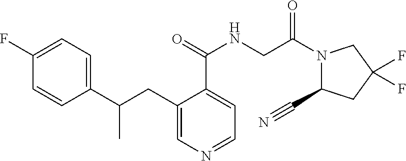 [SMILES: CC(Cc1cnccc1C(=O)NCC(=O)N1CC(F)(F)C[C@H]1C#N)c1ccc(F)cc1] |
| 19 | 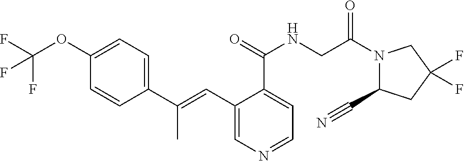 [SMILES: C/C(=C\c1cnccc1C(=O)NCC(=O)N1CC(F)(F)C[C@H]1C#N)c1ccc(OC(F)(F)F)cc1] |
| 20 | 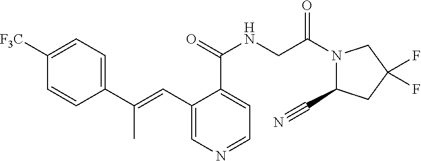 [SMILES: C/C(=C\c1cnccc1C(=O)NCC(=O)N1CC(F)(F)C[C@H]1C#N)c1ccc(C(F)(F)F)cc1] |
| 21 | 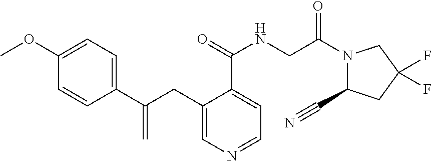 [SMILES: C=C(Cc1cnccc1C(=O)NCC(=O)N1CC(F)(F)C[C@H]1C#N)c1ccc(OC)cc1] |
| 22 | 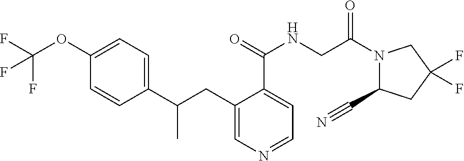 [SMILES: CC(Cc1cnccc1C(=O)NCC(=O)N1CC(F)(F)C[C@H]1C#N)c1ccc(OC(F)(F)F)cc1] |
| 23 | 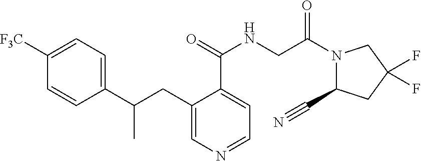 [SMILES: CC(Cc1cnccc1C(=O)NCC(=O)N1CC(F)(F)C[C@H]1C#N)c1ccc(C(F)(F)F)cc1] |
| 24 | 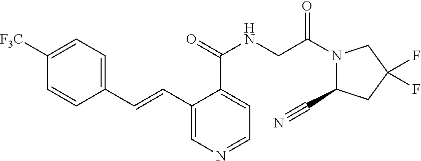 [SMILES: N#C[C@@H]1CC(F)(F)CN1C(=O)CNC(=O)c1ccncc1/C=C/c1ccc(C(F)(F)F)cc1] |
| 25 | 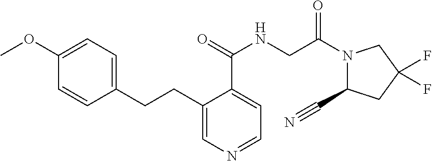 [SMILES: COc1ccc(CCc2cnccc2C(=O)NCC(=O)N2CC(F)(F)C[C@H]2C#N)cc1] |
| 26 | 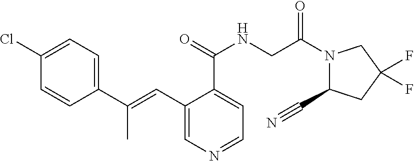 [SMILES: C/C(=C\c1cnccc1C(=O)NCC(=O)N1CC(F)(F)C[C@H]1C#N)c1ccc(Cl)cc1] |
| 27 | 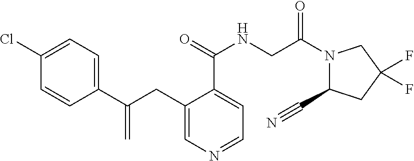 [SMILES: C=C(Cc1cnccc1C(=O)NCC(=O)N1CC(F)(F)C[C@H]1C#N)c1ccc(Cl)cc1] |
| 28 | 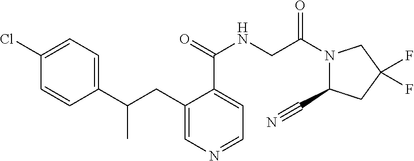 [SMILES: CC(Cc1cnccc1C(=O)NCC(=O)N1CC(F)(F)C[C@H]1C#N)c1ccc(Cl)cc1] |
| 29 | 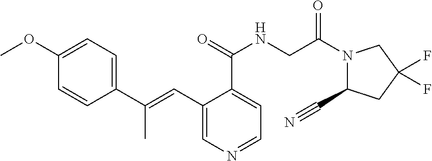 [SMILES: COc1ccc(/C(C)=C/c2cnccc2C(=O)NCC(=O)N2CC(F)(F)C[C@H]2C#N)cc1] |
| 30 | 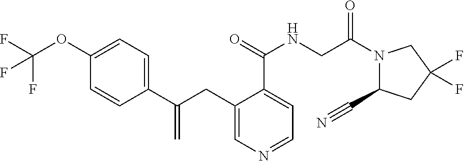 [SMILES: C=C(Cc1cnccc1C(=O)NCC(=O)N1CC(F)(F)C[C@H]1C#N)c1ccc(OC(F)(F)F)cc1] |
| 31 | 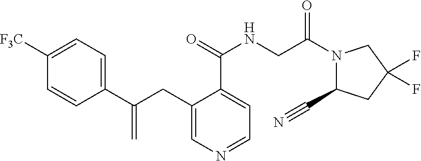 [SMILES: C=C(Cc1cnccc1C(=O)NCC(=O)N1CC(F)(F)C[C@H]1C#N)c1ccc(C(F)(F)F)cc1] |
| 32 | 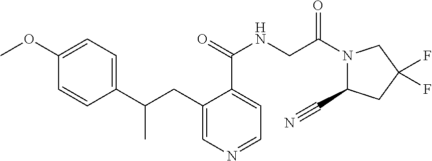 [SMILES: COc1ccc(C(C)Cc2cnccc2C(=O)NCC(=O)N2CC(F)(F)C[C@H]2C#N)cc1] |
| 33 | 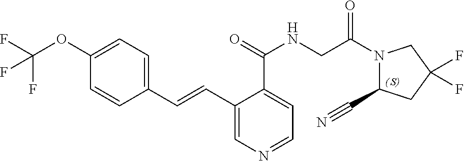 |
| 34 | 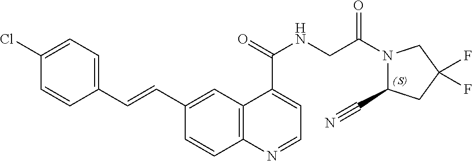 |
| 35 | 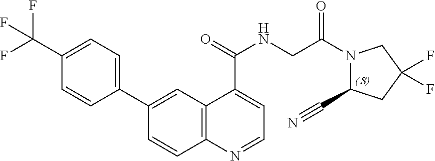 |
| 36 | 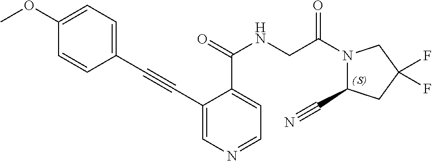 |
| 37 | 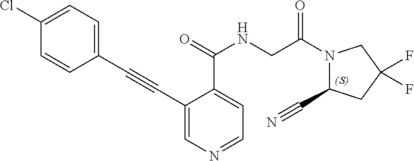 |
| 38 | 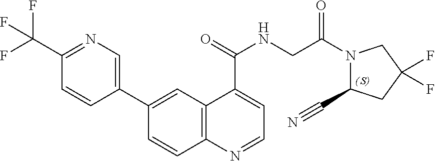 |
| 39 | 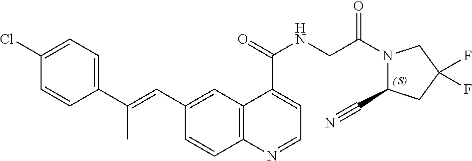 |
| 40 | 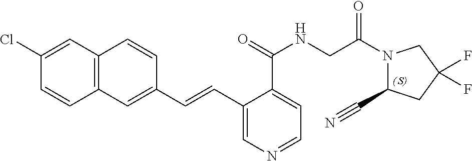 |
| 41 | 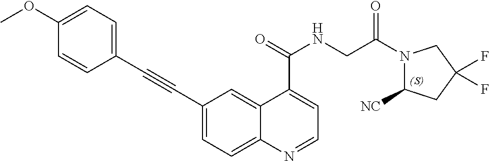 |
| 42 | 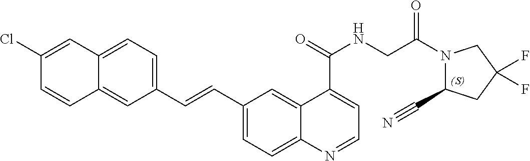 |
| 43 | 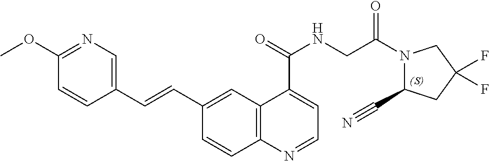 |
| 44 | 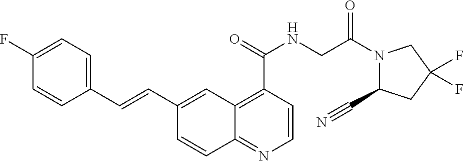 [SMILES: N#C[C@@H]1CC(F)(F)CN1C(=O)CNC(=O)c1ccnc2ccc(/C=C/c3ccc(F)cc3)cc12] |
| 45 | 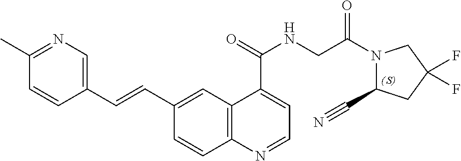 |
| 46 | 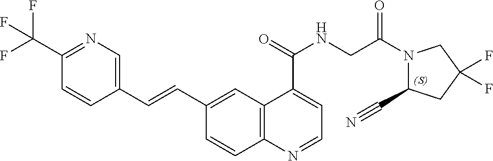 |
| 47 | 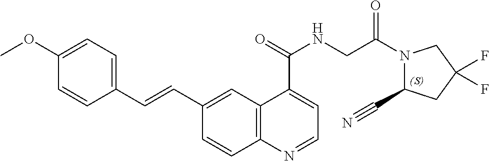 |
| 48 | 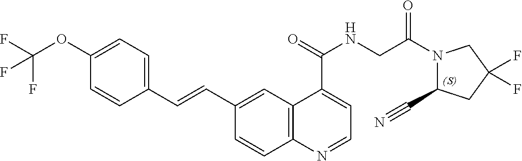 |
| 49 | 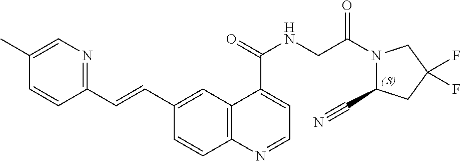 |
| 50 | 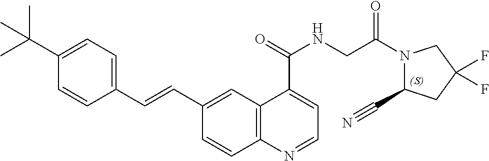 |
| 51 | 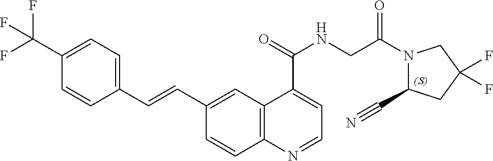 |
| 52 | 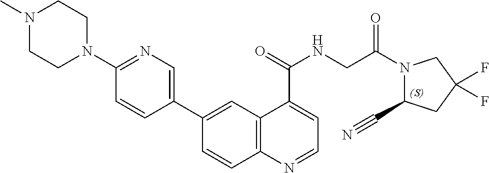 |
| 53 | 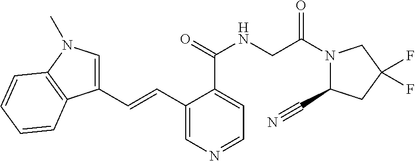 [SMILES: Cn1cc(/C=C/c2cnccc2C(=O)NCC(=O)N2CC(F)(F)C[C@H]2C#N)c2ccccc21] |
| 54 | 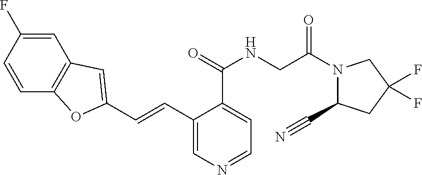 [SMILES: N#C[C@@H]1CC(F)(F)CN1C(=O)CNC(=O)c1ccncc1/C=C/c1cc2cc(F)ccc2o1] |
| 55 | 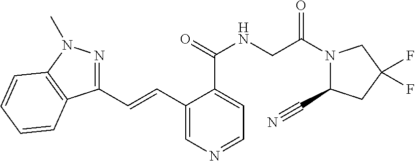 [SMILES: Cn1nc(/C=C/c2cnccc2C(=O)NCC(=O)N2CC(F)(F)C[C@H]2C#N)c2ccccc21] |
| 56 | 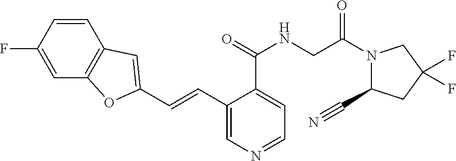 [SMILES: N#C[C@@H]1CC(F)(F)CN1C(=O)CNC(=O)c1ccncc1/C=C/c1cc2ccc(F)cc2o1] |
| 57 | 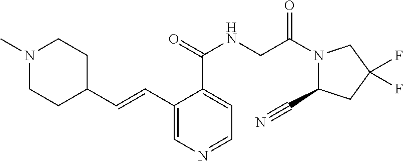 [SMILES: CN1CCC(/C=C/c2cnccc2C(=O)NCC(=O)N2CC(F)(F)C[C@H]2C#N)CC1] |
| 58 | 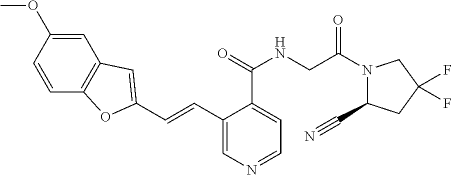 [SMILES: COc1ccc2oc(/C=C/c3cnccc3C(=O)NCC(=O)N3CC(F)(F)C[C@H]3C#N)cc2c1] |
| 59 | 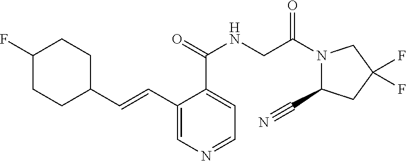 [SMILES: N#C[C@@H]1CC(F)(F)CN1C(=O)CNC(=O)c1ccncc1/C=C/C1CCC(F)CC1] |
| 60 | 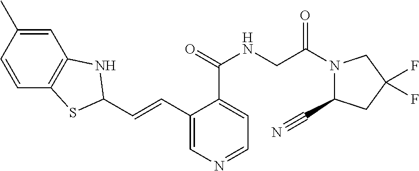 [SMILES: Cc1ccc2c(c1)NC(/C=C/c1cnccc1C(=O)NCC(=O)N1CC(F)(F)C[C@H]1C#N)S2] |
| 61 | 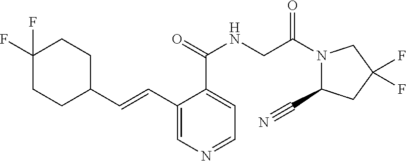 [SMILES: N#C[C@@H]1CC(F)(F)CN1C(=O)CNC(=O)c1ccncc1/C=C/C1CCC(F)(F)CC1] |
| 62 | 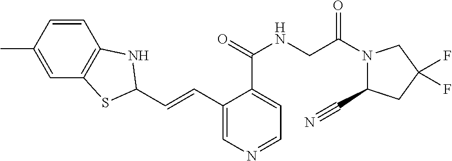 [SMILES: Cc1ccc2c(c1)SC(/C=C/c1cnccc1C(=O)NCC(=O)N1CC(F)(F)C[C@H]1C#N)N2] |
| 63 | 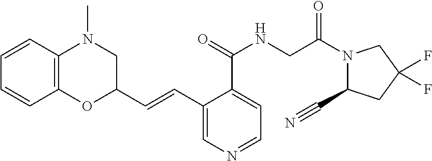 [SMILES: CN1CC(/C=C/c2cnccc2C(=O)NCC(=O)N2CC(F)(F)C[C@H]2C#N)Oc2ccccc21] |
| 64 | 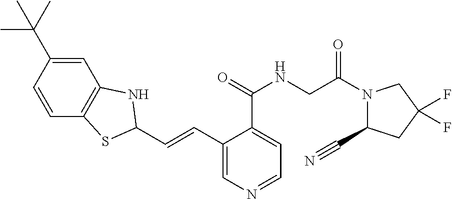 [SMILES: CC(C)(C)c1ccc2c(c1)NC(/C=C/c1cnccc1C(=O)NCC(=O)N1CC(F)(F)C[C@H]1C#N)S2] |
| 65 | 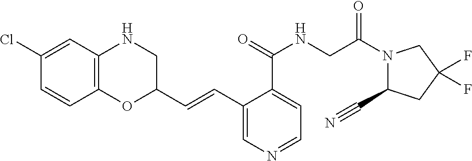 [SMILES: N#C[C@@H]1CC(F)(F)CN1C(=O)CNC(=O)c1ccncc1/C=C/C1CNc2cc(Cl)ccc2O1] |
| 66 | 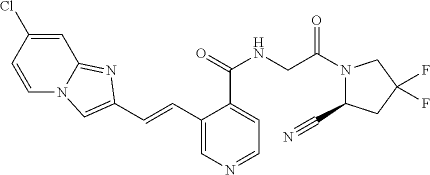 [SMILES: N#C[C@@H]1CC(F)(F)CN1C(=O)CNC(=O)c1ccncc1/C=C/c1cn2ccc(Cl)cc2n1] |
| 67 | 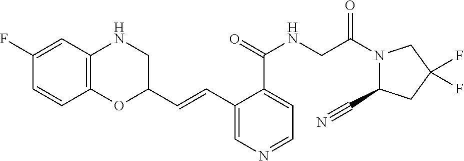 [SMILES: N#C[C@@H]1CC(F)(F)CN1C(=O)CNC(=O)c1ccncc1/C=C/C1CNc2cc(F)ccc2O1] |
| 68 | 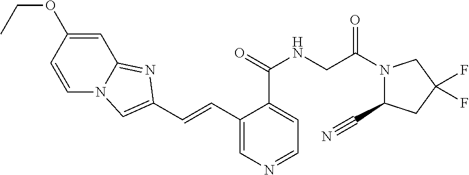 [SMILES: CCOc1ccn2cc(/C=C/c3cnccc3C(=O)NCC(=O)N3CC(F)(F)C[C@H]3C#N)nc2c1] |
| 69 | 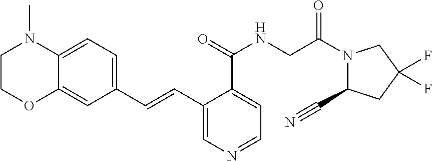 [SMILES: CN1CCOc2cc(/C=C/c3cnccc3C(=O)NCC(=O)N3CC(F)(F)C[C@H]3C#N)ccc21] |
| 70 | 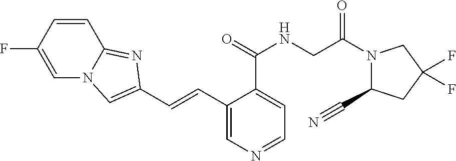 [SMILES: N#C[C@@H]1CC(F)(F)CN1C(=O)CNC(=O)c1ccncc1/C=C/c1cn2cc(F)ccc2n1] |
| 71 | 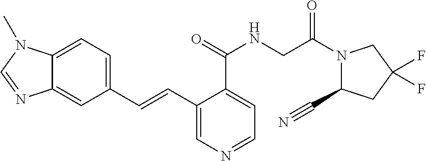 [SMILES: Cn1cnc2cc(/C=C/c3cnccc3C(=O)NCC(=O)N3CC(F)(F)C[C@H]3C#N)ccc21] |
| 72 | 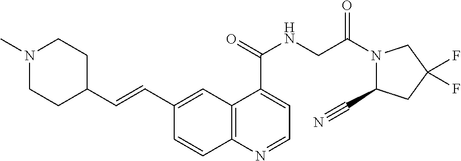 [SMILES: CN1CCC(/C=C/c2ccc3nccc(C(=O)NCC(=O)N4CC(F)(F)C[C@H]4C#N)c3c2)CC1] |
| 73 | 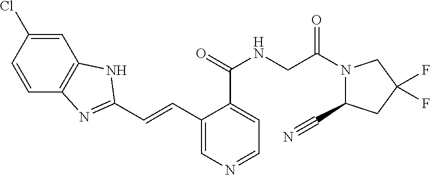 [SMILES: N#C[C@@H]1CC(F)(F)CN1C(=O)CNC(=O)c1ccncc1/C=C/c1nc2ccc(Cl)cc2[nH]1] |
| 74 | 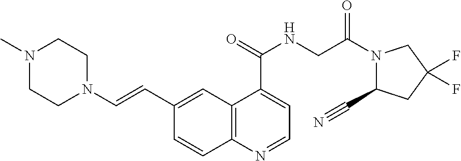 [SMILES: CN1CCN(/C=C/c2ccc3nccc(C(=O)NCC(=O)N4CC(F)(F)C[C@H]4C#N)c3c2)CC1] |
| 75 | 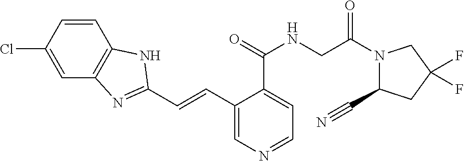 [SMILES: N#C[C@@H]1CC(F)(F)CN1C(=O)CNC(=O)c1ccncc1/C=C/c1nc2cc(Cl)ccc2[nH]1] |
| 76 | 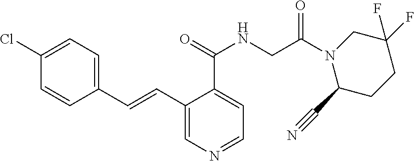 [SMILES: N#C[C@@H]1CCC(F)(F)CN1C(=O)CNC(=O)c1ccncc1/C=C/c1ccc(Cl)cc1] |
| 77 | 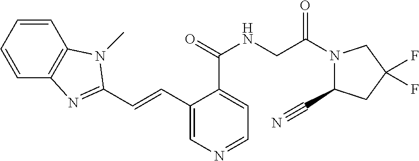 [SMILES: Cn1c(/C=C/c2cnccc2C(=O)NCC(=O)N2CC(F)(F)C[C@H]2C#N)nc2ccccc21] |
| 78 | 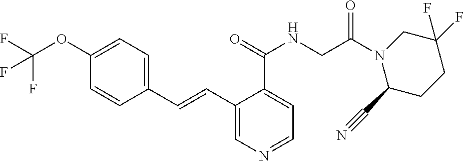 [SMILES: N#C[C@@H]1CCC(F)(F)CN1C(=O)CNC(=O)c1ccncc1/C=C/c1ccc(OC(F)(F)F)cc1] |
| 79 | 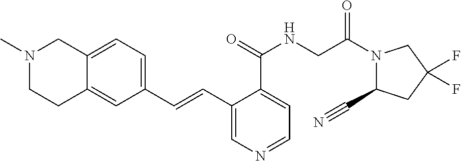 [SMILES: CN1CCc2cc(/C=C/c3cnccc3C(=O)NCC(=O)N3CC(F)(F)C[C@H]3C#N)ccc2C1] |
| 80 | 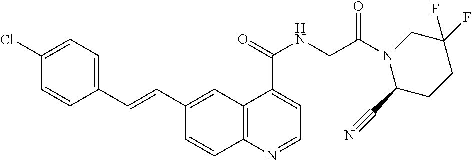 [SMILES: N#C[C@@H]1CCC(F)(F)CN1C(=O)CNC(=O)c1ccnc2ccc(/C=C/c3ccc(Cl)cc3)cc12] |
| 81 | 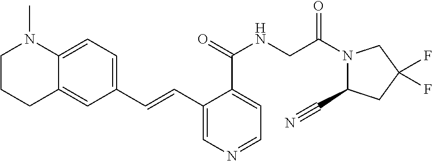 [SMILES: CN1CCCc2cc(/C=C/c3cnccc3C(=O)NCC(=O)N3CC(F)(F)C[C@H]3C#N)ccc21] |
| 82 | 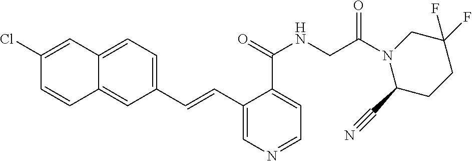 [SMILES: N#C[C@@H]1CCC(F)(F)CN1C(=O)CNC(=O)c1ccncc1/C=C/c1ccc2cc(Cl)ccc2c1] |
| 83 | 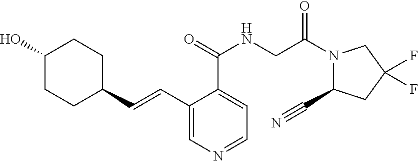 [SMILES: N#C[C@@H]1CC(F)(F)CN1C(=O)CNC(=O)c1ccncc1/C=C/[C@H]1CC[C@H](O)CC1] |
| 84 | 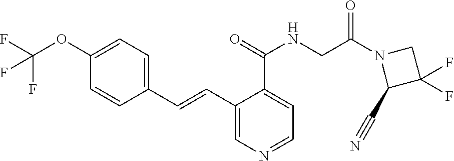 [SMILES: N#C[C@H]1N(C(=O)CNC(=O)c2ccncc2/C=C/c2ccc(OC(F)(F)F)cc2)CC1(F)F] |
| 85 | 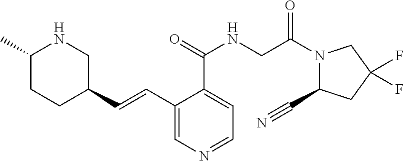 [SMILES: C[C@H]1CC[C@H](/C=C/c2cnccc2C(=O)NCC(=O)N2CC(F)(F)C[C@H]2C#N)CN1] |
| 86 | 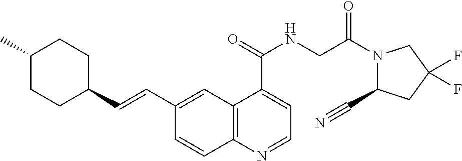 [SMILES: C[C@H]1CC[C@H](/C=C/c2ccc3nccc(C(=O)NCC(=O)N4CC(F)(F)C[C@H]4C#N)c3c2)CC1] |
| 87 | 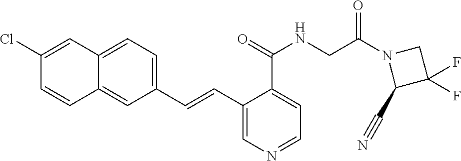 [SMILES: N#C[C@H]1N(C(=O)CNC(=O)c2ccncc2/C=C/c2ccc3cc(Cl)ccc3c2)CC1(F)F] |
| 88 | 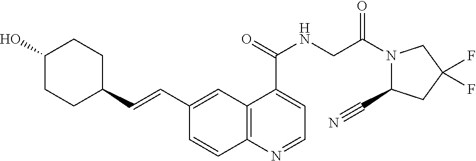 [SMILES: N#C[C@@H]1CC(F)(F)CN1C(=O)CNC(=O)c1ccnc2ccc(/C=C/[C@H]3CC[C@H](O)CC3)cc12] |
| 89 | 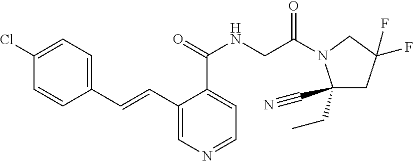 [SMILES: CC[C@@]1(C#N)CC(F)(F)CN1C(=O)CNC(=O)c1ccncc1/C=C/c1ccc(Cl)cc1] |
| 90 | 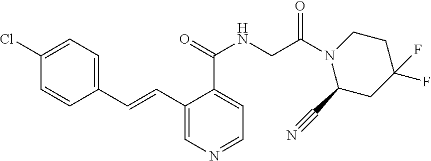 [SMILES: N#C[C@@H]1CC(F)(F)CCN1C(=O)CNC(=O)c1ccncc1/C=C/c1ccc(Cl)cc1] |
| 91 | 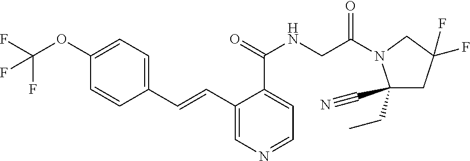 [SMILES: CC[C@@]1(C#N)CC(F)(F)CN1C(=O)CNC(=O)c1ccncc1/C=C/c1ccc(OC(F)(F)F)cc1] |
| 92 | 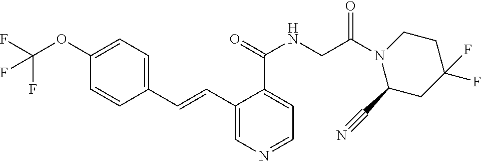 [SMILES: N#C[C@@H]1CC(F)(F)CCN1C(=O)CNC(=O)c1ccncc1/C=C/c1ccc(OC(F)(F)F)cc1] |
| 93 | 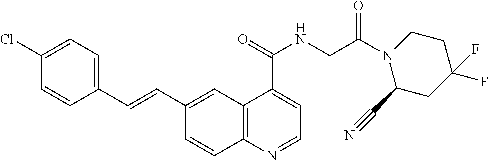 [SMILES: N#C[C@@H]1CC(F)(F)CCN1C(=O)CNC(=O)c1ccnc2ccc(/C=C/c3ccc(Cl)cc3)cc12] |
| 94 | 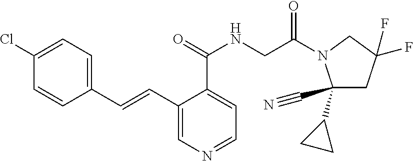 [SMILES: N#C[C@]1(C2CC2)CC(F)(F)CN1C(=O)CNC(=O)c1ccncc1/C=C/c1ccc(Cl)cc1] |
| 95 | 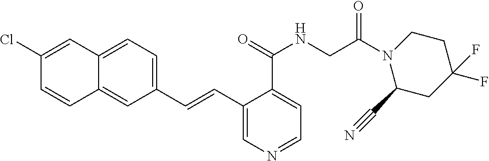 [SMILES: N#C[C@@H]1CC(F)(F)CCN1C(=O)CNC(=O)c1ccncc1/C=C/c1ccc2cc(Cl)ccc2c1] |
| 96 | 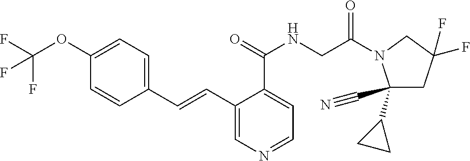 [SMILES: N#C[C@]1(C2CC2)CC(F)(F)CN1C(=O)CNC(=O)c1ccncc1/C=C/c1ccc(OC(F)(F)F)cc1] |
| 97 | 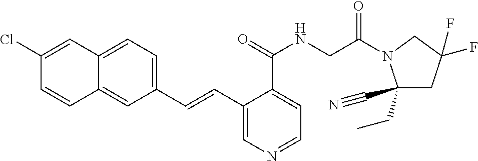 [SMILES: CC[C@@]1(C#N)CC(F)(F)CN1C(=O)CNC(=O)c1ccncc1/C=C/c1ccc2cc(Cl)ccc2c1] |
| 98 | 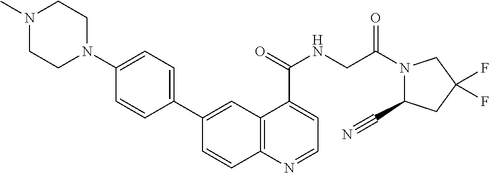 [SMILES: CN1CCN(c2ccc(-c3ccc4nccc(C(=O)NCC(=O)N5CC(F)(F)C[C@H]5C#N)c4c3)cc2)CC1] |
| 99 | 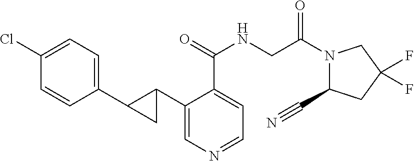 [SMILES: N#C[C@@H]1CC(F)(F)CN1C(=O)CNC(=O)c1ccncc1C1CC1c1ccc(Cl)cc1] |
| 100 | 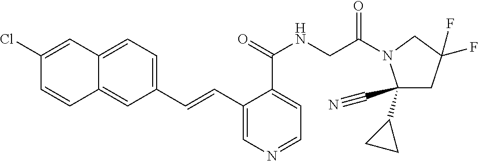 [SMILES: N#C[C@]1(C2CC2)CC(F)(F)CN1C(=O)CNC(=O)c1ccncc1/C=C/c1ccc2cc(Cl)ccc2c1] |
| 101 | 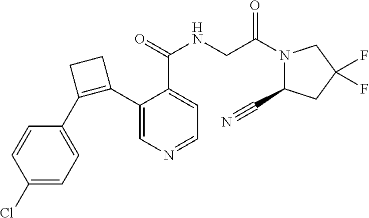 [SMILES: N#C[C@@H]1CC(F)(F)CN1C(=O)CNC(=O)c1ccncc1C1=C(c2ccc(Cl)cc2)CC1] |
| 102 | 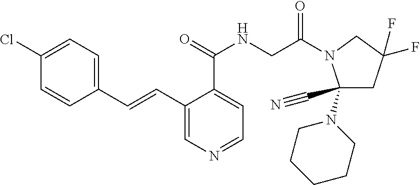 [SMILES: N#C[C@]1(N2CCCCC2)CC(F)(F)CN1C(=O)CNC(=O)c1ccncc1/C=C/c1ccc(Cl)cc1] |
| 103 | 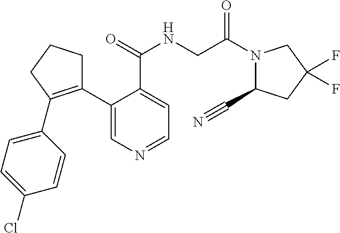 [SMILES: N#C[C@@H]1CC(F)(F)CN1C(=O)CNC(=O)c1ccncc1C1=C(c2ccc(Cl)cc2)CCC1] |
| 104 | 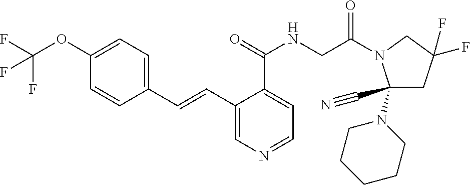 [SMILES: N#C[C@]1(N2CCCCC2)CC(F)(F)CN1C(=O)CNC(=O)c1ccncc1/C=C/c1ccc(OC(F)(F)F)cc1] |
| 105 | 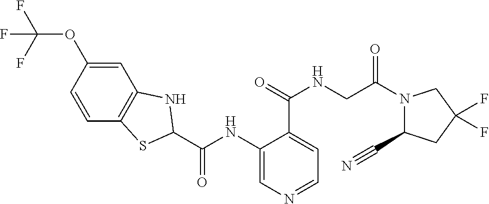 [SMILES: N#C[C@@H]1CC(F)(F)CN1C(=O)CNC(=O)c1ccncc1NC(=O)C1Nc2cc(OC(F)(F)F)ccc2S1] |
| 106 | 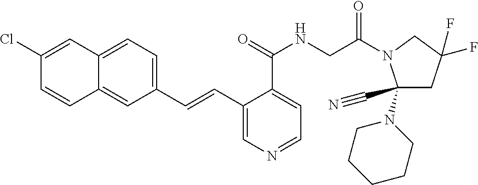 [SMILES: N#C[C@]1(N2CCCCC2)CC(F)(F)CN1C(=O)CNC(=O)c1ccncc1/C=C/c1ccc2cc(Cl)ccc2c1] |
| 107 | 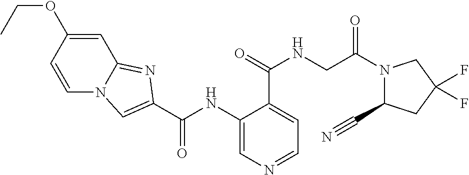 [SMILES: CCOc1ccn2cc(C(=O)Nc3cnccc3C(=O)NCC(=O)N3CC(F)(F)C[C@H]3C#N)nc2c1] |
| 108 | 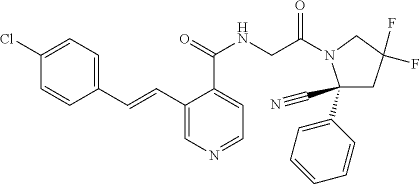 [SMILES: N#C[C@]1(c2ccccc2)CC(F)(F)CN1C(=O)CNC(=O)c1ccncc1/C=C/c1ccc(Cl)cc1] |
| 109 | 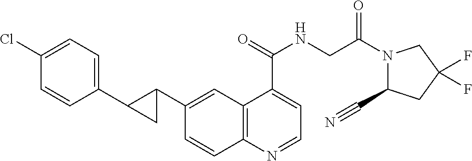 [SMILES: N#C[C@@H]1CC(F)(F)CN1C(=O)CNC(=O)c1ccnc2ccc(C3CC3c3ccc(Cl)cc3)cc12] |
| 110 | 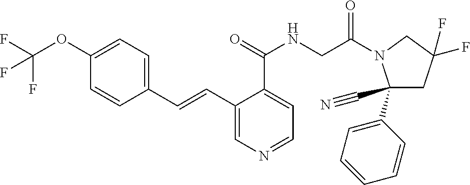 [SMILES: N#C[C@]1(c2ccccc2)CC(F)(F)CN1C(=O)CNC(=O)c1ccncc1/C=C/c1ccc(OC(F)(F)F)cc1] |
| 111 | 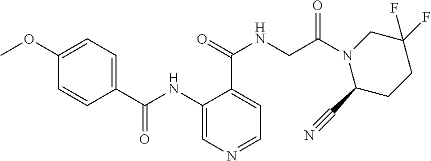 [SMILES: COc1ccc(C(=O)Nc2cnccc2C(=O)NCC(=O)N2CC(F)(F)CC[C@H]2C#N)cc1] |
| 112 | 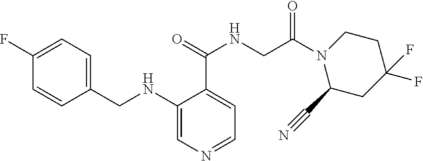 [SMILES: N#C[C@@H]1CC(F)(F)CCN1C(=O)CNC(=O)c1ccncc1NCc1ccc(F)cc1] |
| 113 | 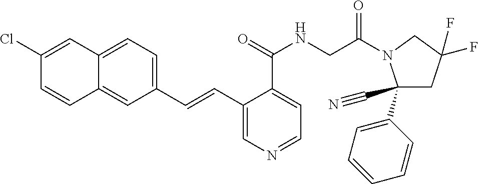 [SMILES: N#C[C@]1(c2ccccc2)CC(F)(F)CN1C(=O)CNC(=O)c1ccncc1/C=C/c1ccc2cc(Cl)ccc2c1] |
| 114 | 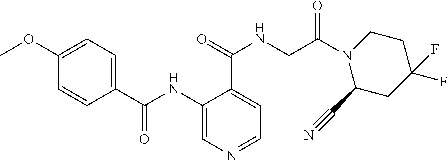 [SMILES: COc1ccc(C(=O)Nc2cnccc2C(=O)NCC(=O)N2CCC(F)(F)C[C@H]2C#N)cc1] |
| 115 | 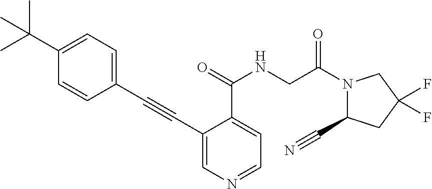 [SMILES: CC(C)(C)c1ccc(C#Cc2cnccc2C(=O)NCC(=O)N2CC(F)(F)C[C@H]2C#N)cc1] |
| 116 | 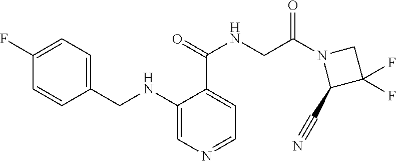 [SMILES: N#C[C@H]1N(C(=O)CNC(=O)c2ccncc2NCc2ccc(F)cc2)CC1(F)F] |
| 117 | 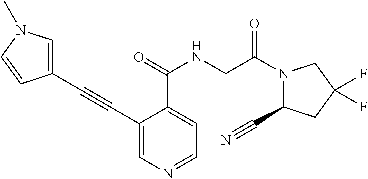 [SMILES: Cn1ccc(C#Cc2cnccc2C(=O)NCC(=O)N2CC(F)(F)C[C@H]2C#N)c1] |
| 118 | 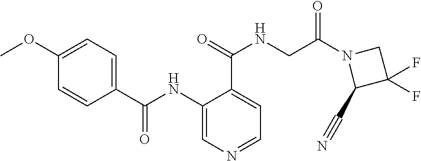 [SMILES: COc1ccc(C(=O)Nc2cnccc2C(=O)NCC(=O)N2CC(F)(F)[C@H]2C#N)cc1] |
| 119 | 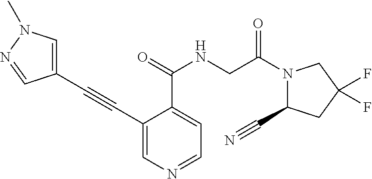 [SMILES: Cn1cc(C#Cc2cnccc2C(=O)NCC(=O)N2CC(F)(F)C[C@H]2C#N)cn1] |
| 120 | 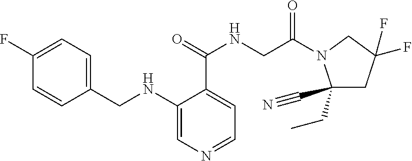 [SMILES: CC[C@@]1(C#N)CC(F)(F)CN1C(=O)CNC(=O)c1ccncc1NCc1ccc(F)cc1] |
| 121 | 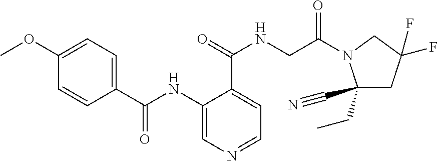 [SMILES: CC[C@@]1(C#N)CC(F)(F)CN1C(=O)CNC(=O)c1ccncc1NC(=O)c1ccc(OC)cc1] |
| 122 | 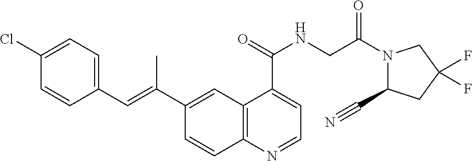 [SMILES: C/C(=C\c1ccc(Cl)cc1)c1ccc2nccc(C(=O)NCC(=O)N3CC(F)(F)C[C@H]3C#N)c2c1] |
| 123 | 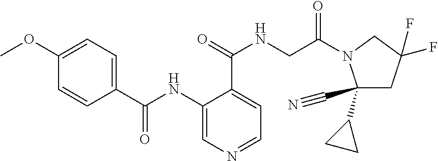 [SMILES: COc1ccc(C(=O)Nc2cnccc2C(=O)NCC(=O)N2CC(F)(F)C[C@]2(C#N)C2CC2)cc1] |
| 124 | 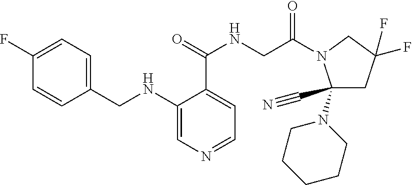 [SMILES: N#C[C@]1(N2CCCCC2)CC(F)(F)CN1C(=O)CNC(=O)c1ccncc1NCc1ccc(F)cc1] |
| 125 | 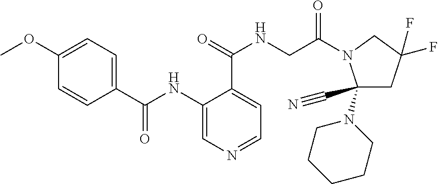 [SMILES: COc1ccc(C(=O)Nc2cnccc2C(=O)NCC(=O)N2CC(F)(F)C[C@]2(C#N)N2CCCCC2)cc1] |
| 126 | 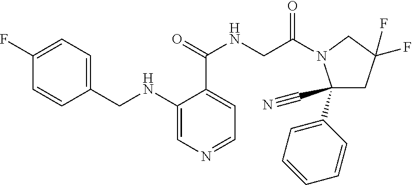 [SMILES: N#C[C@]1(c2ccccc2)CC(F)(F)CN1C(=O)CNC(=O)c1ccncc1NCc1ccc(F)cc1] |
| 127 | 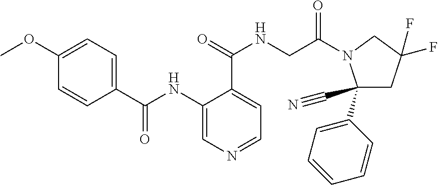 [SMILES: COc1ccc(C(=O)Nc2cnccc2C(=O)NCC(=O)N2CC(F)(F)C[C@]2(C#N)c2ccccc2)cc1] |
| 128 | 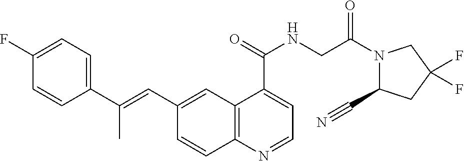 [SMILES: C/C(=C\c1ccc2nccc(C(=O)NCC(=O)N3CC(F)(F)C[C@H]3C#N)c2c1)c1ccc(F)cc1] |
| 129 | 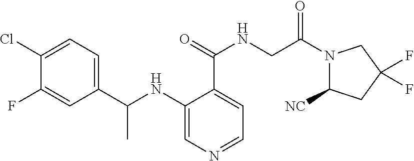 [SMILES: CC(Nc1cnccc1C(=O)NCC(=O)N1CC(F)(F)C[C@H]1C#N)c1ccc(Cl)c(F)c1] |
| 130 | 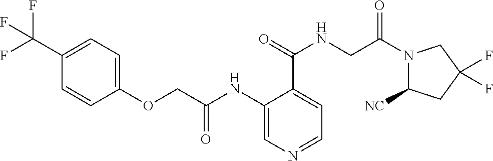 [SMILES: N#C[C@@H]1CC(F)(F)CN1C(=O)CNC(=O)c1ccncc1NC(=O)COc1ccc(C(F)(F)F)cc1] |
| 131 | 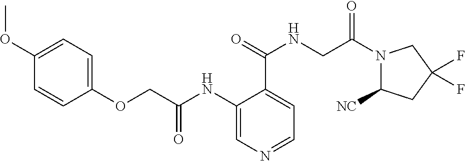 [SMILES: COc1ccc(OCC(=O)Nc2cnccc2C(=O)NCC(=O)N2CC(F)(F)C[C@H]2C#N)cc1] |
| 132 | 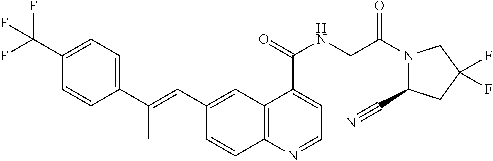 [SMILES: C/C(=C\c1ccc2nccc(C(=O)NCC(=O)N3CC(F)(F)C[C@H]3C#N)c2c1)c1ccc(C(F)(F)F)cc1] |
| 133 | 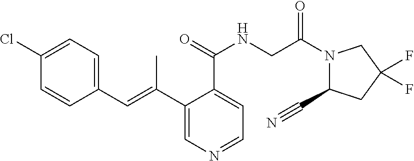 [SMILES: C/C(=C\c1ccc(Cl)cc1)c1cnccc1C(=O)NCC(=O)N1CC(F)(F)C[C@H]1C#N] |
| 134 | 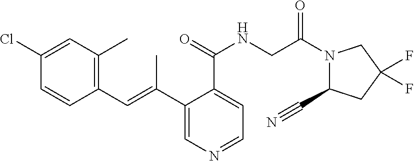 [SMILES: C/C(=C\c1ccc(Cl)cc1C)c1cnccc1C(=O)NCC(=O)N1CC(F)(F)C[C@H]1C#N] |
| 135 | 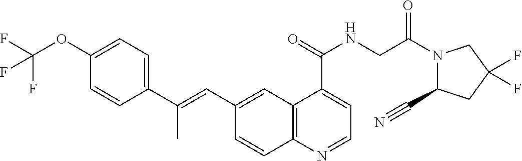 [SMILES: C/C(=C\c1ccc2nccc(C(=O)NCC(=O)N3CC(F)(F)C[C@H]3C#N)c2c1)c1ccc(OC(F)(F)F)cc1] |
| 136 | 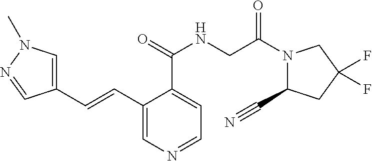 [SMILES: Cn1cc(/C=C/c2cnccc2C(=O)NCC(=O)N2CC(F)(F)C[C@H]2C#N)cn1] |
| 137 | 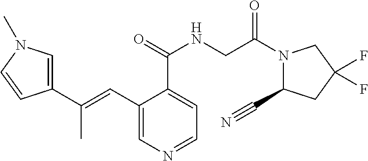 [SMILES: C/C(=C\c1cnccc1C(=O)NCC(=O)N1CC(F)(F)C[C@H]1C#N)c1ccn(C)c1] |
| 138 | 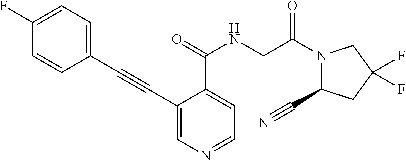 [SMILES: N#C[C@@H]1CC(F)(F)CN1C(=O)CNC(=O)c1ccncc1C#Cc1ccc(F)cc1] |
| 139 | 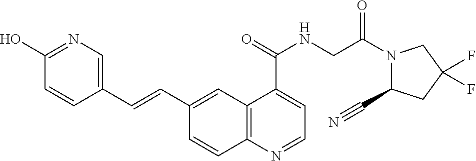 [SMILES: N#C[C@@H]1CC(F)(F)CN1C(=O)CNC(=O)c1ccnc2ccc(/C=C/c3ccc(O)nc3)cc12] |
| 140 | 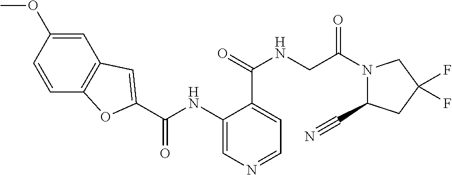 [SMILES: COc1ccc2oc(C(=O)Nc3cnccc3C(=O)NCC(=O)N3CC(F)(F)C[C@H]3C#N)cc2c1] |
| 141 | 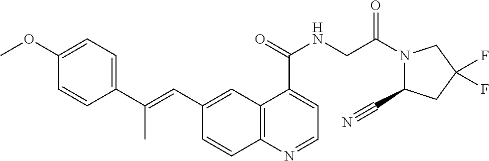 [SMILES: COc1ccc(/C(C)=C/c2ccc3nccc(C(=O)NCC(=O)N4CC(F)(F)C[C@H]4C#N)c3c2)cc1] |
| 142 | 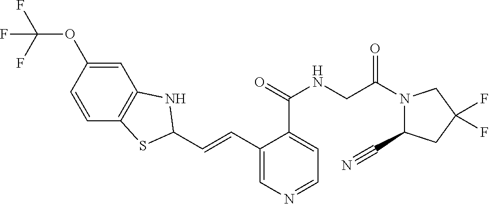 [SMILES: N#C[C@@H]1CC(F)(F)CN1C(=O)CNC(=O)c1ccncc1/C=C/C1Nc2cc(OC(F)(F)F)ccc2S1] |
| 143 | 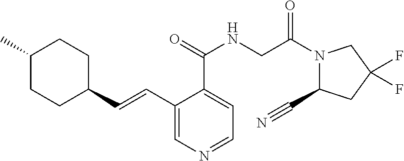 [SMILES: C[C@H]1CC[C@H](/C=C/c2cnccc2C(=O)NCC(=O)N2CC(F)(F)C[C@H]2C#N)CC1] |
| 144 | 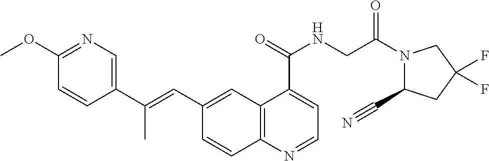 [SMILES: COc1ccc(/C(C)=C/c2ccc3nccc(C(=O)NCC(=O)N4CC(F)(F)C[C@H]4C#N)c3c2)cn1] |
| 145 | 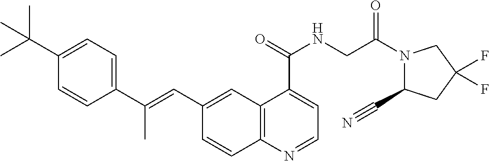 [SMILES: C/C(=C\c1ccc2nccc(C(=O)NCC(=O)N3CC(F)(F)C[C@H]3C#N)c2c1)c1ccc(C(C)(C)C)cc1] |
| 146 | 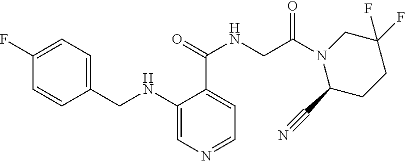 [SMILES: N#C[C@@H]1CCC(F)(F)CN1C(=O)CNC(=O)c1ccncc1NCc1ccc(F)cc1] |
| 147 | 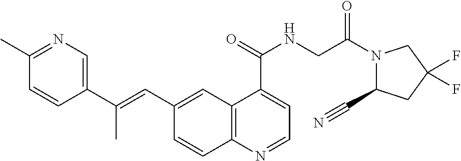 [SMILES: C/C(=C\c1ccc2nccc(C(=O)NCC(=O)N3CC(F)(F)C[C@H]3C#N)c2c1)c1ccc(C)nc1] |
| 148 | 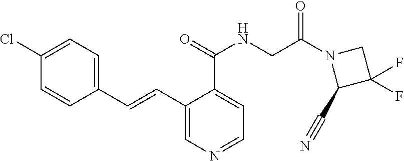 [SMILES: N#C[C@H]1N(C(=O)CNC(=O)c2ccncc2/C=C/c2ccc(Cl)cc2)CC1(F)F] |
| 149 | 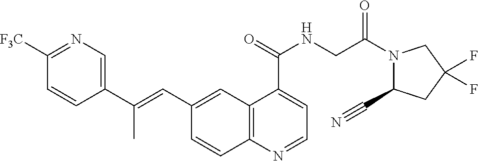 [SMILES: C/C(=C\c1ccc2nccc(C(=O)NCC(=O)N3CC(F)(F)C[C@H]3C#N)c2c1)c1ccc(C(F)(F)F)nc1] |
| 150 | 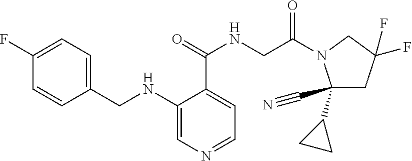 [SMILES: N#C[C@]1(C2CC2)CC(F)(F)CN1C(=O)CNC(=O)c1ccncc1NCc1ccc(F)cc1] |
| 151 | 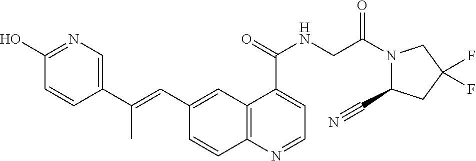 [SMILES: C/C(=C\c1ccc2nccc(C(=O)NCC(=O)N3CC(F)(F)C[C@H]3C#N)c2c1)c1ccc(O)nc1] |
| 152 | 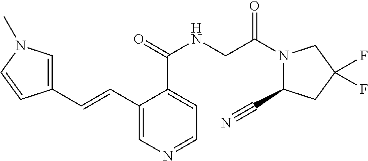 [SMILES: Cn1ccc(/C=C/c2cnccc2C(=O)NCC(=O)N2CC(F)(F)C[C@H]2C#N)c1] |
| 153 | 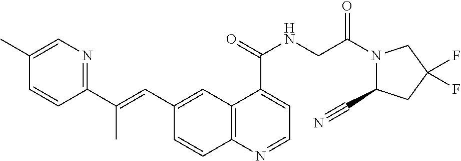 [SMILES: C/C(=C\c1ccc2nccc(C(=O)NCC(=O)N3CC(F)(F)C[C@H]3C#N)c2c1)c1ccc(C)cn1] |
| 154 | 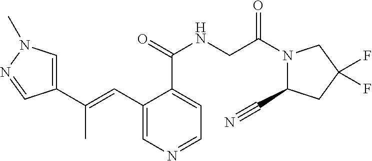 [SMILES: C/C(=C\c1cnccc1C(=O)NCC(=O)N1CC(F)(F)C[C@H]1C#N)c1cnn(C)c1] |
| 155 | 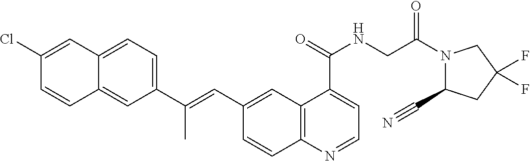 [SMILES: C/C(=C\c1ccc2nccc(C(=O)NCC(=O)N3CC(F)(F)C[C@H]3C#N)c2c1)c1ccc2cc(Cl)ccc2c1] |
| 156 | 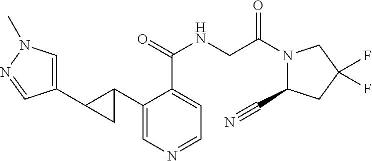 [SMILES: Cn1cc(C2CC2c2cnccc2C(=O)NCC(=O)N2CC(F)(F)C[C@H]2C#N)cn1] |
| 157 | 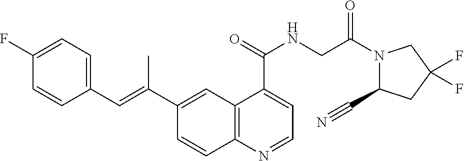 [SMILES: C/C(=C\c1ccc(F)cc1)c1ccc2nccc(C(=O)NCC(=O)N3CC(F)(F)C[C@H]3C#N)c2c1] |
| 158 | 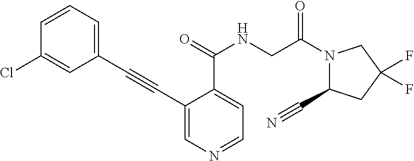 [SMILES: N#C[C@@H]1CC(F)(F)CN1C(=O)CNC(=O)c1ccncc1C#Cc1cccc(Cl)c1] |
| 159 | 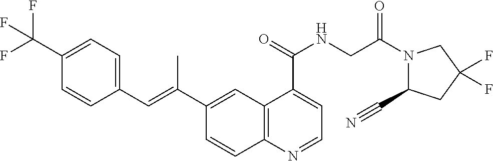 [SMILES: C/C(=C\c1ccc(C(F)(F)F)cc1)c1ccc2nccc(C(=O)NCC(=O)N3CC(F)(F)C[C@H]3C#N)c2c1] |
| 160 | 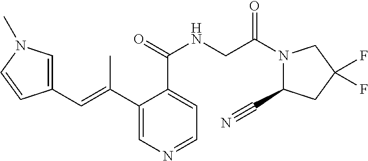 [SMILES: C/C(=C\c1ccn(C)c1)c1cnccc1C(=O)NCC(=O)N1CC(F)(F)C[C@H]1C#N] |
| 161 | 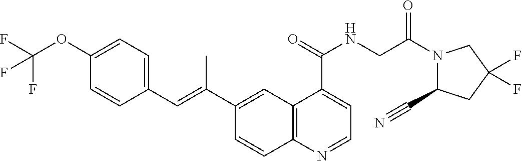 [SMILES: C/C(=C\c1ccc(OC(F)(F)F)cc1)c1ccc2nccc(C(=O)NCC(=O)N3CC(F)(F)C[C@H]3C#N)c2c1] |
| 162 | 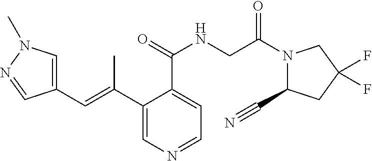 [SMILES: C/C(=C\c1cnn(C)c1)c1cnccc1C(=O)NCC(=O)N1CC(F)(F)C[C@H]1C#N] |
| 163 | 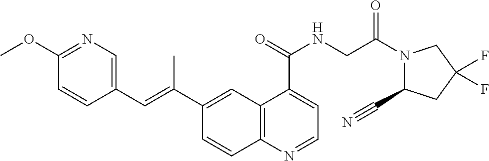 [SMILES: COc1ccc(/C=C(\C)c2ccc3nccc(C(=O)NCC(=O)N4CC(F)(F)C[C@H]4C#N)c3c2)cn1] |
| 164 | 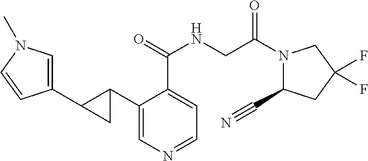 [SMILES: Cn1ccc(C2CC2c2cnccc2C(=O)NCC(=O)N2CC(F)(F)C[C@H]2C#N)c1] |
| 165 | 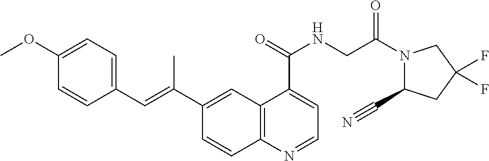 [SMILES: COc1ccc(/C=C(\C)c2ccc3nccc(C(=O)NCC(=O)N4CC(F)(F)C[C@H]4C#N)c3c2)cc1] |
| 166 | 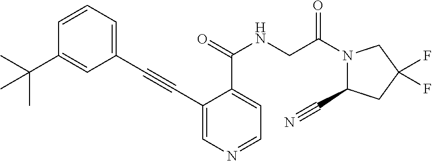 [SMILES: CC(C)(C)c1cccc(C#Cc2cnccc2C(=O)NCC(=O)N2CC(F)(F)C[C@H]2C#N)c1] |
| 167 | 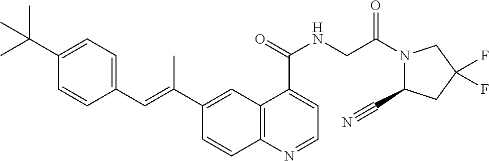 [SMILES: C/C(=C\c1ccc(C(C)(C)C)cc1)c1ccc2nccc(C(=O)NCC(=O)N3CC(F)(F)C[C@H]3C#N)c2c1] |
| 168 | 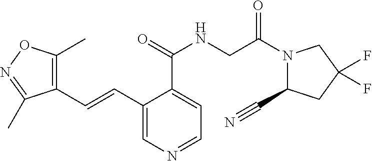 [SMILES: Cc1noc(C)c1/C=C/c1cnccc1C(=O)NCC(=O)N1CC(F)(F)C[C@H]1C#N] |
| 169 | 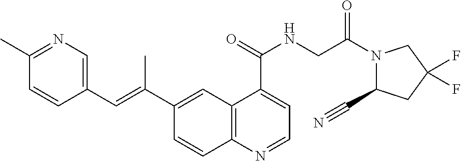 [SMILES: C/C(=C\c1ccc(C)nc1)c1ccc2nccc(C(=O)NCC(=O)N3CC(F)(F)C[C@H]3C#N)c2c1] |
| 170 | 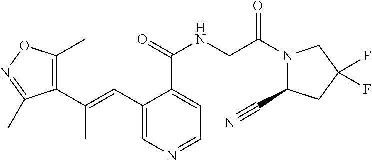 [SMILES: C/C(=C\c1cnccc1C(=O)NCC(=O)N1CC(F)(F)C[C@H]1C#N)c1c(C)noc1C] |
| 171 | 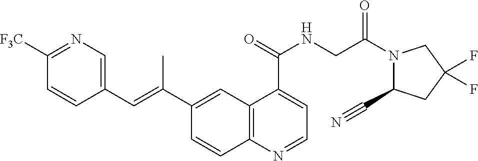 [SMILES: C/C(=C\c1ccc(C(F)(F)F)nc1)c1ccc2nccc(C(=O)NCC(=O)N3CC(F)(F)C[C@H]3C#N)c2c1] |
| 172 | 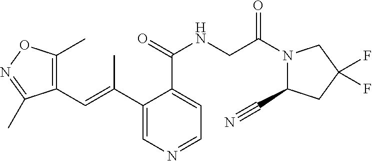 [SMILES: C/C(=C\c1c(C)noc1C)c1cnccc1C(=O)NCC(=O)N1CC(F)(F)C[C@H]1C#N] |
| 173 | 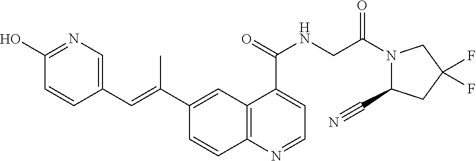 [SMILES: C/C(=C\c1ccc(O)nc1)c1ccc2nccc(C(=O)NCC(=O)N3CC(F)(F)C[C@H]3C#N)c2c1] |
| 174 | 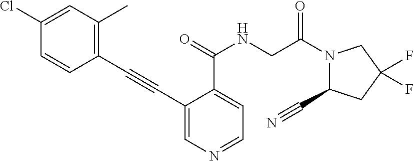 [SMILES: Cc1cc(Cl)ccc1C#Cc1cnccc1C(=O)NCC(=O)N1CC(F)(F)C[C@H]1C#N] |
| 175 | 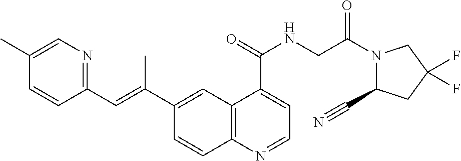 [SMILES: C/C(=C\c1ccc(C)cn1)c1ccc2nccc(C(=O)NCC(=O)N3CC(F)(F)C[C@H]3C#N)c2c1] |
| 176 | 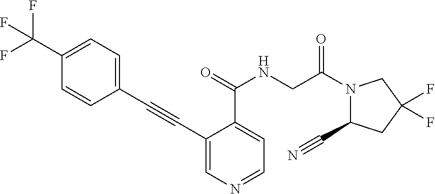 [SMILES: N#C[C@@H]1CC(F)(F)CN1C(=O)CNC(=O)c1ccncc1C#Cc1ccc(C(F)(F)F)cc1] |
| 177 | 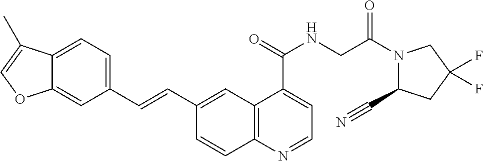 [SMILES: Cc1coc2cc(/C=C/c3ccc4nccc(C(=O)NCC(=O)N5CC(F)(F)C[C@H]5C#N)c4c3)ccc12] |
| 178 | 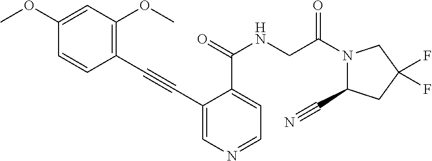 [SMILES: COc1ccc(C#Cc2cnccc2C(=O)NCC(=O)N2CC(F)(F)C[C@H]2C#N)c(OC)c1] |
| 179 | 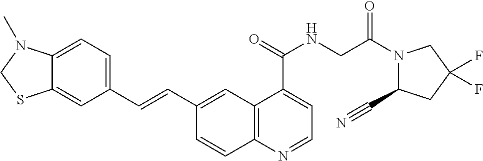 [SMILES: CN1CSc2cc(/C=C/c3ccc4nccc(C(=O)NCC(=O)N5CC(F)(F)C[C@H]5C#N)c4c3)ccc21] |
| 180 | 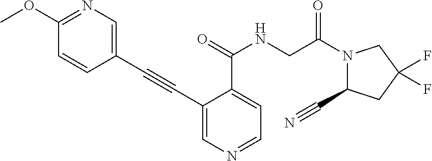 [SMILES: COc1ccc(C#Cc2cnccc2C(=O)NCC(=O)N2CC(F)(F)C[C@H]2C#N)cn1] |
| 181 | 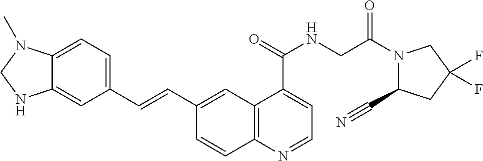 [SMILES: CN1CNc2cc(/C=C/c3ccc4nccc(C(=O)NCC(=O)N5CC(F)(F)C[C@H]5C#N)c4c3)ccc21] |
| 182 | 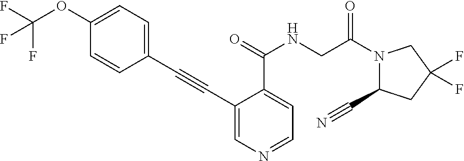 [SMILES: N#C[C@@H]1CC(F)(F)CN1C(=O)CNC(=O)c1ccncc1C#Cc1ccc(OC(F)(F)F)cc1] |
| 183 | 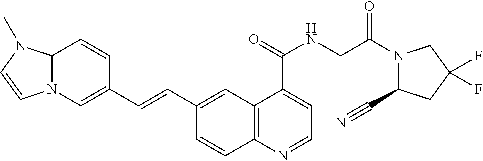 [SMILES: CN1C=CN2C=C(/C=C/c3ccc4nccc(C(=O)NCC(=O)N5CC(F)(F)C[C@H]5C#N)c4c3)C=CC12] |
| 184 | 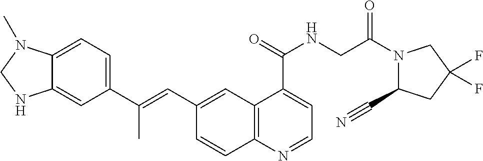 [SMILES: C/C(=C\c1ccc2nccc(C(=O)NCC(=O)N3CC(F)(F)C[C@H]3C#N)c2c1)c1ccc2c(c1)NCN2C] |
| 185 | 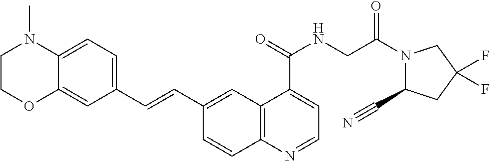 [SMILES: CN1CCOc2cc(/C=C/c3ccc4nccc(C(=O)NCC(=O)N5CC(F)(F)C[C@H]5C#N)c4c3)ccc21] |
| 186 | 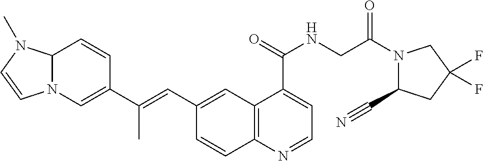 [SMILES: C/C(=C\c1ccc2nccc(C(=O)NCC(=O)N3CC(F)(F)C[C@H]3C#N)c2c1)C1=CN2C=CN(C)C2C=C1] |
| 187 | 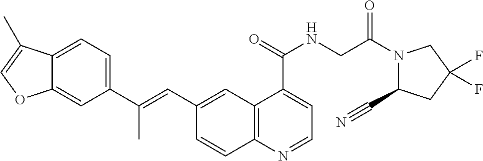 [SMILES: C/C(=C\c1ccc2nccc(C(=O)NCC(=O)N3CC(F)(F)C[C@H]3C#N)c2c1)c1ccc2c(C)coc2c1] |
| 188 | 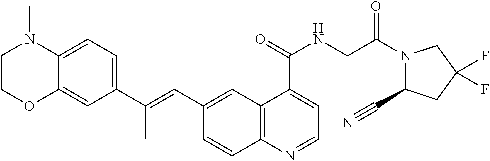 [SMILES: C/C(=C\c1ccc2nccc(C(=O)NCC(=O)N3CC(F)(F)C[C@H]3C#N)c2c1)c1ccc2c(c1)OCCN2C] |
| 189 | 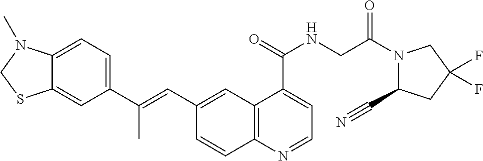 [SMILES: C/C(=C\c1ccc2nccc(C(=O)NCC(=O)N3CC(F)(F)C[C@H]3C#N)c2c1)c1ccc2c(c1)SCN2C] |
| 190 | 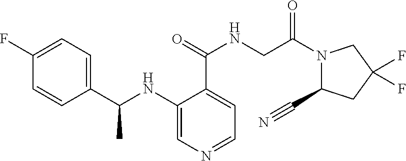 [SMILES: C[C@H](Nc1cnccc1C(=O)NCC(=O)N1CC(F)(F)C[C@H]1C#N)c1ccc(F)cc1] |
| 191 | 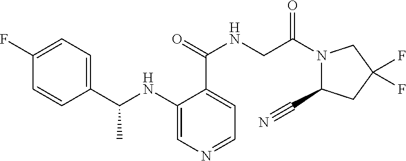 [SMILES: C[C@@H](Nc1cnccc1C(=O)NCC(=O)N1CC(F)(F)C[C@H]1C#N)c1ccc(F)cc1] |
| 192 | 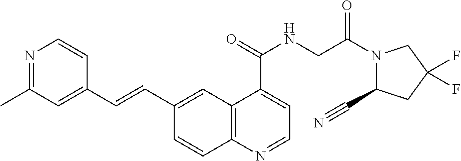 [SMILES: Cc1cc(/C=C/c2ccc3nccc(C(=O)NCC(=O)N4CC(F)(F)C[C@H]4C#N)c3c2)ccn1] |
| 193 | 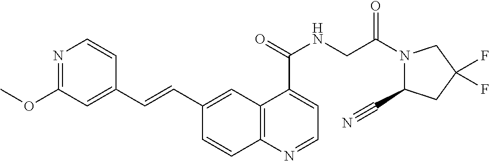 [SMILES: COc1cc(/C=C/c2ccc3nccc(C(=O)NCC(=O)N4CC(F)(F)C[C@H]4C#N)c3c2)ccn1] |
| 194 | 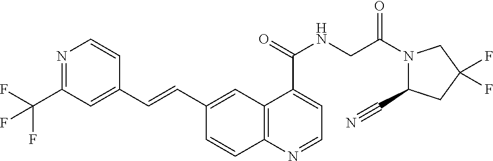 [SMILES: N#C[C@@H]1CC(F)(F)CN1C(=O)CNC(=O)c1ccnc2ccc(/C=C/c3ccnc(C(F)(F)F)c3)cc12] |
| 195 | 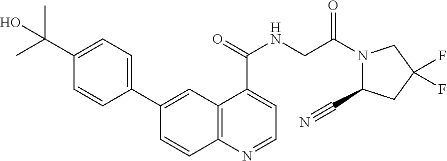 [SMILES: CC(C)(O)c1ccc(-c2ccc3nccc(C(=O)NCC(=O)N4CC(F)(F)C[C@H]4C#N)c3c2)cc1] |
| 196 | 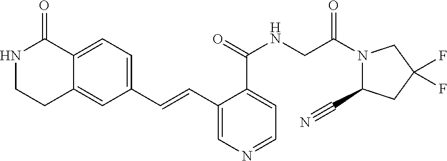 [SMILES: N#C[C@@H]1CC(F)(F)CN1C(=O)CNC(=O)c1ccncc1/C=C/c1ccc2c(c1)CCNC2=O] |
| 197 | 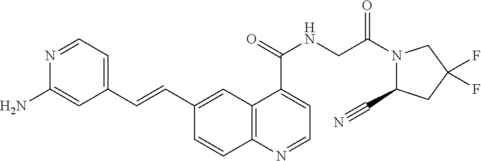 [SMILES: N#C[C@@H]1CC(F)(F)CN1C(=O)CNC(=O)c1ccnc2ccc(/C=C/c3ccnc(N)c3)cc12] |
| 198 | 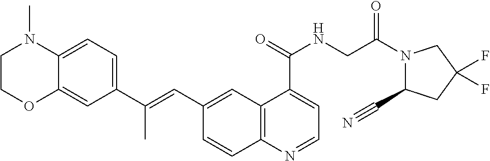 [SMILES: C/C(=C\c1ccc2nccc(C(=O)NCC(=O)N3CC(F)(F)C[C@H]3C#N)c2c1)c1ccc2c(c1)OCCN2C] |
| 199 | 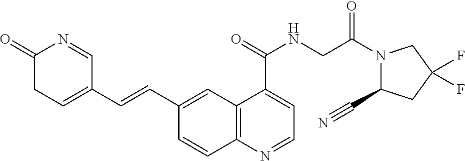 [SMILES: N#C[C@@H]1CC(F)(F)CN1C(=O)CNC(=O)c1ccnc2ccc(/C=C/C3=CCC(=O)N=C3)cc12] |
| 200 | 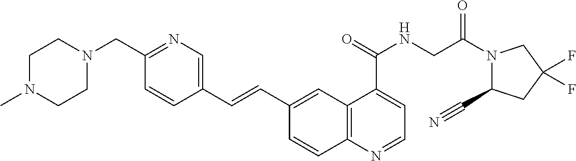 [SMILES: CN1CCN(Cc2ccc(/C=C/c3ccc4nccc(C(=O)NCC(=O)N5CC(F)(F)C[C@H]5C#N)c4c3)cn2)CC1] |
| 201 | 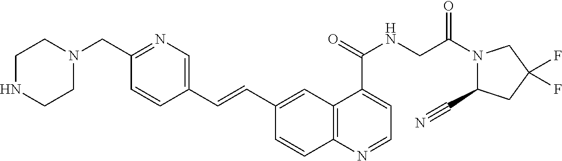 [SMILES: N#C[C@@H]1CC(F)(F)CN1C(=O)CNC(=O)c1ccnc2ccc(/C=C/c3ccc(CN4CCNCC4)nc3)cc12] |
| 202 | 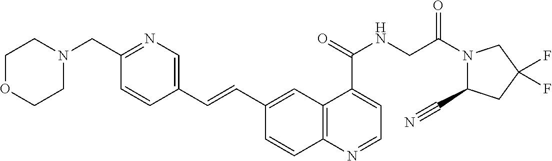 [SMILES: N#C[C@@H]1CC(F)(F)CN1C(=O)CNC(=O)c1ccnc2ccc(/C=C/c3ccc(CN4CCOCC4)nc3)cc12] |
| 203 | 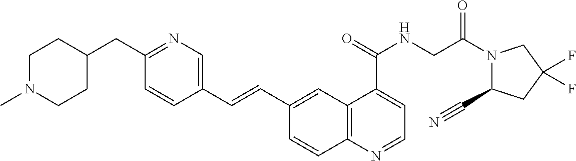 [SMILES: CN1CCC(Cc2ccc(/C=C/c3ccc4nccc(C(=O)NCC(=O)N5CC(F)(F)C[C@H]5C#N)c4c3)cn2)CC1] |
| 204 | 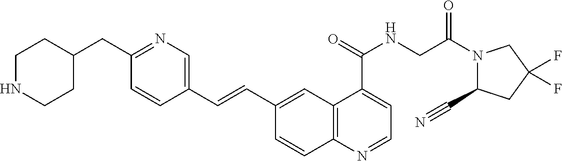 [SMILES: N#C[C@@H]1CC(F)(F)CN1C(=O)CNC(=O)c1ccnc2ccc(/C=C/c3ccc(CC4CCNCC4)nc3)cc12] |
| 205 | 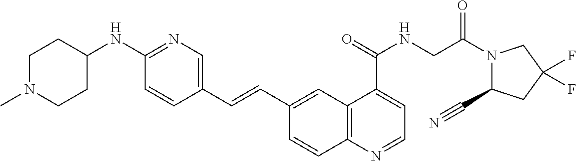 [SMILES: CN1CCC(Nc2ccc(/C=C/c3ccc4nccc(C(=O)NCC(=O)N5CC(F)(F)C[C@H]5C#N)c4c3)cn2)CC1] |
| 206 | 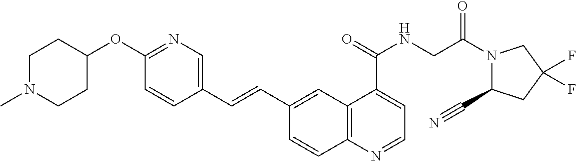 [SMILES: CN1CCC(Oc2ccc(/C=C/c3ccc4nccc(C(=O)NCC(=O)N5CC(F)(F)C[C@H]5C#N)c4c3)cn2)CC1] |
| 207 | 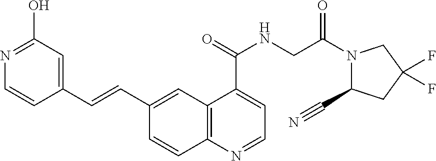 [SMILES: N#C[C@@H]1CC(F)(F)CN1C(=O)CNC(=O)c1ccnc2ccc(/C=C/c3ccnc(O)c3)cc12] |
| 208 | 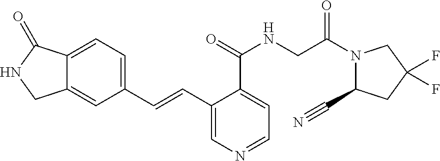 [SMILES: N#C[C@@H]1CC(F)(F)CN1C(=O)CNC(=O)c1ccncc1/C=C/c1ccc2c(c1)CNC2=O] |

Certain compounds depicted in Table 1 exist as tautomers. Regardless of which tautomer is shown, all tautomeric forms are intended.

In some embodiments, provided herein is a compound described in Table 1, or a tautomer thereof, or a salt of any of the foregoing, and uses thereof. In some embodiments, provided herein is a compound described in Table 1, or a pharmaceutically acceptable salt thereof. In some embodiments, provided herein is a compound described in Table 1, or a tautomer thereof, or a salt of any of the foregoing, and uses thereof. In some embodiments, provided herein is a compound described in Table 1, or a pharmaceutically acceptable salt thereof.

In some embodiments, provided is a compound selected from Compound Nos. 1-208 or a stereoisomer thereof (including a mixture of two or more stereoisomers thereof), or a salt thereof.

In some embodiments, the compound is a salt of a compound selected from Compound Nos. 1-208, or a stereoisomer thereof.

In one variation, the compound detailed herein is selected from the group consisting of:
N-(2-(2-cyano-4,4-difluoropyrrolidin-1-yl)-2-oxoethyl)-3-(2-phenylacetamido)isonicotinamide;
N-(2-(2-cyano-4,4-difluoropyrrolidin-1-yl)-2-oxoethyl)-3-((4-fluorobenzyl)amino)isonicotinamide;
N-(2-(2-cyano-4,4-difluoropyrrolidin-1-yl)-2-oxoethyl)-3-(4-methoxybenzamido)isonicotinamide;
N-(2-(2-cyano-4,4-difluoropyrrolidin-1-yl)-2-oxoethyl)-3-((1-(4-fluorophenyl)ethyl)amino)isonicotinamide;
3-(2-(4-chloro-3-fluorophenoxy)acetamido)-N-(2-(2-cyano-4,4-difluoropyrrolidin-1-yl)-2-oxoethyl)isonicotinamide;
2-(4-(bis(4-fluorophenyl)methyl)piperazin-1-yl)-N-(2-(2-cyano-4,4-difluoropyrrolidin-1-yl)-2-oxoethyl)isonicotinamide;
N-(2-(2-cyano-4,4-difluoropyrrolidin-1-yl)-2-oxoethyl)-3-(2-fluorobenzyl)isonicotinamide;
N-(2-(2-cyano-4,4-difluoropyrrolidin-1-yl)-2-oxoethyl)-3-(4-fluorobenzyl)isonicotinamide;
3-(4-chlorostyryl)-N-(2-(2-cyano-4,4-difluoropyrrolidin-1-yl)-2-oxoethyl)isonicotinamide;
3-(4-chlorophenethyl)-N-(2-(2-cyano-4,4-difluoropyrrolidin-1-yl)-2-oxoethyl)isonicotinamide;
N-(2-(2-cyano-4,4-difluoropyrrolidin-1-yl)-2-oxoethyl)-3-(4-fluorophenethyl)isonicotinamide;
N-(2-(2-cyano-4,4-difluoropyrrolidin-1-yl)-2-oxoethyl)-3-(4-fluorostyryl)isonicotinamide;
N-(2-(2-cyano-4,4-difluoropyrrolidin-1-yl)-2-oxoethyl)-3-(2-(4-fluorophenyl)prop-1-en-1-yl)isonicotinamide;
N-(2-(2-cyano-4,4-difluoropyrrolidin-1-yl)-2-oxoethyl)-3-(2-(4-fluorophenyl)allyl)isonicotinamide;
N-(2-(2-cyano-4,4-difluoropyrrolidin-1-yl)-2-oxoethyl)-3-(4-methoxystyryl)isonicotinamide;
N-(2-(2-cyano-4,4-difluoropyrrolidin-1-yl)-2-oxoethyl)-3-(4-(trifluoromethoxy)phenethyl)isonicotinamide;
N-(2-(2-cyano-4,4-difluoropyrrolidin-1-yl)-2-oxoethyl)-3-(4-(trifluoromethyl)phenethyl)isonicotinamide;
N-(2-(2-cyano-4,4-difluoropyrrolidin-1-yl)-2-oxoethyl)-3-(2-(4-fluorophenyl)propyl)isonicotinamide;
N-(2-(2-cyano-4,4-difluoropyrrolidin-1-yl)-2-oxoethyl)-3-(2-(4-(trifluoromethoxy)phenyl)prop-1-en-1-yl)isonicotinamide;
N-(2-(2-cyano-4,4-difluoropyrrolidin-1-yl)-2-oxoethyl)-3-(2-(4-(trifluoromethyl)phenyl)prop-1-en-1-yl)isonicotinamide;
N-(2-(2-cyano-4,4-difluoropyrrolidin-1-yl)-2-oxoethyl)-3-(2-(4-methoxyphenyl)allyl)isonicotinamide;
N-(2-(2-cyano-4,4-difluoropyrrolidin-1-yl)-2-oxoethyl)-3-(2-(4-(trifluoromethoxy)phenyl)propyl)isonicotinamide;
N-(2-(2-cyano-4,4-difluoropyrrolidin-1-yl)-2-oxoethyl)-3-(2-(4-(trifluoromethyl)phenyl)propyl)isonicotinamide;
N-(2-(2-cyano-4,4-difluoropyrrolidin-1-yl)-2-oxoethyl)-3-(4-(trifluoromethyl)styryl)isonicotinamide;
N-(2-(2-cyano-4,4-difluoropyrrolidin-1-yl)-2-oxoethyl)-3-(4-methoxyphenethyl)isonicotinamide;
3-(2-(4-chlorophenyl)prop-1-en-1-yl)-N-(2-(2-cyano-4,4-difluoropyrrolidin-1-yl)-2-oxoethyl)isonicotinamide;
3-(2-(4-chlorophenyl)allyl)-N-(2-(2-cyano-4,4-difluoropyrrolidin-1-yl)-2-oxoethyl)isonicotinamide;
3-(2-(4-chlorophenyl)propyl)-N-(2-(2-cyano-4,4-difluoropyrrolidin-1-yl)-2-oxoethyl)isonicotinamide;
N-(2-(2-cyano-4,4-difluoropyrrolidin-1-yl)-2-oxoethyl)-3-(2-(4-methoxyphenyl)prop-1-en-1-yl)isonicotinamide;
N-(2-(2-cyano-4,4-difluoropyrrolidin-1-yl)-2-oxoethyl)-3-(2-(4-(trifluoromethoxy)phenyl)allyl)isonicotinamide;
N-(2-(2-cyano-4,4-difluoropyrrolidin-1-yl)-2-oxoethyl)-3-(2-(4-(trifluoromethyl)phenyl)allyl)isonicotinamide;
N-(2-(2-cyano-4,4-difluoropyrrolidin-1-yl)-2-oxoethyl)-3-(2-(4-methoxyphenyl)propyl)isonicotinamide;
N-(2-(2-cyano-4,4-difluoropyrrolidin-1-yl)-2-oxoethyl)-3-(4-(trifluoromethoxy)styryl)isonicotinamide;
6-(4-chlorostyryl)-N-(2-(2-cyano-4,4-difluoropyrrolidin-1-yl)-2-oxoethyl)quinoline-4-carboxamide;
N-(2-(2-cyano-4,4-difluoropyrrolidin-1-yl)-2-oxoethyl)-6-(4-(trifluoromethyl)phenyl)quinoline-4-carboxamide;
N-(2-(2-cyano-4,4-difluoropyrrolidin-1-yl)-2-oxoethyl)-3-((4-methoxyphenyl)ethynyl)isonicotinamide;
3-((4-chlorophenyl)ethynyl)-N-(2-(2-cyano-4,4-difluoropyrrolidin-1-yl)-2-oxoethyl)isonicotinamide;
N-(2-(2-cyano-4,4-difluoropyrrolidin-1-yl)-2-oxoethyl)-6-(6-(trifluoromethyl)pyridin-3-yl)quinoline-4-carboxamide;
6-(2-(4-chlorophenyl)prop-1-en-1-yl)-N-(2-(2-cyano-4,4-difluoropyrrolidin-1-yl)-2-oxoethyl)quinoline-4-carboxamide;
3-(2-(6-chloronaphthalen-2-yl)vinyl)-N-(2-(2-cyano-4,4-difluoropyrrolidin-1-yl)-2-oxoethyl)isonicotinamide;
N-(2-(2-cyano-4,4-difluoropyrrolidin-1-yl)-2-oxoethyl)-6-((4-methoxyphenyl)ethynyl)quinoline-4-carb oxamide;
6-(2-(6-chloronaphthalen-2-yl)vinyl)-N-(2-(2-cyano-4,4-difluoropyrrolidin-1-yl)-2-oxoethyl)quinoline-4-carboxamide;
N-(2-(2-cyano-4,4-difluoropyrrolidin-1-yl)-2-oxoethyl)-6-(2-(6-methoxypyridin-3-yl)vinyl)quinoline-4-carb oxamide;
N-(2-(2-cyano-4,4-difluoropyrrolidin-1-yl)-2-oxoethyl)-6-(4-fluorostyryl)quinoline-4-carboxamide;
N-(2-(2-cyano-4,4-difluoropyrrolidin-1-yl)-2-oxoethyl)-6-(2-(6-methylpyridin-3-yl)vinyl)quinoline-4-carboxamide;
N-(2-(2-cyano-4,4-difluoropyrrolidin-1-yl)-2-oxoethyl)-6-(2-(6-(trifluoromethyl)pyridin-3-yl)vinyl)quinoline-4-carboxamide;
N-(2-(2-cyano-4,4-difluoropyrrolidin-1-yl)-2-oxoethyl)-6-(4-methoxystyryl)quinoline-4-carboxamide;
N-(2-(2-cyano-4,4-difluoropyrrolidin-1-yl)-2-oxoethyl)-6-(4-(trifluoromethoxy)styryl)quinoline-4-carboxamide;
N-(2-(2-cyano-4,4-difluoropyrrolidin-1-yl)-2-oxoethyl)-6-(2-(5-methylpyridin-2-yl)vinyl)quinoline-4-carboxamide;
6-(4-(tert-butyl)styryl)-N-(2-(2-cyano-4,4-difluoropyrrolidin-1-yl)-2-oxoethyl)quinoline-4-carboxamide;
N-(2-(2-cyano-4,4-difluoropyrrolidin-1-yl)-2-oxoethyl)-6-(4-(trifluoromethyl)styryl)quinoline-4-carboxamide;
N-(2-(2-cyano-4,4-difluoropyrrolidin-1-yl)-2-oxoethyl)-6-(6-(4-methylpiperazin-1-yl)pyridin-3-yl)quinoline-4-carboxamide;
N-(2-(2-cyano-4,4-difluoropyrrolidin-1-yl)-2-oxoethyl)-3-(2-(1-methyl-1H-indol-3-yl)vinyl)isonicotinamide;
N-(2-(2-cyano-4,4-difluoropyrrolidin-1-yl)-2-oxoethyl)-3-(2-(5-fluorobenzofuran-2-yl)vinyl)isonicotinamide;
N-(2-(2-cyano-4,4-difluoropyrrolidin-1-yl)-2-oxoethyl)-3-(2-(1-methyl-1H-indazol-3-yl)vinyl)isonicotinamide;
N-(2-(2-cyano-4,4-difluoropyrrolidin-1-yl)-2-oxoethyl)-3-(2-(6-fluorobenzofuran-2-yl)vinyl)isonicotinamide;
N-(2-(2-cyano-4,4-difluoropyrrolidin-1-yl)-2-oxoethyl)-3-(2-(1-methylpiperidin-4-yl)vinyl)isonicotinamide;
N-(2-(2-cyano-4,4-difluoropyrrolidin-1-yl)-2-oxoethyl)-3-(2-(5-methoxybenzofuran-2-yl)vinyl)isonicotinamide;
N-(2-(2-cyano-4,4-difluoropyrrolidin-1-yl)-2-oxoethyl)-3-(2-(4-fluorocyclohexyl)vinyl)isonicotinamide;
N-(2-(2-cyano-4,4-difluoropyrrolidin-1-yl)-2-oxoethyl)-3-(2-(5-methyl-2,3-dihydrobenzo[d]thiazol-2-yl)vinyl)isonicotinamide;
N-(2-(2-cyano-4,4-difluoropyrrolidin-1-yl)-2-oxoethyl)-3-(2-(4,4-difluorocyclohexyl)vinyl)isonicotinamide;
N-(2-(2-cyano-4,4-difluoropyrrolidin-1-yl)-2-oxoethyl)-3-(2-(6-methyl-2,3-dihydrobenzo[d]thiazol-2-yl)vinyl)isonicotinamide;
N-(2-(2-cyano-4,4-difluoropyrrolidin-1-yl)-2-oxoethyl)-3-(2-(4-methyl-3,4-dihydro-2H-benzo[b][1,4]oxazin-2-yl)vinyl)isonicotinamide;
3-(2-(5-(tert-butyl)-2,3-dihydrobenzo[d]thiazol-2-yl)vinyl)-N-(2-(2-cyano-4,4-difluoropyrrolidin-1-yl)-2-oxoethyl)isonicotinamide;
3-(2-(6-chloro-3,4-dihydro-2H-benzo[b][1,4]oxazin-2-yl)vinyl)-N-(2-(2-cyano-4,4-difluoropyrrolidin-1-yl)-2-oxoethyl)isonicotinamide;
3-(2-(7-chloroimidazo[1,2-a]pyridin-2-yl)vinyl)-N-(2-(2-cyano-4,4-difluoropyrrolidin-1-yl)-2-oxoethyl)isonicotinamide;
N-(2-(2-cyano-4,4-difluoropyrrolidin-1-yl)-2-oxoethyl)-3-(2-(6-fluoro-3,4-dihydro-2H-benzo[b][1,4]oxazin-2-yl)vinyl)isonicotinamide;
N-(2-(2-cyano-4,4-difluoropyrrolidin-1-yl)-2-oxoethyl)-3-(2-(7-ethoxyimidazo[1,2-a]pyridin-2-yl)vinyl)isonicotinamide;
N-(2-(2-cyano-4,4-difluoropyrrolidin-1-yl)-2-oxoethyl)-3-(2-(4-methyl-3,4-dihydro-2H-benzo[b][1,4]oxazin-7-yl)vinyl)isonicotinamide;
N-(2-(2-cyano-4,4-difluoropyrrolidin-1-yl)-2-oxoethyl)-3-(2-(6-fluoroimidazo[1,2-a]pyridin-2-yl)vinyl)isonicotinamide;
N-(2-(2-cyano-4,4-difluoropyrrolidin-1-yl)-2-oxoethyl)-3-(2-(1-methyl-1H-benzo[d]imidazol-5-yl)vinyl)isonicotinamide;
N-(2-(2-cyano-4,4-difluoropyrrolidin-1-yl)-2-oxoethyl)-6-(2-(1-methylpiperidin-4-yl)vinyl)quinoline-4-carb oxamide;
3-(2-(6-chloro-1H-benzo[d]imidazol-2-yl)vinyl)-N-(2-(2-cyano-4,4-difluoropyrrolidin-1-yl)-2-oxoethyl)isonicotinamide;
N-(2-(2-cyano-4,4-difluoropyrrolidin-1-yl)-2-oxoethyl)-6-(2-(4-methylpiperazin-1-yl)vinyl)quinoline-4-carb oxamide;
3-(2-(5-chloro-1H-benzo[d]imidazol-2-yl)vinyl)-N-(2-(2-cyano-4,4-difluoropyrrolidin-1-yl)-2-oxoethyl)isonicotinamide;
3-(4-chlorostyryl)-N-(2-(2-cyano-5,5-difluoropiperidin-1-yl)-2-oxoethyl)isonicotinamide;
N-(2-(2-cyano-4,4-difluoropyrrolidin-1-yl)-2-oxoethyl)-3-(2-(1-methyl-1H-benzo[d]imidazol-2-yl)vinyl)isonicotinamide;
N-(2-(2-cyano-5,5-difluoropiperidin-1-yl)-2-oxoethyl)-3-(4-(trifluoromethoxy)styryl)isonicotinamide;
N-(2-(2-cyano-4,4-difluoropyrrolidin-1-yl)-2-oxoethyl)-3-(2-(2-methyl-1,2,3,4-tetrahydroisoquinolin-6-yl)vinyl)isonicotinamide;
6-(4-chlorostyryl)-N-(2-(2-cyano-5,5-difluoropiperidin-1-yl)-2-oxoethyl)quinoline-4-carboxamide;
N-(2-(2-cyano-4,4-difluoropyrrolidin-1-yl)-2-oxoethyl)-3-(2-(1-methyl-1,2,3,4-tetrahydroquinolin-6-yl)vinyl)isonicotinamide;
3-(2-(6-chloronaphthalen-2-yl)vinyl)-N-(2-(2-cyano-5,5-difluoropiperidin-1-yl)-2-oxoethyl)isonicotinamide;
N-(2-(2-cyano-4,4-difluoropyrrolidin-1-yl)-2-oxoethyl)-3-(2-(4-hydroxycyclohexyl)vinyl)isonicotinamide;
N-(2-(2-cyano-3,3-difluoroazetidin-1-yl)-2-oxoethyl)-3-(4-(trifluoromethoxy)styryl)isonicotinamide;
N-(2-(2-cyano-4,4-difluoropyrrolidin-1-yl)-2-oxoethyl)-3-(2-(6-methylpiperidin-3-yl)vinyl)isonicotinamide;
N-(2-(2-cyano-4,4-difluoropyrrolidin-1-yl)-2-oxoethyl)-6-(2-(4-methylcyclohexyl)vinyl)quinoline-4-carboxamide;
3-(2-(6-chloronaphthalen-2-yl)vinyl)-N-(2-(2-cyano-3,3-difluoroazetidin-1-yl)-2-oxoethyl)isonicotinamide;
N-(2-(2-cyano-4,4-difluoropyrrolidin-1-yl)-2-oxoethyl)-6-(2-(4-hydroxycyclohexyl)vinyl)quinoline-4-carboxamide;
3-(4-chlorostyryl)-N-(2-(2-cyano-2-ethyl-4,4-difluoropyrrolidin-1-yl)-2-oxoethyl)isonicotinamide;
3-(4-chlorostyryl)-N-(2-(2-cyano-4,4-difluoropiperidin-1-yl)-2-oxoethyl)isonicotinamide;
N-(2-(2-cyano-2-ethyl-4,4-difluoropyrrolidin-1-yl)-2-oxoethyl)-3-(4-(trifluoromethoxy)styryl)isonicotinamide;
N-(2-(2-cyano-4,4-difluoropiperidin-1-yl)-2-oxoethyl)-3-(4-(trifluoromethoxy)styryl)isonicotinamide;
6-(4-chlorostyryl)-N-(2-(2-cyano-4,4-difluoropiperidin-1-yl)-2-oxoethyl)quinoline-4-carboxamide;
3-(4-chlorostyryl)-N-(2-(2-cyano-2-cyclopropyl-4,4-difluoropyrrolidin-1-yl)-2-oxoethyl)isonicotinamide;
3-(2-(6-chloronaphthalen-2-yl)vinyl)-N-(2-(2-cyano-4,4-difluoropiperidin-1-yl)-2-oxoethyl)isonicotinamide;
N-(2-(2-cyano-2-cyclopropyl-4,4-difluoropyrrolidin-1-yl)-2-oxoethyl)-3-(4-(trifluoromethoxy)styryl)isonicotinamide;
3-(2-(6-chloronaphthalen-2-yl)vinyl)-N-(2-(2-cyano-2-ethyl-4,4-difluoropyrrolidin-1-yl)-2-oxoethyl)isonicotinamide;
N-(2-(2-cyano-4,4-difluoropyrrolidin-1-yl)-2-oxoethyl)-6-(4-(4-methylpiperazin-1-yl)phenyl)quinoline-4-carboxamide;
3-(2-(4-chlorophenyl)cyclopropyl)-N-(2-(2-cyano-4,4-difluoropyrrolidin-1-yl)-2-oxoethyl)isonicotinamide;
3-(2-(6-chloronaphthalen-2-yl)vinyl)-N-(2-(2-cyano-2-cyclopropyl-4,4-difluoropyrrolidin-1-yl)-2-oxoethyl)isonicotinamide;
3-(2-(4-chlorophenyl)cyclobut-1-en-1-yl)-N-(2-(2-cyano-4,4-difluoropyrrolidin-1-yl)-2-oxoethyl)isonicotinamide;
3-(4-chlorostyryl)-N-(2-(2-cyano-4,4-difluoro-2-(piperidin-1-yl)pyrrolidin-1-yl)-2-oxoethyl)isonicotinamide;
3-(2-(4-chlorophenyl)cyclopent-1-en-1-yl)-N-(2-(2-cyano-4,4-difluoropyrrolidin-1-yl)-2-oxoethyl)isonicotinamide;
N-(2-(2-cyano-4,4-difluoro-2-(piperidin-1-yl)pyrrolidin-1-yl)-2-oxoethyl)-3-(4-(trifluoromethoxy)styryl)isonicotinamide;
N-(4-((2-(2-cyano-4,4-difluoropyrrolidin-1-yl)-2-oxoethyl)carbamoyl)pyridin-3-yl)-5-(trifluoromethoxy)-2,3-dihydrobenzo[d]thiazole-2-carboxamide;
3-(2-(6-chloronaphthalen-2-yl)vinyl)-N-(2-(2-cyano-4,4-difluoro-2-(piperidin-1-yl)pyrrolidin-1-yl)-2-oxoethyl)isonicotinamide;
N-(4-((2-(2-cyano-4,4-difluoropyrrolidin-1-yl)-2-oxoethyl)carbamoyl)pyridin-3-yl)-7-ethoxyimidazo[1,2-a]pyridine-2-carboxamide;
3-(4-chlorostyryl)-N-(2-(2-cyano-4,4-difluoro-2-phenylpyrrolidin-1-yl)-2-oxoethyl)isonicotinamide;
6-(2-(4-chlorophenyl)cyclopropyl)-N-(2-(2-cyano-4,4-difluoropyrrolidin-1-yl)-2-oxoethyl)quinoline-4-carboxamide;
N-(2-(2-cyano-4,4-difluoro-2-phenylpyrrolidin-1-yl)-2-oxoethyl)-3-(4-(trifluoromethoxy)styryl)isonicotinamide;
N-(2-(2-cyano-5,5-difluoropiperidin-1-yl)-2-oxoethyl)-3-(4-methoxybenzamido)isonicotinamide;
N-(2-(2-cyano-4,4-difluoropiperidin-1-yl)-2-oxoethyl)-3-((4-fluorobenzyl)amino)isonicotinamide;
3-(2-(6-chloronaphthalen-2-yl)vinyl)-N-(2-(2-cyano-4,4-difluoro-2-phenylpyrrolidin-1-yl)-2-oxoethyl)isonicotinamide;
N-(2-(2-cyano-4,4-difluoropiperidin-1-yl)-2-oxoethyl)-3-(4-methoxybenzamido)isonicotinamide;
3-((4-(tert-butyl)phenyl)ethynyl)-N-(2-(2-cyano-4,4-difluoropyrrolidin-1-yl)-2-oxoethyl)isonicotinamide;
N-(2-(2-cyano-3,3-difluoroazetidin-1-yl)-2-oxoethyl)-3-((4-fluorobenzyl)amino)isonicotinamide;
N-(2-(2-cyano-4,4-difluoropyrrolidin-1-yl)-2-oxoethyl)-3-((1-methyl-1H-pyrrol-3-yl)ethynyl)isonicotinamide;
N-(2-(2-cyano-3,3-difluoroazetidin-1-yl)-2-oxoethyl)-3-(4-methoxybenzamido)isonicotinamide;
N-(2-(2-cyano-4,4-difluoropyrrolidin-1-yl)-2-oxoethyl)-3-((1-methyl-1H-pyrazol-4-yl)ethynyl)isonicotinamide;
N-(2-(2-cyano-2-ethyl-4,4-difluoropyrrolidin-1-yl)-2-oxoethyl)-3-((4-fluorobenzyl)amino)isonicotinamide;
N-(2-(2-cyano-2-ethyl-4,4-difluoropyrrolidin-1-yl)-2-oxoethyl)-3-(4-methoxybenzamido)isonicotinamide;
6-(1-(4-chlorophenyl)prop-1-en-2-yl)-N-(2-(2-cyano-4,4-difluoropyrrolidin-1-yl)-2-oxoethyl)quinoline-4-carboxamide;
N-(2-(2-cyano-2-cyclopropyl-4,4-difluoropyrrolidin-1-yl)-2-oxoethyl)-3-(4-methoxybenzamido)isonicotinamide;
N-(2-(2-cyano-4,4-difluoro-2-(piperidin-1-yl)pyrrolidin-1-yl)-2-oxoethyl)-3-((4-fluorobenzyl)amino)isonicotinamide;
N-(2-(2-cyano-4,4-difluoro-2-(piperidin-1-yl)pyrrolidin-1-yl)-2-oxoethyl)-3-(4-methoxybenzamido)isonicotinamide;
N-(2-(2-cyano-4,4-difluoro-2-phenylpyrrolidin-1-yl)-2-oxoethyl)-3-((4-fluorobenzyl)amino)isonicotinamide;
N-(2-(2-cyano-4,4-difluoro-2-phenylpyrrolidin-1-yl)-2-oxoethyl)-3-(4-methoxybenzamido)isonicotinamide;
N-(2-(2-cyano-4,4-difluoropyrrolidin-1-yl)-2-oxoethyl)-6-(2-(4-fluorophenyl)prop-1-en-1-yl)quinoline-4-carboxamide;
3-((1-(4-chloro-3-fluorophenyl)ethyl)amino)-N-(2-(2-cyano-4,4-difluoropyrrolidin-1-yl)-2-oxoethyl)isonicotinamide;
N-(2-(2-cyano-4,4-difluoropyrrolidin-1-yl)-2-oxoethyl)-3-(2-(4-(trifluoromethyl)phenoxy)acetamido)isonicotinamide;
N-(2-(2-cyano-4,4-difluoropyrrolidin-1-yl)-2-oxoethyl)-3-(2-(4-methoxyphenoxy)acetamido)isonicotinamide;
N-(2-(2-cyano-4,4-difluoropyrrolidin-1-yl)-2-oxoethyl)-6-(2-(4-(trifluoromethyl)phenyl)prop-1-en-1-yl)quinoline-4-carboxamide;
3-(1-(4-chlorophenyl)prop-1-en-2-yl)-N-(2-(2-cyano-4,4-difluoropyrrolidin-1-yl)-2-oxoethyl)isonicotinamide;
3-(1-(4-chloro-2-methylphenyl)prop-1-en-2-yl)-N-(2-(2-cyano-4,4-difluoropyrrolidin-1-yl)-2-oxoethyl)isonicotinamide;
N-(2-(2-cyano-4,4-difluoropyrrolidin-1-yl)-2-oxoethyl)-6-(2-(4-(trifluoromethoxy)phenyl)prop-1-en-1-yl)quinoline-4-carb oxamide;
N-(2-(2-cyano-4,4-difluoropyrrolidin-1-yl)-2-oxoethyl)-3-(2-(1-methyl-1H-pyrazol-4-yl)vinyl)isonicotinamide;
N-(2-(2-cyano-4,4-difluoropyrrolidin-1-yl)-2-oxoethyl)-3-(2-(1-methyl-1H-pyrrol-3-yl)prop-1-en-1-yl)isonicotinamide;
N-(2-(2-cyano-4,4-difluoropyrrolidin-1-yl)-2-oxoethyl)-3-((4-fluorophenyl)ethynyl)isonicotinamide;
N-(2-(2-cyano-4,4-difluoropyrrolidin-1-yl)-2-oxoethyl)-6-(2-(6-hydroxypyridin-3-yl)vinyl)quinoline-4-carb oxamide;
N-(2-(2-cyano-4,4-difluoropyrrolidin-1-yl)-2-oxoethyl)-3-(5-methoxybenzofuran-2-carboxamido)isonicotinamide;
N-(2-(2-cyano-4,4-difluoropyrrolidin-1-yl)-2-oxoethyl)-6-(2-(4-methoxyphenyl)prop-1-en-1-yl)quinoline-4-carboxamide;
N-(2-(2-cyano-4,4-difluoropyrrolidin-1-yl)-2-oxoethyl)-3-(2-(5-(trifluoromethoxy)-2,3-dihydrobenzo[d]thiazol-2-yl)vinyl)isonicotinamide;
N-(2-(2-cyano-4,4-difluoropyrrolidin-1-yl)-2-oxoethyl)-3-(2-(4-methylcyclohexyl)vinyl)isonicotinamide;
N-(2-(2-cyano-4,4-difluoropyrrolidin-1-yl)-2-oxoethyl)-6-(2-(6-methoxypyridin-3-yl)prop-1-en-1-yl)quinoline-4-carboxamide;
6-(2-(4-(tert-butyl)phenyl)prop-1-en-1-yl)-N-(2-(2-cyano-4,4-difluoropyrrolidin-1-yl)-2-oxoethyl)quinoline-4-carboxamide;
N-(2-(2-cyano-5,5-difluoropiperidin-1-yl)-2-oxoethyl)-3-((4-fluorobenzyl)amino)isonicotinamide;
N-(2-(2-cyano-4,4-difluoropyrrolidin-1-yl)-2-oxoethyl)-6-(2-(6-methylpyridin-3-yl)prop-1-en-1-yl)quinoline-4-carboxamide;
3-(4-chlorostyryl)-N-(2-(2-cyano-3,3-difluoroazetidin-1-yl)-2-oxoethyl)isonicotinamide;
N-(2-(2-cyano-4,4-difluoropyrrolidin-1-yl)-2-oxoethyl)-6-(2-(6-(trifluoromethyl)pyridin-3-yl)prop-1-en-1-yl)quinoline-4-carboxamide;
N-(2-(2-cyano-2-cyclopropyl-4,4-difluoropyrrolidin-1-yl)-2-oxoethyl)-3-((4-fluorobenzyl)amino)isonicotinamide;
N-(2-(2-cyano-4,4-difluoropyrrolidin-1-yl)-2-oxoethyl)-6-(2-(6-hydroxypyridin-3-yl)prop-1-en-1-yl)quinoline-4-carb oxamide;
N-(2-(2-cyano-4,4-difluoropyrrolidin-1-yl)-2-oxoethyl)-3-(2-(1-methyl-1H-pyrrol-3-yl)vinyl)isonicotinamide;
N-(2-(2-cyano-4,4-difluoropyrrolidin-1-yl)-2-oxoethyl)-6-(2-(5-methylpyridin-2-yl)prop-1-en-1-yl)quinoline-4-carboxamide;
N-(2-(2-cyano-4,4-difluoropyrrolidin-1-yl)-2-oxoethyl)-3-(2-(1-methyl-1H-pyrazol-4-yl)prop-1-en-1-yl)isonicotinamide;
6-(2-(6-chloronaphthalen-2-yl)prop-1-en-1-yl)-N-(2-(2-cyano-4,4-difluoropyrrolidin-1-yl)-2-oxoethyl)quinoline-4-carboxamide;
N-(2-(2-cyano-4,4-difluoropyrrolidin-1-yl)-2-oxoethyl)-3-(2-(1-methyl-1H-pyrazol-4-yl)cyclopropyl)isonicotinamide;
N-(2-(2-cyano-4,4-difluoropyrrolidin-1-yl)-2-oxoethyl)-6-(1-(4-fluorophenyl)prop-1-en-2-yl)quinoline-4-carb oxamide;
3-((3-chlorophenyl)ethynyl)-N-(2-(2-cyano-4,4-difluoropyrrolidin-1-yl)-2-oxoethyl)isonicotinamide;
N-(2-(2-cyano-4,4-difluoropyrrolidin-1-yl)-2-oxoethyl)-6-(1-(4-(trifluoromethyl)phenyl)prop-1-en-2-yl)quinoline-4-carboxamide;
N-(2-(2-cyano-4,4-difluoropyrrolidin-1-yl)-2-oxoethyl)-3-(1-(1-methyl-1H-pyrrol-3-yl)prop-1-en-2-yl)isonicotinamide;
N-(2-(2-cyano-4,4-difluoropyrrolidin-1-yl)-2-oxoethyl)-6-(1-(4-(trifluoromethoxy)phenyl)prop-1-en-2-yl)quinoline-4-carb oxamide;
N-(2-(2-cyano-4,4-difluoropyrrolidin-1-yl)-2-oxoethyl)-3-(1-(1-methyl-1H-pyrazol-4-yl)prop-1-en-2-yl)isonicotinamide;
N-(2-(2-cyano-4,4-difluoropyrrolidin-1-yl)-2-oxoethyl)-6-(1-(6-methoxypyridin-3-yl)prop-1-en-2-yl)quinoline-4-carboxamide;
N-(2-(2-cyano-4,4-difluoropyrrolidin-1-yl)-2-oxoethyl)-3-(2-(1-methyl-1H-pyrrol-3-yl)cyclopropyl)isonicotinamide;
N-(2-(2-cyano-4,4-difluoropyrrolidin-1-yl)-2-oxoethyl)-6-(1-(4-methoxyphenyl)prop-1-en-2-yl)quinoline-4-carb oxamide;
3-((3-(tert-butyl)phenyl)ethynyl)-N-(2-(2-cyano-4,4-difluoropyrrolidin-1-yl)-2-oxoethyl)isonicotinamide;
6-(1-(4-(tert-butyl)phenyl)prop-1-en-2-yl)-N-(2-(2-cyano-4,4-difluoropyrrolidin-1-yl)-2-oxoethyl)quinoline-4-carboxamide;
N-(2-(2-cyano-4,4-difluoropyrrolidin-1-yl)-2-oxoethyl)-3-(2-(3,5-dimethylisoxazol-4-yl)vinyl)isonicotinamide;
N-(2-(2-cyano-4,4-difluoropyrrolidin-1-yl)-2-oxoethyl)-6-(1-(6-methylpyridin-3-yl)prop-1-en-2-yl)quinoline-4-carboxamide;
N-(2-(2-cyano-4,4-difluoropyrrolidin-1-yl)-2-oxoethyl)-3-(2-(3,5-dimethylisoxazol-4-yl)prop-1-en-1-yl)isonicotinamide;
N-(2-(2-cyano-4,4-difluoropyrrolidin-1-yl)-2-oxoethyl)-6-(1-(6-(trifluoromethyl)pyridin-3-yl)prop-1-en-2-yl)quinoline-4-carboxamide;
N-(2-(2-cyano-4,4-difluoropyrrolidin-1-yl)-2-oxoethyl)-3-(1-(3,5-dimethylisoxazol-4-yl)prop-1-en-2-yl)isonicotinamide;
N-(2-(2-cyano-4,4-difluoropyrrolidin-1-yl)-2-oxoethyl)-6-(1-(6-hydroxypyridin-3-yl)prop-1-en-2-yl)quinoline-4-carboxamide;
3-((4-chloro-2-methylphenyl)ethynyl)-N-(2-(2-cyano-4,4-difluoropyrrolidin-1-yl)-2-oxoethyl)isonicotinamide;
N-(2-(2-cyano-4,4-difluoropyrrolidin-1-yl)-2-oxoethyl)-6-(1-(5-methylpyridin-2-yl)prop-1-en-2-yl)quinoline-4-carboxamide;
N-(2-(2-cyano-4,4-difluoropyrrolidin-1-yl)-2-oxoethyl)-3-((4-(trifluoromethyl)phenyl)ethynyl)isonicotinamide;
N-(2-(2-cyano-4,4-difluoropyrrolidin-1-yl)-2-oxoethyl)-6-(2-(3-methylbenzofuran-6-yl)vinyl)quinoline-4-carboxamide;
N-(2-(2-cyano-4,4-difluoropyrrolidin-1-yl)-2-oxoethyl)-3-((2,4-dimethoxyphenyl)ethynyl)isonicotinamide;
N-(2-(2-cyano-4,4-difluoropyrrolidin-1-yl)-2-oxoethyl)-6-(2-(3-methyl-2,3-dihydrobenzo[d]thiazol-6-yl)vinyl)quinoline-4-carb oxamide;
N-(2-(2-cyano-4,4-difluoropyrrolidin-1-yl)-2-oxoethyl)-3-((6-methoxypyridin-3-yl)ethynyl)isonicotinamide;
N-(2-(2-cyano-4,4-difluoropyrrolidin-1-yl)-2-oxoethyl)-6-(2-(1-methyl-2,3-dihydro-1H-benzo[d]imidazol-5-yl)vinyl)quinoline-4-carboxamide;
N-(2-(2-cyano-4,4-difluoropyrrolidin-1-yl)-2-oxoethyl)-3-((4-(trifluoromethoxy)phenyl)ethynyl)isonicotinamide;
N-(2-(2-cyano-4,4-difluoropyrrolidin-1-yl)-2-oxoethyl)-6-(2-(1-methyl-1,8a-dihydroimidazo[1,2-a]pyridin-6-yl)vinyl)quinoline-4-carboxamide;
N-(2-(2-cyano-4,4-difluoropyrrolidin-1-yl)-2-oxoethyl)-6-(2-(1-methyl-2,3-dihydro-1H-benzo[d]imidazol-5-yl)prop-1-en-1-yl)quinoline-4-carboxamide;
N-(2-(2-cyano-4,4-difluoropyrrolidin-1-yl)-2-oxoethyl)-6-(2-(4-methyl-3,4-dihydro-2H-benzo[b][1,4]oxazin-7-yl)vinyl)quinoline-4-carboxamide;
N-(2-(2-cyano-4,4-difluoropyrrolidin-1-yl)-2-oxoethyl)-6-(2-(1-methyl-1,8a-dihydroimidazo[1,2-a]pyridin-6-yl)prop-1-en-1-yl)quinoline-4-carboxamide;
N-(2-(2-cyano-4,4-difluoropyrrolidin-1-yl)-2-oxoethyl)-6-(2-(3-methylbenzofuran-6-yl)prop-1-en-1-yl)quinoline-4-carboxamide;
N-(2-(2-cyano-4,4-difluoropyrrolidin-1-yl)-2-oxoethyl)-6-(2-(4-methyl-3,4-dihydro-2H-benzo[b][1,4]oxazin-7-yl)prop-1-en-1-yl)quinoline-4-carboxamide;
N-(2-(2-cyano-4,4-difluoropyrrolidin-1-yl)-2-oxoethyl)-6-(2-(3-methyl-2,3-dihydrobenzo[d]thiazol-6-yl)prop-1-en-1-yl)quinoline-4-carboxamide;
N-(2-(2-cyano-4,4-difluoropyrrolidin-1-yl)-2-oxoethyl)-6-(2-(2-methylpyridin-4-yl)vinyl)quinoline-4-carboxamide;
N-(2-(2-cyano-4,4-difluoropyrrolidin-1-yl)-2-oxoethyl)-6-(2-(2-methoxypyridin-4-yl)vinyl)quinoline-4-carboxamide;
N-(2-(2-cyano-4,4-difluoropyrrolidin-1-yl)-2-oxoethyl)-6-(2-(2-(trifluoromethyl)pyridin-4-yl)vinyl)quinoline-4-carb oxamide;
N-(2-(2-cyano-4,4-difluoropyrrolidin-1-yl)-2-oxoethyl)-6-(4-(2-hydroxypropan-2-yl)phenyl)quinoline-4-carboxamide;
N-(2-(2-cyano-4,4-difluoropyrrolidin-1-yl)-2-oxoethyl)-3-(2-(1-oxo-1,2,3,4-tetrahydroisoquinolin-6-yl)vinyl)isonicotinamide;
6-(2-(2-aminopyridin-4-yl)vinyl)-N-(2-(2-cyano-4,4-difluoropyrrolidin-1-yl)-2-oxoethyl)quinoline-4-carboxamide;
6-(2-(6-aminopyridin-3-yl)vinyl)-N-(2-(2-cyano-4,4-difluoropyrrolidin-1-yl)-2-oxoethyl)quinoline-4-carboxamide;
N-(2-(2-cyano-4,4-difluoropyrrolidin-1-yl)-2-oxoethyl)-6-(2-(6-oxo-5,6-dihydropyridin-3-yl)vinyl)quinoline-4-carb oxamide;
N-(2-(2-cyano-4,4-difluoropyrrolidin-1-yl)-2-oxoethyl)-6-(2-(6-((4-methylpiperazin-1-yl)methyl)pyridin-3-yl)vinyl)quinoline-4-carboxamide;
N-(2-(2-cyano-4,4-difluoropyrrolidin-1-yl)-2-oxoethyl)-6-(2-(6-(piperazin-1-ylmethyl)pyridin-3-yl)vinyl)quinoline-4-carb oxamide;
N-(2-(2-cyano-4,4-difluoropyrrolidin-1-yl)-2-oxoethyl)-6-(2-(6-(morpholinomethyl)pyridin-3-yl)vinyl)quinoline-4-carboxamide;
N-(2-(2-cyano-4,4-difluoropyrrolidin-1-yl)-2-oxoethyl)-6-(2-(6-((1-methylpiperidin-4-yl)methyl)pyridin-3-yl)vinyl)quinoline-4-carboxamide;
N-(2-(2-cyano-4,4-difluoropyrrolidin-1-yl)-2-oxoethyl)-6-(2-(6-(piperidin-4-ylmethyl)pyridin-3-yl)vinyl)quinoline-4-carboxamide;
N-(2-(2-cyano-4,4-difluoropyrrolidin-1-yl)-2-oxoethyl)-6-(2-(6-((1-methylpiperidin-4-yl)amino)pyridin-3-yl)vinyl)quinoline-4-carboxamide; and
N-(2-(2-cyano-4,4-difluoropyrrolidin-1-yl)-2-oxoethyl)-6-(2-(6-((1-methylpiperidin-4-yl)oxy)pyridin-3-yl)vinyl)quinoline-4-carboxamide,
N-(2-(2-cyano-4,4-difluoropyrrolidin-1-yl)-2-oxoethyl)-6-(2-(2-hydroxypyridin-4-yl)vinyl)quinoline-4-carboxamide,
N-(2-(2-cyano-4,4-difluoropyrrolidin-1-yl)-2-oxoethyl)-3-(2-(1-oxoisoindolin-5-yl)vinyl)isonicotinamide,
   or a pharmaceutically acceptable salt thereof. Also provided herein are, where applicable, any and all stereoisomers of the compounds depicted herein, including geometric isomers (e.g., cis/trans isomers or E/Z isomers), enantiomers, diastereomers, or mixtures thereof in any ratio, including racemic mixtures.

The compounds depicted herein may be present as salts even if salts are not depicted and it is understood that the present disclosure embraces all salts and solvates of the compounds depicted here, as well as the non-salt and non-solvate form of the compound, as is well understood by the skilled artisan. In some embodiments, the salts of the compounds provided herein are pharmaceutically acceptable salts. Where one or more tertiary amine moiety is present in the compound, the N-oxides are also provided and described.

Where tautomeric forms may be present for any of the compounds described herein, each and every tautomeric form is intended even though only one or some of the tautomeric forms may be explicitly depicted. The tautomeric forms specifically depicted may or may not be the predominant forms in solution or when used according to the methods described herein.

The present disclosure also includes any or all of the stereochemical forms, including any enantiomeric or diastereomeric forms of the compounds described, such as the compounds of Table 1. The structure or name is intended to embrace all possible stereoisomers of a compound depicted.

All forms of the compounds are also embraced by the invention, such as crystalline or non-crystalline forms of the compounds. Compositions comprising a compound of the invention are also intended, such as a composition of substantially pure compound, including a specific stereochemical form thereof, or a composition comprising mixtures of compounds of the invention in any ratio, including two or more stereochemical forms, such as in a racemic or non-racemic mixture.

The invention also intends isotopically-labeled and/or isotopically-enriched forms of compounds described herein. The compounds herein may contain unnatural proportions of atomic isotopes at one or more of the atoms that constitute such compounds. In some embodiments, the compound is isotopically-labeled, such as an isotopically-labeled compound of the formula (I) or variations thereof described herein, where a fraction of one or more atoms are replaced by an isotope of the same element. Exemplary isotopes that can be incorporated into compounds of the invention include isotopes of hydrogen, carbon, nitrogen, oxygen, phosphorus, sulfur, chlorine, such as ²H, ³H, ¹¹C, ¹³C, ¹⁴C ¹³N, ¹⁵O, ¹⁷O, ³²P, ³⁵S, ¹⁸F, ³⁶Cl. Certain isotope labeled compounds (e.g. ³H and ¹⁴C) is useful in compound or substrate tissue distribution studies. Incorporation of heavier isotopes such as deuterium (²H) can afford certain therapeutic advantages resulting from greater metabolic stability, for example, increased in vivo half-life, or reduced dosage requirements and, hence may be preferred in some instances.

Isotopically-labeled compounds of the present invention can generally be prepared by standard methods and techniques known to those skilled in the art or by procedures similar to those described in the accompanying Examples substituting appropriate isotopically-labeled reagents in place of the corresponding non-labeled reagent.

Articles of manufacture comprising a compound described herein, or a salt or solvate thereof, in a suitable container are provided. The container may be a vial, jar, ampoule, preloaded syringe, i.v. bag, and the like.

Preferably, the compounds detailed herein are orally bioavailable. However, the compounds may also be formulated for parenteral (e.g., intravenous) administration.

One or several compounds described herein can be used in the preparation of a medicament by combining the compound or compounds as an active ingredient with a pharmacologically acceptable carrier, which are known in the art. Depending on the therapeutic form of the medication, the carrier may be in various forms. In one variation, the manufacture of a medicament is for use in any of the methods disclosed herein, e.g., for the treatment of cancer.

### General Synthetic Methods

The compounds of the invention may be prepared by a number of processes as generally described below and more specifically in the Examples hereinafter (such as the schemes provided in the Examples below). In the following process descriptions, the symbols when used in the formulae depicted are to be understood to represent those groups described above in relation to the formulae herein.

Where it is desired to obtain a particular enantiomer of a compound, this may be accomplished from a corresponding mixture of enantiomers using any suitable conventional procedure for separating or resolving enantiomers. Thus, for example, diastereomeric derivatives may be produced by reaction of a mixture of enantiomers, e.g., a racemate, and an appropriate chiral compound. The diastereomers may then be separated by any convenient means, for example by crystallization and the desired enantiomer recovered. In another resolution process, a racemate may be separated using chiral High Performance Liquid Chromatography. Alternatively, if desired a particular enantiomer may be obtained by using an appropriate chiral intermediate in one of the processes described.

Chromatography, recrystallization and other conventional separation procedures may also be used with intermediates or final products where it is desired to obtain a particular isomer of a compound or to otherwise purify a product of a reaction.

Solvates of a compound provided herein, a pharmaceutically acceptable salt, stereoisomer or tautomer thereof are also contemplated. Solvates contain either stoichiometric or non-stoichiometric amounts of a solvent, and are often formed during the process of crystallization.

Hydrates are formed when the solvent is water, or alcoholates are formed when the solvent is alcohol.

Compounds of the formula (I-1) can be prepared according to Scheme 1, wherein R, R¹, R², Y, m, n and q are as detailed herein for formula (I), or any variation thereof detailed herein; Z and Z¹ are leaving groups; and PG¹ is an amine protecting group.

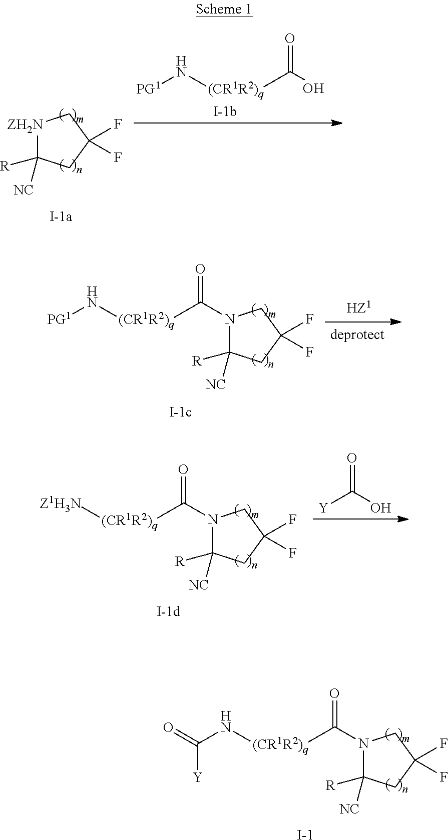

Coupling of a compound of formula (I-1a) with a compound of formula (I-1b) in the presence of a coupling agent (e.g., HATU, HOBt, or PyBOP) yields a compound of formula (I-1c). Deprotection of the amine of the compound of formula (I-1c) under acidic conditions (e.g., HCl or pTsOH) provides the compound of formula (I-1d) as a salt, which is coupled with a carboxylic acid in the presence of a coupling agent (e.g., HATU, HOBt, or PyBOP) to yield a compound of formula (1-1).

An exemplary embodiment of the preparative method in Scheme 1 is shown in Scheme 1a.

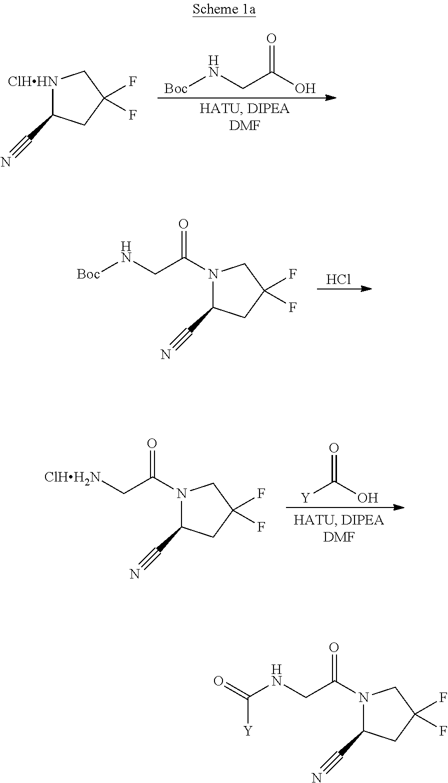

In some embodiments, Y is 6- to 10-membered heteroaryl substituted by R¹². In a further embodiment, Y is pyridin-4-yl substituted by R¹² in the 3-position, wherein

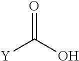

is represented by the compound of formula (II-1).

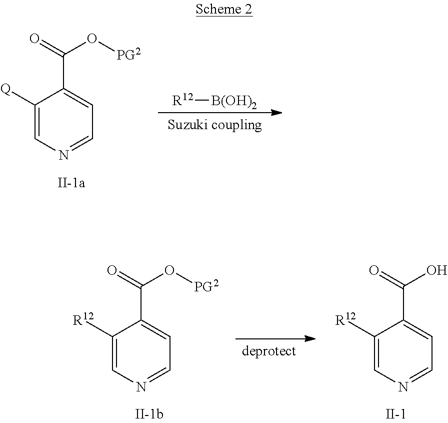

It is understood that the schemes above may be modified to arrive at various compounds of the invention by selection of appropriate reagents and starting materials. For a general description of protecting groups and their use, see P. G. M. Wuts and T. W. Greene, Greene's Protective Groups in Organic Synthesis 4^{th} edition, Wiley-Interscience, New York, 2006.

### Pharmaceutical Compositions and Formulations

Pharmaceutical compositions of any of the compounds detailed herein are embraced by this disclosure. Thus, the present disclosure includes pharmaceutical compositions comprising a compound as detailed herein, or a pharmaceutically acceptable salt, stereoisomer or tautomer thereof and a pharmaceutically acceptable carrier or excipient. In one aspect, the pharmaceutically acceptable salt is an acid addition salt, such as a salt formed with an inorganic or organic acid. Pharmaceutical compositions may take a form suitable for oral, buccal, parenteral, nasal, topical or rectal administration or a form suitable for administration by inhalation.

A compound as detailed herein may in one aspect be in a purified form and compositions comprising a compound in purified forms are detailed herein. Compositions comprising a compound as detailed herein, or a pharmaceutically acceptable salt, stereoisomer or tautomer thereof are provided, such as compositions of substantially pure compounds. In some embodiments, a composition containing a compound as detailed herein, a pharmaceutically acceptable salt, stereoisomer or tautomer thereof is in substantially pure form.

In one variation, the compounds herein are synthetic compounds prepared for administration to an individual. In another variation, compositions are provided containing a compound in substantially pure form. In another variation, the present disclosure embraces pharmaceutical compositions comprising a compound detailed herein and a pharmaceutically acceptable carrier. In another variation, methods of administering a compound are provided. The purified forms, pharmaceutical compositions and methods of administering the compounds are suitable for any compound or form thereof detailed herein.

A compound detailed herein or salt thereof may be formulated for any available delivery route, including an oral, mucosal (e.g., nasal, sublingual, vaginal, buccal or rectal), parenteral (e.g., intramuscular, subcutaneous or intravenous), topical or transdermal delivery form. A compound or salt thereof may be formulated with suitable carriers to provide delivery forms that include, but are not limited to, tablets, caplets, capsules (such as hard gelatin capsules or soft elastic gelatin capsules), cachets, troches, lozenges, gums, dispersions, suppositories, ointments, cataplasms (poultices), pastes, powders, dressings, creams, solutions, patches, aerosols (e.g., nasal spray or inhalers), gels, suspensions (e.g., aqueous or non-aqueous liquid suspensions, oil-in-water emulsions or water-in-oil liquid emulsions), solutions and elixirs.

One or several compounds described herein a pharmaceutically acceptable salt, stereoisomer or tautomer thereof can be used in the preparation of a formulation, such as a pharmaceutical formulation, by combining the compound or compounds, a pharmaceutically acceptable salt, stereoisomer or tautomer thereof, as an active ingredient with a pharmaceutically acceptable carrier, such as those mentioned above. Depending on the therapeutic form of the system (e.g., transdermal patch vs. oral tablet), the carrier may be in various forms. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants. Formulations comprising the compound may also contain other substances which have valuable therapeutic properties. Pharmaceutical formulations may be prepared by known pharmaceutical methods. Suitable formulations can be found, e.g., in Remington's Pharmaceutical Sciences, Mack Publishing Company, Philadelphia, Pa., 20^{th} ed. (2000), which is incorporated herein by reference.

Compounds as described herein may be administered to individuals in a form of generally accepted oral compositions, such as tablets, coated tablets, and gel capsules in a hard or in soft shell, emulsions or suspensions. Examples of carriers, which may be used for the preparation of such compositions, are lactose, corn starch or its derivatives, talc, stearate or its salts, etc. Acceptable carriers for gel capsules with soft shell are, for instance, plant oils, wax, fats, semisolid and liquid poly-ols, and so on. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants.

Compositions comprising a compound provided herein are also described. In one variation, the composition comprises a compound or salt thereof and a pharmaceutically acceptable carrier or excipient. In another variation, a composition of substantially pure compound is provided.

In some embodiments, the composition is for use as a human or veterinary medicament. In some embodiments, the composition is for use in a method described herein. In some embodiments, the composition is for use in the treatment of a disease or disorder described herein.

### Methods of Use and Uses

Compounds and compositions detailed herein, such as a pharmaceutical composition comprising a compound of any formula provided herein a pharmaceutically acceptable salt, stereoisomer or tautomer thereof and a pharmaceutically acceptable carrier or excipient, may be used in methods of administration and treatment as provided herein. The compounds and compositions may also be used in in vitro methods, such as in vitro methods of administering a compound or composition to cells for screening purposes and/or for conducting quality control assays.

Provided herein is a method of treating a disease or disorder in an individual in need thereof comprising administering a compound described herein or any embodiment, variation, or aspect thereof, or a pharmaceutically acceptable salt thereof. In some embodiments, the compound, pharmaceutically acceptable salt thereof, or composition is administered to the individual according to a dosage and/or method of administration described herein.

The compounds or salts thereof described herein and compositions described herein are believed to be effective for treating a variety of diseases and disorders. In some embodiments, a compound or salt thereof described herein or a composition described herein may be used in a method of treating a disease or disorder mediated by fibroblast activation protein (FAP). In some embodiments, the disease or disorder is characterized by proliferation, tissue remodeling, fibrosis, chronic inflammation, excess alcohol consumption, or abnormal metabolism.

In some embodiments, a compound or salt thereof described herein or a composition described herein may be used in a method of treating a disease or disorder mediated by a physiological substrate of FAP peptidase activity. In some embodiments the FAP peptidase activity is endopeptidase activity. In some embodiments, the physiological substrate of FAP endopeptidase activity is α2-antiplasmin, type I collagen, gelatin, and Fibroblast growth factor 21 (FGF21). In some embodiments the FAP peptidase activity is exopeptidase activity. In some embodiments, the physiological substrate of FAP exopeptidase activity is Neuropeptide Y, B-type natriuretic peptide, substance P and peptide YY. In some embodiments, a compound or salt thereof described herein or a composition described herein may be used in a method of treating a disease or disorder mediated by FGF21.

In some embodiments, a compound or salt thereof described herein or a composition described herein may be used in a method of treating a FGF21-associated disorder, such as obesity, type I- and type II diabetes, pancreatitis, dyslipidemia, hyperlipidemia conditions, non-alcoholic fatty liver disease (NAFLD), non-alcoholic steatohepatitis (NASH), insulin resistance, hyperinsulinemia, glucose intolerance, hyperglycemia, metabolic syndrome, acute myocardial infarction, hypertension, cardiovascular diseases, atherosclerosis, peripheral arterial disease, apoplexy, heart failure, coronary artery heart disease, renal disease, diabetic complications, neuropathy, gastroparesis, disorder associated with a serious inactivation mutation in insulin receptor, and other metabolic disorders. In some embodiments, the FGF21-associated disorder is diabetes, obesity, dyslipidemia, metabolic syndrome, non-alcoholic fatty liver disease, non-alcoholic steatohepatitis or cardiovascular diseases.

In some embodiments, a compound or salt thereof described herein or a composition described herein may be used in a method of treating a disease or disorder characterized by proliferation, tissue remodeling, fibrosis, chronic inflammation, excess alcohol consumption, or abnormal metabolism.

In some embodiments, a compound or salt thereof described herein or a composition described herein may be used in a method of treating cancer, such as breast cancer, colorectal cancer, ovarian cancer, prostate cancer, pancreatic cancer, kidney cancer, lung cancer, melanoma, fibrosarcoma, bone sarcoma, connective tissue sarcoma, renal cell carcinoma, giant cell carcinoma, squamous cell carcinoma, leukemia, skin cancer, soft tissue cancer, liver cancer, gastrointestinal carcinoma, or adenocarcinoma. In some embodiments, the compound, salt, or composition may be used in a method of treating metastatic kidney cancer, chronic lymphocytary leukemia, pancreatic adenocarcinoma, or non-small cell lung cancer.

In some embodiments, the administration of the compound, salt, or composition reduces tumor growth, tumor proliferation, or tumorigenicity in the individual. In some embodiments, the compound, salt, or composition may be used in a method of reducing tumor growth, tumor proliferation, or tumorigenicity in an individual in need thereof. In some embodiments, tumor growth is slowed or arrested. In some embodiments, tumor growth is reduced at least about 10%, 20%, 30%, 40%, 50%, 60%, 70%, 80%, 90% or more. In some embodiments, the tumor is reduced in size. In some embodiments, tumor metastasis is prevented or slowed. In some embodiments, the tumor growth, tumor proliferation, or tumorigenicity is compared to the tumor growth, tumor proliferation, or tumorigenicity in the individual prior to the administration of the compound, salt, or composition. In some embodiments, the tumor growth, tumor proliferation, or tumorigenicity is compared to the tumor growth, tumor proliferation, or tumorigenicity in a similar individual or group of individuals. Methods of measuring tumor growth, tumor proliferation, and tumorigenicity are known in the art, for example by repeated imaging of the individual.

In some embodiments, a compound or salt thereof described herein or a composition described herein may be used in in a method of treating fibrotic disease, thrombosis, wound healing, keloid formation, osteoarthritis, rheumatoid arthritis and related disorders involving cartilage degradation, atherosclerotic disease, Crohn's disease, hepatic cirrhosis, idiopathic pulmonary fibrosis, myocardial hypertrophy, diastolic dysfunction, obesity, glucose intolerance, insulin insensitivity, or diabetes mellitus. In some embodiments, the hepatic cirrhosis is viral hepatitis-induced, alcohol-induced, or biliary cirrhosis. In some embodiments, the diabetes mellitus is type II diabetes. In some embodiments, the disease or disorder is fibrotic liver degeneration.

In some embodiments, provided herein is a method of inhibiting FAP. The compounds or salts thereof described herein and compositions described herein are believed to be effective for inhibiting FAP.

In some embodiments, the method of inhibiting FAP comprises inhibiting FAP in a cell by administering or delivering to the cell a compound described herein, or a pharmaceutically acceptable salt thereof, or a pharmaceutical composition described herein. In some embodiments, the cell is a fibroblast, such as a myofibroblast, a keloid fibroblast, a cancer associated fibroblast (CAF), or a reactive stromal fibroblast, among others cells with FAP expression.

In some embodiments, the method of inhibiting FAP comprises inhibiting FAP in a tumor or in plasma by administering or delivering to the tumor or plasma a compound described herein, or a pharmaceutically acceptable salt thereof, or a pharmaceutical composition described herein.

In some embodiments, the inhibition of FAP comprises inhibiting an endopeptidase and/or exopeptidase activity of FAP. In some embodiments, FAP is inhibited by at least about 10%, 20%, 30%, 40%, 50%, 60%, 70%, 80%, 90%, 95%, 98% or more. Inhibition of FAP can be determined by methods known in the art.

In some embodiments, the compound, salt thereof, or composition inhibits FAP with an IC₅₀ of less than about 1 M, such as less than about 750 nM, 600 nM, 500 nM, 300 nM, 200 nM, 100 nM, 80 nM, 60 nM, 40 nM, 25 nM, or less. In some embodiments, the compound, salt thereof, or composition inhibits FAP with an IC₅₀ between about 7 nM and 1 M, such between about 10 nM and 600 nM, 15 nM and 200 nM, or 20 nM and 180 nM. In some aspects, the half maximal inhibitory concentration (IC₅₀) is a measure of the effectiveness of a substance in inhibiting a specific biological or biochemical function. In some aspects, the IC₅₀ is a quantitative measure that indicates how much of an inhibitor is needed to inhibit a given biological process or component of a process such as an enzyme, cell, cell receptor or microorganism by half. Methods of determining IC₅₀ in vitro and in vivo are known in the art.

In some embodiments, the compounds or salts thereof described herein and compositions described herein are administered in an amount wherein DPPII, DPPIV, DPP8, DPP9, and/or PREP activity is not inhibited or is inhibited to a lesser extent. In some embodiments, inhibition of FAP is at least or at least about 2 fold greater than inhibition of DPPII, DPPIV, DPP8, DPP9, and/or PREP activity, for example at least or at least about 3 fold, 4 fold, 5 fold, 8 fold, 10 fold, 15 fold, 30 fold, 50 fold, 60 fold, 75 fold, or 100 fold greater.

By way of example and not wishing to be bound by theory, DPP9 is believed to be a cytoplasmic DPP and belongs to S9B sub-family of proline-selective soluble proteases too. Inhibition of DPP9 activity in macrophages activates the Nlrplb inflammasome. Activation of this pathway leads to pyroptosis, a proinflammatory form of cell death (Okondo M C et al. 2017; Okondo M C et al. 2018) concomitant with the activation of caspase-1 and subsequent activation of pro-IL-10 and pro-IL-18. In MC38 syngeneic mouse model, Val-boroPro a non-selective DPP inhibitor, has shown to inhibit cancer growth with concomitant up-regulation of immune-stimulatory cytokines and tumor infiltration of anti-cancer cell types including CD8+ T cells, Ml-macrophages and NK cells when combined with the immune checkpoint anti-PD1. The cytoplasmic RU134-42 tumor antigen is a natural substrate of DPP9 and endogenous DPP9 limits the presentation of the RU134-42 peptide. These findings suggest a role for DPP9 in antigen presentation (Geiss-Friedlander R et al, 2009). In human myeloid cells, CARD8 mediates DPP8/9 inhibitor-induced procaspase-1β-dependent pyroptosis. DPP8/9 inhibitors induce pyroptosis in the majority of human acute myeloid leukemia (AML) cell lines and primary AML samples, but not in cells from many other lineages, and that these inhibitors inhibit human AML progression in mouse models. Val-boroPro afforded a 97% reduction in tumor burden relative to the vehicle control in a model of disseminated MV4; 11 leukemia cells in NSG mice (Johnson et al., 2018).

Provided herein is a method of enhancing an immune response in an individual comprising administering to the individual a compound described herein, or a pharmaceutically acceptable salt thereof, or a pharmaceutical composition described herein. In some embodiments, the individual has cancer. In some embodiments, the enhanced immune response is directed to a tumor or cancerous cell. By way of example and not wishing to be bound by theory, FAP is believed to suppress immune responses, especially in the context of cancer, therefore inhibiting FAP may enhancing the immune response of an individual. Accordingly, provided herein are methods of treating cancer in an individual in need thereof comprising administering to the individual a compound described herein, or a pharmaceutically acceptable salt thereof, or a pharmaceutical composition described herein, wherein an immune response of the individual is increased.

Provided herein is a method of increasing the level of FGF21 expression in an individual comprising administering to the individual a compound described herein, or a pharmaceutically acceptable salt thereof, or a pharmaceutical composition described herein. Also provided herein is a method of increasing the level of FGF21 or an FGF21 analog in an individual comprising administering to the individual a compound described herein, or a pharmaceutically acceptable salt thereof, or a pharmaceutical composition described herein. In some embodiments, the method further comprises administering FGF21 or an FGF21 analog, such as a mutated FGF21, pegylated FGF21, PF-05231023, or LY2405319.

FGF21 is a peptidic endocrine hormone secreted primarily by the liver (Markan, K. R. et al. Semin Cell Dev Biol, 2016, 53: 85-93). Upon entering circulation, FGF21 functions by signaling to specific tissues regulating carbohydrate and lipid metabolism (Kharitonenkov, A., et al., J Clin Invest, 2005, 115(6): 1627-35). FGF21 stimulates glucose uptake in adipocytes and is believed to protective against obesity and insulin insensitivity. Pharmacological administration of FGF21 to diabetic and obese animal models markedly ameliorates obesity, insulin resistance, dyslipidemia, fatty liver, and hyperglycemia in rodents (Markan, K. R. et al. Semin Cell Dev Biol, 2016, 53: 85-93). Small clinical trials have demonstrated that FGF21 analogs are efficacious in inducing weight loss and correcting hyperinsulinemia, dyslipidemia, and hypoadiponectinemia in obese individuals with type 2 diabetes (Gaich, G., et al., Cell Metab, 2013, 18(3): p. 333-40; Dong, J. Q., et al., Br J Clin Pharmacol, 2015, 80(5): 1051-63.

By way of example and not wishing to be bound by theory, FAP is believed to be the enzyme responsible for cleavage and inactivation of FGF21; therefore inhibiting FAP may increase levels of FGF21 expression and may augment endogenous and/or exogenous FGF21 action. FGF21 interacts with FGFR1 through its N-terminus and with β-Klotho through its C-terminus. This C-terminal region of FGF21 is essential to activate the receptor complex to initiate signaling (Micanovic, R., et al., J Cell Physiol, 2009, 219(2): 227-34; Yie, J., et al., FEBS Lett, 2009, 583(1): 19-24). Recently, FAPα has been identified as the protease responsible for the inactivation of circulating FGF21 through the C-terminal cleavage at Pro171 (Dunshee, D. R., et al., J Biol Chem, 2016, 291(11): 5986-96; Coppage, A. L., et al., PLoS One, 2016, 11(3): e0151269; Zhen, E. Y., et al., Biochem J, 2016, 473(5): 605-14). In rodents and primates, the half-life of exogenously administrated human FGF21 is short (˜0.5-2 h) as result of FAP-mediated enzymatic degradation and susceptibility to renal clearance (Hager, T., et al., Anal Chem, 2013, 85(5): 2731-8; Xu, J., et al., Am J Physiol Endocrinol Metab, 2009, 297(5): E1105-14; Kharitonenkov, A., et al., Endocrinology, 2007, 148(2): 774-81). Common half-life extension strategies have improved significantly the PK properties of these FGF21 analogs in vivo; however, proteolytic processing still persists in these analogs (Hecht, R., et al., PLoS One, 2012, 7(11): e49345; Mu, J., et al., Diabetes, 2012, 61(2): 505-12; Camacho, R. C., et al., Eur J Pharmacol, 2013, 715(1-3): 41-5).

Accordingly, provided herein are methods of treating diabetes mellitus, insulin insensitivity, and/or obesity in an individual in need thereof comprising administering to the individual a compound described herein, or a pharmaceutically acceptable salt thereof, or a pharmaceutical composition described herein. In some embodiments, the method further comprises administering FGF21 or an FGF21 analog. In some embodiments, the FGF21 analog is pegylated FGF21, PF-05231023, or LY2405319. Also provided herein are methods of treating diabetes mellitus, insulin insensitivity, and/or obesity in an individual in need thereof comprising administering to the individual a compound described herein, or a pharmaceutically acceptable salt thereof, or a pharmaceutical composition described herein, wherein FGF21 expression is increased. In some embodiments, the diabetes mellitus is type II diabetes.

In some embodiments, the individual is a mammal. In some embodiments, the individual is a primate, bovine, ovine, porcine, equine, canine, feline, lapine, or rodent. In some embodiments, the individual is a human. In some embodiments, the individual has any of the diseases or disorders disclosed herein. In some embodiments, the individual is a risk for developing any of the diseases or disorders disclosed herein.

In some embodiments, the individual is human. In some embodiments, the human is at least about or is about any of 21, 25, 30, 35, 40, 45, 50, 55, 60, 65, 70, 75, 80, or 85 years old. In some embodiments, the human is a child. In some embodiments, the human is less than about or about any of 21, 18, 15, 12, 10, 8, 6, 5, 4, 3, 2, or 1 years old.

Also provided herein are uses of a compound described herein or a pharmaceutically acceptable salt thereof, or a pharmaceutical composition described herein, in the manufacture of a medicament. In some embodiments, the manufacture of a medicament is for the treatment of a disorder or disease described herein. In some embodiments, the manufacture of a medicament is for the prevention and/or treatment of a disorder or disease mediated by FAP.

### Combination Therapy

As provided herein, compounds or salts thereof described herein and compositions described herein may be administered with an additional agent to treat any of the diseases and disorders disclosed herein.

In some embodiments, (a) a compound described herein, or a pharmaceutically acceptable salt thereof, or a pharmaceutical composition described herein and (b) an additional agent are sequentially administered, concurrently administered or simultaneously administered. In certain embodiments, (a) a compound described herein, or a pharmaceutically acceptable salt thereof, or a pharmaceutical composition described herein and (b) an additional agent are administered with a time separation of about 15 minutes or less, such as about any of 10, 5, or 1 minutes or less. In certain embodiments, (a) a compound described herein, or a pharmaceutically acceptable salt thereof, or a pharmaceutical composition described herein and (b) an additional agent are administered with a time separation of about 15 minutes or more, such as about any of 20, 30, 40, 50, 60, or more minutes. Either (a) a compound described herein, or a pharmaceutically acceptable salt thereof, or a pharmaceutical composition described herein and (b) an additional agent may be administered first. In certain embodiments, (a) a compound described herein, or a pharmaceutically acceptable salt thereof, or a pharmaceutical composition described herein and (b) an additional agent are administered simultaneously.

In some embodiments, the additional agent targets an immune checkpoint protein. In some embodiments, the additional agent is an antibody that targets an immune checkpoint protein.

In some embodiments, the additional agent targets PD-1, PD-L1, PD-L2, CTLA4, TIM3, LAG3, CCR4, OX40, OX40L, IDO, and A2AR. In some embodiments, the additional agent is an anti-PD-1 antibody, an anti-PD-L1 antibody, or an anti-CTLA-4 antibody.

In some embodiments, the additional agent is an inducer of FGF21 expression, such as a PPARα agonist. In some embodiment, the PPARα agonist is fibrate or fenofibrate. In some embodiments, the additional agent is FGF-21 or an FGF-21 analog. In some embodiments, the FGF-21 analog is a mutated FGF21 and/or pegylated FGF21. In some embodiments, the FGF-21 analog is PF-05231023 or LY2405319.

In some embodiments, the additional agent is a KLB/FGFR complex agonist, a DDPIV antagonist, a GLP-1 receptor agonist, or a glucagon receptor agonist.

Provided herein is a method of enhancing an immune response in an individual comprising administering to the individual (a) a compound described herein, or a pharmaceutically acceptable salt thereof, or a pharmaceutical composition described herein and (b) an agent that targets an immune checkpoint protein. In some embodiments, the individual has cancer. In some embodiments, the enhanced immune response is directed to a tumor or cancerous cell.

Also provided herein are methods of treating cancer in an individual in need thereof comprising administering to the individual (a) a compound described herein, or a pharmaceutically acceptable salt thereof, or a pharmaceutical composition described herein and (b) an agent that targets an immune checkpoint protein, wherein an immune response of the individual is increased.

Provided herein is a method of increasing the level of FGF21 expression in an individual comprising administering to the individual (a) a compound described herein, or a pharmaceutically acceptable salt thereof, or a pharmaceutical composition described herein and (b) an agent that induces FGF21 expression.

Also provided herein are methods of treating diabetes mellitus, insulin insensitivity, and/or obesity in an individual in need thereof comprising administering to the individual (a) a compound described herein, or a pharmaceutically acceptable salt thereof, or a pharmaceutical composition described herein and (b) an agent that induces FGF21 expression, wherein FGF21 expression is increased. In some embodiments, the diabetes mellitus is type II diabetes.

As provided herein, compounds or salts thereof described herein and compositions described herein are administered as part of a treatment regimen that includes an exercise regimen, such as strength-training or cardiovascular exercise. In some embodiments, the compounds or salts thereof described herein and compositions described herein are administered with an additional agent and as part of a treatment regimen that includes an exercise regimen, such as strength-training or cardiovascular exercise. In some embodiments, the exercise regimen comprises exercising at least once per week, such as twice per week, 3× per week, 4× per week, 5× per week, 6× per week, or 7× per week. In some embodiments, the exercise regimen comprises exercising at least one day per week, such as two days per week, 3 days per week, 4 days per week, 5 days per week, 6 days per week, or 7 days per week. In some embodiments, the exercise regimen comprises exercising once per day, twice per day, or 3× per day. In some embodiments, the exercise regimen comprises exercising for at least 10 minutes per session, such as for at least 15 min, 20 min, 25 min, 30 min, 35 min, 40 min, 45 min, 50 min, 55 min, 1 hour, 1.25 hours, or 1.5 hours.

### Dosing and Method of Administration

The dose of a compound administered to an individual (such as a human) may vary with the particular compound or salt thereof, the method of administration, and the particular disease, such as type and stage of cancer, being treated. In some embodiments, the amount of the compound or salt thereof is a therapeutically effective amount.

The effective amount of the compound may in one aspect be a dose of between about 0.01 and about 100 mg/kg. Effective amounts or doses of the compounds of the invention may be ascertained by routine methods, such as modeling, dose escalation, or clinical trials, taking into account routine factors, e.g., the mode or route of administration or drug delivery, the pharmacokinetics of the agent, the severity and course of the disease to be treated, the subject's health status, condition, and weight. An exemplary dose is in the range of about from about 0.7 mg to 7 g daily, or about 7 mg to 350 mg daily, or about 350 mg to 1.75 g daily, or about 1.75 to 7 g daily.

Any of the methods provided herein may in one aspect comprise administering to an individual a pharmaceutical composition that contains an effective amount of a compound provided herein a pharmaceutically acceptable salt, stereoisomer or tautomer thereof and a pharmaceutically acceptable excipient.

A compound or composition of the invention may be administered to an individual in accordance with an effective dosing regimen for a desired period of time or duration, such as at least about one month, at least about 2 months, at least about 3 months, at least about 6 months, or at least about 12 months or longer, which in some variations may be for the duration of the individual's life. In one variation, the compound is administered on a daily or intermittent schedule.

The compound can be administered to an individual continuously (for example, at least once daily) over a period of time. The dosing frequency can also be less than once daily, e.g., about a once weekly dosing. The dosing frequency can be more than once daily, e.g., twice or three times daily.

The dosing frequency can also be intermittent, including a ‘drug holiday’ (e.g., once daily dosing for 7 days followed by no doses for 7 days, repeated for any 14 day time period, such as about 2 months, about 4 months, about 6 months or more). Any of the dosing frequencies can employ any of the compounds described herein together with any of the dosages described herein.

### Articles of Manufacture and Kits

The present disclosure further provides articles of manufacture comprising a compound described herein a pharmaceutically acceptable salt, stereoisomer or tautomer thereof, a composition described herein, or one or more unit dosages described herein in suitable packaging. In certain embodiments, the article of manufacture is for use in any of the methods described herein. Suitable packaging is known in the art and includes, for example, vials, vessels, ampules, bottles, jars, flexible packaging and the like. An article of manufacture may further be sterilized and/or sealed.

The present disclosure further provides kits for carrying out the methods of the invention, which comprises one or more compounds described herein or a composition comprising a compound described herein. The kits may employ any of the compounds disclosed herein. In one variation, the kit employs a compound described herein a pharmaceutically acceptable salt, stereoisomer or tautomer thereof. The kits may be used for any one or more of the uses described herein, and, accordingly, may contain instructions for the treatment any disease or described herein, for example for the treatment of cancer.

Kits generally comprise suitable packaging. The kits may comprise one or more containers comprising any compound described herein. Each component (if there is more than one component) can be packaged in separate containers or some components can be combined in one container where cross-reactivity and shelf life permit.

The kits may be in unit dosage forms, bulk packages (e.g., multi-dose packages) or sub-unit doses. For example, kits may be provided that contain sufficient dosages of a compound as disclosed herein and/or an additional pharmaceutically active compound useful for a disease detailed herein to provide effective treatment of an individual for an extended period, such as any of a week, 2 weeks, 3 weeks, 4 weeks, 6 weeks, 8 weeks, 3 months, 4 months, 5 months, 7 months, 8 months, 9 months, or more. Kits may also include multiple unit doses of the compounds and instructions for use and be packaged in quantities sufficient for storage and use in pharmacies (e.g., hospital pharmacies and compounding pharmacies).

The kits may optionally include a set of instructions, generally written instructions, although electronic storage media (e.g., magnetic diskette or optical disk) containing instructions are also acceptable, relating to the use of component(s) of the methods of the present invention. The instructions included with the kit generally include information as to the components and their administration to an individual.

The invention can be further understood by reference to the following examples, which are provided by way of illustration and are not meant to be limiting.

All references throughout, such as publications, patents, patent applications and published patent applications, are incorporated herein by reference in their entireties.

### EXAMPLES

### Synthetic Examples

The chemical reactions in the Synthetic Examples described can be readily adapted to prepare a number of other compounds of the invention, and alternative methods for preparing the compounds of this invention are deemed to be within the scope of this invention. For example, the synthesis of non-exemplified compounds according to the invention can be successfully performed by modifications apparent to those skilled in the art, e.g., by appropriately protecting interfering groups, by utilizing other suitable reagents known in the art other than those described, or by making routine modifications of reaction conditions. Alternatively, other reactions disclosed herein or known in the art will be recognized as having applicability for preparing other compounds of the invention.

### Example S1

### Synthesis of (S)—N-(2-(2-cyano-4,4-difluoropyrrolidin-1-yl)-2-oxoethyl)-3-(2-phenylacetamido)isonicotinamide

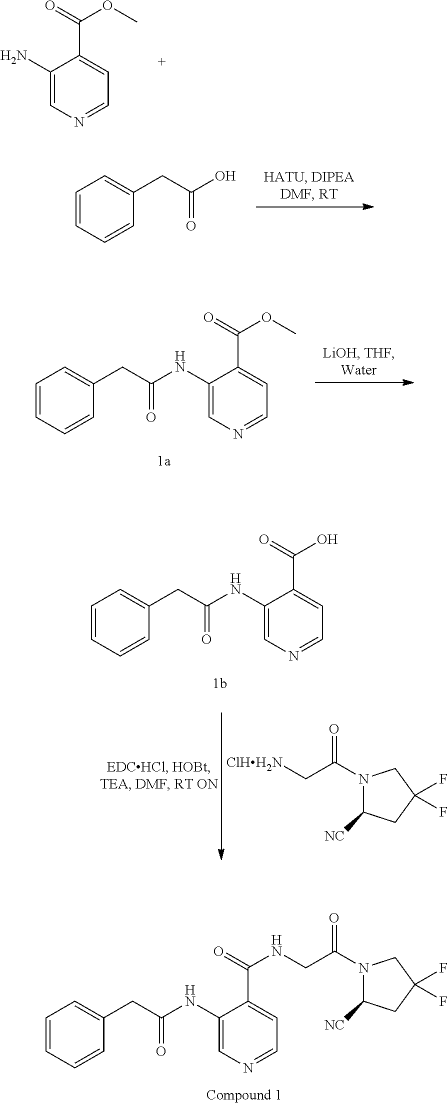

Compound 1a. To a stirred solution of 2-phenylacetic acid (0.500 g, 3.67 mmol, 1.0 equiv) in DMF (10 mL) was added methyl 3-aminoisonicotinate (0.558 g, 3.67 mmol, 1.0 equiv) and HATU (2.8 g, 7.35 mmol, 2.0 equiv) at RT. The resulting reaction mixture was stir for 10 minutes at RT and DIPEA (2.0 mL, 11.50 mmol, 3.0 equiv) was added. The reaction mixture was allowed to stir at RT for overnight. Product formation was confirmed by LCMS. The reaction mixture was diluted with water (50 mL) and extracted with ethyl acetate (100 mL×2). Combined organic extracts were washed with water (20 mL×4), dried over anhydrous Na2SO4 and concentrated. Crude product was purified by flash chromatography (0-40% ethyl acetate in hexane as an eluent) to obtain methyl 3-(2-phenylacetamido)isonicotinate (0.460 g, 46% Yield) as an yellow semisolid.

LCMS 271.2 [M+H]⁺

Compound 1b. To a stirred solution of methyl 3-(2-phenylacetamido)isonicotinate (0.290 g, 1.07 mmol, 1 equiv) in THF and water (6 mL: 3 mL) was added LiOH (0.051 g, 2.14 mmol, 2.0 equiv). The mixture was allowed to stir at ambient temperature for 16 h. Product formation was confirmed by LCMS. The reaction mixture was diluted with water (20 mL). Aqueous layer was washed with ethyl acetate (10 mL), aqueous layer was separated and freeze dried over lyophilizer to obtain 3-(2-phenylacetamido)isonicotinic acid (0.420 g. Quant. Yield) as an off-white solid

LCMS 257.2 [M+H]⁺

Compound 1. To a stirred solution of 3-(2-phenylacetamido)isonicotinic acid (0.200 g, 0.713 mmol, 1 equiv) in DMF (5 mL), was added (S)-4,4-difluoro-1-glycylpyrrolidine-2-carbonitrile hydrochloride (0.161 g, 0.713 mmol, 1.0 equiv), HOBt (0.115 g, 0.856 mmol, 1.2 equiv) and EDC.HCl (0.164 g, 0.856 mmol, 1.2 equiv). The mixture was allowed to stir at RT for 10 min. Triethyl amine (0.145 g, 1.427 mmol, 2.0 equiv) was added and the mixture was allowed to stir at ambient temperature for 16 h. Product formation was confirmed by LCMS. The reaction mixture was diluted with water (10 mL) and extracted with ethyl acetate (40 mL×2). Combined organic extracts were washed with water (25 mL×5). Organic layer was dried over anhydrous Na₂SO₄, filtered and concentrated under reduced pressure to obtain crude product. The crude product obtained was purified by reversed phase HPLC to obtain (S)—N-(2-(2-cyano-4,4-difluoropyrrolidin-1-yl)-2-oxoethyl)-3-(2-phenylacetamido)isonicotinamide (0.070 g, 21% Yield) as an yellow solid.

LCMS 428.3 [M+H]⁺

¹H NMR (400 MHz, DMSO-d₆) δ 10.44 (br. s., 1H), 9.41 (s, 1H), 9.21 (br. s., 1H), 8.42 (d, J=4.8 Hz, 1H), 7.56 (d, J=4.8 Hz, 1H), 7.43-7.15 (m, 4H), 5.16 (d, J=8.8 Hz, 1H), 4.31 (d, J=12.7 Hz, 2H), 4.22-4.08 (m, 3H), 3.76 (s, 2H), 2.93 (br. s., 1H), 2.84 (d, J=15.8 Hz, 2H).

### Example S2

### Synthesis of (S)—N-(2-(2-cyano-4,4-difluoropyrrolidin-1-yl)-2-oxoethyl)-3-((4-fluorobenzyl)amino)isonicotinamide

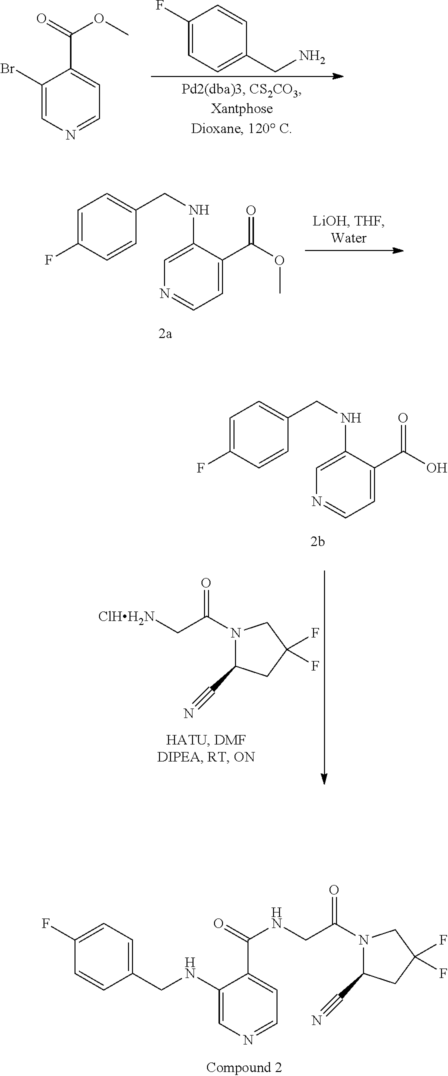

Compound 2a. To a stirred solution of methyl 3-bromoisonicotinate (0.500 gm, 2.3148 mmol, 1 equiv) & (4-fluorophenyl)methanamine (0.289 gm, 2.3148 mmol, 1 equiv) and CS₂CO₃(1.5 gm, 4.6296 mmol, 2 equiv) in dioxane (15.0 mL). The resulting mixture was purged with nitrogen for 10 min followed by addition of Pd₂(dba)₃ (0.106 gm, 0.1157 mmol, 0.05 equiv) and xantphos (0.134 gm, 0.2314 mmol, 0.1 equiv), again purged with nitrogen for 10 min. The reaction mixture was heated at 120° C. for overnight. The progress of reaction was monitored by LCMS. The reaction mixture was diluted with water (30 mL), extracted with EtOAc (2×50 mL). The combined organic layers were washed with water (30 mL), with brine (30 mL), dried over Na₂SO₄, concentrated to afford crude product. Crude was purified by column chromatography (0-50% ethyl acetate in hexane as an eluent) to obtain the desired methyl 3-((4-fluorobenzyl) amino)isonicotinate (200 mg, 33.00%) as an off white solid.

LCMS: 262.1 [M+H]⁺

¹H NMR; (400 MHz, DMSO-d₆) δ8.18 (s, 1H), 7.83 (s, 2H), 7.43 (d, 2H), 7.34 (d, 2H), 7.17 (s, 1H), 4.57 (d, J=5.7 Hz, 2H), 3.86 (s, 3H)

Compound 2b. To a stirred solution of compound methyl 3-((4-fluorobenzyl) amino)isonicotinate (0.200 gm, 0.7692 mmol, 1 equiv) in THF (8 mL) and water (4 mL), was added LiOH (0.040 gm, 1.5384 mmol, 2 equiv). The mixture was allowed to stir at 80° C. for overnight. Product formation was confirmed by LCMS. The reaction mixture was concentrated and diluted with water (10 mL) and washed with ethyl acetate (2×10 ml). Aqueous layer was separated and freeze dried on lyophilyzer to obtain compound 3-((4-fluorobenzyl) amino)isonicotinic acid (170 mg, 89.94%) as a white solid.

LCMS: 247.2 [M+H]⁺

¹H NMR; (400 MHz, DMSO-d₆) δ11.70 (s, 1H), 8.18 (s, 1H), 7.83 (s, 2H), 7.43 (d, 2H), 7.34 (d, 2H), 7.17 (s, 1H), 4.57 (d, J=5.7 Hz, 2H).

Compound 2. To a stirred solution of 3-((4-fluorobenzyl) amino)isonicotinic acid (0.170 gm, 0.8130 mmol, 1 equiv), HATU (0.464 gm, 1.2195 mmol, 1.5 equiv) in DMF (10 ml) after 10 min was added a (S)-4,4-difluoro-1-glycylpyrrolidine-2-carbonitrile hydrochloride (0.219 gm, 0.9756 mmol, 1.2 equiv) and stirred the reaction mixture for 10 min, followed by drop wise addition of DIPEA (0.3 ml, 1.2195 mmol, 1.5 equiv) allowed the reaction for 16 h stirring at rt. reaction progress was monitored by LCMS and TLC, workup done by addition of chilled water (50 mL) to the reaction mass and extracted by ethyl acetate (3×50 mL) and collected all the organic layers, washed by water by three times and once by sodium bicarbonate and once by brine solution. Organic layer was dried over anhydrous Na₂SO₄, concentrated on reduced pressure crude was purified by reverse phase chromatography to afford the desired product (S)—N-(2-(2-cyano-4,4-difluoropyrrolidin-1-yl)-2-oxoethyl)-3-((4-fluorobenzyl) amino)isonicotinamide (30 mg, 11%) as white solid.

LCMS: 418.2 [M+H]⁺

¹H NMR (400 MHz, DMSO-d₆) δ 8.99 (br. s., 1H), 8.10 (s, 1H), 7.88 (d, J=4.8 Hz, 2H), 7.49 (d, J=5.3 Hz, 1H), 7.44-7.35 (m, 2H), 7.17 (t, J=9.0 Hz, 2H), 5.10 (d, J=7.5 Hz, 1H), 4.47 (d, J=5.3 Hz, 4H), 4.12 (d, J=6.6 Hz, 2H), 2.82 (d, J=16.7 Hz, 2H).

### Example S3

### Synthesis of (S)—N-(2-(2-cyano-4,4-difluoropyrrolidin-1-yl)-2-oxoethyl)-3-(4-methoxybenzamido)isonicotinamide

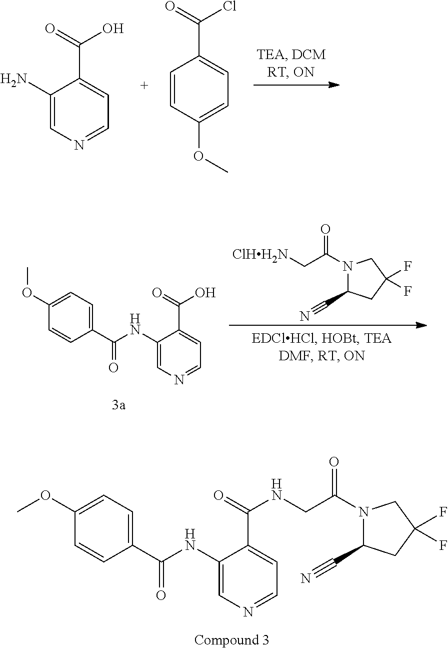

Compound 3a. To a solution of 3-aminoisonicotinic acid (0.5 g, 3.628 mmol, 1.0 equiv) in Dioxan (10 mL) was added 4-methoxybenzoyl chloride (1.0 mL, 7.246 mmol, 2.0 equiv). The resulting reaction mixture was stir at RT for 10 min. TEA (1.5 mL, 10.87 mmol. 3.0 equiv) was added dropwise at RT. The resulting reaction mixture was stirred overnight at RT. Product formation was confirmed by LCMS. After the completion of reaction, reaction mixture was concentrated up to dry and diluted with water (50 mL). Aqueous layer was washed with EtOAc (2×20 mL). Aqueous layer was separated and freeze dried on lyophilyzer to obtain 3-(4-methoxybenzamido)isonicotinic acid (Quant. Yield) as an yellow solid.

LCMS 273.1 [M+H]⁺

Compound 3. To a stirred solution of 3-(4-methoxybenzamido)isonicotinic acid (0.10 g, 0.367 mmol, 1.0 equiv) in DMF (5 mL), was added (S)-4,4-difluoro-1-glycylpyrrolidine-2-carbonitrile hydrochloride (0.082 g, 0.367 mmol, 1.0 equiv), EDCI.HCl (0.141 g, 0.734 mmol, 2.0 equiv) & HOBt (0.099 g, 0.737 mmol, 2.0 equiv). The mixture was allowed to stir at RT for 10 min. TEA (0.4 mL) was added and the mixture was allowed to stir at RT for overnight. Product formation was confirmed by LCMS and TLC. After completion of reaction, the mixture was diluted with water (20 mL) and extracted with ethyl acetate (50 mL×3). Combined organic extracts were washed with water (50 mL×5), dried over anhydrous Na₂SO₄ and concentrated. The crude product obtained was purified by flash chromatography (5% MeOH in DCM as an eluent) to obtain (S)—N-(2-(2-cyano-4,4-difluoropyrrolidin-1-yl)-2-oxoethyl)-3-(4-methoxybenzamido)isonicotinamide (0.015 g, 9.2% Yield) as an off white solid.

LCMS 444.2 [M+H]⁺

¹H NMR (400 MHz, DMSO-d₆) δ 11.59 (s, 1H), 9.75 (s, 1H), 9.44 (br. s., 1H), 8.49 (d, J=4.8 Hz, 1H), 7.99-7.83 (m, J=8.8 Hz, 2H), 7.77 (d, J=5.3 Hz, 1H), 7.25-6.97 (m, J=8.8 Hz, 2H), 5.12 (d, J=6.1 Hz, 1H), 4.33 (br. s., 1H), 4.29-4.07 (m, 2H), 2.91 (br. s., 3H).

### Example S4

### Synthesis of N-(2-((S)-2-cyano-4,4-difluoropyrrolidin-1-yl)-2-oxoethyl)-3-((1-(4-fluorophenyl) ethyl)amino)isonicotinamide

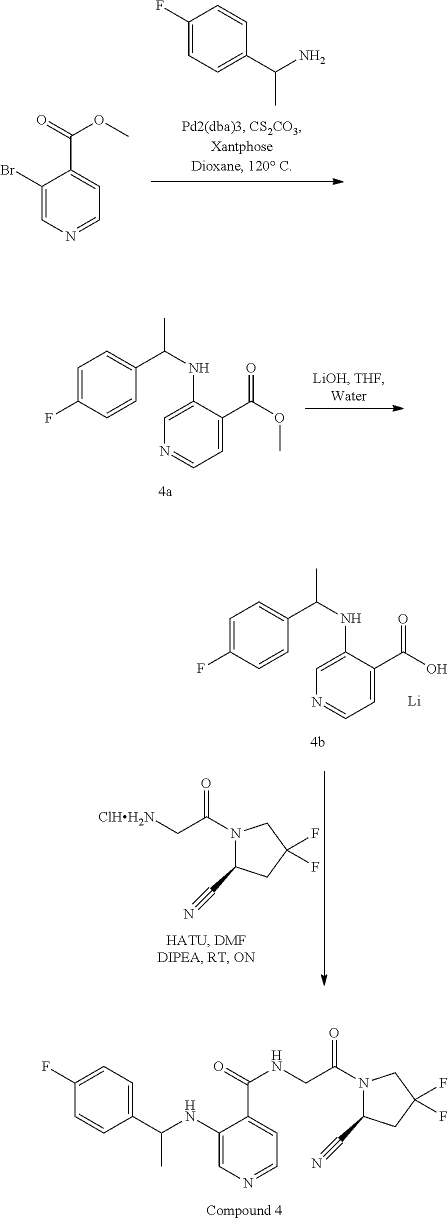

Compound 4a. To a stirred solution of methyl 3-bromoisonicotinate (0.500 gm, 2.3148 mmol, 1.0 equiv) & 1-(4-fluorophenyl)ethan-1-amine (0.350 gm, 2.540 mmol, 1.0 equiv) and CS₂CO₃(1.5 gm, 4.6296 mmol, 2 equiv) in Dioxan (20 mL). The resulting mixture was purged with nitrogen for 10 min followed by addition of Pd₂(dba)₃ (0.110 gm, 0.115 mmol, 0.05 equiv) and xantphos (0.135 gm, 0.231 mmol, 0.1 equiv), again purged with nitrogen for 10 min. The reaction mixture was heated at 120° C. for overnight. The progress of reaction was monitored by LCMS. The reaction mixture was diluted with water (30 mL), extracted with EtOAc (2×50 mL). The combined organic layer was washed with water (30 mL), with brine (30 mL), dried over Na₂SO₄, concentrated. Crude was purified by flash chromatography (0-50% ethyl acetate in hexane as an eluent) to obtain methyl 3-((1-(4-fluorophenyl)ethyl)amino)isonicotinate (200 mg, 31.0%) as an off white solid.

LCMS: 275.1 [M+H]⁺

¹H NMR (400 MHz, CHLOROFORM-d) δ 7.98 (s, 1H), 7.87 (d, J=5.3 Hz, 1H), 7.62 (d, J=5.3 Hz, 1H), 7.38-7.29 (m, 1H), 7.00 (t, J=8.6 Hz, 2H), 4.67 (t, J=6.4 Hz, 1H), 3.93 (s, 3H), 1.63-1.49 (m, 3H).

Compound 4b. To a stirred solution of methyl 3-((1-(4-fluorophenyl)ethyl)amino)isonicotinate (0.200 gm, 0.727 mmol, 1.0 equiv) in THF (10 mL) and water (4 mL), was added LiOH (0.035 gm, 1.45 mmol, 2 equiv). The mixture was allowed to stir at 80° C. for overnight. Product formation was confirmed by LCMS. The reaction mixture was concentrated and diluted with water (10 mL) and washed with ethyl acetate (2×10 mL). Aqueous layer was separated and freeze dried on lyophilyzer to obtain 3-((1-(4-fluorophenyl)ethyl)amino)isonicotinic acid, lithium salt (0.250 gm, Quant. Yield) as a white solid.

LCMS: 261.2 [M+H]⁺

¹H NMR (400 MHz, DMSO-d₆) δ 9.56 (d, J=7.5 Hz, 1H), 7.65-7.57 (m, 2H), 7.52 (d, J=4.8 Hz, 1H), 7.38 (dd, J=5.7, 8.8 Hz, 2H), 7.11 (t, J=9.0 Hz, 2H), 4.64 (t, J=6.8 Hz, 1H), 1.42 (d, J=7.0 Hz, 3H).

Compound 4. To a stirred solution of 3-((1-(4-fluorophenyl)ethyl)amino)isonicotinic acid, lithium salt (0.250 gm, 0.936 mmol, 1.0 equiv) in DMF (5 mL) was added EDC.HCl (0.269 gm, 1.404 mmol, 1.5 equiv), HOBt (0.152 gm, 1.123, 1.2 equiv), (S)-4,4-difluoro-1-glycylpyrrolidine-2-carbonitrile hydrochloride (0.219 gm, 0.9756 mmol, 1.2 equiv), DMAP (0.002 gm, 0.009 mmols, 0.01 equiv) followed by the addition of triethyl amine (0.4 mL, 2.808 mmols, 3.0 equiv). The resulting reaction mixture was allowed stir at RT for 16 h. The reaction progress was monitored by LCMS and TLC, reaction mixture was diluted with water extracted by ethyl acetate (2×50 mL). Combined organic layer was washed with water (20 mL×4). Organic layer was dried over anhydrous Na₂SO₄, concentrated under reduced pressure. Crude product was purified by reversed phase chromatography to obtain N-(2-((S)-2-cyano-4,4-difluoropyrrolidin-1-yl)-2-oxoethyl)-3-((1-(4-fluorophenyl)ethyl)amino)isonicotinamide (0.012 gm, 3%) as white solid.

LCMS: 432.2 [M+H]⁺

¹H NMR (400 MHz, DMSO-d₆) δ 9.02 (br. s., 1H), 7.96-7.89 (m, 1H), 7.83 (d, J=4.8 Hz, 1H), 7.52-7.33 (m, 2H), 7.19-7.05 (m, 1H), 5.12 (d, J=8.3 Hz, 1H), 4.88-4.72 (m, 1H), 4.31 (br. s., 1H), 4.22-4.02 (m, 2H), 2.92 (br. s., 1H), 2.83 (d, J=18.9 Hz, 1H), 1.45 (d, J=6.6 Hz, 3H).

### Example S5

### Synthesis of (S)-3-(2-(4-chloro-3-fluorophenoxy)acetamido)-N-(2-(2-cyano-4,4-difluoropyrrolidin-1-yl)-2-oxoethyl)isonicotinamide

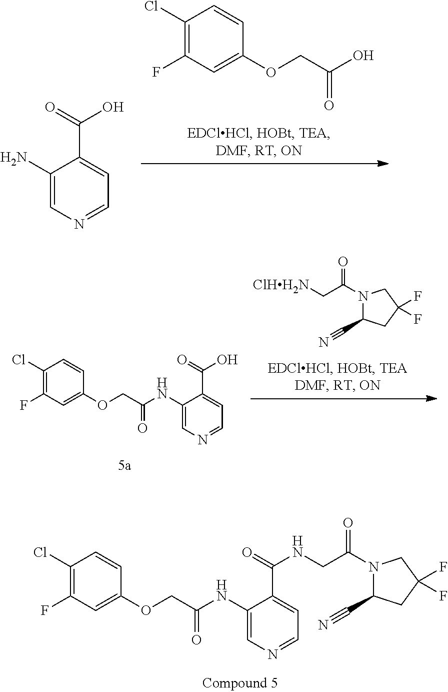

Compound 5a. To a stirred solution of 3-aminoisonicotinic acid (0.2 g, 1.45 mmol, 1.0 equiv) in DMF (5 mL), was added 2-(4-chloro-3-fluorophenoxy)acetic acid (0.59 g, 2.898 mmol, 2.0 equiv), EDCI.HCl (0.556 g, 2.898 mmol, 2.0 equiv) & HOBt (0.391 g, 2.898 mmol, 2.0 equiv). The mixture was allowed to stir at RT for 10 min. TEA (0.4 mL) was added and the mixture was allowed to stir at RT for overnight. Product formation was confirmed by LCMS and TLC. After completion of reaction, the mixture was diluted with water (20 mL) and extracted with ethyl acetate (50 mL×3). Aqueous layer was separated and freeze dried on lyophilyzer to obtain 3-(2-(4-chloro-3-fluorophenoxy)acetamido)isonicotinic acid (Quant. Yield) as an off white solid.

LCMS 325.1 [M+H]⁺

Compound 5. To a stirred solution of 3-(2-(4-chloro-3-fluorophenoxy)acetamido)isonicotinic acid (0.20 g, 0.617 mmol, 1.0 equiv) in DMF (15 mL), was added (S)-4,4-difluoro-1-glycylpyrrolidine-2-carbonitrile hydrochloride (0.138 g, 0.617 mmol, 1.0 equiv), EDCI.HCl (0.141 g, 1.234 mmol, 2.0 equiv) & HOBt (0.166 g, 1.234 mmol, 2.0 equiv). The mixture was allowed to stir at RT for 10 min. TEA (2.0 mL) was added and the mixture was allowed to stir at RT for overnight. Product formation was confirmed by LCMS and TLC. After completion of reaction, the mixture was diluted with water (20 mL) and extracted with ethyl acetate (50 mL×3). Combined organic extracts were washed with water (50 mL×5), dried over anhydrous Na₂SO₄ and concentrated. The crude product obtained was purified by flash chromatography (5% MeOH in DCM as an eluent) to obtain (S)-3-(2-(4-chloro-3-fluorophenoxy)acetamido)-N-(2-(2-cyano-4,4-difluoropyrrolidin-1-yl)-2-oxoethyl)isonicotinamide (0.025 g, 8.1% Yield) as an off white solid.

LCMS 444.2 [M+H]⁺

¹H NMR ¹H NMR (400 MHz, DMSO-d₆) δ 11.37 (s, 1H), 9.63 (s, 1H), 9.35 (br. s., 1H), 8.50 (d, J=4.8 Hz, 1H), 7.73 (d, J=5.3 Hz, 1H), 7.52 (t, J=8.8 Hz, 1H), 7.19 (d, J=11.0 Hz, 1H), 6.96 (d, J=6.1 Hz, 1H), 5.11 (d, J=6.6 Hz, 1H), 4.81 (s, 2H), 4.32 (br. s., 1H), 4.27-3.99 (m, 2H), 2.92 (br. s., 2H), 2.84 (d, J=11.4 Hz, 1H)

### Example S6

### Synthesis of (S)-2-(4-(bis(4-fluorophenyl)methyl)piperazin-1-yl)-N-(2-(2-cyano-4,4-difluoropyrrolidin-1-yl)-2-oxoethyl)isonicotinamide

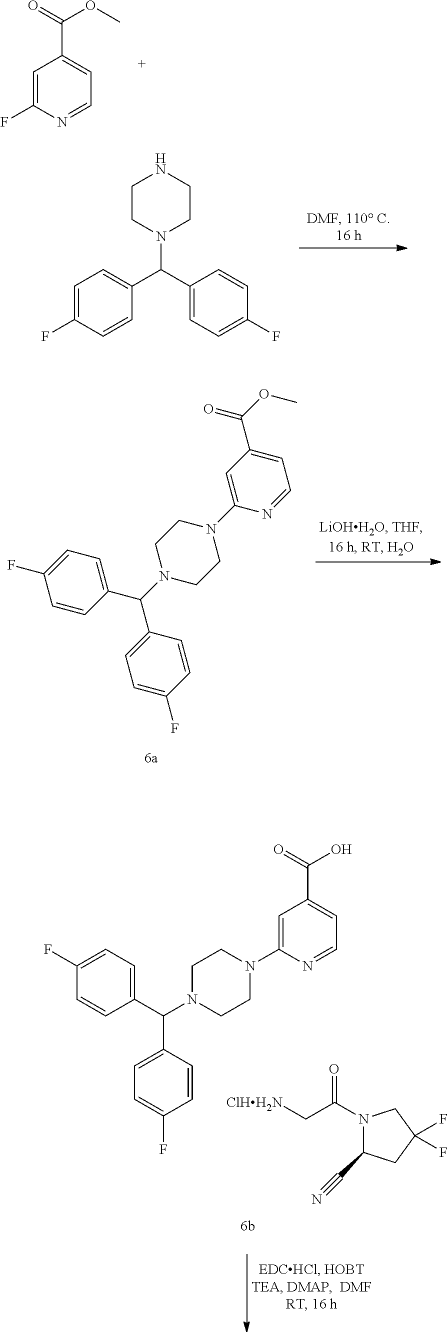

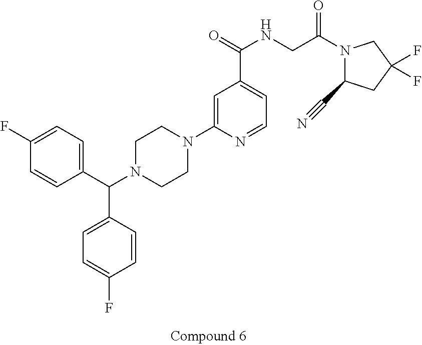 [SMILES: N#C[C@@H]1CC(F)(F)CN1C(=O)CNC(=O)c1ccnc(N2CCN(C(c3ccc(F)cc3)c3ccc(F)cc3)CC2)c1]

Compound 6a. To a stirred solution of methyl 2-fluoroisonicotinate (0.050 g, 0.32 mmol, 1.0 equiv) in DMF (3 ml) was added 1-(bis(4-fluorophenyl)methyl)piperazine (0.093 g, 0.32 mmol, 1.0 equiv). The reaction mixture was allowed to heat at 100° C. for 16 h. Product formation was confirmed by TLC and LCMS. The reaction mixture was diluted with water (15 mL) and extracted with ethyl acetate (20 mL×3). Combined organic layer was washed with water (12 mL×6). Organic layer was dried over anhydrous sodium sulphate, filtered and concentrated under reduced pressure. Crude compound was purified by normal phase combi-flash chromatography to obtain methyl 2-(4-(bis(4-fluorophenyl)methyl)piperazin-1-yl)isonicotinate (0.015 g, 11% yield) as an off-white solid.

LCMS 424.2 [M+H]⁺

Compound 6b. To a stirred solution of methyl 2-(4-(bis(4-fluorophenyl)methyl)piperazin-1-yl)isonicotinate (0.120 g, 0.28 mmol, 1 equiv) in THF and water (1:1)(6 mL), was added LiOH.H₂O (0.018 g, 0.42 mmol, 2.0 equiv). The mixture was allowed to stir at RT for 16 h. Product formation was confirmed by LCMS. The reaction mixture was diluted with water (20 mL), washed with ethyl acetate (15 mL×2). Aqueous layer was separated and freeze dried on lyophilizer to obtain 2-(4-(bis(4-fluorophenyl)methyl)piperazin-1-yl)isonicotinic acid (0.100 g) as an off-white solid.

LCMS 410.2 [M+H]⁺

Compound 6. To a stirring solution of 2-(4-(bis(4-fluorophenyl)methyl)piperazin-1-yl)isonicotinic acid (0.100 g, 0.24 mmol, 1.0 equiv) in DMF (3 ml) were added (S)-4,4-difluoro-1-glycylpyrrolidine-2-carbonitrile hydrochloride (0.068 g, 0.3 mmol, 1.2 equiv), EDC.HCl (0.057 g, 0.3 mmol, 1.2 equiv), HOBT (0.041 g, 0.3 mmol, 1.2 equiv), DMAP (0.001 g) and stirred the reaction mixture at RT for 10 min followed by addition of TEA (0.073 g, 0.72 mmol, 3.0 equiv). The reaction mixture was allowed to stir at RT for 16 h. Product formation was confirmed by LCMS. After completion of reaction, reaction mixture was diluted with water which results into precipitate. The resulting solid was filtered off and washed with ice-cold water, dried under vacuum. Crude product was purified by flash chromatography to obtain (S)-2-(4-(bis(4-fluorophenyl)methyl)piperazin-1-yl)-N-(2-(2-cyano-4,4-difluoropyrrolidin-1-yl)-2-oxoethyl)isonicotinamide (0.006 g, 4.26% Yield) as off-white solid.

LCMS 581.4 [M+H]⁺

¹H NMR (400 MHz, DMSO-d₆) δ 8.91 (t, J=6.0 Hz, 1H), 8.21 (d, J=5.2 Hz, 1H), 7.48 (dd, J=8.5, 5.5 Hz, 4H), 7.19-7.10 (m, 5H), 7.01 (d, J=5.3 Hz, 1H), 5.08 (dd, J=9.4, 2.8 Hz, 1H), 4.43 (s, 1H), 4.28 (td, J=11.3, 10.4, 5.9 Hz, 1H), 4.11 (qd, J=14.2, 11.8, 4.4 Hz, 3H), 3.55 (t, J=4.8 Hz, 4H), 3.31 (s, 2H), 2.98-2.72 (m, 2H), 2.40 (t, J=4.9 Hz, 5H), 1.54 (s, 1H), 1.38-1.16 (m, 9H), 0.85 (t, J=6.5 Hz, 1H).

### Example S7

### Synthesis of (S)—N-(2-(2-cyano-4,4-difluoropyrrolidin-1-yl)-2-oxoethyl)-3-(2-fluorobenzyl) isonicotinamide

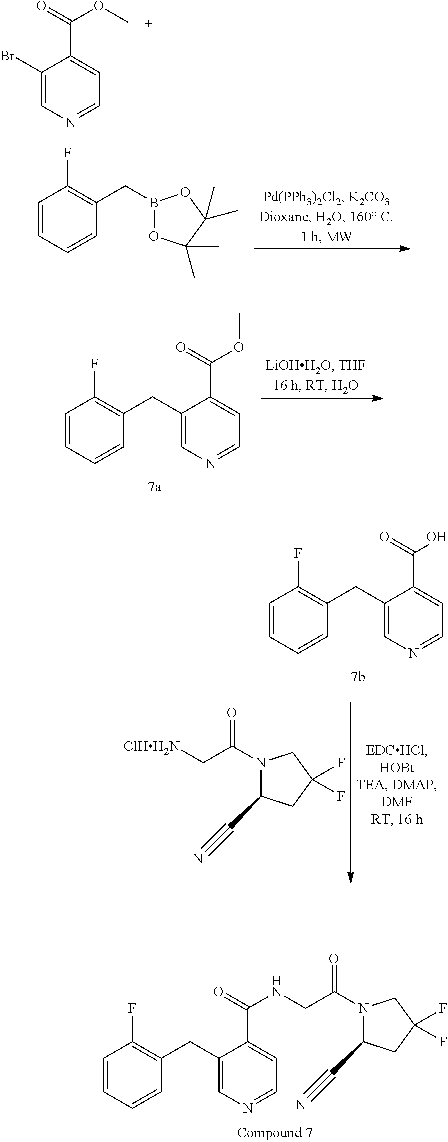

Compound 7a. To a stirred solution of methyl 3-bromoisonicotinate (0.100 g, 0.46 mmol, 1.0 equiv) in Dioxan (4 mL) and water (1 mL) were added K₂CO₃ (0.032 g, 0.23 mmol, 0.5 equiv), 2-(2-fluorobenzyl)-4,4,5,5-tetramethyl-1,3,2-dioxaborolane (0.163 g, 0.69 mmol, 1.5 equiv) and Pd(PPh₃)Cl₂ (0.016 g, 0.023 mmol, 0.05 equiv). The reaction mixture was allowed to heat at 160° C. for 1 h in microwave. Product formation was confirmed by TLC and LCMS. The reaction mixture was diluted with water (10 mL) and extracted with ethyl acetate (15 mL×3). Combined organic layer was washed with brine (30 mL). Organic layer was dried over anhydrous sodium sulphate, filtered and concentrated under reduced pressure. Crude product was purified by normal phase combi-flash chromatography to obtain methyl 3-(2-fluorobenzyl)isonicotinate (0.025 g, 22% Yield) as a yellow semisolid.

LCMS 246.0 [M+H]⁺

Compound 7b. To a stirred solution of methyl 3-(2-fluorobenzyl)isonicotinate (0.130 g, 0.53 mmol, 1 equiv) in THF and water (3:1)(4 mL), was added LiOH.H₂O (0.045 g, 1.06 mmol, 2.0 equiv). The mixture was allowed to stir at RT for 16 h. Product formation was confirmed by LCMS.

The reaction mixture was diluted with water (25 mL), washed with ethyl acetate (20 mL). Aqueous layer was separated and freeze dried on lyophilizer to obtain 3-(2-fluorobenzyl)isonicotinic acid (0.120 g, Quant. Yield).

LCMS 231.9 [M+H]⁺

Compound 7. To a stirring solution of 3-(2-fluorobenzyl)isonicotinic acid (0.130 g, 0.56 mmol, 1.0 equiv) in DMF (4 ml) were added (S)-4,4-difluoro-1-glycylpyrrolidine-2-carbonitrile hydrochloride (0.153 g, 0.68 mmol, 1.2 equiv), EDC.HCl (0.129 g, 0.68 mmol, 1.2 equiv), HOBT (0.092 g, 0.68 mmol, 1.2 equiv), DMAP (0.001 g) and stirred the reaction mixture at RT for 10 min followed by addition of TEA (0.170 g, 1.68 mmol, 3.0 equiv). The reaction mixture was allowed to stir at RT for 16 h. Product formation was confirmed by LCMS. The reaction mixture was diluted with water (20 mL) and extracted with ethyl acetate (30 mL×2). Combined organic layer was washed with water (20 mL×6) and brine (40 mL). Organic layer was dried over anhydrous sodium sulphate, filtered and concentrated under reduced pressure. Crude product was purified by reversed phase HPLC to obtain (S)—N-(2-(2-cyano-4,4-difluoropyrrolidin-1-yl)-2-oxoethyl)-3-(2-fluorobenzyl)isonicotinamide (0.002 g) as an off white solid.

LCMS 403.3 [M+H]⁺

¹H NMR (400 MHz, DMSO-d₆) δ 8.95 (t, J=5.8 Hz, 1H), 8.54 (d, J=5.0 Hz, 1H), 8.41 (s, 1H), 7.38 (d, J=4.9 Hz, 1H), 7.19 (ddt, J=39.1, 20.6, 7.2 Hz, 4H), 5.21-5.03 (m, 1H), 4.43-3.99 (m, 6H), 3.02-2.73 (m, 2H).

### Example S8

### Synthesis of (S)—N-(2-(2-cyano-4,4-difluoropyrrolidin-1-yl)-2-oxoethyl)-3-(4-fluorobenzyl) isonicotinamide

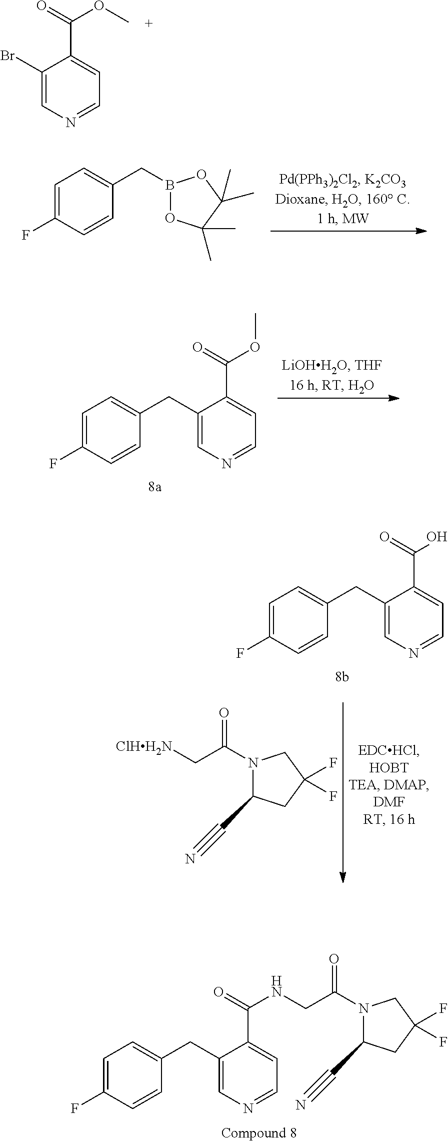

Compound 8a. To a stirred solution of methyl 3-bromoisonicotinate (0.100 g, 0.46 mmol, 1.0 equiv) in Dioxane (4 ml) and water (1 ml) were added K₂CO₃ (0.032 g, 0.23 mmol, 0.5 equiv), 2-(4-fluorobenzyl)-4,4,5,5-tetramethyl-1,3,2-dioxaborolane (0.163 g, 0.69 mmol, 1.5 equiv) and Pd(PPh₃)Cl₂ (0.016 g, 0.023 mmol, 0.05 equiv). The reaction mixture was allowed to heat at 160° C. for 1 h in microwave. Product formation was confirmed by TLC and LCMS. The reaction mixture was diluted with water (10 mL) and extracted with ethyl acetate (15 mL×3). Combined organic layer was washed with brine (30 mL). Organic layer was dried over anhydrous sodium sulphate, filtered and concentrated under reduced pressure. Crude product was purified by normal phase combi-flash chromatography to obtain to obtain methyl 3-(4-fluorobenzyl)isonicotinate (0.025 g, 22% Yield) as a yellow semi solid.

LCMS 246.0 [M+H]⁺

Compound 8b. To a stirred solution of methyl 3-(4-fluorobenzyl)isonicotinate (0.200 g, 0.82 mmol, 1 equiv) in THF and water (4:1)(5 mL), was added LiOH.H₂O (0.069 g, 1.64 mmol, 2.0 equiv). The mixture was allowed to stir at RT for 16 h. Product formation was confirmed by LCMS. The reaction mixture was diluted with water (25 mL), washed with ethyl acetate (20 mL). Aqueous layer was separated and freeze dried on lyophilizer to obtain 3-(4-fluorobenzyl)isonicotinic acid (0.180 g) as an off-white solid.

LCMS 231.9 [M+H]⁺

Compound 8. To a stirring solution of 3-(4-fluorobenzyl)isonicotinic acid (0.250 g, 1.08 mmol, 1.0 equiv) in DMF (4 ml) were added (S)-4,4-difluoro-1-glycylpyrrolidine-2-carbonitrile hydrochloride (0.293 g, 1.3 mmol, 1.2 equiv), EDC.HCl (0.247 g, 1.3 mmol, 1.2 equiv), HOBT (0.176 g, 1.3 mmol, 1.2 equiv), DMAP (0.001 g) and stirred the reaction mixture at RT for 10 min followed by addition of TEA (0.327 g, 3.24 mmol, 3.0 equiv). The reaction mixture was allowed to stir at RT for 16 h. Product formation was confirmed by LCMS. The reaction mixture was diluted with water (20 mL) and extracted with ethyl acetate (30 mL×2). Combined organic layer was washed with water (20 mL×5) and brine (40 mL). Organic layer was dried over anhydrous sodium sulphate, filtered and concentrated under reduced pressure. Crude product was purified by reverse phase HPLC to obtain pure (S)—N-(2-(2-cyano-4,4-difluoropyrrolidin-1-yl)-2-oxoethyl)-3-(4-fluorobenzyl)isonicotinamide (0.005 g) as an off white solid.

LCMS 403.3 [M+H]⁺

¹H NMR (400 MHz, DMSO-d₆) δ 8.96 (t, J=5.8 Hz, 1H), 8.54 (s, 1H), 8.51 (d, J=5.0 Hz, 1H), 7.39-7.27 (m, 3H), 7.07 (t, J=8.8 Hz, 2H), 5.13 (dd, J=9.4, 2.7 Hz, 1H), 4.28 (td, J=12.4, 6.4 Hz, 1H), 4.21-4.03 (m, 5H), 2.99-2.74 (m, 2H).

### Example S9

### Synthesis of (S,E)-3-(4-chlorostyryl)-N-(2-(2-cyano-4,4-difluoropyrrolidin-1-yl)-2-oxoethyl)isonicotinamide

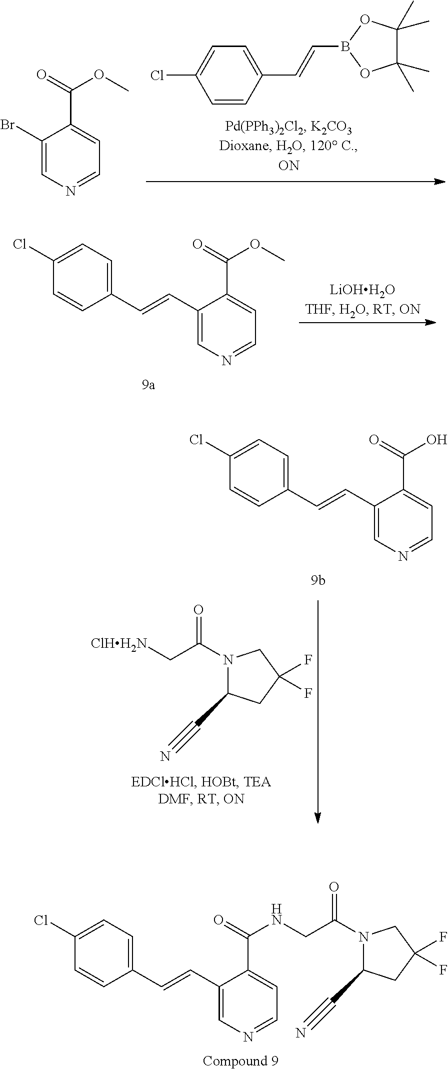

Compound 9a. To a solution of ethyl 3-bromoisonicotinate (0.2 g, 0.925 mmol, 1.0 equiv) in Dioxan (8 mL) and water (2 mL) was added (E)-2-(4-chlorostyryl)-4,4,5,5-tetramethyl-1,3,2-dioxaborolane (0.366 g, 1.39 mmol, 1.5 equiv), K₂CO₃ (0.384 g, 2.78 mmol, 3.0 equiv) and resulting reaction mixture was purged with N₂ gas for 10 minute, followed by the addition of Pd(PPh₃)Cl₂ (0.033 g, 0.046 mmol. 0.05 equiv). The resulting reaction mixture was heated at 120° C. for overnight. Product formation was confirmed by LCMS. After the completion of reaction, the mixture was filtered through celite bed, washed with ethyl acetate (100 mL). Filtrate was concentrated under reduced pressure. The crude product obtained was purified by flash chromatography (0-20% ethyl acetate in hexane as an eluent) to methyl (E)-3-(4-chlorostyryl)isonicotinate (0.120 g, 47.43% yield) as an yellow solid.

LCMS 274.5 [M+H]⁺

¹H NMR (400 MHz, DMSO-d₆) δ 9.11 (s, 1H), 8.64 (d, J=5.3 Hz, 1H), 7.78-7.67 (m, 2H), 7.63 (d, J=8.3 Hz, 2H), 7.48 (d, J=8.3 Hz, 2H), 7.36 (d, J=16.7 Hz, 1H), 3.91 (s, 3H).

Compound 9b. To a stirred solution of methyl (E)-3-(4-chlorostyryl)isonicotinate (0.1 g, 0.366 mmol, 1.0 equiv) in THF (5 mL) and water (5 mL), was added LiOH (0.023 g, 0.0.549 mmol, 1.5 equiv). The mixture was allowed to stir at RT for overnight. Product formation was confirmed by LCMS and ¹H NMR Spectroscopy. The reaction mixture was concentrated and diluted with water (10 mL) and washed with ethyl acetate (10 mL×2). Aqueous layer was separated and freeze dried on lyophilyzer to obtain (E)-3-(4-chlorostyryl)isonicotinic acid (Quant. Yield) as an off white solid.

LCMS 260.0 [M+H]⁺

¹H NMR (400 MHz, DMSO-d₆) δ 8.80 (s, 1H), 8.30 (d, J=4.8 Hz, 1H), 7.87 (d, J=16.7 Hz, 1H), 7.63-7.49 (m, J=8.3 Hz, 2H), 7.47-7.35 (m, J=8.8 Hz, 2H), 7.28 (d, J=4.8 Hz, 1H), 7.19 (d, J=16.7 Hz, 1H).

Compound 9. To a stirred solution of (E)-3-(4-chlorostyryl)isonicotinic acid (0.11 g, 0.424 mmol, 1.0 equiv) in DMF (3 mL), was added (S)-4,4-difluoro-1-glycylpyrrolidine-2-carbonitrile hydrochloride (0.096 g, 0.424 mmol, 1.0 equiv), EDCI.HCl (0.122 g, 0.636 mmol, 1.5 equiv) & HOBt (0.086 g, 0.48 mmol, 1.5 equiv). The mixture was allowed to stir at RT for 10 min. Triethylamine (0.25 mL) was added and the mixture was allowed to stir at RT for overnight. Product formation was confirmed by LCMS and TLC. After completion of reaction, the mixture was diluted with water (20 mL) and extracted with ethyl acetate (20 mL×2). Combined organic extracts were washed with water (20 mL×4), dried over anhydrous Na₂SO₄ and concentrated. The crude product obtained was crystallized with ether to obtain Synthesis of (S,E)-3-(4-chlorostyryl)-N-(2-(2-cyano-4,4-difluoropyrrolidin-1-yl)-2-oxoethyl)isonicotinamide (0.18 g, 98.36% Yield) as an off white solid.

LCMS 431.2 [M+H]⁺

¹H NMR (400 MHz, DMSO-d₆) δ 9.14 (s, 1H), 9.06-8.94 (m, 1H), 8.54 (d, J=4.8 Hz, 1H), 7.83-7.61 (m, 3H), 7.60-7.41 (m, 3H), 7.36 (d, J=4.8 Hz, 1H), 5.25-5.10 (m, 1H), 4.31 (d, J=11.8 Hz, 1H), 4.24-3.96 (m, 3H), 2.94 (br. s., 1H), 2.92-2.78 (m, 1H).

### Example S10

### Synthesis of (S)-3-(4-chlorophenethyl)-N-(2-(2-cyano-4,4-difluoropyrrolidin-1-yl)-2-oxoethyl)isonicotinamide

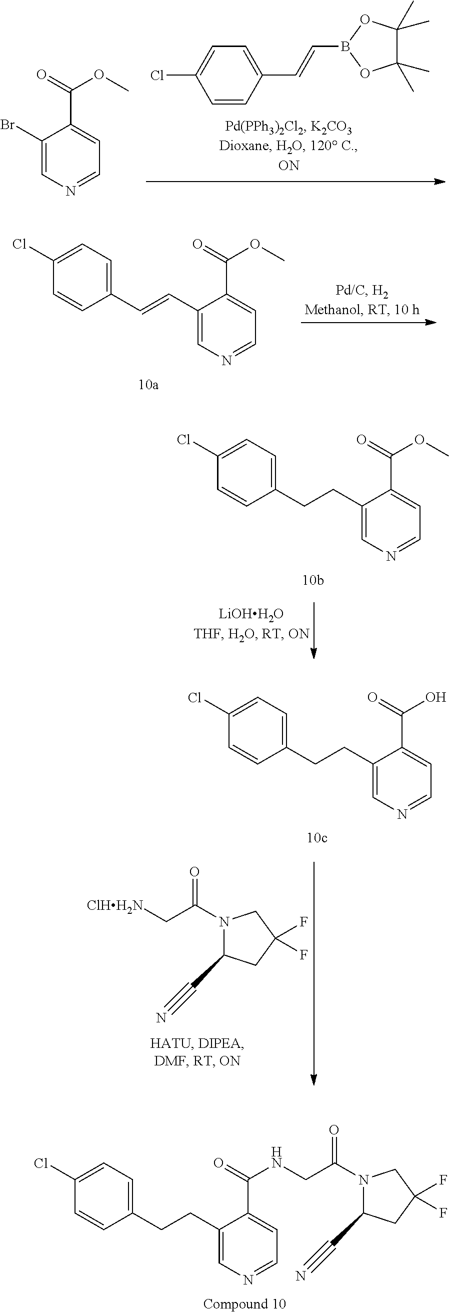

Compound 10a. To a solution of ethyl 3-bromoisonicotinate (0.2 g, 0.925 mmol, 1.0 equiv) in dioxane (8 mL) and water (2 mL) was added (E)-2-(4-chlorostyryl)-4,4,5,5-tetramethyl-1,3,2-dioxaborolane (0.366 g, 1.39 mmol, 1.5 equiv), K₂CO₃ (0.384 g, 2.78 mmol, 3.0 equiv) and resulting reaction mixture purged with N₂ gas for 10 minute, followed by the addition of Pd(PPh₃)Cl₂ (0.033 g, 0.046 mmol. 0.05 equiv). The resulting reaction mixture was heated at 120° C. for overnight. Product formation was confirmed by LCMS. After the completion of reaction, the mixture was filtered through celite bed, washed with ethyl acetate (100 mL). Filtrate was concentrated under reduced pressure. The crude product obtained was purified by flash chromatography (0-20% ethyl acetate in hexane as an eluent) to methyl (E)-3-(4-chlorostyryl)isonicotinate (0.120 g, 47.43% yield) as an yellow solid.

LCMS 274.5 [M+H]⁺

¹H NMR (400 MHz, DMSO-d₆) δ 9.11 (s, 1H), 8.64 (d, J=5.3 Hz, 1H), 7.78-7.67 (m, 2H), 7.63 (d, J=8.3 Hz, 2H), 7.48 (d, J=8.3 Hz, 2H), 7.36 (d, J=16.7 Hz, 1H), 3.91 (s, 3H).

Compound 10b. To a solution of methyl (E)-3-(4-chlorostyryl)isonicotinate (0.12 g, 0.44 mmol, 1.0 equiv) in methanol (15 mL) was purged with N₂ gas for 10 minute, followed by the addition of Pd/C (0.030 g). Then resulting reaction mixture was purged with H₂ gas for 10 h. Product formation was confirmed by LCMS. After the completion of reaction, the mixture was filtered through celite bed, washed with methanol (30 mL). Filtrate was concentrated under reduced pressure to obtain methyl 3-(4-chlorophenethyl)isonicotinate (0.105 g, 86.77% yield) as a white solid.

LCMS 276.2 [M+H]⁺

¹H NMR (400 MHz, DMSO-d₆) δ 8.63-8.51 (m, 2H), 7.67 (d, J=4.9 Hz, 1H), 7.33 (d, J=8.3 Hz, 2H), 7.20 (d, J=8.3 Hz, 2H), 3.96-3.78 (m, 3H), 3.15 (dd, J=6.6, 9.0 Hz, 2H), 2.90-2.79 (m, 2H).

Compound 10c. To a stirred solution of methyl 3-(4-chlorophenethyl)isonicotinate (0.105 g, 0.38 mmol, 1.0 equiv) in THF (5 mL) and water (5 mL), was added LiOH.H₂O (0.024 g, 0.0.57 mmol, 1.5 equiv). The mixture was allowed to stir at RT for overnight. Product formation was confirmed by LCMS and ¹H NMR Spectroscopy. The reaction mixture was concentrated and diluted with water (10 mL) and washed with ethyl acetate (10 mL×2). Aqueous layer was separated and freeze dried on lyophilyzer to obtain (E)-3-(4-chlorostyryl)isonicotinic acid (Quant. Yield) as a white solid.

LCMS 262.1 [M+H]⁺

Compound 10. To a stirred solution of (E)-3-(4-chlorostyryl)isonicotinic acid (0.07 g, 0.268 mmol, 1.0 equiv) in DMF (3 mL), was added (S)-4,4-difluoro-1-glycylpyrrolidine-2-carbonitrile hydrochloride (0.06 g, 0.268 mmol, 1.0 equiv), HATU (0.152 g, 0.402 mmol, 1.5 equiv). The mixture was allowed to stir at RT for 10 min. DIPEA (0.2 mL) was added and the mixture was allowed to stir at RT for overnight. Product formation was confirmed by LCMS and TLC. After completion of reaction, the mixture was diluted with water (20 mL) and extracted with ethyl acetate (20 mL×2). Combined organic extracts were washed with water (20 mL×4), dried over anhydrous Na₂SO₄ and concentrated. The crude product obtained was purified by flash chromatography (5% MeOH in DCM as an eluent) to obtain (S)-3-(4-chlorophenethyl)-N-(2-(2-cyano-4,4-difluoropyrrolidin-1-yl)-2-oxoethyl)isonicotinamide (0.015 g, 13% Yield) as an off white solid.

LCMS 433.2 [M+H]⁺

¹H NM (DMSO-d₆, 400 MHz): δ 9.01 (br. s., 1H), 8.57 (d, J=4.9 Hz, 1H), 8.53 (s, 1H), 7.46 (d, J=4.9 Hz, 1H), 7.30 (q, J=8.3 Hz, 4H), 5.13 (d, J=9.8 Hz, 1H), 4.32 (br. s., 1H), 4.12-4.22 (m, 2H), 4.10 (br. s., 1H), 2.97-3.13 (m, 2H), 2.84-2.95 (m, 2H), 2.81 (br. s., 2H).

### Example S11

### Synthesis of (S)—N-(2-(2-cyano-4,4-difluoropyrrolidin-1-yl)-2-oxoethyl)-3-(4-fluorophenethyl)isonicotinamide

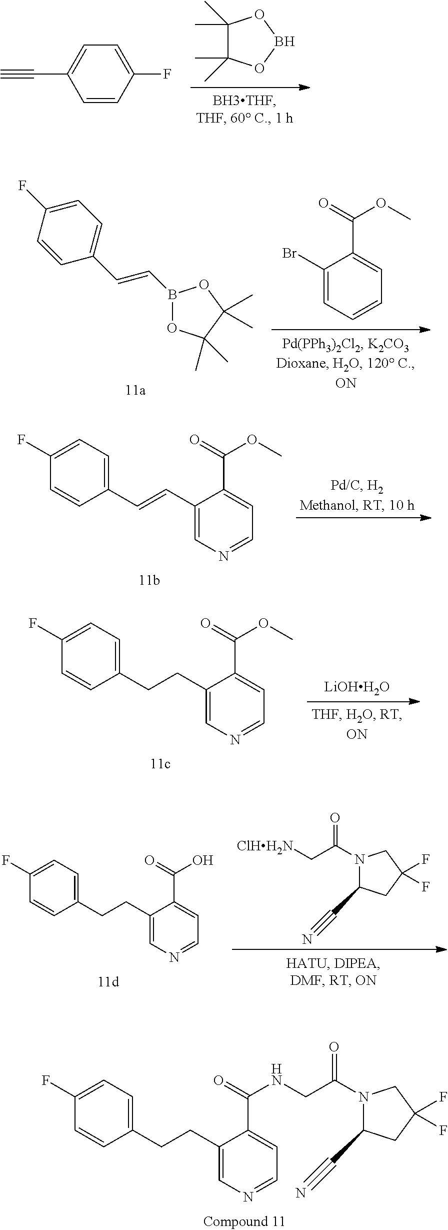

Compound 11a. To a stirred solution 1-ethynyl-4-fluorobenzene (500 mg, 4.16 mmol, 1.0 equiv) in THF (10 mL), was added 4,4,5,5-tetramethyl-1,3,2-dioxaborolane (800 mg, 6.25 mmol, 1.5 equiv), BH₃.THF (2 ml, 2.08 mmol, 0.5 equiv) flushed with an nitrogen atmosphere at RT. The mixture was allowed to heat for 1 hour at 60 degree. Product formation was confirmed by TLC. After completion of reaction, the mixture was diluted with NH₄Cl solution (20 mL) and extracted with ethyl acetate (20 mL×2). Combined organic extracts were washed with water (20 mL×4), dried over anhydrous Na₂SO₄ and concentrated. The crude product obtained was purified by flash chromatography (10% ethyl acetate) to obtain (E)-2-(4-fluorostyryl)-4,4,5,5-tetramethyl-1,3,2-dioxaborolane (350 mg, 13% Yield) as an yellow oil.

LCMS 249.2 [M+H]⁺

¹HNMR (DMSO-d₆, 400 MHz) δ 7.65 (dd, J=8.3, 5.7 Hz, 2H), 7.29 (d, J=18.4 Hz, 1H), 7.20 (t, J=8.8 Hz, 2H), 6.09 (d, J=18.4 Hz, 1H), 1.13-1.32 ppm (m, 12H).

Compound 11b. To a solution of methyl 3-bromoisonicotinate (0.2 g, 0.921 mmol, 1.0 equiv) in Dioxan (4 mL) was added (E)-2-(4-fluorostyryl)-4,4,5,5-tetramethyl-1,3,2-dioxaborolane (0.3 g, 1.38 mmol, 1.5 equiv), K₂CO₃ (0.384 g, 2.78 mmol, 3.0 equiv) and resulting reaction mixture purged with N₂ gas for 10 minute, followed by the addition of Pd(PPh₃)Cl₂ (0.032 g, 0.046 mmol. 0.05 equiv). The resulting reaction mixture was heated at 120° C. for overnight. Product formation was confirmed by LCMS. After the completion of reaction, the mixture was filtered through celite bed, washed with ethyl acetate (100 mL). Filtrate was concentrated under reduced pressure. The crude product obtained was purified by flash chromatography (0-20% ethyl acetate in hexane as an eluent) to methyl (E)-3-(4-flurostyryl)isonicotinate (0.05 g, 47.43% yield) as an yellow solid.

LCMS 258.0 [M+H]⁺

¹H NMR (DMSO-d₆, 400 MHz) δ 9.07 (s, 1H), 8.59 (d, J=4.9 Hz, 1H), 7.48-7.76 (m, 4H), 7.33 (d, J=16.6 Hz, 1H), 7.22 (t, J=8.6 Hz, 2H), 3.87 ppm (s, 3H).

Compound 11c. To a solution of methyl (E)-3-(4-flurostyryl)isonicotinate (0.10 g, 0.389 mmol, 1.0 equiv) in methanol (15 mL) was purged with N₂ gas for 10 minute, followed by the addition of Pd/C (0.030 g). Then resulting reaction mixture was purged with H₂ gas for 10 h. Product formation was confirmed by LCMS. After the completion of reaction, the mixture was filtered through celite bed, washed with methanol (30 mL). Filtrate was concentrated under reduced pressure to obtained methyl 3-(4-flurophenethyl)isonicotinate (0.1 g, 86.77% yield) as a white solid.

LCMS 260.2 [M+H]⁺

¹H NMR (DMSO-d₆, 400 MHz) δ 8.57 (br. s., 2H), 7.65 (d, J=4.9 Hz, 1H), 7.16-7.29 (m, 2H), 7.00-7.12 (m, 2H), 3.88 (s, 3H), 3.06-3.19 (m, 2H), 2.77-2.88 ppm (m, 2H).

Compound 11d. To a stirred solution of methyl 3-(4-flurophenethyl)isonicotinate (0.100 g, 0.38 mmol, 1.0 equiv) in THF (5 mL) and water (5 mL), was added LiOH.H₂O (0.024 g, 0.583 mmol, 1.5 equiv). The mixture was allowed to stir at RT for overnight. Product formation was confirmed by LCMS and ¹H NMR Spectroscopy. The reaction mixture was concentrated and diluted with water (10 mL) and washed with ethyl acetate (10 mL×2). Aqueous layer was separated and freeze dried on lyophilyzer to obtain 3-(4-fluorophenethyl)isonicotinic acid (Quant. Yield) as white solid.

LCMS 246.2 [M+H]⁺

¹H NMR (DMSO-d₆, 400 MHz) δ 8.21 (s, 1H), 8.13 (s, 1H), 7.13-7.29 (m, 3H), 7.06 (t, J=8.8 Hz, 2H), 2.90-3.03 (m, 2H), 2.72-2.87 ppm (m, 2H).

Compound 11. To a stirred solution of (E)-3-(4-flurostyryl)isonicotinic acid (0.09 g, 0.367 mmol, 1.0 equiv) in DMF (3 mL), was added (S)-4,4-difluoro-1-glycylpyrrolidine-2-carbonitrile hydrochloride (0.08 g, 0.367 mmol, 1.0 equiv), HATU (0.209 g, 0.550 mmol, 1.5 equiv). The mixture was allowed to stir at RT for 10 min. DIPEA (0.2 mL) was added and the mixture was allowed to stir at RT for overnight. Product formation was confirmed by LCMS and TLC. After completion of reaction, the mixture was diluted with water (20 mL) and extracted with ethyl acetate (20 mL×2). Combined organic extracts were washed with water (20 mL×4), dried over anhydrous Na₂SO₄ and concentrated. The crude product obtained was purified by flash chromatography (5% MeOH in DCM as an eluent) to obtain (S)—N-(2-(2-cyano-4,4-difluoropyrrolidin-1-yl)-2-oxoethyl)-3-(4-fluorophenethyl)isonicotinamide (0.060 g, 13% Yield) as an off white solid.

LCMS 417.3 [M+H]⁺

¹H NMR (DMSO-d₆, 400 MHz) δ 8.94 (br. s., 1H), 8.35-8.55 (m, 2H), 7.34 (d, J=4.4 Hz, 1H), 7.16-7.31 (m, 2H), 7.07 (t, J=8.8 Hz, 2H), 5.13 (d, J=7.8 Hz, 1H), 4.31 (br. s., 1H), 3.95-4.20 (m, 3H), 3.01 (d, J=9.3 Hz, 2H), 2.87 (d, J=8.8 Hz, 2H), 2.80 ppm (br. s., 2H).

### Example S12

### Synthesis of (S,E)-N-(2-(2-cyano-4,4-difluoropyrrolidin-1-yl)-2-oxoethyl)-3-(4-fluorostyryl)isonicotinamide

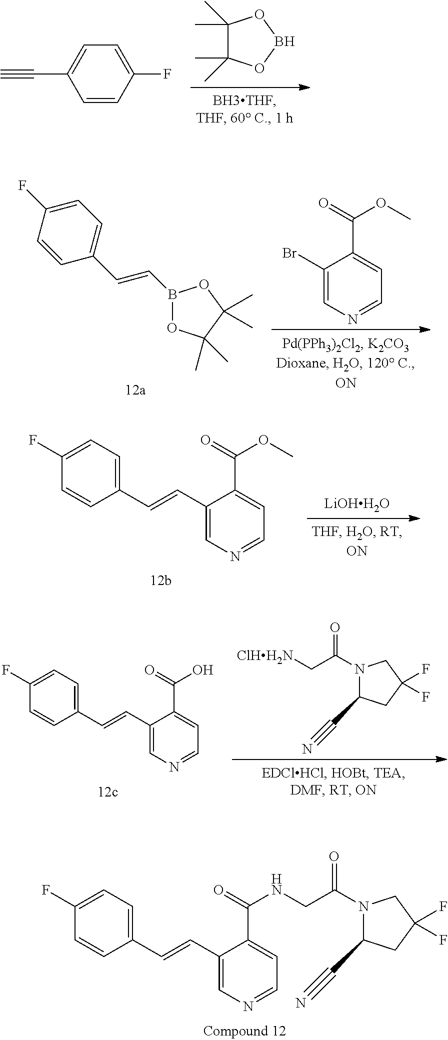

Compound 12a. To a stirred solution 1-ethynyl-4-fluorobenzene (500 mg, 4.16 mmol, 1.0 equiv) in THF (10 mL), was added 4,4,5,5-tetramethyl-1,3,2-dioxaborolane (800 mg, 6.25 mmol, 1.5 equiv), BH₃.THF (2 ml, 2.08 mmol, 0.5 equiv) flushed with an nitrogen atmosphere at RT. The mixture was allowed to heat for 1 hour at 60 degree. Product formation was confirmed by TLC. After completion of reaction, the mixture was diluted with NH₄Cl solution (20 mL) and extracted with ethyl acetate (20 mL×2). Combined organic extracts were washed with water (20 mL×4), dried over anhydrous Na₂SO₄ and concentrated. The crude product obtained was purified by flash chromatography (10% ethyl acetate) to obtain desired (E)-2-(4-fluorostyryl)-4,4,5,5-tetramethyl-1,3,2-dioxaborolane (350 mg, 13% Yield) as an yellow oil.

LCMS 249.2 [M+H]⁺

¹H NMR (DMSO-d₆, 400 MHz) δ 7.65 (dd, J=8.3, 5.7 Hz, 2H), 7.29 (d, J=18.4 Hz, 1H), 7.20 (t, J=8.8 Hz, 2H), 6.09 (d, J=18.4 Hz, 1H), 1.13-1.32 ppm (m, 12H).

Compound 12b. To a solution of methyl 3-bromoisonicotinate (0.2 g, 0.921 mmol, 1.0 equiv) in dioxane (4 mL) was added (E)-2-(4-flurostyryl)-4,4,5,5-tetramethyl-1,3,2-dioxaborolane (0.3 g, 1.38 mmol, 1.5 equiv), K₂CO₃ (0.384 g, 2.78 mmol, 3.0 equiv) and resulting reaction mixture purged with N₂ gas for 10 minute, followed by the addition of Pd(PPh₃)Cl₂ (0.032 g, 0.046 mmol. 0.05 equiv). The resulting reaction mixture was heated at 120° C. for overnight. Product formation was confirmed by LCMS. After the completion of reaction, the mixture was filtered through celite bed, washed with ethyl acetate (100 mL). Filtrate was concentrated under reduced pressure. The crude product obtained was purified by flash chromatography (0-20% ethyl acetate in hexane as an eluent) to methyl (E)-3-(4-flurostyryl)isonicotinate (0.05 g, 47.43% yield) as an yellow solid.

LCMS 258.0 [M+H]⁺

¹H NMR (DMSO-d₆, 400 MHz) δ 9.07 (s, 1H), 8.59 (d, J=4.9 Hz, 1H), 7.48-7.76 (m, 4H), 7.33 (d, J=16.6 Hz, 1H), 7.22 (t, J=8.6 Hz, 2H), 3.87 ppm (s, 3H).

Compound 12c. To a stirred solution of methyl (E)-3-(4-flurostyryl)isonicotinate (0.300 g, 1.167 mmol, 1.0 equiv) in THF (4 mL) and water (4 mL), was added LiOH.H₂O (0.074 g, 1.751 mmol, 1.5 equiv). The mixture was allowed to stir at RT for overnight. Product formation was confirmed by LCMS and ¹H NMR Spectroscopy. The reaction mixture was concentrated and diluted with water (20 mL) and washed with ethyl acetate (10 mL×2). Aqueous layer was separated and freeze dried on lyophilyzer to obtain (E)-3-(4-fluorostyryl)isonicotinic acid (Quant. Yield) as a white solid.

LCMS 244.0 [M+H]⁺

Compound 12. To a stirred solution of (E)-3-(4-fluorostyryl)isonicotinic acid (0.1 g, 0.412 mmol, 1.0 equiv) in DMF (5 mL), was added (S)-4,4-difluoro-1-glycylpyrrolidine-2-carbonitrile hydrochloride (0.093 g, 0.412 mmol, 1.0 equiv), EDCI.HCl (0.119 g, 0.618 mmol, 1.5 equiv) & HOBt (0.084 g, 0.618 mmol, 1.5 equiv). The mixture was allowed to stir at RT for 10 min. TEA (0.3 mL) was added and the mixture was allowed to stir at RT for overnight. Product formation was confirmed by LCMS and TLC. After completion of reaction, the mixture was diluted with water (20 mL) and extracted with ethyl acetate (20 mL×2). Combined organic extracts were washed with water (20 mL×4), dried over anhydrous Na₂SO₄ and concentrated. The crude product obtained was purified by flash chromatography (5% MeOH in DCM as an eluent) to obtain (S,E)-N-(2-(2-cyano-4,4-difluoropyrrolidin-1-yl)-2-oxoethyl)-3-(4-fluorostyryl)isonicotinamide (0.1 g, 58.8% Yield) as an white solid.

LCMS 415.2 [M+H]⁺

¹H NMR (DMSO-d₆, 400 MHz) δ 9.13 (s, 1H), 8.99 (t, J=5.9 Hz, 1H), 8.53 (d, J=4.8 Hz, 1H), 7.76 (dd, J=8.6, 5.5 Hz, 2H), 7.64 (d, J=16.7 Hz, 1H), 7.48 (d, J=16.7 Hz, 1H), 7.36 (d, J=5.3 Hz, 1H), 7.11-7.26 (m, 2H), 5.19 (dd, J=9.4, 2.9 Hz, 1H), 4.25-4.36 (m, 1H), 4.00-4.24 (m, 3H), 3.92 (d, J=5.7 Hz, 1H), 2.78-2.97 ppm (m, 2H).

### Example S13

### Synthesis of (S,E)-N-(2-(2-cyano-4,4-difluoropyrrolidin-1-yl)-2-oxoethyl)-3-(2-(4-fluorophenyl)prop-1-en-1-yl)isonicotinamide and (S)—N-(2-(2-cyano-4,4-difluoropyrrolidin-1-yl)-2-oxoethyl)-3-(2-(4-fluorophenyl)allyl)isonicotinamide

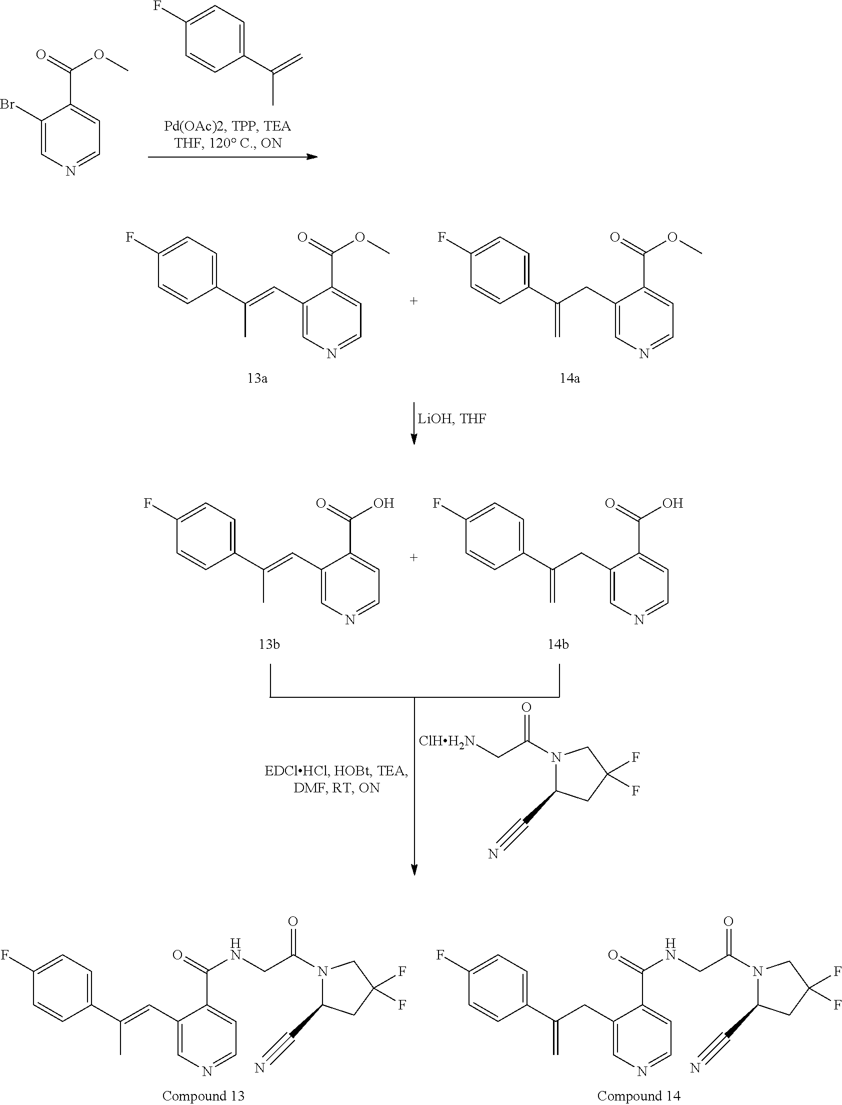

Compounds 13a and 14a. To a solution of methyl 3-bromoisonicotinate (1.0 g, 4.629 mmol, 1.0 equiv) in THF (20 mL) was added 1-fluoro-4-(prop-1-en-2-yl)benzene (1.0 g, 6.94 mmol, 1.5 equiv), TEA (1.4 mL, 10.18 mmol, 2.2 equiv), followed by the addition of Pd(OAc)₂ (0.52 g, 0.23 mmol. 0.05 equiv) & Triphenylphospine (0.12 g, 0.4629 mmol. 0.1 equiv) The resulting reaction mixture was heated at 120° C. for overnight. Product formation was confirmed by LCMS and TLC. After the completion of reaction, the mixture was filtered through celite bed, washed with ethyl acetate (100 mL). Filtrate was concentrated under reduced pressure. The crude product obtained was purified by flash chromatography (0-20% ethyl acetate in hexane as an eluent) to obtain the mixture of methyl (E)-3-(2-(4-fluorophenyl)prop-1-en-1-yl)isonicotinate and methyl 3-(2-(4-fluorophenyl)allyl)isonicotinate (0.05 g, 4%) as an yellow oil.

LCMS 272.1 [M+H]⁺

Compounds 13b and 14b. To a stirred solution of mixture of methyl (E)-3-(2-(4-fluorophenyl)prop-1-en-1-yl)isonicotinate and methyl 3-(2-(4-fluorophenyl)allyl)isonicotinate (0.067 g, 0.24 mmol, 1.0 equiv) in THF (5 mL) and water (5 mL), was added LiOH.H₂O (0.016 g, 0.371 mmol, 1.5 equiv). The mixture was allowed to stir at RT for overnight. Product formation was confirmed by LCMS. The reaction mixture was concentrated and diluted with water (10 mL) and washed with ethyl acetate (5 mL×2). Aqueous layer was acidify with 6N HCl (pH-5 to 6), solid precipitate was filtered off and dried under vacuum to obtain mixture of (E)-3-(2-(4-fluorophenyl)prop-1-en-1-yl)isonicotinic acid and 3-(2-(4-fluorophenyl)allyl)isonicotinic acid (45 mg, 73%) as off white solid.

LCMS 258.0 [M+H]⁺

Compounds 13 and 14. To a stirred solution of (E)-3-(2-(4-fluorophenyl)prop-1-en-1-yl)isonicotinic acid and 3-(2-(4-fluorophenyl)allyl)isonicotinic acid (0.045 g, 0.175 mmol, 1.0 equiv) in DMF (2 mL), was added (S)-4,4-difluoro-1-glycylpyrrolidine-2-carbonitrile hydrochloride (0.040 g, 0.175 mmol, 1.0 equiv), EDCI.HCl (0.05 g, 0.263 mmol, 1.5 equiv) & HOBt (0.036 g, 0.263 mmol, 1.5 equiv). The mixture was allowed to stir at RT for 10 min. TEA (0.2 mL) was added and the mixture was allowed to stir at RT for overnight. Product formation was confirmed by LCMS and TLC. After completion of reaction, the mixture was diluted with water (10 mL) and extracted with ethyl acetate (10 mL×2). Combined organic extracts were washed with water (10 mL×4), dried over anhydrous Na₂SO₄ and concentrated. The crude product obtained was purified by flash chromatography (5% MeOH in DCM as an eluent) to obtain mixture of compounds which is further purified by reversed HPLC to obtain (S,E)-N-(2-(2-cyano-4,4-difluoropyrrolidin-1-yl)-2-oxoethyl)-3-(2-(4-fluorophenyl)prop-1-en-1-yl)isonicotinamide (0.022 g) as yellow solid and (S)—N-(2-(2-cyano-4,4-difluoropyrrolidin-1-yl)-2-oxoethyl)-3-(2-(4-fluorophenyl)allyl)isonicotinamide (0.008 g) as white solid.

LCMS 429.2 [M+H]⁺

¹H NMR (Compound 13) (400 MHz, DMSO-d₆) δ 8.92 (br. s., 1H), 8.73 (br. s., 1H), 8.64 (br. s., 1H), 7.74-7.57 (m, 2H), 7.55 (d, J=4.8 Hz, 1H), 7.23 (t, J=8.8 Hz, 2H), 7.07-6.97 (m, 1H), 5.12 (d, J=9.2 Hz, 1H), 4.30 (d, J=11.0 Hz, 1H), 4.15 (d, J=4.4 Hz, 1H), 4.08 (d, J=9.6 Hz, 2H), 3.01-2.73 (m, 3H), 2.17 (s, 3H).

¹H NMR (Compound 14) (400 MHz, DMSO-d₆) δ 8.89 (br. s., 1H), 8.49 (d, J=4.8 Hz, 1H), 8.43 (s, 1H), 7.54 (dd, J=5.5, 8.6 Hz, 2H), 7.35 (d, J=5.3 Hz, 1H), 7.12 (t, J=8.8 Hz, 2H), 5.47 (s, 1H), 5.13 (d, J=6.1 Hz, 1H), 4.99 (s, 1H), 4.27 (d, J=11.8 Hz, 1H), 4.20-3.95 (m, 3H), 3.17 (d, J=5.7 Hz, 1H), 2.90 (br. s., 1H), 2.82 (d, J=17.5 Hz, 1H), 2.67 (br. s., 1H).

### Example S14

### Synthesis of (S,E)-N-(2-(2-cyano-4,4-difluoropyrrolidin-1-yl)-2-oxoethyl)-3-(4-methoxystyryl)isonicotinamide

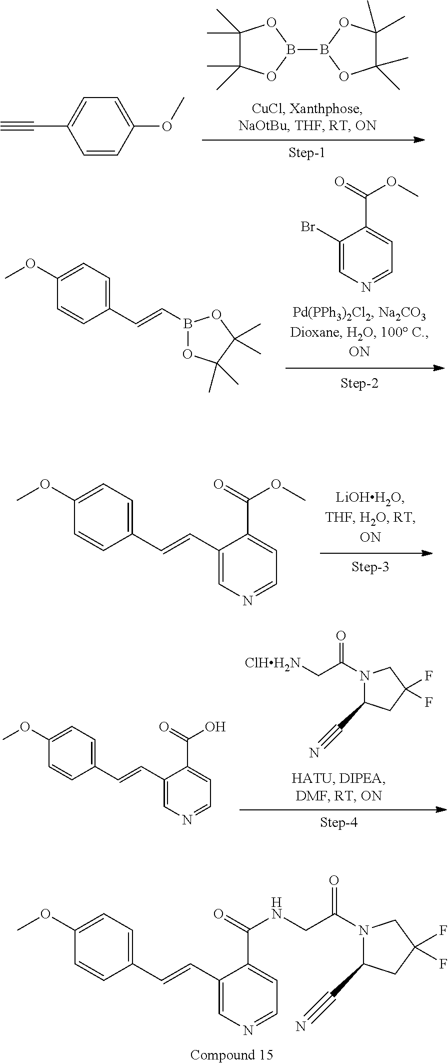

Step 1: Synthesis of (E)-2-(4-methoxystyryl)-4,4,5,5-tetramethyl-1,3,2-dioxaborolane. To a stirred solution 4,4,4′,4′,5,5,5′,5′-octamethyl-2,2′-bi(1,3,2-dioxaborolane) (1.0 g, 4.16 mmol, 1.1 equiv) in dried THF (20 mL), was added CuCl (3.7 mg, 0.038 mmol, 0.01 equiv), Xanthphos (22 mg, 0.038 mmol, 0.01 equiv) and NaOtBu (0.435 g, 4.536 mmol, 1.2 equiv) at RT. The mixture was allowed to stir at RT for 30 minute. 1-ethynyl-4-methoxybenzene (0.5 g, 3.78 mmol, 1.0 equiv) dissolved in THF (5 mL) was added into above reaction mixture and stirred at RT overnight. Product formation was confirmed by TLC. After completion of reaction, the mixture was diluted with water (50 mL) and extracted with ethyl acetate (50 mL×2). Combined organic extracts were washed with water (20 mL×2), dried over anhydrous Na₂SO₄ and concentrated. The crude product was purified by flash chromatography (5% EtOAc in hexane as an eluent) to obtain (E)-2-(4-methoxystyryl)-4,4,5,5-tetramethyl-1,3,2-dioxaborolane (0.8 g, 86.5% Yield) as a yellow oil.

LCMS 261.2 [M+H]⁺

¹H NMR (DMSO-d₆, 400 MHz) δ 7.46-7.60 (m, J=8.8 Hz, 2H), 7.25 (d, J=18.4 Hz, 1H), 6.84-6.97 (m, J=8.8 Hz, 2H), 5.96 (d, J=18.4 Hz, 1H), 3.77 (s, 3H), 1.23 (s, 12H).

Step 2: Synthesis of methyl (E)-3-(4-methoxystyryl)isonicotinate. To a solution of methyl 3-bromoisonicotinate (0.2 g, 0.926 mmol, 1.0 equiv) in dioxane (20 mL) and water (1 mL) was added (E)-2-(4-methoxystyryl)-4,4,5,5-tetramethyl-1,3,2-dioxaborolane (0.361 g, 1.39 mmol, 1.5 equiv), Na₂CO₃ (0.2 g, 1.85 mmol, 2.0 equiv) and resulting reaction mixture purged with N₂ gas for 10 minute, followed by the addition of Pd(PPh₃)Cl₂ (0.033 g, 0.0463 mmol. 0.05 equiv). The resulting reaction mixture was heated at 100° C. for overnight. Product formation was confirmed by LCMS. After the completion of reaction, the mixture was filtered through Celite®, washed with ethyl acetate (100 mL). Filtrate was concentrated under reduced pressure. The crude product was purified by flash chromatography (0-20% ethyl acetate in hexane as an eluent) to obtain methyl (E)-3-(4-methoxystyryl)isonicotinate (0.25 g, 99% yield) as a yellow solid.

LCMS 270.0 [M+H]⁺

¹H NMR (DMSO-d₆, 400 MHz) δ 9.10 (s, 1H), 8.59 (d, J=4.8 Hz, 1H), 7.68 (d, J=4.8 Hz, 1H), 7.45-7.60 (m, 3H), 7.33 (d, J=16.2 Hz, 1H), 6.99 (d, J=8.3 Hz, 2H), 3.91 (s, 2H), 3.67-3.83 (m, 3H).

Step 3: Synthesis of (E)-3-(4-methoxystyryl)isonicotinic acid. To a stirred solution of methyl (E)-3-(4-methoxystyryl)isonicotinate (0.250 g, 0.929 mmol, 1.0 equiv) in THF (10 mL) and water (10 mL), was added LiOH.H₂O (0.056 g, 1.39 mmol, 1.5 equiv). The mixture was allowed to stir at RT for overnight. Product formation was confirmed by LCMS and ¹H NMR Spectroscopy. The reaction mixture was concentrated and diluted with water (20 mL) and washed with ethyl acetate (10 mL×2). Aqueous layer acidify with 6N HCl (pH˜5 to 6), solid precipitate formed was filtered off and dried under vacuum to obtain (E)-3-(4-methoxystyryl)isonicotinic acid (0.15 g, 63.5%) as an yellow solid.

LCMS 255.9 [M+H]⁺

Step 4: Synthesis of (S,E)-N-(2-(2-cyano-4,4-difluoropyrrolidin-1-yl)-2-oxoethyl)-3-(4-methoxystyryl)isonicotinamide. To a stirred solution of (E)-3-(4-methoxystyryl)isonicotinic acid (0.12 g, 0.47 mmol, 1.0 equiv) in DMF (5 mL), was added (S)-4,4-difluoro-1-glycylpyrrolidine-2-carbonitrile hydrochloride (0.158 g, 0.705 mmol, 1.5 equiv), HATU (0.268 g, 0.705 mmol, 1.5 equiv). The mixture was allowed to stir at RT for 10 min. DIPEA (0.4 mL) was added and the mixture was allowed to stir at RT for overnight. Product formation was confirmed by LCMS and TLC. After completion of reaction, the mixture was diluted with water (20 mL) and extracted with ethyl acetate (20 mL×2). Combined organic extracts were washed with water (20 mL×4), dried over anhydrous Na₂SO₄ and concentrated. The crude product was purified by flash chromatography (5% MeOH in DCM as an eluent) followed by reverse phase HPLC purification to obtain (S,E)-N-(2-(2-cyano-4,4-difluoropyrrolidin-1-yl)-2-oxoethyl)-3-(4-methoxystyryl)isonicotinamide (0.06 g, 30% Yield) as a white solid.

LCMS 427.3 [M+H]⁺

¹H NMR (DMSO-d₆, 400 MHz) δ 9.11 (s, 1H), 8.97 (t, J=5.9 Hz, 1H), 8.49 (d, J=4.8 Hz, 1H), 7.57-7.68 (m, J=8.8 Hz, 2H), 7.51 (d, J=16.2 Hz, 1H), 7.41 (d, J=16.7 Hz, 1H), 7.33 (d, J=5.3 Hz, 1H), 6.85-7.03 (m, J=8.8 Hz, 2H), 5.18 (dd, J=9.2, 2.2 Hz, 1H), 4.27-4.38 (m, 1H), 3.99-4.21 (m, 3H), 3.66-3.87 (m, 3H), 2.75-2.98 (m, 2H).

### Example S15

### Synthesis of (S,E)-N-(2-(2-cyano-4,4-difluoropyrrolidin-1-yl)-2-oxoethyl)-3-(2-(4-(trifluoromethoxy)phenyl)prop-1-en-1-yl)isonicotinamide

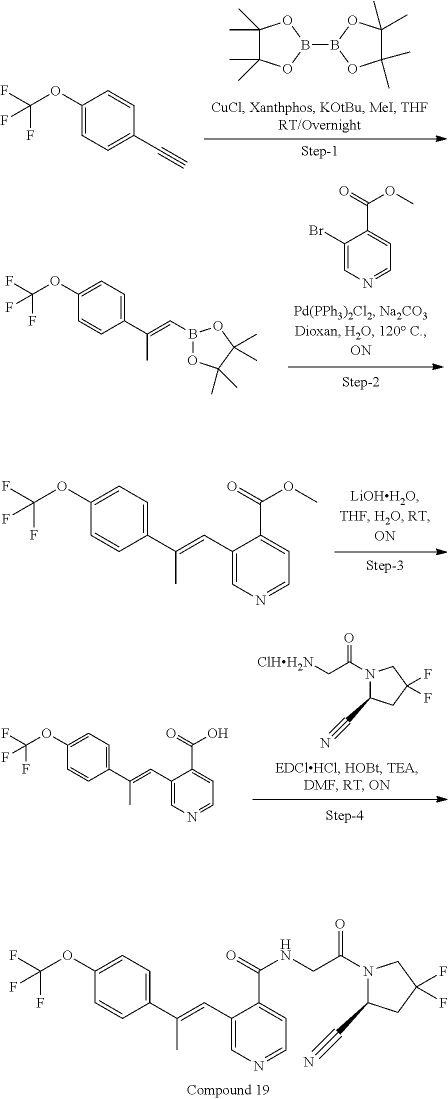

Step 1: Synthesis of (E)-4,4,5,5-tetramethyl-2-(2-(4-(trifluoromethoxy)phenyl)prop-1-en-1-yl)-1,3,2-dioxaborolane. To a stirred solution 1-ethynyl-4-(trifluoromethoxy)benzene (1.0 g, 5.37 mmol, 1.0 equiv) in THF (10 mL), was added 4,4,4′,4′,5,5,5′,5′-octamethyl-2,2′-bi(1,3,2-dioxaborolane) (1.49 g, 5.90 mmol, 1.1 equiv), CuCl (0.005 g, 0.050 mmol, 0.01 equiv), Xanthphos (0.030 g, 0.050 mmol, 0.01 equiv), KOtBu (0.71 g, 0.63 mmol, 1.2 equiv) followed by the addition of methyl iodide (1.3 ml, 21.2 mmol, 4.0 equiv). The resulting reaction mixture was allowed to stir at RT for overnight. Product formation was confirmed by TLC. After the completion of reaction, the reaction mixture was diluted with water solution (50 mL) and extracted with ethyl acetate (100 mL×2). Combined organic extracts were washed with water (20 mL×4), dried over anhydrous Na₂SO₄ and concentrated. The crude product obtained was purified by flash chromatography (0-10% ethyl acetate in hexane) to obtain (E)-4,4,5,5-tetramethyl-2-(2-(4-(trifluoromethoxy)phenyl)prop-1-en-1-yl)-1,3,2-dioxaborolane (0.700 g, 39% Yield) as a yellow oil.

LCMS 328.1 [M+H]⁺

¹H NMR (400 MHz, CHLOROFORM-d) δ 7.40-7.58 (m, 2H) 7.16 (m, J=8.33 Hz, 2H) 5.73 (d, J=0.88 Hz, 1H) 2.29-2.44 (m, 3H) 1.14-1.42 (m, 12H).

Step 2: Synthesis of methyl (E)-3-(2-(4-(trifluoromethoxy)phenyl)prop-1-en-1-yl)isonicotinate. To a solution of methyl 3-bromoisonicotinate (0.220 g, 1.018 mmol, 1.0 equiv) in dioxane:water (4:2 mL) was added (E)-4,4,5,5-tetramethyl-2-(2-(4-(trifluoromethoxy)phenyl)prop-1-en-1-yl)-1,3,2-dioxaborolane (0.400 g, 1.22 mmol, 1.2 equiv), K₂CO₃ (0.284 g, 2.037 mmol, 2.0 equiv) and the mixture was purged with N₂ gas for 10 min, followed by the addition of Pd(PPh₂)Cl₂ (0.035 g, 0.050 mmol. 0.05 equiv). The resulting reaction mixture was heated at 120° C. for overnight. Product formation was confirmed by LCMS. After the completion of reaction, the reaction mixture was diluted with water (20 mL) and extracted with ethyl acetate (50 mL×2). Combined organic extracts were washed with water (10 mL×2), dried over anhydrous Na₂SO₄ and concentrated. The crude product was purified by flash chromatography (0-20% ethyl acetate in hexane as an eluent) to obtain methyl (E)-3-(2-(4-(trifluoromethoxy)phenyl)prop-1-en-1-yl)isonicotinate (0.110 g, 35% yield) as a yellow solid.

LCMS 338.1 [M+H]⁺

¹H NMR (400 MHz, DMSO-d₆) δ 8.57-8.82 (m, 2H) 7.64-7.78 (m, 2H) 7.42 (d, J=7.89 Hz, 2H) 7.16 (s, 1H) 3.75-3.99 (m, 3H) 2.09 (d, J=1.32 Hz, 3H).

Step 3: Synthesis of (E)-3-(2-(4-(trifluoromethoxy)phenyl)prop-1-en-1-yl)isonicotinic acid. To a stirred solution of methyl (E)-3-(2-(4-(trifluoromethoxy)phenyl)prop-1-en-1-yl)isonicotinate (0.100 g, 0.296 mmol, 1.0 equiv) in THF (4 mL) and water (4 mL), was added LiOH.H₂O (0.024 g, 0.593 mmol, 2.0 equiv). The mixture was allowed to stir at RT for overnight. Product formation was confirmed by LCMS and ¹H NMR Spectroscopy. The reaction mixture was diluted with water (20 mL) and washed with ethyl acetate (10 mL×2). Aqueous layer was separated and freeze dried on lyophilyzer to obtain (E)-3-(2-(4-(trifluoromethoxy)phenyl)prop-1-en-1-yl)isonicotinic acid (0.100 g, Quant. Yield) as a white solid.

LCMS 324.0 [M+H]⁺

Step 4: Synthesis of (S,E)-N-(2-(2-cyano-4,4-difluoropyrrolidin-1-yl)-2-oxoethyl)-3-(2-(4-(trifluoromethoxy)phenyl)prop-1-en-1-yl)isonicotinamide. To a stirred solution of (E)-3-(2-(4-(trifluoromethoxy)phenyl)prop-1-en-1-yl)isonicotinic acid (0.1 g, 0.308 mmol, 1.0 equiv) in DMF (5 mL), was added (S)-4,4-difluoro-1-glycylpyrrolidine-2-carbonitrile hydrochloride (0.069 g, 0.308 mmol, 1.0 equiv), EDCI.HCl (0.070 g, 0.370 mmol, 1.2 equiv) and HOBt (0.049 g, 0.370 mmol, 1.2 equiv) followed by the addition of TEA (0.2 mL). The resulting reaction mixture was allowed to stir at RT for overnight. Product formation was confirmed by LCMS and TLC. After completion of reaction, the mixture was diluted with water (20 mL) and extracted with ethyl acetate (25 mL×2). Combined organic extracts were washed with water (10 mL×4), dried over anhydrous Na₂SO₄ and concentrated. The crude product was purified by flash chromatography (5% MeOH in DCM as an eluent) to obtain (S,E)-N-(2-(2-cyano-4,4-difluoropyrrolidin-1-yl)-2-oxoethyl)-3-(2-(4-(trifluoromethoxy)phenyl)prop-1-en-1-yl)isonicotinamide (0.030 g, 20.0% Yield) as a white solid.

LCMS 495.3 [M+H]⁺

¹H NMR (400 MHz, DMSO-d₆) δ 8.89 (t, J=5.92 Hz, 1H) 8.69 (s, 1H) 8.60 (d, J=4.82 Hz, 1H) 7.74 (m, J=8.77 Hz, 2H) 7.47 (d, J=4.82 Hz, 1H) 7.39 (m, J=8.33 Hz, 2H) 7.01-7.13 (m, 1H) 5.13 (d, J=6.58 Hz, 1H) 4.24-4.35 (m, 1H) 3.92-4.20 (m, 3H) 2.69-2.95 (m, 2H) 2.18 (s, 3H).

### Example S16

### Synthesis of (S,E)-N-(2-(2-cyano-4,4-difluoropyrrolidin-1-yl)-2-oxoethyl)-3-(2-(4-(trifluoromethyl)phenyl)prop-1-en-1-yl)isonicotinamide

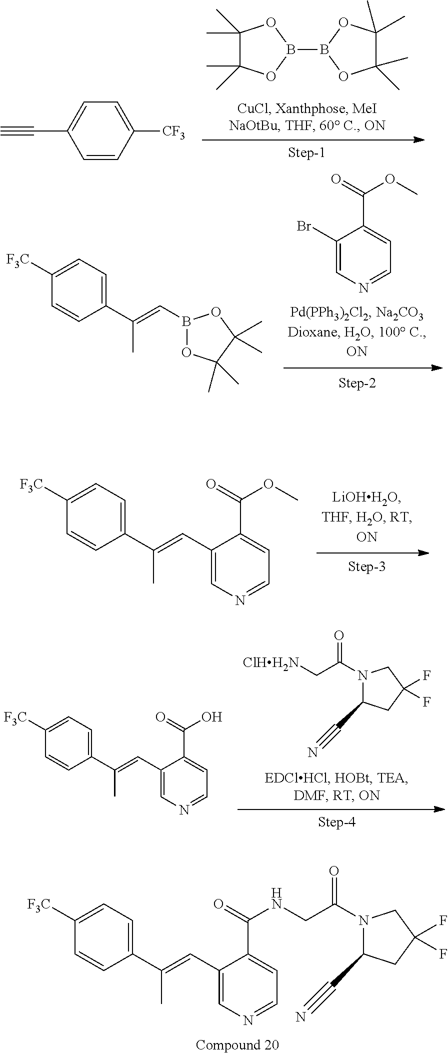

Step 1: Synthesis of (E)-4,4,5,5-tetramethyl-2-(2-(4-(trifluoromethyl)phenyl)prop-1-en-1-yl)-1,3,2-dioxaborolane. To a stirred solution 4,4,4′,4′,5,5,5′,5′-octamethyl-2,2′-bi(1,3,2-dioxaborolane) (0.818 g, 3.23 mmol, 1.1 equiv) in dried THF (20 mL), was added CuCl (3.0 mg, 0.0294 mmol, 0.01 equiv), Xanthphos (0.017 mg, 0.0294 mmol, 0.01 equiv) and NaOtBu (0.34 g, 3.53 mmol, 1.2 equiv) at RT. The mixture was allowed to stir at RT for 30 minute. 1-ethynyl-4-(trifluoromethyl)benzene (0.5 g, 2.94 mmol, 1.0 equiv) dissolved in THF (5 mL) and Methyl iodide (0.8 mL, 11.764 mmol, 4.0 equiv) was added into above reaction mixture, resultant reaction mixture stir at RT overnight. Product formation was confirmed by TLC. After completion of reaction, the mixture was diluted with water (50 mL) and extracted with ethyl acetate (50 mL×2). Combined organic extracts were washed with water (20 mL×2), dried over anhydrous Na₂SO₄ and concentrated. The crude product was purified by flash chromatography (5% EtOAc in Hexane as an eluent) to obtain (E)-4,4,5,5-tetramethyl-2-(2-(4-(trifluoromethyl)phenyl)prop-1-en-1-yl)-1,3,2-dioxaborolane (0.8 g, 87.2% Yield) as a yellow oil.

¹H NMR (DMSO-d₆, 400 MHz) δ 7.81 (d, J=7.9 Hz, 2H), 7.59-7.74 (m, 2H), 5.75 (s, 1H), 2.30-2.43 (m, 3H), 1.18-1.31 (m, 12H).

Step 2: Synthesis of methyl (E)-3-(2-(4-(trifluoromethyl)phenyl)prop-1-en-1-yl)isonicotinate. To a solution of methyl 3-bromoisonicotinate (0.1 g, 0.463 mmol, 1.0 equiv) in dioxane (5 mL) and water (1 mL) was added (E)-4,4,5,5-tetramethyl-2-(2-(4-(trifluoromethyl)phenyl)prop-1-en-1-yl)-1,3,2-dioxaborolane (0.216 g, 0.694 mmol, 1.5 equiv), Na₂CO₃ (0.1 g, 0.936 mmol, 2.0 equiv) and resulting reaction mixture purged with N₂ gas for 10 minute, followed by the addition of Pd(PPh₃)Cl₂ (0.016 g, 0.0235 mmol. 0.05 equiv). The resulting reaction mixture was heated at 100° C. for overnight. Product formation was confirmed by LCMS. After the completion of reaction, the mixture was filtered through celite bed, washed with ethyl acetate (50 mL). Filtrate was concentrated under reduced pressure. The crude product was purified by flash chromatography (0-20% ethyl acetate in hexane as an eluent) to obtained methyl (E)-3-(2-(4-(trifluoromethyl)phenyl)prop-1-en-1-yl)isonicotinate (0.06 g, 40.26% yield) as a yellow oil.

LCMS 322.2 [M+H]⁺

¹H NMR (DMSO-d₆, 400 MHz) δ 8.63-8.73 (m, 2H), 7.71-7.84 (m, 5H), 7.25 (s, 1H), 3.79-3.95 (m, 3H), 2.06-2.20 (m, 3H)

Step 3: Synthesis of (E)-3-(2-(4-(trifluoromethyl)phenyl)prop-1-en-1-yl)isonicotinic acid. To a stirred solution of methyl (E)-3-(2-(4-(trifluoromethyl)phenyl)prop-1-en-1-yl)isonicotinate (0.06 g, 0.186 mmol, 1.0 equiv) in THF (2 mL) and water (2 mL), was added LiOH.H₂O (0.012 g, 0.28 mmol, 1.5 equiv). The mixture was allowed to stir at RT for overnight. Product formation was confirmed by LCMS and ¹H NMR Spectroscopy. The reaction mixture was concentrated and diluted with water (20 mL) and washed with ethyl acetate (10 mL×2). Aqueous layer acidify with 6N HCl (pH˜5 to 6), solid precipitate was filtered off and dried under vacuum to obtain (E)-3-(2-(4-(trifluoromethyl)phenyl)prop-1-en-1-yl)isonicotinic acid (0.035 g, 61.4%) as an off white solid.

LCMS 308.1 [M+H]⁺

Step 4: Synthesis of (S,E)-N-(2-(2-cyano-4,4-difluoropyrrolidin-1-yl)-2-oxoethyl)-3-(2-(4-(trifluoromethyl)phenyl)prop-1-en-1-yl)isonicotinamide. To a stirred solution of (E)-3-(2-(4-(trifluoromethyl)phenyl)prop-1-en-1-yl)isonicotinic acid (0.035 g, 0.114 mmol, 1.0 equiv) in DMF (3 mL), was added (S)-4,4-difluoro-1-glycylpyrrolidine-2-carbonitrile hydrochloride (0.026 g, 0.114 mmol, 1.5 equiv), EDCI.HCl (0.033 g, 0.171 mmol, 1.5 equiv) and HOBt (0.023 g, 0.171 mmol, 1.5 equiv). The mixture was allowed to stir at RT for 10 min. TEA (0.1 mL) was added and the mixture was allowed to stir at RT for overnight. Product formation was confirmed by LCMS and TLC. After completion of reaction, the mixture was diluted with water (10 mL) and extracted with ethyl acetate (10 mL×2). Combined organic extracts were washed with water (10 mL×4), dried over anhydrous Na₂SO₄ and concentrated. The crude product was purified by reversed phase HPLC to obtain (S,E)-N-(2-(2-cyano-4,4-difluoropyrrolidin-1-yl)-2-oxoethyl)-3-(2-(4-(trifluoromethyl)phenyl)prop-1-en-1-yl)isonicotinamide (0.007 g, 12.86% Yield) as a white solid.

LCMS 479.2[M+H]⁺

¹H NMR (DMSO-d₆, 400 MHz) δ 8.91 (d, J=5.7 Hz, 1H), 8.72 (s, 1H), 8.63 (d, J=4.8 Hz, 1H), 7.84 (d, J=8.3 Hz, 2H), 7.76 (d, J=8.3 Hz, 2H), 7.50 (d, J=4.8 Hz, 1H), 7.13-7.20 (m, 1H), 5.14 (d, J=7.5 Hz, 1H), 4.23-4.34 (m, 1H), 4.03-4.23 (m, 2H), 2.68-2.94 (m, 3H), 2.21 (s, 3H).

### Example S17

### Synthesis of (S,E)-N-(2-(2-cyano-4,4-difluoropyrrolidin-1-yl)-2-oxoethyl)-3-(4-(trifluoromethyl)styryl)isonicotinamide

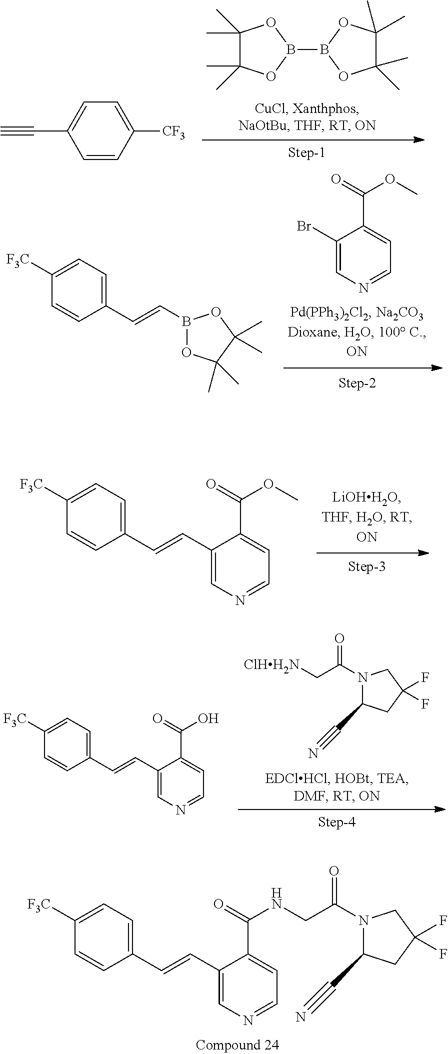

Step 1: Synthesis of (E)-4,4,5,5-tetramethyl-2-(4-(trifluoromethyl)styryl)-1,3,2-dioxaborolane. To a stirred solution 4,4,4′,4′,5,5,5′,5′-octamethyl-2,2′-bi(1,3,2-dioxaborolane) (0.818 g, 3.23 mmol, 1.1 equiv) in dried THF (20 mL), was added CuCl (3.0 mg, 0.0294 mmol, 0.01 equiv), Xanthphos (0.017 g, 0.0294 mmol, 0.01 equiv) and NaOtBu (0.34 g, 3.53 mmol, 1.2 equiv) at RT. The mixture was allowed to stir at RT for 30 min. 1-ethynyl-4-(trifluoromethyl)benzene (0.5 g, 2.94 mmol, 1.0 equiv) in THF (5 mL) was added into above reaction mixture and resultant reaction mixture stir at RT overnight. Product formation was confirmed by TLC. After completion of reaction, the mixture was diluted with water (50 mL) and extracted with ethyl acetate (50 mL×2). Combined organic extracts were washed with water (20 mL×2), dried over anhydrous Na₂SO₄ and concentrated. The crude product was purified by flash chromatography (5% EtOAc in Hexane as an eluent) to obtain (E)-4,4,5,5-tetramethyl-2-(4-(trifluoromethyl)styryl)-1,3,2-dioxaborolane (0.6 g, 68.5% Yield) as a brown solid.

¹H NMR (DMSO-d₆, 400 MHz) δ 7.81 (d, J=9.2 Hz, 2H), 7.66-7.79 (m, 2H), 7.35 (s, 1H), 7.40 (s, 1H), 6.32 (d, J=18.4 Hz, 1H), 1.22-1.35 (m, 10H), 1.13-1.22 (m, 2H).

Step 2: Synthesis of methyl (E)-3-(4-(trifluoromethyl)styryl)isonicotinate. To a solution of methyl 3-bromoisonicotinate (0.3 g, 1.39 mmol, 1.0 equiv) in dioxane (20 mL) and water (2 mL) was added (E)-4,4,5,5-tetramethyl-2-(4-(trifluoromethyl)styryl)-1,3,2-dioxaborolane (0.496 g, 1.67 mmol, 1.2 equiv), Na₂CO₃ (0.3 g, 2.78 mmol, 2.0 equiv) and resulting reaction mixture purged with N₂ gas for 10 minute, followed by the addition of Pd(PPh₃)Cl₂ (0.049 g, 0.0694 mmol. 0.05 equiv). The resulting reaction mixture was heated at 100° C. for overnight. Product formation was confirmed by LCMS. After the completion of reaction, the mixture was filtered through celite bed, washed with ethyl acetate (100 mL). Filtrate was concentrated under reduced pressure. The crude product was purified by flash chromatography (0-20% ethyl acetate in hexane as an eluent) to obtain methyl (E)-3-(4-(trifluoromethyl)styryl)isonicotinate (0.4 g, 93.9% yield) as a yellow oil.

LCMS 308.1 [M+H]⁺

¹H NMR (DMSO-d₆, 400 MHz) δ 9.15 (s, 1H), 8.68 (d, J=4.8 Hz, 1H), 7.71-7.87 (m, 5H), 7.43 (s, 1H), 7.48 (s, 1H), 3.83-3.96 (m, 3H).

Step 3: Synthesis of (E)-3-(4-(trifluoromethyl)styryl)isonicotinic acid. To a stirred solution of methyl (E)-3-(4-(trifluoromethyl)styryl)isonicotinate (0.4 g, 1.303 mmol, 1.0 equiv) in THF (5 mL) and water (5 mL), was added LiOH.H₂O (0.092 g, 2.21 mmol, 1.5 equiv). The mixture was allowed to stir at RT for overnight. Product formation was confirmed by LCMS and ¹H NMR Spectroscopy. The reaction mixture was concentrated and diluted with water (20 mL) and washed with ethyl acetate (10 mL×2). Aqueous layer acidify with 6N HCl (pH˜5 to 6), solid precipitate was filtered off and dried under vacuum to obtained (E)-3-(4-(trifluoromethyl)styryl)isonicotinic acid (0.355 g, 93%) as a yellow solid.

LCMS 294.1 [M+H]⁺

Step 4: Synthesis of (S,E)-N-(2-(2-cyano-4,4-difluoropyrrolidin-1-yl)-2-oxoethyl)-3-(4-(trifluoromethyl)styryl)isonicotinamide. To a stirred solution of (E)-3-(4-(trifluoromethyl)styryl)isonicotinic acid (0.07 g, 0.239 mmol, 1.0 equiv) in DMF (5 mL), was added (S)-4,4-difluoro-1-glycylpyrrolidine-2-carbonitrile hydrochloride (0.54 g, 0.239 mmol, 1.5 equiv), HOBt (0.05 g, 0.359 mmol, 1.5 equiv) and EDCI.HCl (0.07 g, 0.359 mmol, 1.5 equiv). The mixture was allowed to stir at RT for 10 min. TEA (0.2 mL) was added and the mixture was allowed to stir at RT for overnight. Product formation was confirmed by LCMS and TLC. After completion of reaction, the mixture was diluted with water (20 mL) and extracted with ethyl acetate (20 mL×2). Combined organic layer was washed with water (20 mL×4), dried over anhydrous Na₂SO₄ and concentrated under reduced pressure. The crude product was purified by flash chromatography (5% MeOH in DCM as an eluent) to obtain (S,E)-N-(2-(2-cyano-4,4-difluoropyrrolidin-1-yl)-2-oxoethyl)-3-(4-(trifluoromethyl)styryl)isonicotinamide (0.05 g, 45% Yield) as an off white solid.

LCMS 465.2 [M+H]⁺

¹H NMR (DMSO-d₆, 400 MHz) δ (s, 1H), 8.92-9.05 (m, 1H), 8.57 (d, J=4.8 Hz, 1H), 7.82-7.97 (m, 3H), 7.75 (d, J=8.3 Hz, 2H), 7.59 (d, J=17.1 Hz, 1H), 7.39 (d, J=5.3 Hz, 1H), 5.21 (d, J=6.1 Hz, 1H), 4.28-4.37 (m, 1H), 4.03-4.27 (m, 2H), 2.73-3.02 (m, 3H).

### Example S18

### Synthesis of (S,E)-3-(2-(4-chlorophenyl)prop-1-en-1-yl)-N-(2-(2-cyano-4,4-difluoropyrrolidin-1-yl)-2-oxoethyl)isonicotinamide

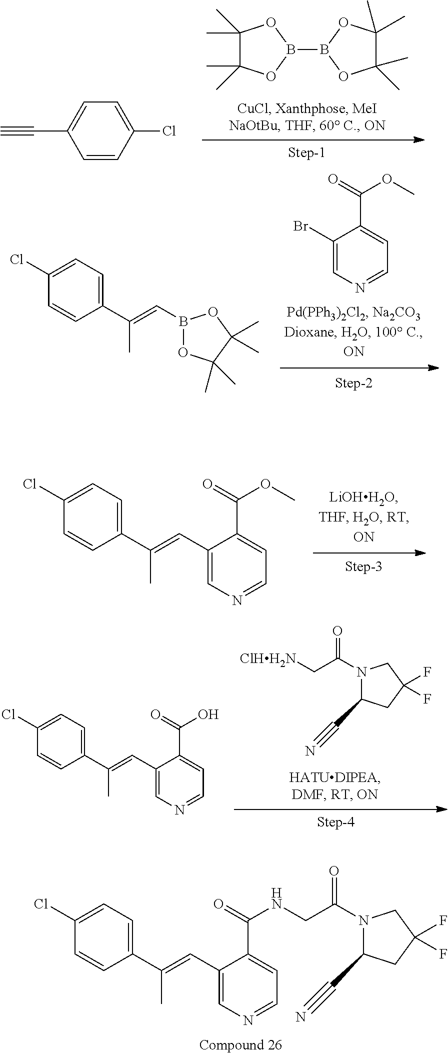

Step 1: Synthesis of (E)-2-(2-(4-chloroyphenyl)prop-1-en-1-yl)-4,4,5,5-tetramethyl-1,3,2-dioxaborolane. To a stirred solution 4,4,4′,4′,5,5,5′,5′-octamethyl-2,2′-bi(1,3,2-dioxaborolane) (1.4 g, 5.514 mmol, 1.5 equiv) in dried THF (10 mL), was added CuCl (18 mg, 0.183 mmol, 0.05 equiv), Xanthphos (0.106 g, 0.183 mmol, 0.05 equiv) & KOtBu (0.5 g, 4.4 mmol, 1.2 equiv) at RT. The reaction mixture was allowed to stir at RT for 30 min. 1-chloro-4-ethynylbenzene (0.5 g, 3.78 mmol, 1.0 equiv) in THF (5 mL) was added followed by the addition of methyl iodide (1 mL, 15.12 mmol, 4.0 equiv). The resulting reaction mixture was heated at 60° C. overnight. Product formation was confirmed by TLC. After completion of reaction, the mixture was diluted with water (50 mL) and extracted with ethyl acetate (50 mL×2). Combined organic extracts were washed with water (20 mL×2), dried over anhydrous Na₂SO₄ and concentrated. The crude product was purified by flash chromatography (2% EtOAc in hexane as an eluent) to obtain (E)-2-(2-(4-chloroyphenyl)prop-1-en-1-yl)-4,4,5,5-tetramethyl-1,3,2-dioxaborolane (0.57 g, 33.81% Yield) as a white solid.

LCMS 279.0 [M+H]⁺

¹H NMR (DMSO-d₆, 400 MHz) δ 7.46-7.60 (m, J=8.8 Hz, 2H), 7.35-7.45 (m, J=8.8 Hz, 2H), 5.67 (s, 1H), 2.32 (s, 3H), 1.06-1.32 ppm (m, 12H).

Step 2: Synthesis of methyl (E)-3-(2-(4-chlorophenyl)prop-1-en-1-yl)isonicotinate. To a solution of methyl 3-bromoisonicotinate (0.15 g, 0.694 mmol, 1.0 equiv) in dioxane (10 mL) and water (0.5 mL) was added (E)-2-(2-(4-chloroyphenyl)prop-1-en-1-yl)-4,4,5,5-tetramethyl-1,3,2-dioxaborolane (0.29 g, 1.04 mmol, 1.5 equiv), Na₂CO₃ (0.15 g, 1.38 mmol, 2.0 equiv) and resulting reaction mixture was purged with N₂ gas for 10 minute, followed by the addition of Pd(PPh₃)Cl₂ (0.024 g, 0.035 mmol. 0.05 equiv). The resulting reaction mixture was heated at 100° C. for overnight. Product formation was confirmed by LCMS. After the completion of reaction, the mixture was filtered through Celite®, washed with ethyl acetate (50 mL). Filtrate was concentrated under reduced pressure. The crude product was purified by flash chromatography (0-20% ethyl acetate in hexane as an eluent) to obtain methyl (E)-3-(2-(4-chlorophenyl)prop-1-en-1-yl)isonicotinate (0.050 g, 25% yield) as a yellow oil.

LCMS 288.2 [M+H]⁺

Step 3: Synthesis of (E)-3-(2-(4-chlorophenyl)prop-1-en-1-yl)isonicotinic acid. To a stirred solution of methyl (E)-3-(2-(4-chlorophenyl)prop-1-en-1-yl)isonicotinate (0.050 g, 0.174 mmol, 1.0 equiv) in THF (2 mL) and water (2 mL), was added LiOH.H₂O (0.011 g, 0.26 mmol, 1.5 equiv). The mixture was allowed to stir at RT for overnight. Product formation was confirmed by LCMS and ¹H NMR Spectroscopy. The reaction mixture was concentrated and diluted with water (4 mL) and washed with ethyl acetate (2 mL×2). Aqueous layer acidify with 6N HCl (pH˜5 to 6), solid precipitate formed was filtered off and dried under vacuum to obtain (E)-3-(2-(4-chlorophenyl)prop-1-en-1-yl)isonicotinic acid (0.04 g, 84.21%) as a yellow solid.

LCMS 274.2 [M+H]⁺

¹H NMR (DMSO-d₆, 400 MHz) δ 13.61 (br. s., 1H), 8.57-8.70 (m, 2H), 7.77 (d, J=5.3 Hz, 1H), 7.55-7.67 (m, J=8.3 Hz, 2H), 7.41-7.52 (m, J=8.8 Hz, 2H), 7.18 (s, 1H), 2.08 (s, 3H).

Step 4: (S,E)-3-(2-(4-chlorophenyl)prop-1-en-1-yl)-N-(2-(2-cyano-4,4-difluoropyrrolidin-1-yl)-2-oxoethyl)isonicotinamide. To a stirred solution of (E)-3-(2-(4-chlorophenyl)prop-1-en-1-yl)isonicotinic acid (0.04 g, 0.146 mmol, 1.0 equiv) in DMF (2 mL), was added (S)-4,4-difluoro-1-glycylpyrrolidine-2-carbonitrile hydrochloride (0.05 g, 0.219 mmol, 1.5 equiv), HATU (0.09 g, 0.219 mmol, 1.5 equiv). The mixture was allowed to stir at RT for 10 min. DIPEA (0.1 mL) was added and the mixture was allowed to stir at RT for overnight. Product formation was confirmed by LCMS and TLC. After completion of reaction, the mixture was diluted with water (10 mL) and extracted with ethyl acetate (10 mL×2). Combined organic extracts were washed with water (5 mL×4), dried over anhydrous Na₂SO₄ and concentrated. The crude product was purified by reverse phase HPLC to obtain (S,E)-3-(2-(4-chlorophenyl)prop-1-en-1-yl)-N-(2-(2-cyano-4,4-difluoropyrrolidin-1-yl)-2-oxoethyl)isonicotinamide (0.008 g, 13.84% Yield) as a white solid.

LCMS 445.2 [M+H]⁺

¹H NMR (DMSO-d₆, 400 MHz) δ 8.88 (t, J=5.9 Hz, 1H), 8.68 (s, 1H), 8.60 (d, J=4.8 Hz, 1H), 7.64 (d, J=8.3 Hz, 2H), 7.32-7.55 (m, 3H), 7.10 (s, 1H), 5.06-5.19 (m, 1H), 4.28 (br. s., 1H), 3.95-4.17 (m, 2H), 2.91 (br. s., 2H), 2.82 (d, J=15.8 Hz, 2H), 2.17 (s, 3H).

### Example S19

### Synthesis of (S,E)-N-(2-(2-cyano-4,4-difluoropyrrolidin-1-yl)-2-oxoethyl)-3-(2-(4-methoxyphenyl)prop-1-en-1-yl)isonicotinamide

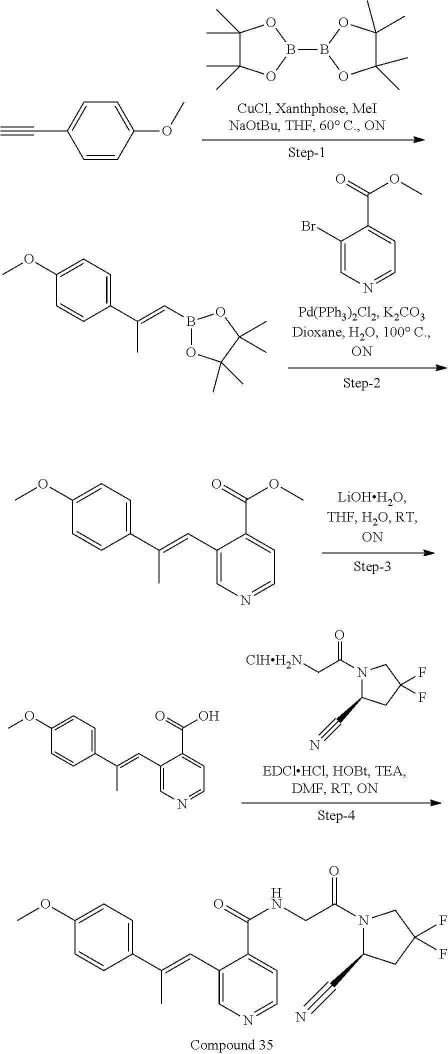

Step 1: Synthesis of (E)-2-(2-(4-methoxyphenyl)prop-1-en-1-yl)-4,4,5,5-tetramethyl-1,3,2-dioxaborolane. To a stirred solution 4,4,4′,4′,5,5,5′,5′-octamethyl-2,2′-bi(1,3,2-dioxaborolane) (1.4 g, 4.16 mmol, 1.5 equiv) in dried THF (10 mL), was added CuCl (0.019 g, 0.189 mmol, 0.05 equiv), Xanthphos (0.109 g, 0.189 mmol, 0.05 equiv) and KOtBu (0.509 g, 4.536 mmol, 1.2 equiv) at RT. The mixture was allowed to stir at RT for 30 min. 1-ethynyl-4-methoxybenzene (0.5 g, 3.78 mmol, 1.0 equiv) in THF (5 mL) was added followed by the addition of methyl iodide (1 mL, 15.12 mmol, 4.0 equiv) was added into above reaction mixture, resultant reaction mixture was heated at 60° C. overnight. Product formation was confirmed by TLC. After completion of reaction, the mixture was diluted with water (50 mL) and extracted with ethyl acetate (50 mL×2). Combined organic extracts were washed with water (20 mL×2), dried over anhydrous Na₂SO₄ and concentrated. The crude product was purified by flash chromatography (2% EtOAc in Hexane as an eluent) to obtain (E)-2-(2-(4-methoxyphenyl)prop-1-en-1-yl)-4,4,5,5-tetramethyl-1,3,2-dioxaborolane (0.35 g, 33.81% Yield) as a white solid.

LCMS 261.2 [M+H]⁺

¹H NMR (DMSO-d₆, 400 MHz) δ7.38-7.57 (m, J=8.8 Hz, 2H), 6.76-6.94 (m, J=8.8 Hz, 2H), 5.58 (s, 1H), 3.67-3.80 (m, 3H), 3.33 (s, 3H), 1.17-1.32 (m, 12H).

Step 2: Synthesis of methyl (E)-3-(2-(4-methoxyphenyl)prop-1-en-1-yl)isonicotinate. To a solution of methyl 3-bromoisonicotinate (0.15 g, 0.694 mmol, 1.0 equiv) in dioxane (10 mL) and water (0.5 mL) was added (E)-2-(2-(4-methoxyphenyl)prop-1-en-1-yl)-4,4,5,5-tetramethyl-1,3,2-dioxaborolane (0.285 g, 1.04 mmol, 1.5 equiv), K₂CO₃ (0.2 g, 1.85 mmol, 2.0 equiv) and resulting reaction mixture purged with N₂ gas for 10 minute, followed by the addition of Pd(PPh₃)Cl₂ (0.024 g, 0.035 mmol. 0.05 equiv). The resulting reaction mixture was heated at 100° C. for overnight.

Product formation was confirmed by LCMS. After the completion of reaction, the mixture was filtered through Celite®, washed with ethyl acetate (50 mL). Filtrate was concentrated under reduced pressure. The crude product was purified by flash chromatography (0-20% ethyl acetate in hexane as an eluent) to obtain methyl (E)-3-(2-(4-methoxyphenyl)prop-1-en-1-yl)isonicotinate (0.150 g, 76.53% yield) as a yellow oil.

LCMS 284.1 [M+H]⁺

Step 3: Synthesis of (E)-3-(2-(4-methoxyphenyl)prop-1-en-1-yl)isonicotinic acid. To a stirred solution of methyl (E)-3-(2-(4-methoxyphenyl)prop-1-en-1-yl)isonicotinate (0.250 g, 0.929 mmol, 1.0 equiv) in THF (10 mL) and water (10 mL), was added LiOH.H₂O (0.056 g, 1.39 mmol, 1.5 equiv). The mixture was allowed to stir at RT for overnight. Product formation was confirmed by LCMS and ¹H NMR Spectroscopy. The reaction mixture was concentrated and diluted with water (20 mL) and washed with ethyl acetate (10 mL×2). Aqueous layer was acidified with 6N HCl (pH˜5 to 6), solid precipitate formed was filtered off and dried under vacuum to obtain (E)-3-(2-(4-methoxyphenyl)prop-1-en-1-yl)isonicotinic acid (0.08 g, 38.27%) as a yellow solid.

LCMS 270.1 [M+H]⁺

¹H NMR (DMSO-d₆, 400 MHz) δ13.59 (br. s., 1H), 8.51-8.67 (m, 2H), 7.73 (d, J=4.8 Hz, 1H), 7.42-7.57 (m, J=8.8 Hz, 2H), 7.08 (s, 1H), 6.87-7.04 (m, J=8.3 Hz, 2H), 3.78 (s, 3H), 2.07 (s, 3H).

Step 4: (S,E)-N-(2-(2-cyano-4,4-difluoropyrrolidin-1-yl)-2-oxoethyl)-3-(2-(4-methoxyphenyl)prop-1-en-1-yl)isonicotinamide. To a stirred solution of (E)-3-(2-(4-methoxyphenyl)prop-1-en-1-yl)isonicotinic acid (0.08 g, 0.297 mmol, 1.0 equiv) in DMF (5 mL), was added (S)-4,4-difluoro-1-glycylpyrrolidine-2-carbonitrile hydrochloride (0.1 g, 0.446 mmol, 1.5 equiv), EDCI.HCl (0.09 g, 0.446 mmol, 1.5 equiv) and HOBt (0.06 g, 0.446 mmol, 1.5 equiv). The mixture was allowed to stir at RT for 10 min. TEA (0.2 mL) was added and the mixture was allowed to stir at RT for overnight. Product formation was confirmed by LCMS and TLC. After completion of reaction, the mixture was diluted with water (20 mL) and extracted with ethyl acetate (20 mL×2). Combined organic extracts were washed with water (20 mL×4), dried over anhydrous Na₂SO₄ and concentrated. The crude product was purified by reversed phase HPLC to obtain (S,E)-N-(2-(2-cyano-4,4-difluoropyrrolidin-1-yl)-2-oxoethyl)-3-(2-(4-methoxyphenyl)prop-1-en-1-yl)isonicotinamide (0.042 g, 32.3% Yield) as a white solid.

LCMS 441.3 [M+H]⁺

¹H NMR (DMSO-d₆, 400 MHz) δ 8.84 (br. s., 1H), 8.66 (s, 1H), 8.57 (d, J=4.8 Hz, 1H), 7.55 (d, J=8.8 Hz, 2H), 7.44 (d, J=5.3 Hz, 1H), 6.81-7.08 (m, 3H), 5.11 (d, J=9.6 Hz, 1H), 4.27 (br. s., 1H), 4.13 (d, J=6.1 Hz, 2H), 3.77 (s, 3H), 2.83 (br. s., 2H), 2.67 (d, J=1.8 Hz, 2H), 2.15 (s, 3H).

### Example S20

### Synthesis of (S,E)-N-(2-(2-cyano-4,4-difluoropyrrolidin-1-yl)-2-oxoethyl)-3-(4-(trifluoromethoxy)styryl)isonicotinamide

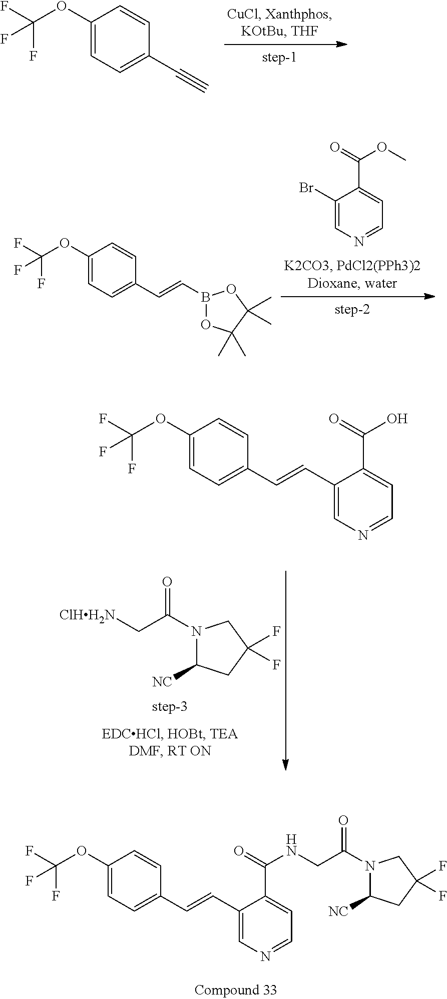

Step 1: Synthesis of (E)-4,4,5,5-tetramethyl-2-(4-(trifluoromethoxy)styryl)-1,3,2-dioxaborolane. To a stirred solution 4,4,4′,4′,5,5,5′,5′-octamethyl-2,2′-bi(1,3,2-dioxaborolane) (0.750 g, 2.92 mmol, 1.1 equiv) in dried THF (10 mL), was added CuCl (0.002 g, 0.020 mmol, 0.01 equiv), Xanthphos (0.015 g, 0.020 mmol, 0.01 equiv) and KOtBu (0.361 g, 3.22 mmol, 1.2 equiv) at RT. The mixture was allowed to stir at RT for 30 min. 1-ethynyl-4-(trifluoromethoxy)benzene (0.5 g, 2.368 mmol, 1.0 equiv) in THF (5 mL) was added into above reaction mixture, resultant reaction mixture was heated at 60° C. overnight. Product formation was confirmed by TLC. After completion of reaction, the mixture was diluted with water (50 mL) and extracted with ethyl acetate (50 mL×2). Combined organic extracts were washed with water (20 mL×2), dried over anhydrous Na₂SO₄ and concentrated. The crude product was purified by flash chromatography (2% EtOAc in Hexane as an eluent) to obtain (E)-4,4,5,5-tetramethyl-2-(4-(trifluoromethoxy)styryl)-1,3,2-dioxaborolane (0.35 g, 41% Yield) as an off-white solid.

LCMS 315.0 [M+H]⁺

¹H NMR (400 MHz, DMSO-d₆) δ 7.73 (d, J=8.77 Hz, 1H) 7.27-7.43 (m, 2H) 6.18 (d, J=18.86 Hz, 1H) 1.24 (s, 9H).

Step 2: Synthesis of (E)-3-(4-(trifluoromethoxy)styryl)isonicotinic acid. To a solution of methyl 3-bromoisonicotinate (0.200 g, 1.62 mmol, 1.0 equiv) in dioxane (5 mL) and water (2 mL) was added (E)-4,4,5,5-tetramethyl-2-(4-(trifluoromethoxy)styryl)-1,3,2-dioxaborolane (0.350 g, 1.94 mmol, 1.2 equiv), K₂CO₃ (0.256 g, 3.24 mmol, 2.0 equiv) and resulting reaction mixture purged with N₂ gas for 10 minute, followed by the addition of Pd(PPh₃)Cl₂ (0.033 g, 0.081 mmol. 0.05 equiv). The resulting reaction mixture was heated at 120° C. for overnight. Product formation was confirmed by LCMS. After the completion of reaction, the reaction mixture was diluted with water (10 mL). Aqueous layer was washed with ethyl acetate (5 mL×2). Aqueous layer was separated and freeze dried to obtain (E)-3-(4-(trifluoromethoxy)styryl)isonicotinic acid (0.150 g, 50% yield) as an off-white solid.

LCMS 310.1 [M+H]⁺

Step 3: (S,E)-N-(2-(2-cyano-4,4-difluoropyrrolidin-1-yl)-2-oxoethyl)-3-(4-(trifluoromethoxy)styryl)isonicotinamide. To a stirred solution of (E)-3-(4-(trifluoromethoxy)styryl)isonicotinic acid (0.150 g, 0.483 mmol, 1.0 equiv) in DMF (5 mL), was added (S)-4,4-difluoro-1-glycylpyrrolidine-2-carbonitrile hydrochloride (0.108 g, 0.483 mmol, 1.1 equiv), EDCI.HCl (0.185 g, 0.966 mmol, 2.0 equiv) and HOBt (0.136 g, 0.966 mmol, 2.0 equiv). The resulting reaction mixture was allowed to stir at RT for 10 min. TEA (0.3 mL) was added and the mixture was allowed to stir at RT for overnight. Product formation was confirmed by LCMS and TLC. After completion of reaction, the mixture was diluted with water (50 mL) and extracted with ethyl acetate (75 mL×2). Combined organic extracts were washed with water (25 mL×4), dried over anhydrous Na₂SO₄ and concentrated. The crude product was purified by reversed phase HPLC to obtain (S,E)-N-(2-(2-cyano-4,4-difluoropyrrolidin-1-yl)-2-oxoethyl)-3-(4-(trifluoromethoxy)styryl)isonicotinamide (0.020 g, 8% Yield) as a white solid.

LCMS 481.2 [M+H]⁺

¹H NMR (400 MHz, DMSO-d₆) δ 9.15 (s, 1H) 9.02 (t, J=5.70 Hz, 1H) 8.54 (d, J=5.26 Hz, 1H) 7.84 (d, J=8.33 Hz, 2H) 7.75 (d, J=16.66 Hz, 1H) 7.53 (d, J=16.66 Hz, 1H) 7.30-7.46 (m, 3H) 5.20 (dd, J=9.21, 2.63 Hz, 1H) 4.27-4.38 (m, 1H) 3.99-4.27 (m, 3H) 2.78-3.07 (m, 2H).

### Example S21

### Synthesis of (S,E)-6-(4-chlorostyryl)-N-(2-(2-cyano-4,4-difluoropyrrolidin-1-yl)-2-oxoethyl)quinoline-4-carboxamide

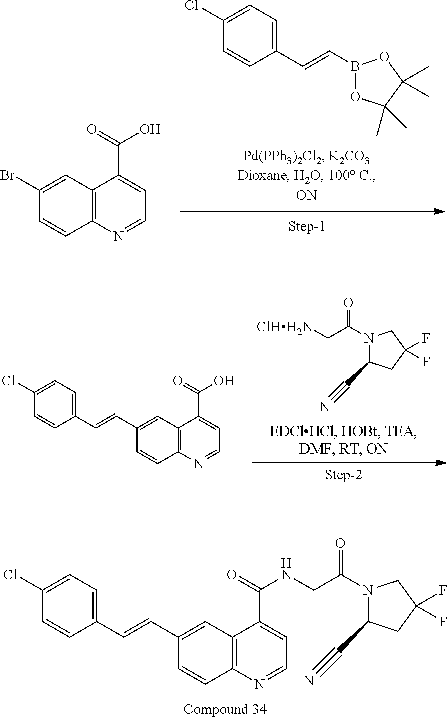

Step 1: Synthesis of (E)-6-(4-chlorostyryl)quinoline-4-carboxylic acid. To a solution of 6-bromoquinoline-4-carboxylic acid (0.05 g, 0.199 mmol, 1.0 equiv) in dioxane (4 mL) and water (2 mL) was added (E)-2-(4-chlorostyryl)-4,4,5,5-tetramethyl-1,3,2-dioxaborolane (0.079 g, 0.298 mmol, 1.5 equiv), K₂CO₃ (0.055 g, 0.397 mmol, 2.0 equiv) and the resulting reaction mixture was purged with N₂ gas for 10 min, followed by the addition of Pd(PPh₂)Cl₂ (0.008 g, 0.01 mmol. 0.05 equiv). The resulting reaction mixture was heated at 100° C. for overnight. Product formation was confirmed by LCMS. After the completion of reaction, the reaction mixture was quenched with water (10 mL) and aqueous layer washed with ethyl acetate (10 mL). Aqueous layer was separated and acidified with 6N HCl (pH˜5 to 6), solid precipitate formed which was filtered off and dried under vacuum to obtain (E)-6-(4-chlorostyryl)quinoline-4-carboxylic acid (0.06 g, 96% Yield) as a yellow solid.

LCMS 310.1 [M+H]⁺

¹H NMR (400 MHz, DMSO-d₆) δ 8.98 (d, J=4.38 Hz, 1H), 8.75 (s, 1H), 8.24 (d, J=8.33 Hz, 1H), 8.10 (d, J=8.77 Hz, 1H), 7.91 (d, J=4.38 Hz, 1H), 7.75 (d, J=8.33 Hz, 2H), 7.54-7.67 (m, 1H), 7.33-7.54 (m, 3H).

Step 2: Synthesis of (S,E)-6-(4-chlorostyryl)-N-(2-(2-cyano-4,4-difluoropyrrolidin-1-yl)-2-oxoethyl)quinoline-4-carboxamide. To a stirred solution of (E)-6-(4-chlorostyryl)quinoline-4-carboxylic acid (0.06 g, 0.194 mmol, 1.0 equiv) in DMF (3 mL), was added (S)-4,4-difluoro-1-glycylpyrrolidine-2-carbonitrile hydrochloride (0.065 g, 0.291 mmol, 1.5 equiv), EDCI.HCl (0.056 g, 0.291 mmol, 1.5 equiv) and HOBt (0.040 g, 0.291 mmol, 1.5 equiv). The mixture was allowed to stir at RT for 10 min. Triethylamine (0.1 mL) was added and the mixture was allowed to stir at RT for overnight. Product formation was confirmed by LCMS and TLC. After completion of reaction, the reaction mixture was diluted with water (10 mL) and extracted with ethyl acetate (20 mL×2). Combined organic extracts were washed with water (10 mL×4), dried over anhydrous Na₂SO₄ and concentrated. The crude product was enriched by flash chromatography (5% Methanol in DCM as an eluent) followed by reversed phase HPLC to obtain (S,E)-6-(4-chlorostyryl)-N-(2-(2-cyano-4,4-difluoropyrrolidin-1-yl)-2-oxoethyl)quinoline-4-carboxamide (0.020 g, 21.5% Yield) as a yellow solid.

LCMS 481.2 [M+H]⁺

¹H NMR (400 MHz, DMSO-d₆) δ 9.18 (br. s., 1H), 8.94 (d, J=3.95 Hz, 1H), 8.60 (s, 1H), 7.96-8.15 (m, 2H), 7.73 (d, J=8.77 Hz, 2H), 7.51-7.61 (m, 2H), 7.30-7.49 (m, 3H), 5.23 (d, J=6.14 Hz, 1H), 4.11-4.40 (m, 3H), 2.87 (d, J=17.10 Hz, 3H).

### Example S22

### Synthesis of (S)—N-(2-(2-cyano-4,4-difluoropyrrolidin-1-yl)-2-oxoethyl)-6-(4-(trifluoromethyl)phenyl)quinoline-4-carboxamide

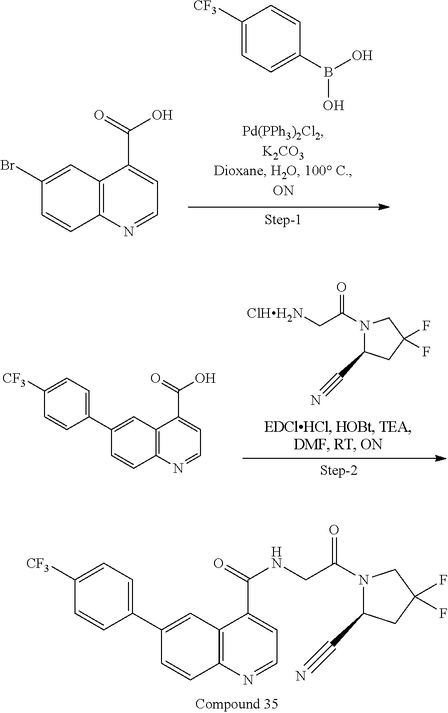

Step 1: Synthesis of 6-(4-(trifluoromethyl)phenyl)quinoline-4-carboxylic acid. To a solution of 6-bromoquinoline-4-carboxylic acid (0.05 g, 0.199 mmol, 1.0 equiv) in dioxane (4 mL) and water (2 mL) was added (4-(trifluoromethyl)phenyl)boronic acid (0.057 g, 0.298 mmol, 1.5 equiv), K₂CO₃ (0.055 g, 0.397 mmol, 2.0 equiv) and the resulting reaction mixture was purged with N₂ gas for 10 min, followed by the addition of Pd(PPh₂)Cl₂ (0.008 g, 0.009 mmol. 0.05 equiv). The resulting reaction mixture was heated at 100° C. for overnight. Product formation was confirmed by LCMS. After the completion of reaction, the mixture was quenched with water (10 mL) and aqueous layer washed with ethyl acetate (10 mL). Aqueous layer was separated and acidify with 6N HCl (pH˜5 to 6), solid precipitate formed was filtered off and dried under vacuum to obtain 6-(4-(trifluoromethyl)phenyl)quinoline-4-carboxylic acid (0.054 g, 82.53% Yield) as an off white solid.

LCMS 318 [M+H]⁺

¹H NMR (DMSO-d₆, 400 MHz) δ 8.98-9.19 (m, 2H), 8.12-8.35 (m, 2H), 7.97-8.09 (m, 3H), 7.92 (d, J=8.3 Hz, 2H).

Step 2: Synthesis of (S)—N-(2-(2-cyano-4,4-difluoropyrrolidin-1-yl)-2-oxoethyl)-6-(4-(trifluoromethyl)phenyl)quinoline-4-carboxamide. To a stirred solution of 6-(4-(trifluoromethyl)phenyl)quinoline-4-carboxylic acid (0.05 g, 0.158 mmol, 1.0 equiv) in DMF (3 mL), was added (S)-4,4-difluoro-1-glycylpyrrolidine-2-carbonitrile hydrochloride (0.053 g, 0.236 mmol, 1.5 equiv), EDCI.HCl (0.045 g, 0.236 mmol, 1.5 equiv) and HOBt (0.032 g, 0.236 mmol, 1.5 equiv). The mixture was allowed to stir at RT for 10 min. Triethylamine (0.1 mL) was added and the resulting reaction mixture was allowed to stir at RT for overnight. Product formation was confirmed by LCMS and TLC. After completion of reaction, the reaction mixture was diluted with water (10 mL) and extracted with ethyl acetate (20 mL×2). Combined organic extracts were washed with water (10 mL×4), dried over anhydrous Na₂SO₄ and concentrated. The crude product was enriched by flash chromatography (5% Methanol in DCM as an eluent) followed by reversed phase HPLC to obtain (S)—N-(2-(2-cyano-4,4-difluoropyrrolidin-1-yl)-2-oxoethyl)-6-(4-(trifluoromethyl)phenyl)quinoline-4-carboxamide (0.016 g, 23.37% Yield) as a white solid.

LCMS 489.3 [M+H]⁺

¹H NMR (400 MHz, DMSO-d₆) δ 9.18-9.31 (m, 1H), 9.02 (d, J=3.95 Hz, 1H), 8.97 (d, J=1.75 Hz, 1H), 8.27-8.36 (m, 2H), 8.11-8.27 (m, 3H), 7.90 (d, J=8.33 Hz, 1H), 7.62 (d, J=4.38 Hz, 1H), 5.15-5.30 (m, 1H), 4.24-4.40 (m, 2H), 4.05-4.24 (m, 1H), 2.95 (br. s., 1H), 2.87 (d, J=16.66 Hz, 2H).

### Example S23

### Synthesis of (S)—N-(2-(2-cyano-4,4-difluoropyrrolidin-1-yl)-2-oxoethyl)-3-((4-methoxyphenyl)ethynyl)isonicotinamide

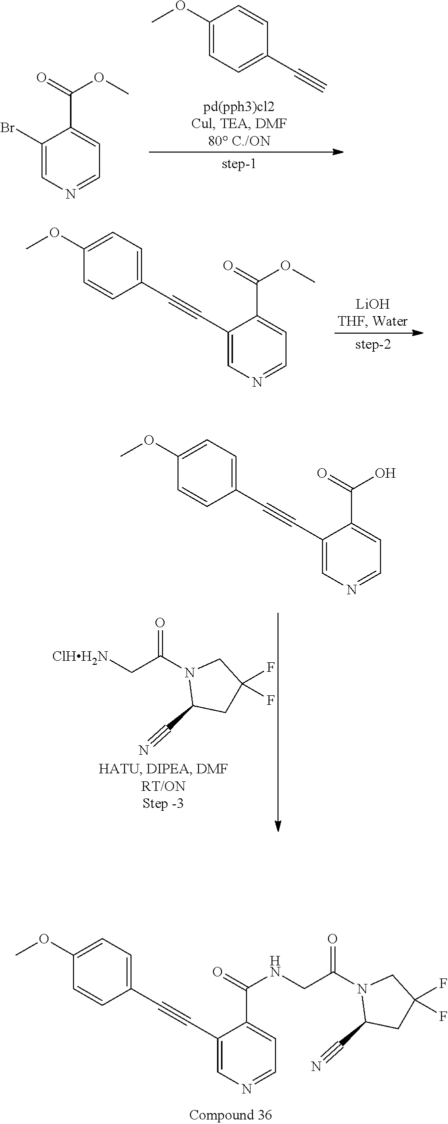

Step 1: Synthesis of methyl 3-((4-methoxyphenyl)ethynyl)isonicotinate. To a solution of methyl 3-bromoisonicotinate (1000 mg, 4.6 mmol, 1.0 equiv) in DMF (10 mL) was added 1-ethynyl-4-methoxybenzene (611 mg, 4.6 mmol, 1.0 equiv), CuI (87 mg, 0.46 mmol, 0.1 equiv), DIPEA (1186 mg, 9.2 mmol, 2.0 equiv), followed by the addition of pd(pph3)₂Cl₂ (161 mg, 0.23 mmol. 0.05 equiv). The resulting reaction mixture was heated at 80° C. for overnight. Product formation was confirmed by LCMS. After completion of reaction, the mixture was diluted with water (50 mL) and extracted with ethyl acetate (100 mL×2). Combined organic extracts were washed with water (50 mL×2), dried over anhydrous Na₂SO₄ and concentrated. The crude product obtained was purified by flash chromatography (0-30% EtOAc in Hexane as an eluent) to obtain methyl 3-((4-methoxyphenyl)ethynyl)isonicotinate (300 mg, 24% Yield) as a yellow solid.

LCMS 267.0 [M+H]⁺

Step 2: Synthesis of 3-((4-methoxyphenyl)ethynyl)isonicotinic acid. To a stirred solution of methyl 3-((4-methoxyphenyl)ethynyl)isonicotinate (300 mg, 1.11 mmol, 1.0 equiv) in THF (10 mL) and water (05 mL), was added LiOH (53 mg, 2.23 mmol, 2.0 equiv). The mixture was allowed to stir at RT for overnight. Product formation was confirmed by LCMS and ¹H NMR spectroscopy. The reaction mixture was diluted with water (15 mL) and washed with ethyl acetate (15 mL). Aqueous layer was separated and freeze dried on lyophilyzer to obtain 3-((4-methoxyphenyl)ethynyl)isonicotinic acid (400 mg, Quant. Yield) as a white solid.

LCMS 253.0 [M+H]⁺

Step 3: Synthesis of (S)—N-(2-(2-cyano-4,4-difluoropyrrolidin-1-yl)-2-oxoethyl)-3-((4-methoxyphenyl)ethynyl)isonicotinamide. To a stirred solution of 3-((4-methoxyphenyl)ethynyl)isonicotinic acid (100 mg, 0.39 mmol, 1.0 equiv) in DMF (05 mL), was added HATU (296 mg, 0.78 mmol, 2.0 equiv), (S)-4,4-difluoro-1-glycylpyrrolidine-2-carbonitrile hydrochloride (89 mg, 0.39 mmol, 1.0 equiv) followed by the addition of DIPEA (151 mg, 1.17 mmol, 3.0 equiv). The resulting reaction mixture was allowed to stir for overnight at RT. The product formation was confirmed by TLC and LCMS. After completion of the reaction, the reaction mixture was diluted with water (20 mL) and extracted with ethyl acetate (40 mL×2). Organic layer was washed with water (20 mL×4), dried over anhydrous sodium sulphate and concentrated under reduced pressure. The crude product was purified by reversed phase HPLC to obtain (S)—N-(2-(2-cyano-4,4-difluoropyrrolidin-1-yl)-2-oxoethyl)-3-((4-methoxyphenyl)ethynyl)isonicotinamide (05 mg, 04% Yield) as a yellow solid.

LCMS 425.2 [M+H]⁺

¹H NMR (400 MHz, DMSO-d₆) δ 8.92 (br. s., 1H), 8.82 (s, 1H), 8.64 (d, J=5.26 Hz, 1H), 7.45-7.65 (m, 3H), 6.99 (d, J=8.77 Hz, 2H), 5.13 (d, J=7.89 Hz, 1H), 4.29 (d, J=15.35 Hz, 2H), 4.21 (br. s., 2H), 3.80 (s, 3H), 2.79-2.98 (m, 2H).

### Example S24

### Synthesis of (S)-3-((4-chlorophenyl)ethynyl)-N-(2-(2-cyano-4,4-difluoropyrrolidin-1-yl)-2-oxoethyl)isonicotinamide

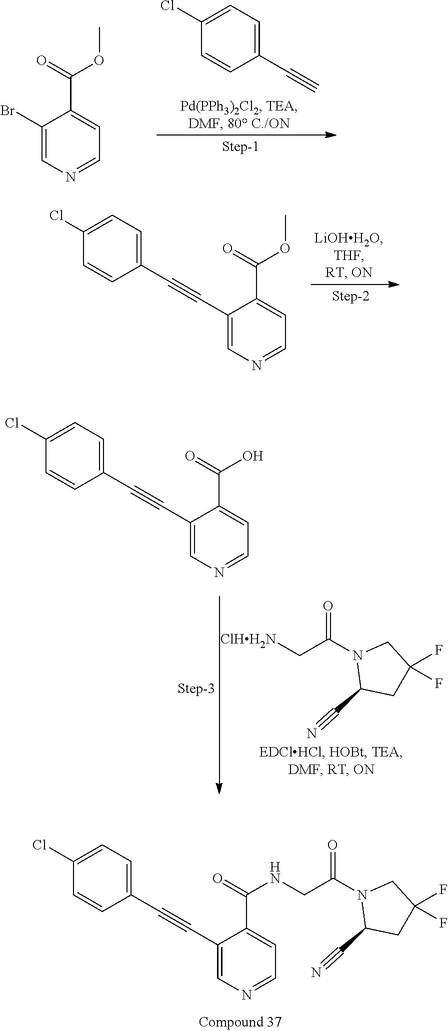

Step 1: Synthesis of methyl 3-((4-chlorophenyl)ethynyl)isonicotinate. To a solution of methyl-3-bromoisonicotinate (1 g, 4.62 mmol, 1.0 equiv) in DMF (10 mL) was added 1-chloro-4-ethynylbenzene (0.935 g, 6.88 mmol, 1.5 equiv) and TEA (1.38 g, 2.28 mmol, 3.0 equiv) at RT. The resulting reaction mixture was purged with N₂ gas for 5 min, followed by the addition of Pd(PPh₂)Cl₂ (0.160 g, 0.229 mmol. 0.05 equiv). The reaction mixture was heated at 80° C. for overnight. Product formation was confirmed by LCMS and TLC. After completion of reaction, the reaction mixture was diluted with water (50 mL) and extracted with ethyl acetate (100 mL×2). Combined organic extracts were washed with water (50 mL×4), dried over anhydrous Na₂SO₄ and concentrated. The crude product obtained was purified by flash chromatography (0-30% ethyl acetate in hexane as an eluent) to obtain methyl 3-((4-chlorophenyl)ethynyl)isonicotinate (0.408 g, 32% Yield) as a yellow solid.

LCMS 272.1 [M+H]⁺

Step 2: Synthesis of 3-((4-chlorophenyl)ethynyl)isonicotinic acid. To a stirred solution of methyl 3-((4-chlorophenyl)ethynyl)isonicotinate (0.05 g, 0.183 mmol, 1.0 equiv) in THF (3 ml) was added LiOH.H₂O (0.009 g, 0.220 mmol, 1.2 equiv) and H₂O (1 ml) at RT. The mixture was allowed to stir at RT for overnight. Product formation was confirmed by LCMS. After completion of reaction, the reaction mixture was diluted with water (10 mL) and washed with ethyl acetate (10 mL). The aqueous layer was separated and freeze dried on lyophilyzer to obtain 3-((4-chlorophenyl)ethynyl)isonicotinic acid (0.050 g, Quant. Yield) as a yellow solid.

LCMS 258.1 [M+H]⁺

Step 3: Synthesis of (S)-3-((4-chlorophenyl) ethynyl)-N-(2-(2-cyano-4,4-difluoropyrrolidin-1-yl)-2-oxoethyl)isonicotinamide. To a stirred solution of 3-((4-chlorophenyl)ethynyl)isonicotinic acid (0.150 g, 0.583 mmol, 1.0 equiv) in DMF (7 ml) was added EDCI.HCl (0.167 g, 0.875 mmol, 1.5 equiv), HOBt (0.118 g, 0.875 mmol, 1.5 equiv) and (S)-4,4-difluoro-1-glycylpyrrolidine-2-carbonitrile hydrochloride (0.196 g, 0.875 mmol, 1.5 equiv) followed by the addition of TEA (0.176 g, 1.749 mmol, 3.0 equiv). The resulting reaction mixture was allowed to stir at RT for overnight. Product formation was confirmed by LCMS. After completion of reaction, the reaction mixture was diluted with water (20 mL) and extracted with ethyl acetate (50 mL×2). Combined organic extracts were washed with water (20 mL×4), dried over anhydrous Na₂SO₄ and concentrated under reduced pressure. The crude product was purified by reversed phase HPLC to obtain (S)-3-((4-chlorophenyl)ethynyl)-N-(2-(2-cyano-4,4-difluoropyrrolidin-1-yl)-2-oxoethyl)isonicotinamide (0.024 g, 9.6% Yield) as a white solid.

LCMS 429.3 [M+H]⁺

¹H NMR (400 MHz, DMSO-d₆) δ 8.97 (br. s., 1H) 8.86 (s, 1H) 8.68 (d, J=4.82 Hz, 1H) 7.64 (d, J=8.77 Hz, 2H) 7.43-7.60 (m, 3H) 5.14 (d, J=7.02 Hz, 1H) 4.06-4.36 (m, 4H) 2.76-3.00 (m, 2H).

### Example S25

### Synthesis of (S)—N-(2-(2-cyano-4,4-difluoropyrrolidin-1-yl)-2-oxoethyl)-6-(6-(trifluoromethyl)pyridin-3-yl)quinoline-4-carboxamide

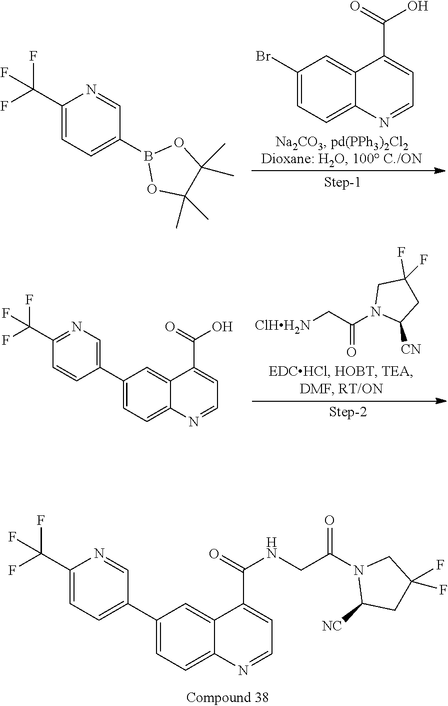

Step 1: Synthesis of 6-(6-(trifluoromethyl)pyridin-3-yl)quinoline-4-carboxylic acid. To the solution of 5-(4,4,5,5-tetramethyl-1,3,2-dioxaborolan-2-yl)-2-(trifluoromethyl)pyridine (100 mg, 0.36 mmol, 1.0 equiv) in Dioxane:water (05:02 mL), was added 6-bromoquinoline-4-carboxylic acid (93 mg, 0.36 mmol, 1.0 equiv), Na₂CO₃ (77 mg, 0.72 mmol, 2.0 equiv) followed by the addition of Pd(PPh3)2Cl₂ (13 mg, 0.018 mmol, 0.05 equiv). The resulting reaction mixture was heated at 100° C. for overnight. Product formation was confirmed by LCMS. After completion of reaction the reaction mixture was diluted with water (30 mL). Aqueous layer was washed with extracted with ethyl acetate (50 mL×2) separated and freeze dried over lyophilizer to obtain 6-(6-(trifluoromethyl)pyridin-3-yl)quinoline-4-carboxylic acid (100 mg) as an off white solid.

LCMS 319.0 [M+H]⁺

Step 2: Synthesis of (S)—N-(2-(2-cyano-4,4-difluoropyrrolidin-1-yl)-2-oxoethyl)-6-(6-(trifluoromethyl)pyridin-3-yl)quinoline-4-carboxamide. To a stirred solution of 6-(6-(trifluoromethyl)pyridin-3-yl)quinoline-4-carboxylic acid (100 mg, 0.31 mmol, 1.0 equiv) in DMF (05 mL), was added (S)-4,4-difluoro-1-glycylpyrrolidine-2-carbonitrile hydrochloride (71 mg, 0.31 mmol, 1.0 equiv), HOBT (63 mg, 0.46 mmol, 1.5 equiv) and EDC.HCl (89 mg, 0.46 mmol, 1.5 equiv). The mixture was allowed to stir at RT for 10 min. Triethyl amine (0.10 mL) was added and the mixture was allowed to stir at RT for overnight. Product formation was confirmed by LCMS and TLC. After completion of reaction, the mixture was diluted with water (50 mL) and extracted with ethyl acetate (50 mL×2). Combined organic extracts were washed with water (20 mL×4), dried over anhydrous Na₂SO₄ and concentrated. The crude product obtained was purified reversed phase HPLC to obtain (S)—N-(2-(2-cyano-4,4-difluoropyrrolidin-1-yl)-2-oxoethyl)-6-(6-(trifluoromethyl)pyridin-3-yl)quinoline-4-carboxamide (15 mg, 10% Yield) as an off-white solid.

LCMS 490.3 [M+H]⁺

¹H NMR (400 MHz, DMSO-d₆) δ 9.30 (s, 1H), 9.23 (br. s., 1H), 9.05 (d, J=4.39 Hz, 1H), 8.99 (s, 1H), 8.65 (d, J=10.96 Hz, 1H), 8.37 (br. s., 1H), 8.26 (d, J=8.77 Hz, 1H), 8.06 (d, J=8.33 Hz, 2H), 7.65 (d, J=4.38 Hz, 1H), 5.21 (d, J=7.02 Hz, 1H), 4.27-4.39 (m, 2H), 4.12 (d, J=11.40 Hz, 2H), 2.91 (d, J=17.10 Hz, 2H).

### Example S26

### Synthesis of (S,E)-6-(2-(4-chlorophenyl)prop-1-en-1-yl)-N-(2-(2-cyano-4,4-difluoropyrrolidin-1-yl)-2-oxoethyl)quinoline-4-carboxamide

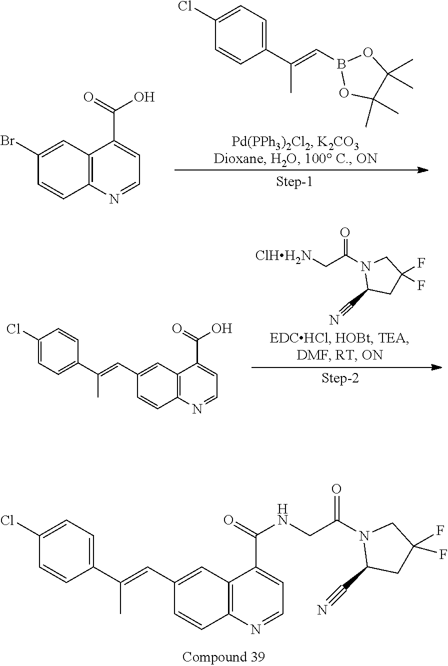

Step 1: Synthesis of (E)-6-(2-(4-chlorophenyl)prop-1-en-1-yl)quinoline-4-carboxylic acid. To a solution of 6-bromoquinoline-4-carboxylic acid (0.3 g, 1.14 mmol, 1.0 equiv) in dioxane (4 mL) and water (2 mL) was added (E)-2-(2-(4-chlorophenyl)prop-1-en-1-yl)-4,4,5,5-tetramethyl-1,3,2-dioxaborolane (0.288 g, 0.91 mmol, 0.8 equiv), K₂CO₃ (0.241 g, 2.28 mmol, 2.0 equiv) and the mixture purged with N₂ gas for 10 min, followed by the addition of Pd(PPh₂)Cl₂ (0.042 g, 0.057 mmol. 0.05 equiv). The resulting reaction mixture was heated at 100° C. for overnight. Product formation was confirmed by LCMS. After the completion of reaction the reaction mixture was diluted with water (30 mL) and washed with ethyl acetate (50 mL×2). Aqueous layer was separated and dried over lyophilizer to obtain (E)-6-(2-(4-chlorophenyl)prop-1-en-1-yl)quinoline-4-carboxylic acid (0.350 g, Quant. Yield) as an off-white solid.

LCMS 324.1 [M+H]⁺

Step 2: Synthesis of (S,E)-6-(2-(4-chlorophenyl)prop-1-en-1-yl)-N-(2-(2-cyano-4,4-difluoropyrrolidin-1-yl)-2-oxoethyl)quinoline-4-carboxamide. To a stirred solution of (E)-6-(4-chlorostyryl)quinoline-4-carboxylic acid (0.35 g, 1.08 mmol, 1.0 equiv) in DMF (5 mL) was added EDCI.HCl (0.311 g, 1.62 mmol, 1.5 equiv), HOBt (0.218 g, 1.62 mmol, 1.5 equiv) and (S)-4,4-difluoro-1-glycylpyrrolidine-2-carbonitrile hydrochloride (0.364 g, 1.62 mmol, 1.5 equiv) followed by the addition of triethylamine (0.327 g, 3.24 mmol, 3.0 equiv). The resulting reaction mixture was allowed to stir at RT for overnight. Product formation was confirmed by LCMS. After completion of reaction, the reaction mixture was diluted with water (25 mL) and extracted with ethyl acetate (50 mL×2). Combined organic extracts were washed with water (20 mL×4), dried over anhydrous Na₂SO₄ and concentrated under reduced pressure. The crude product was purified by flash chromatography (0-5% MeOH in DCM as an eluent) followed by reversed phase HPLC purification to obtain (S,E)-6-(2-(4-chlorophenyl)prop-1-en-1-yl)-N-(2-(2-cyano-4,4-difluoropyrrolidin-1-yl)-2-oxoethyl)quinoline-4-carboxamide (0.02 g, 3.7% Yield) as a brown solid.

LCMS 495.4 [M+H]⁺

¹H NMR (400 MHz, DMSO-d₆) δ 9.16 (t, J=5.92 Hz, 1H) 8.97 (d, J=4.38 Hz, 1H) 8.41 (s, 1H) 8.09 (d, J=8.77 Hz, 1H) 7.87 (d, J=8.33 Hz, 1H) 7.67 (d, J=8.77 Hz, 2H) 7.58 (d, J=4.38 Hz, 1H) 7.47 (d, J=8.77 Hz, 2H) 7.11 (s, 1H) 5.16 (d, J=7.02 Hz, 1H) 4.08-4.38 (m, 4H) 2.78-2.99 (m, 2H) 2.32 (s, 3H).

### Example S27

### Synthesis of (S,E)-3-(2-(6-chloronaphthalen-2-yl)vinyl)-N-(2-(2-cyano-4,4-difluoropyrrolidin-1-yl)-2-oxoethyl)isonicotinamide

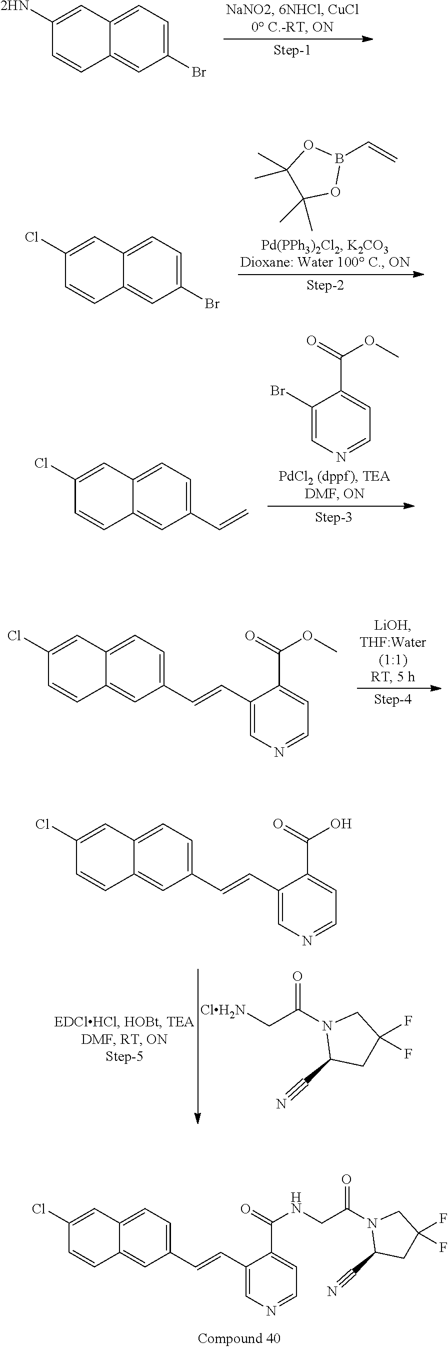

Step 1: Synthesis of 2-bromo-6-chloronaphthalene. To a stirred solution of 6-chloronaphthalen-2-amine (1.0 g, 4.504 mmol, 1.0 equiv) in 6N HCl (10 mL) was added NaNO₂ (0.372 g, 5.405 mmol, 1.2 equiv) in water (10 mL) at 0° C. and resulting reaction mixture was stir at RT for one hour. A solution of CuCl (2.2 g, 22.522 mmol, 5.0 equiv) in 6N HCl (10 mL) was added and again stirred at RT for overnight. After the completion of reaction (monitored by TLC), the reaction mixture was diluted with water (100 mL) and extracted with ethyl acetate (150 mL×3). Combined organic extracts were washed with water (50 mL×2) & brine (50 mL), dried over anhydrous Na₂SO₄ and concentrated. The crude product was purified by flash chromatography (hexane as an eluent) to obtain 2-bromo-6-chloronaphthalene (0.600 g, 55.5% Yield) as a white solid.

¹H NMR (400 MHz, DMSO-d₆) δ 8.27 (s, 1H) 8.10 (s, 1H) 7.97 (d, J=8.77 Hz, 1H) 7.90 (d, J=8.77 Hz, 1H) 7.70 (dd, J=8.77, 1.75 Hz, 1H) 7.59 (dd, J=8.99, 1.97 Hz, 1H).

Step 2: Synthesis of 2-chloro-6-vinylnaphthalene. To a stirred solution of 2-bromo-6-chloronaphthalene (0.250 g, 1.061 mmol, 1.0 equiv) in Dioxane (9 mL) was added 4,4,5,5-tetramethyl-2-vinyl-1,3,2-dioxaborolane (0.395 g, 1.562 mmol, 1.5 equiv) and a solution of K₂CO₃ (0.287 g, 2.083 mmol, 2.0 equiv) in water (3 mL), the mixture purged with N₂ gas for 10 min, followed by the addition of Pd(PPh₃)Cl₂ (0.036 g, 0.0520 mmol. 0.05 equiv). The resulting reaction mixture was heated at 100° C. for overnight. Product formation was confirmed by TLC. After completion of reaction the reaction mixture was diluted with water (50 mL) extracted with ethyl acetate (100 mL×2). Combined organic extracts were washed with water (50 mL×2) and brine (50 mL), dried over anhydrous Na₂SO₄ and concentrated. The crude product was purified by flash chromatography (hexane as an eluent) to obtain 2-chloro-6-vinylnaphthalene (0.150 g, 76.92% Yield) as a white solid.

¹H NMR (400 MHz, CHLOROFORM-d) δ 7.52-7.82 (m, 4H) 7.40 (dd, J=8.77, 2.19 Hz, 1H) 7.26 (s, 1H) 6.86 (dd, J=17.54, 10.96 Hz, 1H) 5.88 (d, J=17.54 Hz, 1H) 5.36 (d, J=10.96 Hz, 1H).

Step 3: Synthesis of methyl (E)-3-(2-(6-chloronaphthalen-2-yl)vinyl)isonicotinate. To a stirred solution of 2-chloro-6-vinylnaphthalene (0.250 g, 1.32 mmol, 1.0 equiv) in DMF (10 mL) was added methyl 3-bromoisonicotinate (0.574 g, 2.65 mmol, 2.0 equiv) and triethyl amine (0.575 ml, 3.983 mmol, 3.0 equiv). The resulting reaction mixture was purged with N₂ gas for 5 min followed by addition of pd(dppf)Cl₂ (0.097 g, 0.132 mmol, 0.1 equiv). The reaction mixture was heated at 100° C. for overnight. Product formation was confirmed by TLC. After completion of reaction the reaction mixture was diluted with water (50 mL) extracted with ethyl acetate (100 mL×2). Combined organic extracts were washed with water (50 mL×2) & brine (50 mL), dried over anhydrous Na₂SO₄ and concentrated. The crude product was purified by flash chromatography (0-15% EA/Hexane as an eluent) to obtain methyl (E)-3-(2-(6-chloronaphthalen-2-yl)vinyl)isonicotinate (0.120 g, 28% Yield) as a white solid.

LCMS 324.2 [M+H]⁺

¹H NMR (400 MHz, CHLOROFORM-d) δ 9.06 (s, 1H) 8.63 (d, J=5.26 Hz, 1H) 8.00 (d, J=16.22 Hz, 1H) 7.89 (s, 1H) 7.66-7.84 (m, 4H) 7.44 (dd, J=8.77, 2.19 Hz, 1H) 7.19-7.28 (m, 2H) 3.99 (s, 3H).

Step 4: Synthesis of (E)-3-(2-(6-chloronaphthalen-2-yl)vinyl)isonicotinic acid. To a stirred solution of methyl (E)-3-(2-(6-chloronaphthalen-2-yl)vinyl)isonicotinate (0.120 g, 0.370 mmol, 1.0 equiv) in THF (6 ml) was added a solution of LiOH.H₂O (0.031 g, 0.741 mmol, 2.0 equiv) in water (6 ml). The reaction mixture was stirred at RT for 5 h. Product formation was confirmed by TLC. Reaction mixture was diluted with water (20 ml) and washed with ethyl acetate (25 mL×3). Aqueous layer was separated and freeze dried over lyophilizer to obtain compound (E)-3-(2-(6-chloronaphthalen-2-yl)vinyl)isonicotinic acid (0.100 g, 87% Yield) as a white solid.

LCMS 310.0 [M+H]⁺

¹H NMR (400 MHz, DMSO-d₆) δ 8.85 (s, 1H) 8.31 (d, J=4.82 Hz, 1H) 8.02 (s, 1H) 7.98 (br. s., 1H) 7.87-7.98 (m, 2H) 7.79 (d, J=7.45 Hz, 1H) 7.52 (dd, J=8.77, 1.75 Hz, 1H) 7.35 (d, J=16.66 Hz, 1H) 7.27 (d, J=4.82 Hz, 1H).

Step 5: Synthesis of (S,E)-3-(2-(6-chloronaphthalen-2-yl)vinyl)-N-(2-(2-cyano-4,4-difluoropyrrolidin-1-yl)-2-oxoethyl)isonicotinamide. To a stirred solution of (E)-3-(2-(6-chloronaphthalen-2-yl)vinyl)isonicotinic acid (0.100 g, 0.323 mmol, 1.0 equiv) in DMF (3 mL), was added (S)-4,4-difluoro-1-glycylpyrrolidine-2-carbonitrile hydrochloride (0.109 g, 0.485 mmol, 1.5 equiv), HOBt (0.065 g, 0.485 mmol, 1.5 equiv) and EDCI.HCl (0.092 g, 0.485 mmol, 1.5 equiv) followed by the addition of TEA (0.09 mL, 0.647 mmol, 2.0 equiv). The resulting reaction mixture was allowed to stir at RT for overnight. Product formation was confirmed by LCMS and TLC. After completion of reaction, the reaction mixture was diluted with water (20 mL) and extracted with ethyl acetate (50 mL×2). Combined organic extracts were washed with water (20 mL×2) and brine (20 mL). Organic layer was separated and dried over anhydrous Na₂SO₄ and concentrated under reduced pressure. The crude product was purified by flash chromatography (0-5% MeOH in DCM as an eluent) to obtain (S,E)-3-(2-(6-chloronaphthalen-2-yl)vinyl)-N-(2-(2-cyano-4,4-difluoropyrrolidin-1-yl)-2-oxoethyl)isonicotinamide (0.030 g, 20% Yield) as an off white solid.

LCMS 481.1 [M+H]⁺

¹H NMR (400 MHz, DMSO-d₆) δ 9.20 (s, 1H) 9.06 (t, J=5.70 Hz, 1H) 8.55 (d, J=4.82 Hz, 1H) 8.18 (s, 1H) 7.97-8.07 (m, 2H) 7.92 (d, J=8.77 Hz, 1H) 7.82 (d, J=16.66 Hz, 1H) 7.65 (d, J=16.66 Hz, 1H) 7.48-7.61 (m, 1H) 7.38 (d, J=4.82 Hz, 1H) 5.23 (d, J=6.58 Hz, 1H) 4.34 (t, J=11.62 Hz, 1H) 4.23 (br. s., 1H) 4.03-4.23 (m, 2H) 2.76-3.05 (m, 3H).

### Example S28

### Synthesis of (S)—N-(2-(2-cyano-4,4-difluoropyrrolidin-1-yl)-2-oxoethyl)-6-((4-methoxyphenyl)ethynyl)quinoline-4-carboxamide

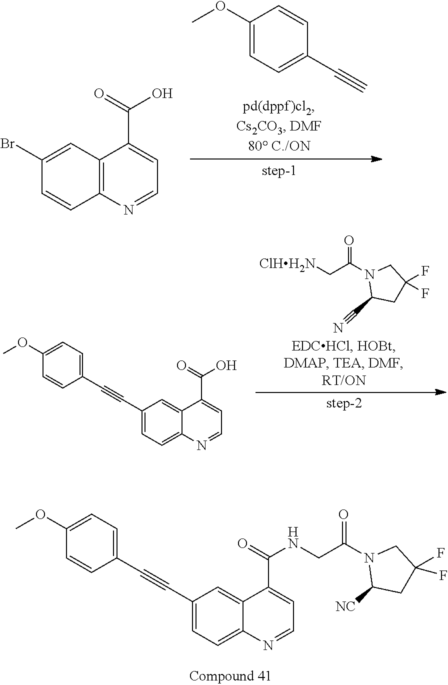

Step 1: Synthesis of 6-((4-methoxyphenyl)ethynyl)quinoline-4-carboxylic acid. To a solution of 6-bromoquinoline-4-carboxylic acid (250 mg, 1.0 mmol, 1.0 equiv) in DMF (07 mL) was added 1-ethynyl-4-methoxybenzene (204 mg, 1.2 mmol, 1.5 equiv), Cs2CO3 (487 mg, 1.5 mmol, 1.5 equiv), Pd(dppf)Cl₂ (35 mg, 0.05 mmol, 0.05 equiv). The resulting reaction mixture was stirred at 80° C. for overnight. Product formation was confirmed by LCMS. After completion of reaction the reaction mixture was diluted with water (30 mL) and washed with ethyl acetate (10 mL×2). Aqueous layer was separated and freeze dried over lyophilizer to obtain 6-((4-methoxyphenyl)ethynyl)quinoline-4-carboxylic acid (100 mg, 33% Yield) as an off white solid.

LCMS 304.0 [M+H]⁺

Step 2: Synthesis of (S)—N-(2-(2-cyano-4,4-difluoropyrrolidin-1-yl)-2-oxoethyl)-6-((4-methoxyphenyl)ethynyl)quinoline-4-carboxamide. To a stirred solution of 6-((4-methoxyphenyl)ethynyl)quinoline-4-carboxylic acid (100 mg, 0.33 mmol, 1.0 equiv) in DMF (05 mL), was added (S)-4,4-difluoro-1-glycylpyrrolidine-2-carbonitrile hydrochloride (75 mg, 0.33 mmol, 1.0 equiv), EDC.HCl (95 mg, 0.49 mmol, 1.5 equiv), HOBT (67 mg, 0.49 mmol, 1.5 equiv) and TEA (0.2 mL, 0.99 mmol, 3.0 equiv). The resulting reaction mixture was allowed to stir at RT for overnight. Product formation was confirmed by TLC and LCMS. After completion of the reaction, the reaction mixture was diluted with water (20 mL) and extracted with ethyl acetate (50 mL×2). Organic layer was washed with water (20 mL×4), brine solution (20 mL), dried over anhydrous sodium sulphate and concentrated under reduced pressure. The crude product was purified by reversed phase HPLC to obtain (S)—N-(2-(2-cyano-4,4-difluoropyrrolidin-1-yl)-2-oxoethyl)-6-((4-methoxyphenyl)ethynyl)quinoline-4-carboxamide (05 mg, 4%) as a yellow solid.

LCMS 475.3 [M+H]⁺

¹H NMR (400 MHz, DMSO-d₆) δ 9.20 (br. s., 1H), 9.01 (d, J=4.4 Hz, 1H), 8.55 (s, 1H), 8.10 (d, J=8.8 Hz, 1H), 7.89 (d, J=10.5 Hz, 1H), 7.67-7.51 (m, 3H), 7.01 (d, J=8.8 Hz, 2H), 5.20 (d, J=9.2 Hz, 1H), 4.36-4.19 (m, 3H), 4.16 (br. s., 1H), 3.81 (s, 3H), 2.86 (d, J=17.1 Hz, 2H).

### Example S29

### Synthesis of (S,E)-6-(2-(6-chloronaphthalen-2-yl)vinyl)-N-(2-(2-cyano-4,4-difluoropyrrolidin-1-yl)-2-oxoethyl)quinoline-4-carboxamide

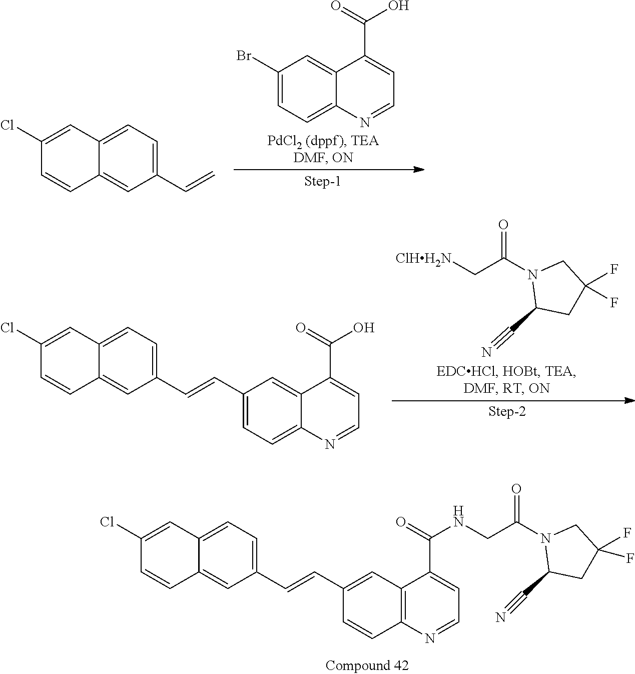

Step 1: Synthesis of (E)-6-(2-(6-chloronaphthalen-2-yl)vinyl)quinoline-4-carboxylic acid. To a stirred solution of 2-chloro-6-vinylnaphthalene (0.100 g, 0.531 mmol, 1.0 equiv) in DMF (5 mL) was added 6-bromoquinoline-4-carboxylic acid (0.134 g, 0.531 mmol, 1.0 equiv) and triethyl amine (0.230 ml, 1.595 mmol, 3.0 equiv). The resulting reaction mixture was purged with N₂ gas for 5 min followed by the addition of Pd(dppf)Cl₂ (0.038 g, 0.0531 mmol, 0.1 equiv). The reaction mixture was heated at 100° C. for overnight. Product formation was confirmed by TLC. Reaction mixture was cool to RT, diluted with water (50 mL) and washed with ethyl acetate (50 mL×3). The aqueous layer was separated and freeze dried over lyophilizer to obtain (E)-6-(2-(6-chloronaphthalen-2-yl)vinyl)quinoline-4-carboxylic acid (0.100 g, 52% Yield) as a yellow solid.

LCMS 360.0 [M+H]⁺

¹H NMR (400 MHz, DMSO-d₆) δ 8.94 (d, J=4.38 Hz, 1H) 8.83 (br. s., 1H) 8.24 (d, J=8.33 Hz, 1H) 8.17 (br. s., 1H) 8.09 (d, J=8.33 Hz, 1H) 8.04 (d, J=7.89 Hz, 2H) 7.76-8.00 (m, 2H) 7.48-7.75 (m, 1H) 6.90 (dd, J=17.32, 11.18 Hz, 1H) 6.00 (d, J=17.54 Hz, 1H) 5.39 (d, J=10.52 Hz, 1H).

Step 2: Synthesis of (S,E)-6-(2-(6-chloronaphthalen-2-yl)vinyl)-N-(2-(2-cyano-4,4-difluoropyrrolidin-1-yl)-2-oxoethyl)quinoline-4-carboxamide. To a stirred solution of (E)-6-(2-(6-chloronaphthalen-2-yl)vinyl)quinoline-4-carboxylic acid (0.100 g, 0.278 mmol, 1.0 equiv) in DMF (5 mL), was added (S)-4,4-difluoro-1-glycylpyrrolidine-2-carbonitrile hydrochloride (0.094 g, 0.417 mmol, 1.5 equiv), HOBt (0.056 g, 0.417 mmol, 1.5 equiv) and EDC.HCl (0.079 g, 0.417 mmol, 1.5 equiv) followed by the addition of TEA (0.1 mL). The resulting reaction mixture was allowed to stir at RT for overnight. Product formation was confirmed by LCMS and TLC. After completion of reaction, the reaction mixture was diluted with water (50 mL) and extracted with ethyl acetate (100 mL×2). Combined organic extracts were washed with water (50 mL×3) and brine (50 mL), dried over anhydrous Na₂SO₄ and concentrated. The crude product was purified by flash chromatography (0-5% MeOH in DCM as an eluent) followed by the reversed phase HPLC purification to obtained (S,E)-6-(2-(6-chloronaphthalen-2-yl)vinyl)-N-(2-(2-cyano-4,4-difluoropyrrolidin-1-yl)-2-oxoethyl)quinoline-4-carboxamide (0.015 g, 10% Yield) as a yellow solid.

LCMS 531.4 [M+H]⁺

¹H NMR (400 MHz, DMSO-d₆) δ 9.20 (t, J=5.70 Hz, 1H) 8.93 (d, J=4.38 Hz, 1H) 8.70 (s, 1H) 8.14 (br. s., 2H) 8.05-8.12 (m, 1H) 7.87-8.05 (m, 4H) 7.76 (d, J=16.66 Hz, 1H) 7.43-7.67 (m, 3H) 5.17-5.36 (m, 1H) 4.12-4.41 (m, 4H) 2.96 (br. s., 1H) 2.89 (d, J=9.21 Hz, 1H).

### Example S30

### Synthesis of (S,E)-N-(2-(2-cyano-4,4-difluoropyrrolidin-1-yl)-2-oxoethyl)-6-(2-(6-methoxypyridin-3-yl)vinyl)quinoline-4-carboxamide

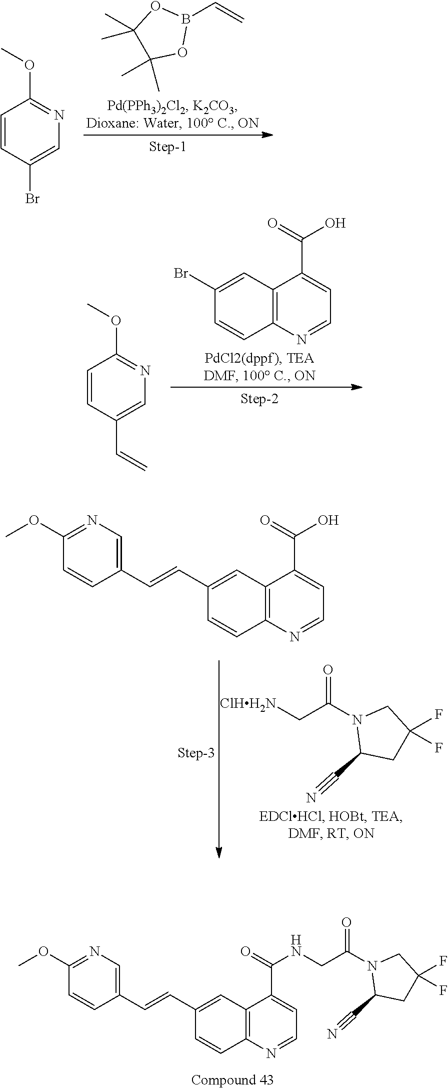

Step 1: Synthesis of 5-ethenyl-2-methoxy-pyridine. To a stirred solution of 5-bromo-2-methoxy-pyridine (0.500 g, 2.659 mmol, 1.0 equiv) in Dioxane (8 mL) was added 2-Vinyl-4,4,5,5-tetramethyl-1,3,2-dioxaoborolane (0.614 g, 3.989 mmol, 1.5 equiv) and a solution of K₂CO₃ (0.734 g, 5.319 mmol, 2.0 equiv) in water (4 mL), and resulting reaction mixture was purged with N₂ gas for 10 min, followed by the addition of Pd(PPh₃)Cl₂ (0.093 g, 0.132 mmol. 0.05 equiv). The resulting reaction mixture was heated at 100° C. for overnight. Product formation was confirmed by TLC. Reaction mixture was cooled to RT, diluted with water (50 mL) extracted with ethyl acetate (100 mL×2). Combined organic extracts were washed with water (50 mL×2) & brine (50 mL), dried over anhydrous Na₂SO₄ and concentrated. The crude product was purified by flash chromatography (0-5% EA in hexane as an eluent) to obtain 5-ethenyl-2-methoxy-pyridine (0.250 g, 89% Yield) as a yellow semi solid.

¹H NMR (400 MHz, CHLOROFORM-d) δ 8.12 (d, J=2.63 Hz, 1H) 7.69 (dd, J=8.77, 2.63 Hz, 1H) 6.72 (d, J=8.77 Hz, 1H) 6.55-6.68 (m, 1H) 5.64 (d, J=17.54 Hz, 1H) 5.21 (d, J=10.96 Hz, 1H) 3.87-4.04 (m, 3H).

Step 2: Synthesis of (E)-6-(2-(6-methoxypyridin-3-yl)vinyl)quinoline-4-carboxylic acid. To a stirred solution of 6-bromoquinoline-4-carboxylic acid (0.200 g, 0.793 mmol, 1.0 equiv) in DMF (5 mL) was added 5-ethenyl-2-methoxy-pyridine (0.125 g, 1.190 mmol, 1.5 equiv) and triethyl amine (0.34 ml, 2.380 mmol, 3.0 equiv). The resulting reaction mixture was purged with N₂ gas for 5 min followed by addition of Pd(dppf)Cl₂ (0.058 g, 0.079 mmol, 0.1 equiv). The reaction mixture was heated at 100° C. for overnight. Product formation was confirmed by LCMS. Reaction mixture was cooled to RT, diluted with water (20 mL) washed with ethyl acetate (10 mL×2). The aqueous layer was separated and freeze dried over lyophilyzer to obtain (E)-6-(2-(6-methoxypyridin-3-yl)vinyl)quinoline-4-carboxylic acid (0.200 g, 82% Yield) as a yellow solid.

LCMS 307.1 [M+H]⁺

¹H NMR (400 MHz, DMSO-d₆) δ 10.28 (s, 1H) 8.89 (d, J=4.38 Hz, 1H) 8.73 (s, 1H) 8.40 (d, J=2.19 Hz, 1H) 8.09-8.22 (m, 2H) 8.03 (d, J=9.21 Hz, 1H) 7.76 (d, J=3.95 Hz, 1H) 7.28-7.54 (m, 2H) 6.87 (d, J=8.77 Hz, 1H) 3.88 (s, 3H).

Step 3: Synthesis of (S,E)-N-(2-(2-cyano-4,4-difluoropyrrolidin-1-yl)-2-oxoethyl)-6-(2-(6-methoxypyridin-3-yl)vinyl)quinoline-4-carboxamide. To a stirred solution of (E)-6-(2-(6-methoxypyridin-3-yl)vinyl)quinoline-4-carboxylic acid (0.200 g, 0.653 mmol, 1.0 equiv) in DMF (5 mL), was added (S)-4,4-difluoro-1-glycylpyrrolidine-2-carbonitrile hydrochloride (0.220 g, 0.980 mmol, 1.5 equiv), HOBt (0.132 g, 0.980 mmol, 1.5 equiv) and EDC.HCl (0.187 g, 0.980 mmol, 1.5 equiv) followed by the addition of TEA (0.18 mL). The resulting reaction mixture was allowed to stir at RT for overnight. Product formation was confirmed by LCMS and TLC. After completion of reaction, the reaction mixture was diluted with water (50 mL) and extracted with ethyl acetate (100 mL×2). Combined organic extracts were washed with water (50 mL×3) and brine (50 mL), dried over anhydrous Na₂SO₄ and concentrated. The crude product was purified by flash chromatography (0-5% MeOH in DCM as an eluent) to obtain (S,E)-N-(2-(2-cyano-4,4-difluoropyrrolidin-1-yl)-2-oxoethyl)-6-(2-(6-methoxypyridin-3-yl)vinyl)quinoline-4-carboxamide (0.070 g, 22.43% Yield) as a yellow solid.

LCMS 478.4 [M+H]⁺

¹H NMR (400 MHz, DMSO-d₆) δ 9.17 (t, J=5.92 Hz, 1H) 8.93 (d, J=4.38 Hz, 1H) 8.52 (s, 1H) 8.41 (d, J=2.19 Hz, 1H) 8.03-8.20 (m, 3H) 7.56 (d, J=4.38 Hz, 1H) 7.53 (d, J=16.66 Hz, 1H) 7.39 (d, J=16.66 Hz, 1H) 6.88 (d, J=8.77 Hz, 1H) 5.23 (dd, J=9.21, 3.07 Hz, 1H) 4.11-4.40 (m, 4H) 3.89 (s, 3H) 2.97 (d, J=8.77 Hz, 1H) 2.87 (d, J=14.03 Hz, 1H).

### Example S31

### Synthesis of (S,E)-N-(2-(2-cyano-4,4-difluoropyrrolidin-1-yl)-2-oxoethyl)-6-(4-fluorostyryl)quinoline-4-carboxamide

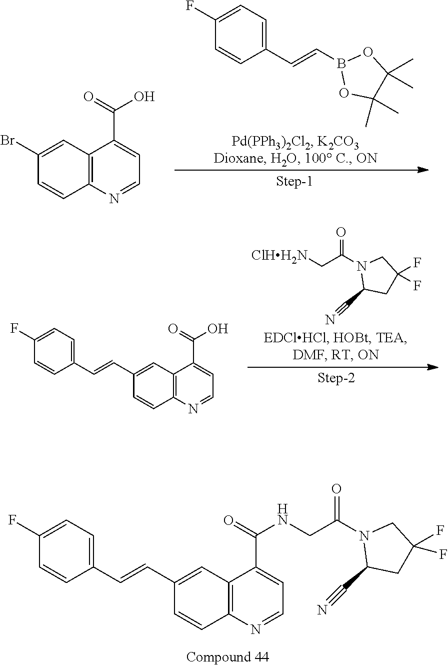

Step 1: Synthesis of (E)-6-(4-Flurostyryl)quinoline-4-carboxylic acid. To a solution of 6-bromoquinoline-4-carboxylic acid (0.341 g, 1.36 mmol, 0.8 equiv) and (E)-2-(4-flurostyryl)-4,4,5,5-tetramethyl-1,3,2-dioxaborolane (0.5 g, 1.70 mmol, 1.0 equiv) in dioxane (10 ml) and water (1 ml) was added in K₂CO₃ (0.357 g, 3.4 mmol, 2.0 equiv) and resulting reaction mixture was purged with N₂ gas for 10 min, followed by the addition of Pd(PPh₂)Cl₂ (0.059 g, 0.085 mmol. 0.05 equiv). The resulting reaction mixture was heated at 100° C. for overnight. Product formation was confirmed by LCMS. After the completion of reaction, the reaction mixture was quenched with water (10 mL) and aqueous layer was washed with ethyl acetate (10 mL×2). The aqueous layer was separated and freeze dried over lyophilizer obtain (E)-6-(4-flurostyryl) quinoline-4-carboxylic acid (0.250 g, Quant. Yield) as a yellow solid.

LCMS 294.2 [M+H]⁺

Step 2: Synthesis of (S,E)-6-(4-flurostyryl)-N-(2-(2-cyano-4,4-difluoropyrrolidin-1-yl)-2-oxoethyl)quinoline-4-carboxamide. To a stirred solution of (E)-6-(4-chlorostyryl)quinoline-4-carboxylic acid (0.1 g, 0.34 mmol, 1.0 equiv) in DMF (5 mL), was added (S)-4,4-difluoro-1-glycylpyrrolidine-2-carbonitrile hydrochloride (0.114 g, 0.51 mmol, 1.5 equiv), EDC.HCl (0.097 g, 0.51 mmol, 1.5 equiv) and HOBt (0.068 g, 0.51 mmol, 1.5 equiv) followed by the addition of TEA (0.1 mL). The resulting reaction mixture was allowed to stir at RT for overnight. Product formation was confirmed by LCMS and TLC. After completion of reaction, the reaction mixture was diluted with water (50 mL) and extracted with ethyl acetate (100 mL×2). Combined organic extracts were washed with water (50 mL×3) and brine (50 mL), dried over anhydrous Na₂SO₄ and concentrated.

The crude product was purified by flash chromatography (0-5% MeOH in DCM as an eluent) followed by reversed phase HPLC purification to obtain (S,E)-6-(4-flurostyryl)-N-(2-(2-cyano-4,4-difluoropyrrolidin-1-yl)-2-oxoethyl)quinoline-4-carboxamide (0.015 g, 10% Yield) as an off-white solid.

LCMS 465.4 [M+H]⁺

¹H NMR (400 MHz, DMSO-d₆) δ 9.18 (br. s., 1H) 8.94 (d, J=4.39 Hz, 1H) 8.58 (s, 1H) 8.09 (t, J=9.43 Hz, 2H) 7.67-7.82 (m, 2H) 7.48-7.64 (m, 2H) 7.42 (s, 1H) 7.24 (t, J=8.77 Hz, 2H) 5.22 (br. s., 1H) 4.29 (t, J=5.92 Hz, 4H) 3.17 (d, J=5.26 Hz, 1H) 2.90 (br. s., 2H).

### Example S32

### Synthesis of (S,E)-N-(2-(2-cyano-4,4-difluoropyrrolidin-1-yl)-2-oxoethyl)-6-(2-(6-methylpyridin-3-yl) vinyl)quinoline-4-carboxamide

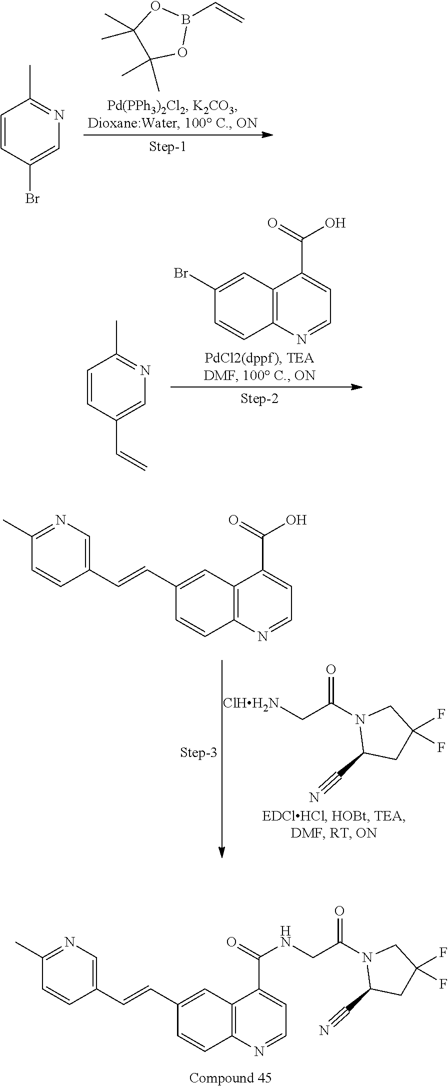

Step 1: Synthesis of 2-(methyl)-5-vinylpyridine. To a stirred solution of 5-Bromo-2-(methyl)pyridine (0.500 g, 2.906 mmol, 1.0 equiv) in Dioxane (8 mL) was added 2-Vinyl-4,4,5,5-tetramethyl-1,3,2-dioxaoborolane (0.671 g, 4.360 mmol, 1.5 equiv) and a solution of K₂CO₃ (0.802 g, 5.813 mmol, 2.0 equiv) in water (4 mL), and resulting reaction mixture was purged with N₂ gas for 10 min, followed by the addition of Pd(PPh₃)Cl₂ (0.102 g, 0.145 mmol. 0.05 equiv). The resulting reaction mixture was heated at 100° C. for overnight. Product formation was confirmed by TLC. Reaction mixture was cooled to RT, diluted with water (50 mL) extracted with ethyl acetate (100 mL×2). Combined organic extracts were washed with water (50 mL×2) & brine (50 mL), dried over anhydrous Na₂SO₄ and concentrated. The crude product was purified by flash chromatography (0-15% EA in hexane as an eluent) to obtain 2-(methyl)-5-vinylpyridine (0.170 g, 49.27% Yield) as a yellow semi solid.

¹H NMR (400 MHz, DMSO-d₆) δ 8.49 (d, J=2.19 Hz, 1H) 7.82 (dd, J=7.89, 2.19 Hz, 1H) 7.23 (d, J=8.33 Hz, 1H) 6.73 (dd, J=17.76, 11.18 Hz, 1H) 5.78-5.98 (m, 1H) 5.32 (d, J=10.96 Hz, 1H) 2.37-2.48 (m, 3H).

Step 2: Synthesis of (E)-6-(2-(6-methylpyridin-3-yl)vinyl)quinoline-4-carboxylic acid. To a stirred solution of 6-bromoquinoline-4-carboxylic acid (0.150 g, 0.595 mmol, 1.0 equiv) in Dioxane (5 mL) was added 2-(methyl)-5-vinylpyridine (0.106 g, 0.892 mmol, 1.5 equiv) and triethyl amine (0.257 ml, 1.785 mmol, 3.0 equiv). The resulting reaction mixture was purged with N₂ gas for 5 min followed by addition of Pd(dppf)Cl₂ (0.043 g, 0.059 mmol, 0.1 equiv). The reaction mixture was heated at 100° C. for overnight. Product formation was confirmed by LCMS. Reaction mixture was cooled to RT, diluted with water (50 mL) washed with ethyl acetate (20 mL×2). The aqueous layer was separated and freeze dried over lyophilyzer to obtain (E)-6-(2-(6-methylpyridin-3-yl)vinyl)quinoline-4-carboxylic acid (0.150 g, 87% Yield) as a yellow solid.

LCMS 291.1 [M+H]⁺

¹H NMR (400 MHz, DMSO-d₆) δ 9.56 (br. s., 1H) 8.91 (d, J=3.95 Hz, 1H) 8.76 (s, 1H) 8.70 (br. s., 1H) 8.18 (d, J=9.65 Hz, 2H) 8.06 (d, J=8.33 Hz, 1H) 7.79 (d, J=3.95 Hz, 1H) 7.57 (d, J=16.66 Hz, 1H) 7.43 (d, J=16.22 Hz, 1H) 7.29 (d, J=7.89 Hz, 1H) 2.33 (br. s., 3H).

Step 3: Synthesis of (S,E)-N-(2-(2-cyano-4,4-difluoropyrrolidin-1-yl)-2-oxoethyl)-6-(2-(6-methylpyridin-3-yl)vinyl)quinoline-4-carboxamide. To a stirred solution of (E)-6-(2-(6-methylpyridin-3-yl)vinyl)quinoline-4-carboxylic acid (0.200 g, 0.689 mmol, 1.0 equiv) in DMF (5 mL), was added (S)-4,4-difluoro-1-glycylpyrrolidine-2-carbonitrile hydrochloride (0.232 g, 1.034 mmol, 1.5 equiv), HOBt (0.139 g, 1.034 mmol, 1.5 equiv) & EDC.HCl (0.197 g, 1.034 mmol, 1.5 equiv) followed by the addition of TEA (0.19 mL). The resulting reaction mixture was allowed to stir at RT for overnight. Product formation was confirmed by LCMS and TLC. After completion of reaction, the reaction mixture was diluted with water (50 mL) and extracted with ethyl acetate (100 mL×2). Combined organic extracts were washed with water (50 mL×3) and brine (50 mL), dried over anhydrous Na₂SO₄ and concentrated. The crude product was purified by flash chromatography (0-5% MeOH in DCM as an eluent) to obtain (S,E)-N-(2-(2-cyano-4,4-difluoropyrrolidin-1-yl)-2-oxoethyl)-6-(2-(6-methylpyridin-3-yl)vinyl)quinoline-4-carboxamide (0.070 g, 22.08% Yield) as an off white solid.

LCMS 462.4 [M+H]⁺

¹H NMR (400 MHz, DMSO-d₆) δ 9.18 (t, J=5.92 Hz, 1H) 8.94 (d, J=4.38 Hz, 1H) 8.71 (d, J=1.75 Hz, 1H) 8.58 (s, 1H) 7.94-8.23 (m, 3H) 7.40-7.65 (m, 3H) 7.29 (d, J=7.89 Hz, 1H) 5.23 (dd, J=9.21, 3.07 Hz, 1H) 4.11-4.41 (m, 4H) 2.96 (br. s., 1H) 2.87 (d, J=15.35 Hz, 1H) 2.49 (s, 3H).

### Example S33

### Synthesis of (S,E)-N-(2-(2-cyano-4,4-difluoropyrrolidin-1-yl)-2-oxoethyl)-6-(2-(6-(trifluoromethyl)pyridin-3-yl)vinyl)quinoline-4-carboxamide

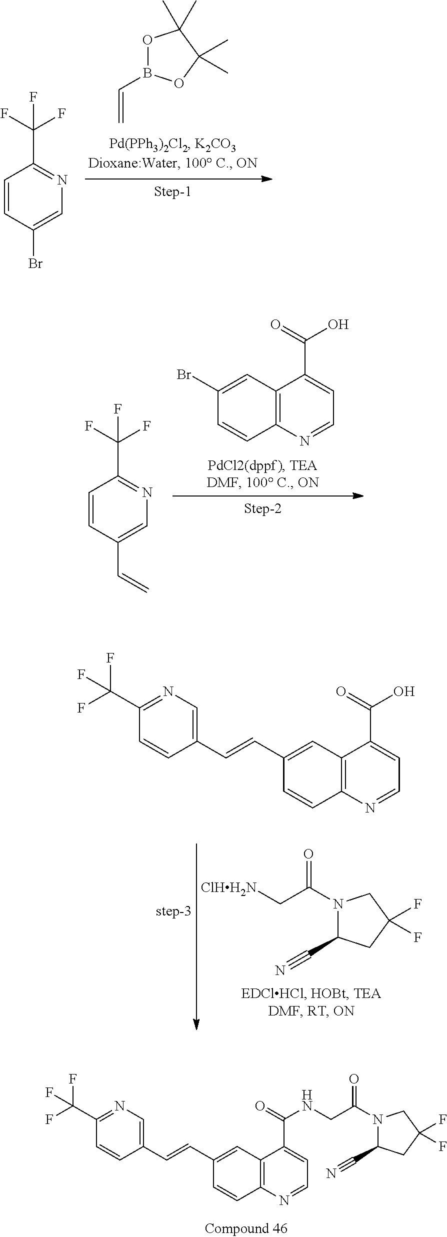

Step 1: Synthesis of 2-(trifluoromethyl)-5-vinylpyridine. To a stirred solution of 5-Bromo-2-(trifluoromethyl)pyridine (0.500 g, 2.212 mmol, 1.0 equiv) in Dioxane (8 mL) was added 2-Vinyl-4,4,5,5-tetramethyl-1,3,2-dioxaoborolane (0.511 g, 3.312 mmol, 1.5 equiv) and a solution of K₂CO₃ (0.616 g, 4.424 mmol, 2.0 equiv) in water (4 mL), and resulting reaction mixture was purged with N₂ gas for 10 minute, followed by the addition of Pd(PPh₃)Cl₂ (0.077 g, 0.110 mmol. 0.05 equiv). The resulting reaction mixture was heated at 100° C. for overnight. Product formation was confirmed by TLC. Reaction mixture was cooled to RT, diluted with water (50 mL) extracted with ethyl acetate (100 mL×2). Combined organic extracts were washed with water (50 mL×2) & brine (50 mL), dried over anhydrous Na₂SO₄ and concentrated. The crude product was purified by flash chromatography (0-5% EA in hexane as an eluent) to obtain 2-(trifluoromethyl)-5-vinylpyridine (0.200 g, 52.35% Yield) as a yellow semi solid.

¹H NMR (400 MHz, DMSO-d₆) δ 8.77-8.97 (m, 1H) 8.14-8.26 (m, 1H) 7.88 (d, J=7.89 Hz, 1H) 6.88 (dd, J=17.76, 11.18 Hz, 1H) 6.17 (d, J=17.98 Hz, 1H) 5.58 (d, J=11.40 Hz, 1H).

Step 2: Synthesis of (E)-6-(2-(6-(trifluoromethyl)pyridin-3-yl)vinyl)quinoline-4-carboxylic acid. To a stirred solution of 6-bromoquinoline-4-carboxylic acid (0.100 g, 0.396 mmol, 1.0 equiv) in Dioxane (10 mL) was added 2-(trifluoromethyl)-5-vinylpyridine (0.102 g, 0.595 mmol, 1.5 equiv) and triethyl amine (0.171 ml, 1.190 mmol, 3.0 equiv). The resulting reaction mixture was purged with N₂ gas for 5 min followed by addition of Pd(dppf)Cl₂ (0.029 g, 0.0396 mmol, 0.1 equiv). The reaction mixture was heated at 100° C. for overnight. Product formation was confirmed by LCMS. Reaction mixture was cooled to RT, diluted with water (50 mL) washed with ethyl acetate (20 mL×2), The aqueous layer was acidified with 1N HCl and extracted with ethyl acetate (100 mL×2). Combined organic extracts were washed with water (20 mL×2) and brine (20 mL), dried over anhydrous Na₂SO₄ and concentrated under reduced pressure to obtain (E)-6-(2-(6-(trifluoromethyl)pyridin-3-yl)vinyl)quinoline-4-carboxylic acid (0.100 g, 73.52% Yield) as a yellow solid.

LCMS 345.1 [M+H]⁺

¹H NMR (400 MHz, DMSO-d₆) δ 13.91 (br. s., 1H) 9.06 (s, 1H) 9.00 (d, J=4.38 Hz, 1H) 8.82 (s, 1H) 8.41 (d, J=8.77 Hz, 1H) 8.26 (d, J=9.21 Hz, 1H) 8.13 (d, J=8.77 Hz, 1H) 7.81-7.99 (m, 3H) 7.59 (d, J=16.66 Hz, 1H).

Step 3: Synthesis of (S,E)-N-(2-(2-cyano-4,4-difluoropyrrolidin-1-yl)-2-oxoethyl)-6-(2-(6-(trifluoromethyl)pyridin-3-yl)vinyl)quinoline-4-carboxamide. To a stirred solution of (E)-6-(2-(6-(trifluoromethyl)pyridin-3-yl)vinyl)quinoline-4-carboxylic acid (0.100 g, 0.290 mmol, 1.0 equiv) in DMF (5 mL), was added (S)-4,4-difluoro-1-glycylpyrrolidine-2-carbonitrile hydrochloride (0.098 g, 0.436 mmol, 1.5 equiv), HOBt (0.058 g, 0.436 mmol, 1.5 equiv) and EDC.HCl (0.083 g, 0.436 mmol, 1.5 equiv) followed by the addition of TEA (0.08 mL). The resulting reaction mixture was allowed to stir at RT for overnight. Product formation was confirmed by LCMS and TLC. After completion of reaction, the reaction mixture was diluted with water (50 mL) and extracted with ethyl acetate (100 mL×2). Combined organic extracts were washed with water (50 mL×3) and brine (50 mL), dried over anhydrous Na₂SO₄ and concentrated. The crude product was purified by flash chromatography (0-5% MeOH in DCM as an eluent) to obtain (S,E)-N-(2-(2-cyano-4,4-difluoropyrrolidin-1-yl)-2-oxoethyl)-6-(2-(6-(trifluoromethyl)pyridin-3-yl)vinyl)quinoline-4-carboxamide (0.095 g, 63% Yield) as a white solid.

LCMS 516.4 [M+H]⁺

¹H NMR (400 MHz, DMSO-d₆) δ 9.20 (t, J=5.70 Hz, 1H) 9.05 (s, 1H) 8.98 (d, J=3.95 Hz, 1H) 8.68 (s, 1H) 8.40 (d, J=7.45 Hz, 1H) 8.03-8.27 (m, 2H) 7.93 (d, J=8.33 Hz, 1H) 7.81 (s, 1H) 7.66-7.74 (m, 1H) 7.60 (d, J=4.39 Hz, 1H) 5.24 (d, J=7.02 Hz, 1H) 4.26-4.50 (m, 2H) 4.00-4.26 (m, 2H) 2.76-3.03 (m, 2H).

### Example S34

### Synthesis of (S,E)-N-(2-(2-cyano-4,4-difluoropyrrolidin-1-yl)-2-oxoethyl)-6-(4-methoxystyryl)quinoline-4-carboxamide

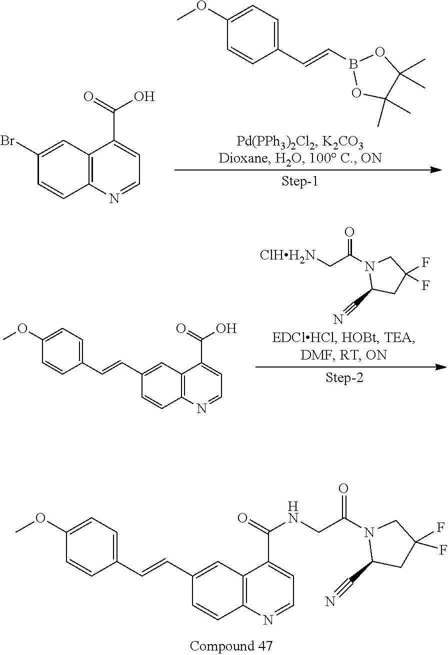

Step 1: Synthesis of (E)-6-(4-methoxystyryl)quinoline-4-carboxylic acid. To a solution of 6-bromoquinoline-4-carboxylic acid (0.383 g, 1.52 mmol, 0.8 equiv) and (E)-2-(4-methoxystyryl)-4,4,5,5-tetramethyl-1,3,2-dioxaborolane (0.5 g, 1.91 mmol, 1.0 equiv) in dioxane (10 ml) and water (1 ml) was added in K₂CO₃ (0.404 g, 3.82 mmol, 2.0 equiv) and the resulting reaction mixture was purged with N₂ gas for 10 min, followed by the addition of Pd(PPh₂)Cl₂ (0.067 g, 0.095 mmol. 0.05 equiv). The resulting reaction mixture was heated at 100° C. for overnight. Product formation was confirmed by LCMS. After the completion of reaction, the reaction mixture was quenched with water (20 mL) and aqueous layer was washed with ethyl acetate (10 mL×2). The aqueous layer was separated and freeze dried over lyophilizer to obtain (E)-6-(4-methoxystyryl) quinoline-4-carboxylic acid (0.200 g, 44% Yield) as an yellow solid.

LCMS 306.0 [M+H]⁺

Step 2: Synthesis of (S,E)-6-(4-methoxystyryl)-N-(2-(2-cyano-4,4-difluoropyrrolidin-1-yl)-2-oxoethyl)quinoline-4-carboxamide. To a stirred solution of (E)-6-(4-chlorostyryl)quinoline-4-carboxylic acid (0.2 g, 0.65 mmol, 1.0 equiv) in DMF (5 mL), was added (S)-4,4-difluoro-1-glycylpyrrolidine-2-carbonitrile hydrochloride (0.220 g, 0.98 mmol, 1.5 equiv), EDC.HCl (0.188 g, 0.98 mmol, 1.5 equiv) and HOBt (0.132 g, 0.98 mmol, 1.5 equiv) followed by the addition of TEA (0.2 mL). The resulting reaction mixture was allowed to stir at RT for overnight. Product formation was confirmed by LCMS and TLC. After completion of reaction, the reaction mixture was diluted with water (50 mL) and extracted with ethyl acetate (100 mL×2). Combined organic extracts were washed with water (50 mL×3) and brine (50 mL), dried over anhydrous Na₂SO₄ and concentrated.

The crude product was purified by flash chromatography (0-5% MeOH in DCM as an eluent) followed by reversed phase HPLC purification to obtain (S,E)-6-(4-methoxystyryl)-N-(2-(2-cyano-4,4-difluoropyrrolidin-1-yl)-2-oxoethyl)quinoline-4-carboxamide (0.045 g, 14% Yield) as an off-white solid.

LCMS 477.4 [M+H]⁺

¹H NMR (400 MHz, DMSO-d₆) δ 9.17 (t, J=5.92 Hz, 1H) 8.91 (d, J=3.95 Hz, 1H) 8.52 (s, 1H) 8.10 (dd, J=8.99, 1.53 Hz, 1H) 8.05 (d, J=8.77 Hz, 1H) 7.65 (m, J=8.33 Hz, 2H) 7.47-7.59 (m, 2H) 7.28 (d, J=16.22 Hz, 1H) 6.97 (m, J=8.77 Hz, 2H) 5.23 (dd, J=9.21, 2.63 Hz, 1H) 4.11-4.41 (m, 4H) 3.79 (s, 3H) 2.78-3.05 (m, 2H).

### Example S35

### Synthesis of (S,E)-N-(2-(2-cyano-4,4-difluoropyrrolidin-1-yl)-2-oxoethyl)-6-(4-(trifluoromethoxy)styryl)quinoline-4-carboxamide

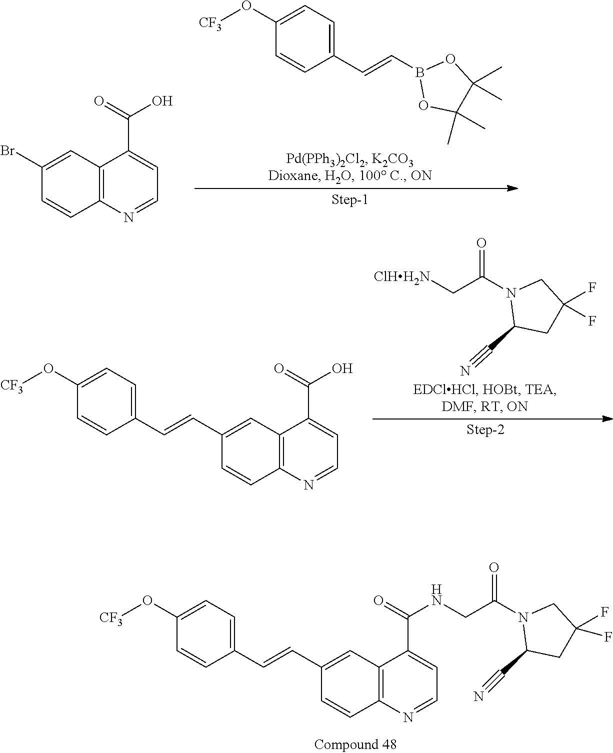

Step 1: Synthesis of (E)-6-(4-trifluromethoxystyryl)quinoline-4-carboxylic acid. To a solution of 6-bromoquinoline-4-carboxylic acid (0.140 g, 0.56 mmol, 0.8 equiv) and (E)-2-(4-trifluromethoxystyryl)-4,4,5,5-tetramethyl-1,3,2-dioxaborolane (0.220 g, 0.70 mmol, 1.0 equiv) in dioxane (10 ml) and water (1 ml) was added in K₂CO₃ (0.147 g, 1.4 mmol, 2.0 equiv) and resulting reaction mixture was purged with N₂ gas for 10 min, followed by the addition of Pd(PPh₂)Cl₂ (0.024 g, 0.035 mmol. 0.05 equiv). The resulting reaction mixture was heated at 100° C. for overnight. Product formation was confirmed by LCMS. After the completion of reaction, the reaction mixture was quenched with water (20 mL) and aqueous layer was washed with ethyl acetate (10 mL×2). The aqueous layer was separated and freeze dried over lyophilizer to obtain (E)-6-(4-methoxystyryl) quinoline-4-carboxylic acid (0.100 g, 50% Yield) as a yellow solid.

LCMS 360.1 [M+H]⁺

Step 2: Synthesis of (S,E)-6-(4-methoxystyryl)-N-(2-(2-cyano-4,4-difluoropyrrolidin-1-yl)-2-oxoethyl)quinoline-4-carboxamide. To a stirred solution of (E)-6-(4-chlorostyryl)quinoline-4-carboxylic acid (0.1 g, 0.27 mmol, 1.0 equiv) in DMF (5 mL), was added (S)-4,4-difluoro-1-glycylpyrrolidine-2-carbonitrile hydrochloride (0.093 g, 0.41 mmol, 1.5 equiv), EDCI.HCl (0.078 g, 0.41 mmol, 1.5 equiv) and HOBt (0.055 g, 0.41 mmol, 1.5 equiv) followed by the addition of TEA (0.1 mL). The resulting reaction mixture was allowed to stir at RT for overnight. Product formation was confirmed by LCMS and TLC. After completion of reaction, the reaction mixture was diluted with water (50 mL) and extracted with ethyl acetate (100 mL×2). Combined organic extracts were washed with water (50 mL×3) and brine (50 mL), dried over anhydrous Na₂SO₄ and concentrated.

The crude product was purified by flash chromatography (0-5% MeOH in DCM as an eluent) followed by reversed phase HPLC purification to obtain (S,E)-6-(4-methoxystyryl)-N-(2-(2-cyano-4,4-difluoropyrrolidin-1-yl)-2-oxoethyl)quinoline-4-carboxamide (0.045 g, 32% Yield) as an off white solid.

LCMS 531.4 [M+H]⁺

¹H NMR (400 MHz, DMSO-d₆) δ 9.19 (t, J=5.70 Hz, 1H) 8.94 (d, J=4.38 Hz, 1H) 8.62 (s, 1H) 8.04-8.17 (m, 2H) 7.83 (m, J=8.77 Hz, 2H) 7.53-7.70 (m, 2H) 7.45-7.53 (m, 1H) 7.39 (m, J=8.33 Hz, 2H) 5.23 (dd, J=9.21, 2.19 Hz, 1H) 4.11-4.41 (m, 4H) 2.96 (br. s., 2H).

### Example S36

### Synthesis of (S,E)-N-(2-(2-cyano-4,4-difluoropyrrolidin-1-yl)-2-oxoethyl)-6-(2-(5-methylpyridin-2-yl) vinyl)quinoline-4-carboxamide

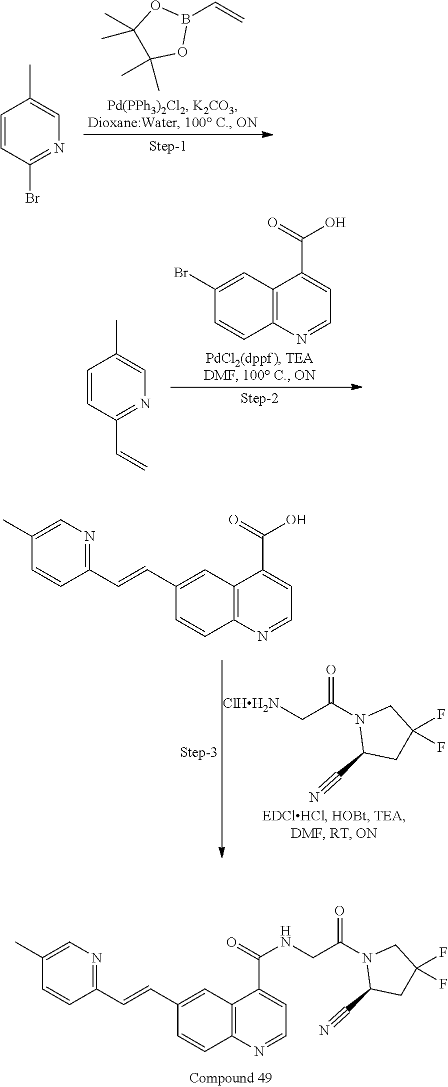

Step 1: Synthesis of 5-(methyl)-2-vinylpyridine. To a stirred solution of 2-Bromo-5-methylpyridine (0.500 g, 2.906 mmol, 1.0 equiv) in Dioxane (8 mL) was added 2-Vinyl-4,4,5,5-tetramethyl-1,3,2-dioxaoborolane (0.671 g, 4.360 mmol, 1.5 equiv) and a solution of K₂CO₃ (0.802 g, 5.813 mmol, 2.0 equiv) in water (4 mL), and resulting reaction mixture was purged with N₂ gas for 10 minute, followed by the addition of Pd(PPh₃)Cl₂ (0.102 g, 0.145 mmol. 0.05 equiv). The resulting reaction mixture was heated at 100° C. for overnight. Product formation was confirmed by TLC. Reaction mixture was cool to RT, diluted with water (50 mL) extracted with ethyl acetate (100 mL×2). Combined organic extracts were washed with water (50 mL×2) and brine (50 mL), dried over anhydrous Na₂SO₄ and concentrated. The crude product was purified by flash chromatography (0-15% EA in hexane as an eluent) to obtain 5-(methyl)-2-vinylpyridine (0.300 g, 86.95% Yield) as a yellow semi-solid.

¹H NMR (400 MHz, CHLOROFORM-d) δ 8.40 (s, 1H) 7.45 (d, J=7.02 Hz, 1H) 7.17-7.34 (m, 1H) 6.79 (dd, J=17.54, 10.96 Hz, 1H) 6.12 (d, J=17.54 Hz, 1H) 5.42 (d, J=10.96 Hz, 1H) 2.20-2.45 (m, 3H).

Step 2: Synthesis of (E)-6-(2-(5-methylpyridin-2-yl)vinyl)quinoline-4-carboxylic acid. To a stirred solution of 6-bromoquinoline-4-carboxylic acid (0.100 g, 0.396 mmol, 1.0 equiv) in DMF (5 mL) was added 5-(methyl)-2-vinylpyridine (0.070 g, 0.595 mmol, 1.5 equiv) and triethyl amine (0.171 ml, 1.190 mmol, 3.0 equiv). The resulting reaction mixture was purged with N₂ gas for 5 min followed by addition of Pd(dppf)Cl₂ (0.014 g, 0.019 mmol, 0.1 equiv). The reaction mixture was heated at 100° C. for overnight. Product formation was confirmed by LCMS. Reaction mixture was cooled to RT, diluted with water (60 mL) washed with ethyl acetate (20 mL×2). The aqueous layer was separated and freeze dried over lyophilyzer to obtain (E)-6-(2-(5-methylpyridin-2-yl)vinyl)quinoline-4-carboxylic acid (0.100 g, 86% Yield) as a yellow solid.

LCMS 291.0 [M+H]⁺

¹H NMR (400 MHz, DMSO-d₆) δ 9.37 (br. s., 1H) 8.90-9.03 (m, 1H) 8.82 (s, 1H) 8.45 (s, 1H) 8.20 (d, J=7.89 Hz, 1H) 8.01-8.13 (m, 1H) 7.75-7.90 (m, 2H) 7.54-7.75 (m, 2H) 7.45 (d, J=16.22 Hz, 1H) 2.33 (s, 3H).

Step 3: Synthesis of (S,E)-N-(2-(2-cyano-4,4-difluoropyrrolidin-1-yl)-2-oxoethyl)-6-(2-(5-methylpyridin-2-yl)vinyl)quinoline-4-carboxamide. To a stirred solution of (E)-6-(2-(5-methylpyridin-2-yl)vinyl)quinoline-4-carboxylic acid (0.100 g, 0.344 mmol, 1.0 equiv) in DMF (2 mL), was added (S)-4,4-difluoro-1-glycylpyrrolidine-2-carbonitrile hydrochloride (0.116 g, 0.517 mmol, 1.5 equiv), HOBt (0.069 g, 0.517 mmol, 1.5 equiv) and EDC.HCl (0.098 g, 0.517 mmol, 1.5 equiv) followed by the addition of TEA (0.09 mL). The resulting reaction mixture was allowed to stir at RT for overnight. Product formation was confirmed by LCMS and TLC. After completion of reaction, the reaction mixture was diluted with water (50 mL) and extracted with ethyl acetate (100 mL×2). Combined organic extracts were washed with water (50 mL×3) and brine (50 mL), dried over anhydrous Na₂SO₄ and concentrated. The crude product was purified by flash chromatography (0-5% MeOH in DCM as an eluent) to obtain (S,E)-N-(2-(2-cyano-4,4-difluoropyrrolidin-1-yl)-2-oxoethyl)-6-(2-(5-methylpyridin-2-yl)vinyl)quinoline-4-carboxamide (0.070 g, 44.30% Yield) as an off-white solid.

LCMS 462.4 [M+H]⁺

¹H NMR (400 MHz, DMSO-d₆) δ 9.19 (t, J=5.92 Hz, 1H) 8.95 (d, J=3.95 Hz, 1H) 8.58 (s, 1H) 8.47 (s, 1H) 8.17 (d, J=10.09 Hz, 1H) 8.08 (d, J=8.77 Hz, 1H) 7.79 (d, J=16.22 Hz, 1H) 7.44-7.65 (m, 3H) 5.25 (d, J=7.45 Hz, 1H) 4.11-4.40 (m, 4H) 2.77-3.05 (m, 2H) 2.33 (s, 3H).

### Example S37

### Synthesis of (S,E)-6-(4-(tert-butyl)styryl)-N-(2-(2-cyano-4,4-difluoropyrrolidin-1-yl)-2-oxoethyl)quinoline-4-carboxamide

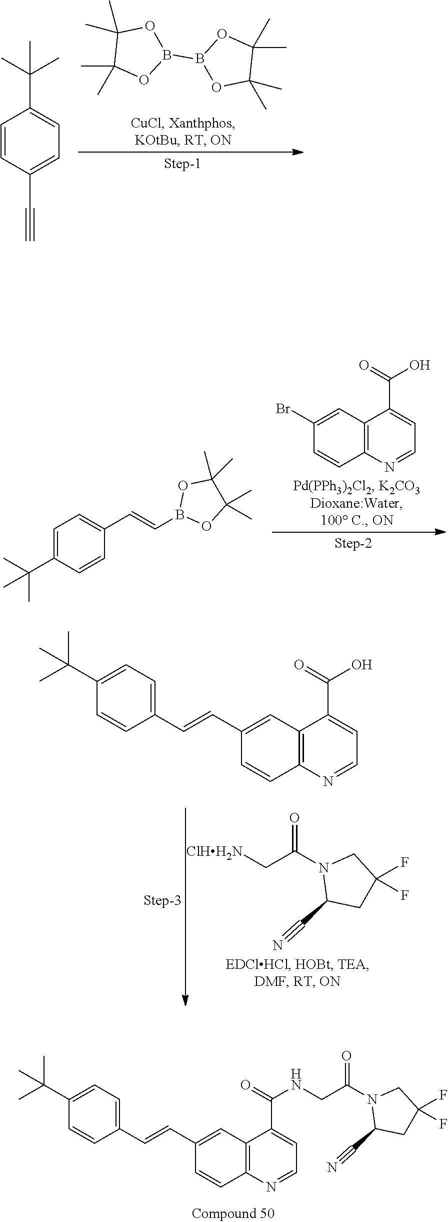

Step 1: Synthesis of 2-[2-(4-tert-butylphenyl)ethenyl]-4,4,5,5-tetramethyl-1,3,2-dioxaborolane. To a stirred solution of 4,4,4′,4′,5,5,5′,5′-octamethyl-2,2′-bi(1,3,2-dioxaborolane (0.200 g, 1.265 mmol, 1.0 equiv) in THF (5 mL) was added CuCl(I) (0.001 g, 0.012 mmol, 0.01 equiv) and Xanthphos (0.007 g, 0.012 mmol, 0.01 equiv). The resulting reaction mixture was allowed to stir at RT for 15 min. After 15 min Potassium tert-butoxide (0.170 g, 1.518 mmol, 1.2 equiv) and 1-tert-butyl-4-ethynylbenzene (0.200 g, 1.265 mmol, 1.0 equiv) was added. The resulting reaction mixture was stirred at RT for overnight. Product formation was confirmed by TLC. After completion of reaction, the reaction mixture was diluted with water (50 mL) and extracted with ethyl acetate (80 mL×3). Combined organic extracts were washed with water (20 mL×3) and brine (20 mL), dried over anhydrous Na₂SO₄ and concentrated. The crude product was purified by flash chromatography (5% EA-Hexane as an eluent) to obtained 2-[2-(4-tert-butylphenyl)ethenyl]-4,4,5,5-tetramethyl-1,3,2-dioxaborolane (0.100 g, 27.62% Yield) as a yellow solid.

LCMS 287.0 [M+H]⁺

¹H NMR (400 MHz, CHLOROFORM-d) δ 7.41-7.48 (m, 2H) 7.31-7.41 (m, 2H) 7.26 (s, 1H) 6.12 (d, J=18.42 Hz, 1H) 1.31 (s, 12H).

Step 2: Synthesis of (E)-6-(4-(tert-butyl)styryl)quinoline-4-carboxylic acid. To a stirred solution of 6-bromoquinoline-4-carboxylic acid (0.100 g, 0.396 mmol, 1.0 equiv) in Dioxane (3 mL) was added 2-[2-(4-tert-butylphenyl)ethenyl]-4,4,5,5-tetramethyl-1,3,2-dioxaborolane (0.136 g, 0.476 mmol, 1.2 equiv) and a solution of K₂CO₃ (0.109 g, 0.792 mmol, 2.0 equiv) in water (1 mL), and resulting reaction mixture was purged with N₂ gas for 10 minute, followed by the addition of Pd(PPh₃)Cl₂ (0.013 g, 0.019 mmol. 0.05 equiv). The resulting reaction mixture was heated at 100° C. for overnight. Product formation was confirmed by LCMS. Reaction mixture was cooled to RT, diluted with water (50 mL) washed with ethyl acetate (20 mL×3). The aqueous layer was separated and freeze dried over lyophilyzer to obtain (E)-6-(4-(tert-butyl)styryl)quinoline-4 carboxylic acid (0.100 g, 76% Yield) as a yellow solid.

LCMS 332.1 [M+H]⁺

¹H NMR (400 MHz, DMSO-d₆) δ 8.64-8.78 (m, 2H) 8.55 (s, 1H) 8.01 (d, J=7.89 Hz, 1H) 7.89 (d, J=8.33 Hz, 1H) 7.59 (d, J=8.33 Hz, 2H) 7.37-7.51 (m, 2H) 7.33 (d, J=6.58 Hz, 2H) 1.30 (s, 9H).

Step 3: Synthesis of (S,E)-6-(4-(tert-butyl)styryl)-N-(2-(2-cyano-4,4-difluoropyrrolidin-1-yl)-2-oxoethyl)quinoline-4-carboxamide. To a stirred solution of (E)-6-(4-(tert-butyl)styryl)quinoline-4 carboxylic acid (0.100 g, 0.302 mmol, 1.0 equiv) in DMF (2 mL), was added (S)-4,4-difluoro-1-glycylpyrrolidine-2-carbonitrile hydrochloride (0.101 g, 0.453 mmol, 1.5 equiv), HOBt (0.061 g, 0.453 mmol, 1.5 equiv) and EDC.HCl (0.086 g, 0.453 mmol, 1.5 equiv) followed by the addition of TEA (0.09 mL). The resulting reaction mixture was allowed to stir at RT for overnight. Product formation was confirmed by LCMS and TLC. After completion of reaction, the reaction mixture was diluted with water (50 mL) and extracted with ethyl acetate (100 mL×2). Combined organic extracts were washed with water (50 mL×3) and brine (50 mL), dried over anhydrous Na₂SO₄ and concentrated. The crude product was purified by flash chromatography (0-5% MeOH in DCM as an eluent) to obtain (S,E)-6-(4-(tert-butyl)styryl)-N-(2-(2-cyano-4,4-difluoropyrrolidin-1-yl)-2-oxoethyl)quinoline-4-carboxamide (0.060 g, 39.73% Yield) as an off-white solid.

LCMS 503.4 [M+H]⁺

¹H NMR (400 MHz, DMSO-d₆) δ 9.18 (br. s., 1H) 8.92 (d, J=3.95 Hz, 1H) 8.57 (s, 1H) 8.13 (d, J=8.77 Hz, 1H) 8.06 (d, J=9.21 Hz, 1H) 7.63 (d, J=8.33 Hz, 2H) 7.49-7.60 (m, 2H) 7.29-7.49 (m, 3H) 5.23 (d, J=8.77 Hz, 1H) 4.26-4.46 (m, 2H) 4.05-4.26 (m, 2H) 2.96 (br. s., 1H) 2.88 (d, J=15.79 Hz, 1H) 1.30 (s, 9H).

### Example S38

### Synthesis of (S,E)-N-(2-(2-cyano-4,4-difluoropyrrolidin-1-yl)-2-oxoethyl)-6-(4-(trifluoromethyl)styryl)quinoline-4-carboxamide

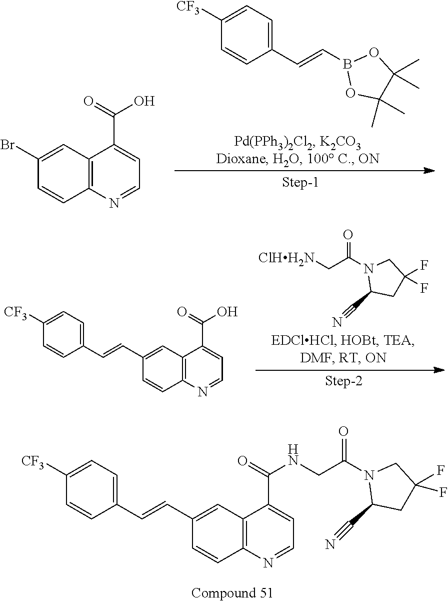

Step 1: Synthesis of (E)-6-(4-trifluromethyl)styryl)quinoline-4-carboxylic acid. To a solution of 6-bromoquinoline-4-carboxylic acid (0.140 g, 0.56 mmol, 0.8 equiv) and (E)-2-(4-trifluromethyl)styryl)-4,4,5,5-tetramethyl-1,3,2-dioxaborolane (0.220 g, 0.70 mmol, 1.0 equiv) in dioxane (10 ml) and water (1 ml) was added in K₂CO₃ (0.147 g, 1.4 mmol, 2.0 equiv) and resulting reaction mixture was purged with N₂ gas for 10 min, followed by the addition of Pd(PPh₂)Cl₂ (0.024 g, 0.035 mmol. 0.05 equiv). The resulting reaction mixture was heated at 100° C. for overnight.

Product formation was confirmed by LCMS. After the completion of reaction, the mixture was quenched with water (20 mL) and aqueous layer washed with ethyl acetate (10 mL×2). The aqueous layer was separated and freeze dried over lyophilyzer to obtain (E)-6-(4-trifluromethylstyryl) quinoline-4-carboxylic acid (0.250 g, Quant. Yield) as a yellow solid.

LCMS 360.1 [M+H]⁺

Step 2: Synthesis of (S,E)-6-(4-trifluromethylstyryl)-N-(2-(2-cyano-4,4-difluoropyrrolidin-1-yl)-2-oxoethyl)quinoline-4-carboxamide. To a stirred solution of (E)-6-(4-trifluromethyl)styryl)quinoline-4-carboxylic acid (0.250 g, 0.726 mmol, 1.0 equiv) in DMF (5 mL), was added (S)-4,4-difluoro-1-glycylpyrrolidine-2-carbonitrile hydrochloride (0.245 g, 1.09 mmol, 1.5 equiv), EDC.HCl (0.209 g, 1.09 mmol, 1.5 equiv) and HOBt (0.145 g, 1.09 mmol, 1.5 equiv) followed by the addition of TEA (0.2 mL). The resulting reaction mixture was allowed to stir at RT for overnight. Product formation was confirmed by LCMS and TLC. After completion of reaction, the reaction mixture was diluted with water (50 mL) and extracted with ethyl acetate (100 mL×2). Combined organic extracts were washed with water (50 mL×3) and brine (50 mL), dried over anhydrous Na₂SO₄ and concentrated. The crude product was purified by flash chromatography (0-5% MeOH in DCM as an eluent) followed by reversed phase HPLC purification to obtain (S,E)-6-(4-trifluromethyl styryl)-N-(2-(2-cyano-4,4-difluoropyrrolidin-1-yl)-2-oxoethyl)quinoline-4-carboxamide (0.008 g, 32% Yield) as an off white solid.

LCMS 515.3[M+H]⁺

¹H NMR (400 MHz, DMSO-d₆) δ 9.21 (s, 1H) 8.96 (d, J=4.38 Hz, 1H) 8.69 (s, 1H) 8.16 (d, J=8.77 Hz, 1H) 8.10 (d, J=8.77 Hz, 1H) 7.93 (m, J=8.33 Hz, 2H) 7.72-7.80 (m, 2H) 7.66 (d, J=14.47 Hz, 1H) 7.53-7.61 (m, 1H) 5.24 (d, J=8.77 Hz, 1H) 4.18-4.36 (br. s., 4H) 2.96 (br. s., 2H).

### Example S39

### Synthesis of (S)—N-(2-(2-cyano-4,4-difluoropyrrolidin-1-yl)-2-oxoethyl)-6-(6-(4-methylpiperazin-1-yl)pyridin-3-yl)quinoline-4-carboxamide

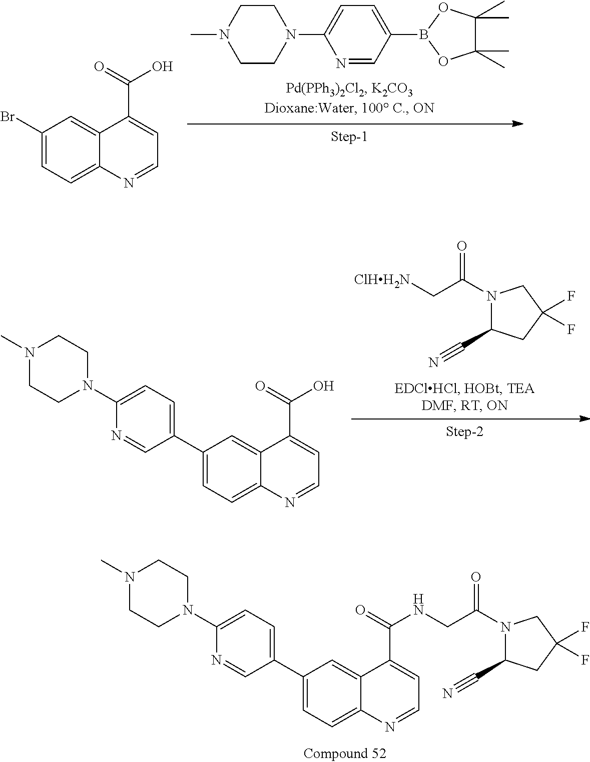

Step 1: Synthesis of 6-(6-(4-methylpiperazin-1-yl)pyridin-3-yl)quinoline-4-carboxylic acid. To a stirred solution of 6-bromoquinoline-4-carboxylic acid (0.250 g, 0.992 mmol, 1.0 equiv) in Dioxane (5 mL) was added 2-(4-Methylpiperazin-1-yl)pyridine-5-boronic acid pinacol ester (0.360 g, 1.190 mmol, 1.5 equiv), K₂CO₃ (0.273 g, 1.984 mmol, 2.0 equiv) in water (2 mL) was added and the mixture was purged with N₂ gas for 10 min followed by the addition of Pd(PPh₃)Cl₂ (0.034 g, 0.049 mmol. 0.05 equiv). The resulting reaction mixture was heated at 100° C. for overnight. Product formation was confirmed by LCMS. Reaction mixture was cooled to RT, diluted with water (60 mL) washed with ethyl acetate (25 mL×3). The aqueous layer was separated and freeze dried over lyophilyzer to obtain 6-(6-(4-methylpiperazin-1-yl)pyridin-3-yl)quinoline-4-carboxylic acid (0.250 g, 72% Yield) as a yellow solid.

LCMS 349.0 [M+H]⁺

¹H NMR (400 MHz, DMSO-d₆) δ 8.94 (s, 1H) 8.73 (d, J=4.38 Hz, 1H) 8.53 (d, J=2.19 Hz, 1H) 7.96 (s, 2H) 7.88-7.93 (m, 1H) 7.47 (d, J=4.38 Hz, 1H) 6.98 (d, J=8.77 Hz, 1H) 3.55 (d, J=5.26 Hz, 4H) 2.39-2.44 (m, 4H) 2.23 (s, 3H).

Step 2: Synthesis of (S)—N-(2-(2-cyano-4,4-difluoropyrrolidin-1-yl)-2-oxoethyl)-6-(6-(4-methylpiperazin-1-yl)pyridin-3-yl)quinoline-4-carboxamide. To a stirred solution of 6-(6-(4-methylpiperazin-1-yl)pyridin-3-yl)quinoline-4-carboxylic acid (0.100 g, 0.287 mmol, 1.0 equiv) in DMF (2 mL), was added (S)-4,4-difluoro-1-glycylpyrrolidine-2-carbonitrile hydrochloride (0.082 g, 0.431 mmol, 1.5 equiv), HOBt (0.058 g, 0.431 mmol, 1.5 equiv) and EDC.HCl (0.082 g, 0.431 mmol, 1.5 equiv) followed by the addition of TEA (0.1 mL). The resulting reaction mixture was allowed to stir at RT for overnight. Product formation was confirmed by LCMS and TLC. After completion of reaction, the reaction mixture was diluted with water (50 mL) and extracted with ethyl acetate (100 mL×2). Combined organic extracts were washed with water (50 mL×3) and brine (50 mL), dried over anhydrous Na₂SO₄ and concentrated. The crude product was purified by flash chromatography (0-5% MeOH in DCM as an eluent) followed by reversed phase HPLC purification to obtain (S)—N-(2-(2-cyano-4,4-difluoropyrrolidin-1-yl)-2-oxoethyl)-6-(6-(4-methylpiperazin-1-yl)pyridin-3-yl)quinoline-4-carboxamide (0.115 g, 77% Yield) as an off-white solid.

LCMS 520.5 [M+H]⁺

¹H NMR (400 MHz, DMSO-d₆) δ 9.17 (t, J=5.92 Hz, 1H) 8.94 (d, J=4.38 Hz, 1H) 8.65 (dd, J=6.14, 2.19 Hz, 2H) 8.16-8.23 (m, 1H) 8.07-8.16 (m, 2H) 7.56 (d, J=4.38 Hz, 1H) 6.98 (d, J=9.21 Hz, 1H) 5.19 (dd, J=9.21, 2.63 Hz, 1H) 4.22-4.42 (m, 3H) 4.08-4.22 (m, 1H) 3.50-3.65 (m, 4H) 2.78-3.01 (m, 2H) 2.36-2.46 (m, 4H) 2.23 (s, 3H).

### Example S40

### Synthesis of (S,E)-N-(2-(2-cyano-4,4-difluoropyrrolidin-1-yl)-2-oxoethyl)-6-(2-(6-hydroxypyridin-3-yl) vinyl)quinoline-4-carboxamide

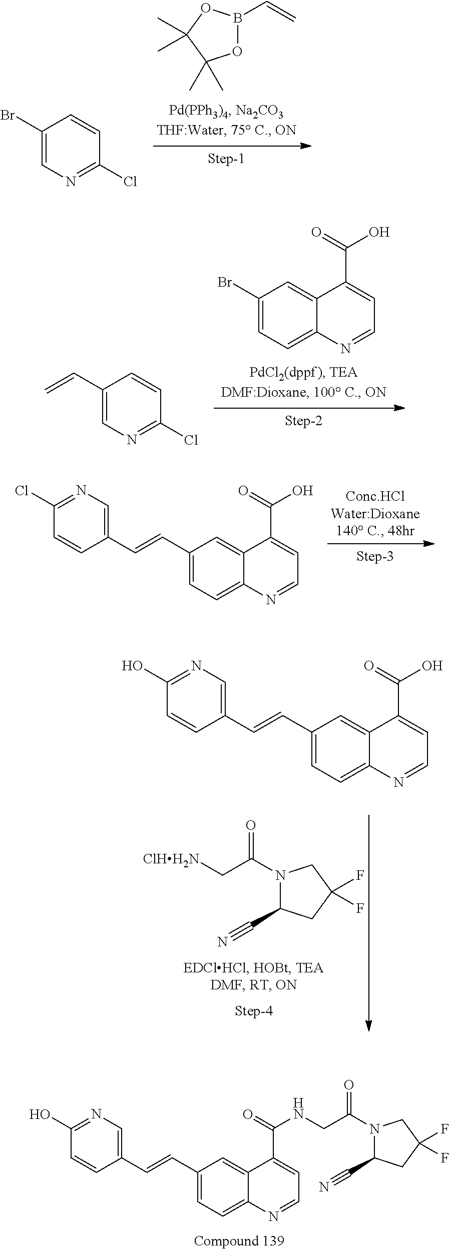

Step 1: Synthesis of 2-chloro-5-vinylpyridine. To a stirred solution of 5-bromo-2-chloropyridine (1.0 g, 5.19 mmol, 1.0 equiv) in THF (16 mL) was added 2-Vinyl-4,4,5,5-tetramethyl-1,3,2-dioxaoborolane (1.20 g, 7.79 mmol, 1.5 equiv) and a solution of Na₂CO₃ (2.75 g, 25.98 mmol, 5.0 equiv) in water (4 mL). The resulting mixture was purged with N₂ gas for 10 min. followed by the addition of Pd(PPh₃)₄(0.120 g, 0.103 mmol. 0.2 equiv). The resulting reaction mixture was heated at 75° C. for overnight. Product formation was confirmed by TLC. Reaction mixture was cooled to RT, diluted with water (50 mL) extracted with ethyl acetate (80 mL×3). Combined organic extracts were washed with water (80 mL×2) & brine (80 mL), dried over anhydrous Na₂SO₄ and concentrated. The crude product was purified by flash chromatography (0-5% EA in hexane as an eluent) to obtain 2-chloro-5-vinylpyridine (0.600 g, 83% Yield) as an oil.

¹H NMR (400 MHz, CHLOROFORM-d) δ 8.37 (d, J=2.63 Hz, 1H) 7.70 (dd, J=8.33, 2.63 Hz, 1H) 7.23-7.32 (m, 1H) 6.67 (dd, J=17.54, 10.96 Hz, 1H) 5.81 (d, J=17.54 Hz, 1H) 5.41 (d, J=11.40 Hz, 1H).

Step 2: Synthesis of (E)-6-(2-(6-chloropyridin-3-yl)vinyl)quinoline-4-carboxylic acid. To a stirred solution of 6-bromoquinoline-4-carboxylic acid (0.300 g, 1.190 mmol, 1.0 equiv) in mixture of DMF (4 mL) and Dioxane (4 mL) was added 2-chloro-5-vinylpyridine (0.248 g, 1.785 mmol, 1.5 equiv) and triethylamine (0.5 ml, 3.57 mmol, 3.0 equiv). The resulting reaction mixture was purged with N₂ gas for 5 min followed by addition of Pd(dppf)Cl₂ (0.043 g, 0.059 mmol, 0.05 equiv) and the reaction mixture was heated at 120° C. for overnight. Product formation was confirmed by LCMS. Reaction mixture was cool to RT, diluted with ethyl acetate (100 mL), filtered through Celite® and filtrate was washed with water (100 mL×2), Aqueous layer was separated and freeze dried over lyophilizer to obtain (E)-6-(2-(6-chloropyridin-3-yl)vinyl)quinoline-4-carboxylic acid (0.250 g, 67% Yield) as a yellow solid.

LCMS 311.0 [M+H]⁺

Step 3: Synthesis of (E)-6-(2-(6-hydroxypyridin-3-yl)vinyl)quinoline-4-carboxylic acid. To a stirred solution of (E)-6-(2-(6-chloropyridin-3-yl)vinyl)quinoline-4-carboxylic acid (0.200 g, 0.643 mmol, 1.0 equiv) in Dioxane (2 mL) and water (2 mL) was added Con. HCl (0.5 mL). The resulting reaction mixture was heated at 140° C. for 48 hr. Product formation was confirmed by LCMS. Reaction mixture was cooled to RT and concentrated under reduced pressure to obtain which was freeze dried over lyophilizer to obtain (E)-6-(2-(6-hydroxypyridin-3-yl)vinyl)quinoline-4-carboxylic acid (0.110 g, 58% Yield) as a yellow solid.

LCMS 293.0 [M+H]⁺

Step 4: Synthesis of (S,E)-N-(2-(2-cyano-4,4-difluoropyrrolidin-1-yl)-2-oxoethyl)-6-(2-(6-hydroxypyridin-3-yl)vinyl)quinoline-4-carboxamide. To a stirred solution of (E)-6-(2-(6-hydroxypyridin-3-yl)vinyl)quinoline-4-carboxylic acid (0.100 g, 0.342 mmol, 1.0 equiv) in DMF (4 mL), was added (S)-4,4-difluoro-1-glycylpyrrolidine-2-carbonitrile hydrochloride (0.092 g, 0.410 mmol, 1.2 equiv), HOBt (0.069 g, 0.513 mmol, 1.5 equiv) & EDCI.HCl (0.098 g, 0.513 mmol, 1.5 equiv). The mixture was allowed to stir at RT for 5 min. triethylamine (0.09 mL) was added and the mixture was allowed to stir at RT for overnight. Product formation was confirmed by LCMS and TLC. After completion of reaction, the mixture was diluted with water (40 mL) and extracted with ethyl acetate (50 mL×3). Combined organic extracts were washed with water (60 mL×3) and brine (60 mL), dried over anhydrous Na₂SO₄ and concentrated. The crude product was purified by reversed phase HPLC to obtain (S,E)-N-(2-(2-cyano-4,4-difluoropyrrolidin-1-yl)-2-oxoethyl)-6-(2-(6-hydroxypyridin-3-yl)vinyl)quinoline-4-carboxamide (0.040 g, 15% Yield) as a yellow solid.

LCMS 464.4 [M+H]⁺

¹H NMR (400 MHz, DMSO-d₆) δ 9.14 (t, J=5.92 Hz, 1H) 8.90 (d, J=4.38 Hz, 1H) 8.39 (s, 1H) 8.04 (s, 1H) 7.96 (d, J=7.02 Hz, 1H) 7.62 (br. s., 1H) 7.54 (d, J=3.95 Hz, 1H) 7.33 (d, J=16.22 Hz, 1H) 7.12 (d, J=16.22 Hz, 1H) 6.41 (d, J=9.65 Hz, 1H) 5.20 (d, J=6.58 Hz, 1H) 4.23-4.40 (m, 3H) 4.09-4.23 (m, 1H) 2.95 (br. s., 1H) 2.87 (d, J=17.10 Hz, 2H).

### Example S41

### Synthesis of N-(2-((S)-2-cyano-4,4-difluoropyrrolidin-1-yl)-2-oxoethyl)-3-(((S)-1-(4-fluorophenyl)ethyl)amino)isonicotinamide

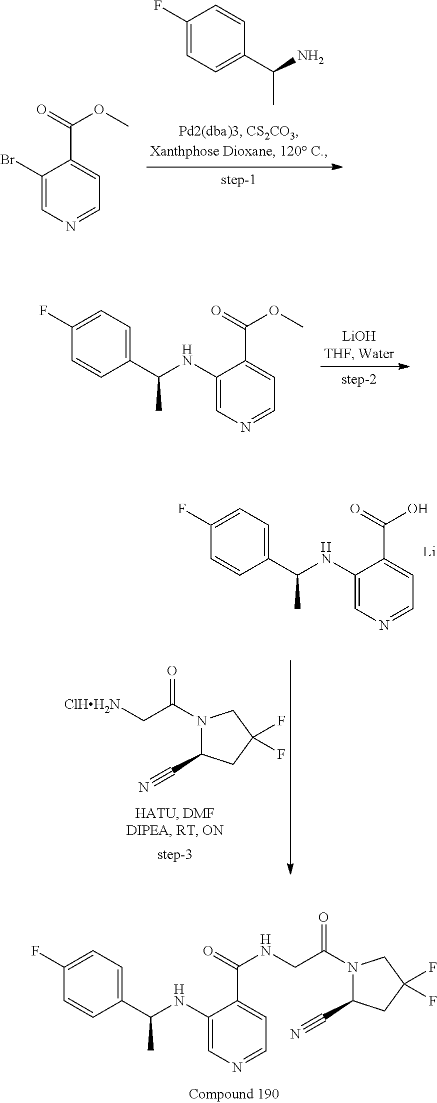

Step 1: Synthesis of methyl (S)-3-((1-(4-fluorophenyl)ethyl)amino)isonicotinate. To a stirred solution of methyl 3-bromoisonicotinate (0.500 g, 2.31 mmol, 1.0 equiv) and (S)-1-(4-fluorophenyl)ethan-1-amine (0.350 g, 2.54 mmol, 1.0 equiv) in Dioxan (10 mL) was added CS₂CO₃(1.5 g, 4.62 mmol, 2 equiv). The resulting mixture was purged with nitrogen for 10 min followed by addition of Pd₂(dba)₃ (0.110 g, 0.115 mmol, 0.05 equiv) and xanthphos (0.135 g, 0.231 mmol, 0.1 equiv). The reaction mixture was heated at 120° C. for overnight. The progress of reaction was monitored by TLC & LCMS. The reaction mixture was diluted with water (50 mL), extracted with EtOAc (3×80 mL). The combined organic layer was washed with water (2×60 mL), with brine (60 mL), dried over Na₂SO₄, concentrated. Crude product was purified by flash chromatography (0-50% ethyl acetate in hexane as an eluent) to obtain methyl (S)-3-((1-(4-fluorophenyl)ethyl)amino)isonicotinate (0.210 g, 34% Yield) as an oil.

LCMS: 275.0 [M+H]⁺

¹H NMR: (400 MHz, CHLOROFORM-d) δ 7.97 (s, 1H) 7.78-7.90 (m, 2H) 7.61 (d, J=5.26 Hz, 1H) 7.22-7.36 (m, 2H) 6.95-7.07 (m, 2H) 4.60-4.76 (m, 1H) 3.87-4.00 (s, 3H) 1.59 (d, J=7.02 Hz, 3H).

Step 2: Synthesis of (S)-3-((1-(4-fluorophenyl)ethyl)amino)isonicotinic acid, lithium salt. To a stirred solution of methyl (S)-3-((1-(4-fluorophenyl)ethyl)amino)isonicotinate (0.200 g, 0.727 mmol, 1.0 equiv) in THF (10 mL) and water (4 mL), was added LiOH (0.035 g, 1.45 mmol, 2 equiv). The mixture was allowed to stir at 80° C. for overnight. Product formation was confirmed by LCMS. The reaction mixture was concentrated and diluted with water (50 mL) and washed with ethyl acetate (2×50 mL). Aqueous layer was separated and freeze dried over lyophilyzer to obtain (S)-3-((1-(4-fluorophenyl)ethyl)amino)isonicotinic acid, lithium salt (0.200 g, Quant. Yield) as a yellow solid.

LCMS: 261.0 [M+H]⁺

Step 3: Synthesis of N-(2-((S)-2-cyano-4,4-difluoropyrrolidin-1-yl)-2-oxoethyl)-3-(((S)-1-(4-fluorophenyl)ethyl)amino)isonicotinamide. To a stirred solution of (S)-3-((1-(4-fluorophenyl)ethyl)amino)isonicotinic acid, lithium salt (0.200 g, 0.769 mmol, 1.0 equiv) in DMF (5 mL) was added (S)-4,4-difluoro-1-glycylpyrrolidine-2-carbonitrile hydrochloride (0.259 g, 1.153 mmol, 1.5 equiv), HATU (0.438 g, 1.153 mmols, 1.5 equiv) and DIPEA (0.198 g, 1.538 mmols, 2.0 equiv). The resulting reaction mixture was allowed stir at RT for overnight. The reaction progress was monitored by LCMS and TLC, reaction mixture was diluted with water (30 mL) extracted with ethyl acetate (3×40 mL). Combined organic layer was washed with water (40 mL×4), dried over anhydrous Na₂SO₄ and concentrated under reduced pressure. Crude product was purified by reversed phase chromatography to obtain N-(2-((S)-2-cyano-4,4-difluoropyrrolidin-1-yl)-2-oxoethyl)-3-(((S)-1-(4-fluorophenyl)ethyl)amino)isonicotinamide (0.050 g, 15%) as off white solid.

LCMS: 432.4 [M+H]⁺

¹H NMR: (400 MHz, DMSO-d₆) δ 9.03 (t, J=5.70 Hz, 1H) 7.89-7.95 (m, 2H) 7.83 (d, J=5.26 Hz, 1H) 7.48 (d, J=5.26 Hz, 1H) 7.40 (dd, J=8.55, 5.48 Hz, 2H) 7.15 (t, J=8.77 Hz, 2H) 5.13 (dd, J=9.21, 2.63 Hz, 1H) 4.81 (t, J=6.58 Hz, 1H) 4.25-4.39 (m, 1H) 3.99-4.24 (m, 3H) 2.89-3.02 (m, 1H) 2.83 (d, J=18.42 Hz, 1H) 1.45 (d, J=6.58 Hz, 3H).

### Example S42

### Synthesis of N-(2-((S)-2-cyano-4,4-difluoropyrrolidin-1-yl)-2-oxoethyl)-3-(((R)-1-(4-fluorophenyl)ethyl)amino)isonicotinamide

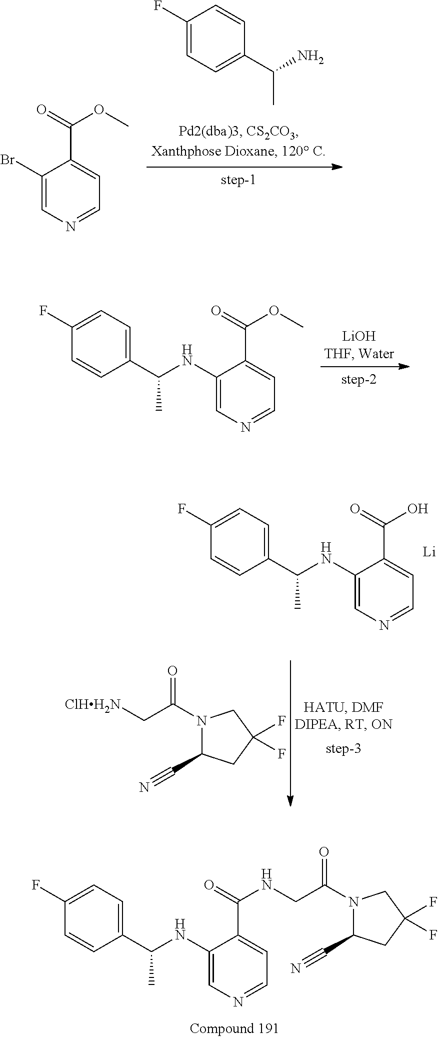

Step 1: Synthesis of methyl (R)-3-((1-(4-fluorophenyl)ethyl)amino)isonicotinate. To a stirred solution of methyl 3-bromoisonicotinate (0.500 g, 2.31 mmol, 1.0 equiv) and (S)-1-(4-fluorophenyl)ethan-1-amine (0.350 g, 2.54 mmol, 1.0 equiv) in Dioxan (10 mL) was added CS₂CO₃(1.5 g, 4.62 mmol, 2 equiv). The resulting mixture was purged with nitrogen for 10 min followed by addition of Pd₂(dba)₃ (0.110 g, 0.115 mmol, 0.05 equiv) and xanthphos (0.135 g, 0.231 mmol, 0.1 equiv). The reaction mixture was heated at 120° C. for overnight. The progress of reaction was monitored by TLC & LCMS. The reaction mixture was diluted with water (50 mL), extracted with EtOAc (3×80 mL). The combined organic layer was washed with water (2×60 mL), with brine (60 mL), dried over Na₂SO₄, concentrated. Crude product was purified by flash chromatography (0-50% ethyl acetate in hexane as an eluent) to obtain methyl (R)-3-((1-(4-fluorophenyl)ethyl)amino)isonicotinate (0.220 g, 35% Yield) as an oil.

LCMS: 275.0 [M+H]⁺

¹H NMR (400 MHz, CHLOROFORM-d) δ 7.97 (s, 1H) 7.78-7.90 (m, 2H) 7.61 (d, J=5.26 Hz, 1H) 7.22-7.36 (m, 2H) 6.95-7.07 (m, 2H) 4.60-4.76 (m, 1H) 3.87-4.00 (s, 3H) 1.59 (d, J=7.02 Hz, 3H).

Step 2: Synthesis of (R)-3-((1-(4-fluorophenyl)ethyl)amino)isonicotinic acid lithium salt. To a stirred solution of methyl (R)-3-((1-(4-fluorophenyl)ethyl)amino)isonicotinate (0.200 g, 0.727 mmol, 1.0 equiv) in THF (10 mL) and water (4 mL), was added LiOH (0.035 g, 1.45 mmol, 2 equiv). The mixture was allowed to stir at 80° C. for overnight. Product formation was confirmed by LCMS. The reaction mixture was concentrated and diluted with water (50 mL) and washed with ethyl acetate (2×50 mL). Aqueous layer was separated and freeze dried over lyophilyzer to obtain (R)-3-((1-(4-fluorophenyl)ethyl)amino)isonicotinic acid, lithium salt (0.200 g, Quant. Yield) as a yellow solid.

LCMS: 261.0 [M+H]⁺

Step 3: Synthesis of N-(2-((S)-2-cyano-4,4-difluoropyrrolidin-1-yl)-2-oxoethyl)-3-(((R)-1-(4-fluorophenyl)ethyl)amino)isonicotinamide. To a stirred solution of (R)-3-((1-(4-fluorophenyl)ethyl)amino)isonicotinic acid, lithium salt (0.200 g, 0.769 mmol, 1.0 equiv) in DMF (5 mL) was added (S)-4,4-difluoro-1-glycylpyrrolidine-2-carbonitrile hydrochloride (0.259 g, 1.15 mmol, 1.5 equiv), HATU (0.438 g, 1.15 mmol, 1.5 equiv) and DIPEA (0.198 g, 1.53 mmol, 2.0 equiv). The resulting reaction mixture was allowed stir at RT for overnight. The reaction progress was monitored by LCMS and TLC, reaction mixture was diluted with water (30 mL) extracted by ethyl acetate (3×40 mL). Combined organic layer was washed with water (40 mL×2). Organic layer was dried over anhydrous Na₂SO₄, concentrated under reduced pressure. Crude product was purified by reversed phase chromatography to obtain N-(2-((S)-2-cyano-4,4-difluoropyrrolidin-1-yl)-2-oxoethyl)-3-(((R)-1-(4-fluorophenyl)ethyl)amino)isonicotinamide (0.050 g, 15% Yield) as an off white solid.

LCMS: 432.4 [M+H]⁺

¹H NMR (400 MHz, DMSO-d₆) δ 9.03 (t, J=5.70 Hz, 1H) 7.89-7.95 (m, 2H) 7.83 (d, J=5.26 Hz, 1H) 7.48 (d, J=5.26 Hz, 1H) 7.40 (dd, J=8.55, 5.48 Hz, 2H) 7.15 (t, J=8.77 Hz, 2H) 5.13 (dd, J=9.21, 2.63 Hz, 1H) 4.81 (t, J=6.58 Hz, 1H) 4.25-4.39 (m, 1H) 3.99-4.24 (m, 3H) 2.89-3.02 (m, 1H) 2.83 (d, J=18.42 Hz, 1H) 1.45 (d, J=6.58 Hz, 3H).

### Example S43

### Synthesis of (S,E)-N-(2-(2-cyano-4,4-difluoropyrrolidin-1-yl)-2-oxoethyl)-6-(2-(2-methylpyridin-4-yl) vinyl)quinoline-4-carboxamide

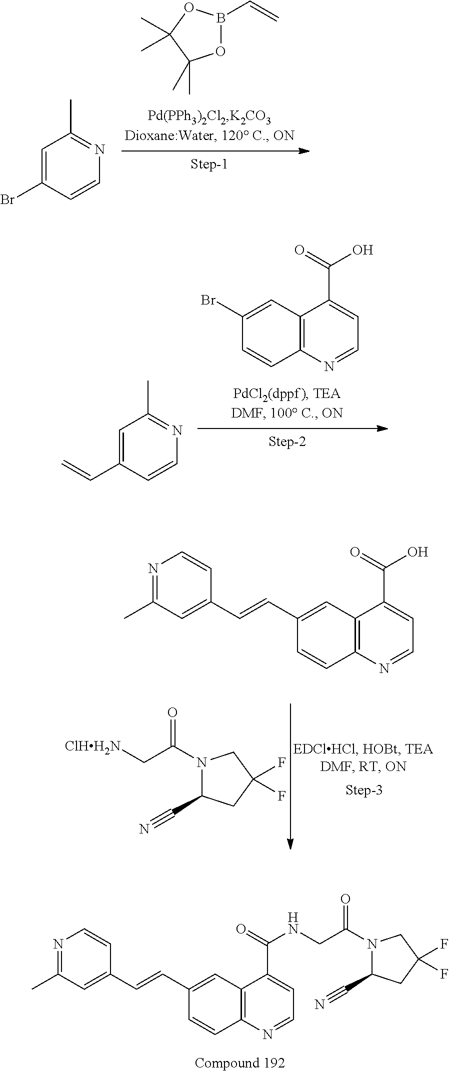

Step 1: Synthesis of 2-(methyl)-4-vinylpyridine. To a stirred solution of 4-Bromo-2-(methyl)pyridine (0.500 g, 2.90 mmol, 1.0 equiv) in Dioxane (8 mL) was added 2-Vinyl-4,4,5,5-tetramethyl-1,3,2-dioxaoborolane (0.671 g, 4.36 mmol, 1.5 equiv) and K₂CO₃ (0.802 g, 5.81 mmol, 2.0 equiv) in water (4 mL). The resulting reaction mixture was purged with N₂ gas for 10 minute, followed by the addition of Pd(PPh₃)Cl₂ (0.102 g, 0.145 mmol. 0.05 equiv). The reaction mixture was heated at 120° C. for overnight. Product formation was confirmed by TLC. Reaction mixture was cool to RT, diluted with water (50 mL) extracted with ethyl acetate (100 mL×3). Combined organic extracts were washed with water (50 mL×3), brine (50 mL), dried over anhydrous Na₂SO₄ and concentrated. The crude product obtained was purified by flash chromatography (0-15% ethyl acetate in hexane as an eluent) to obtain 2-(methyl)-4-vinylpyridine (0.170 g, 49% Yield) as a yellow semi-solid.

¹H NMR (400 MHz, CHLOROFORM-d) δ 8.44 (d, J=5.26 Hz, 1H) 7.05-7.19 (m, 2H) 6.63 (dd, J=17.76, 10.74 Hz, 1H) 5.94 (d, J=17.54 Hz, 1H) 5.45 (d, J=10.96 Hz, 1H) 2.55 (s, 3H).

Step 2: Synthesis of (E)-6-(2-(6-methylpyridin-3-yl)vinyl)quinoline-4-carboxylic acid. To a stirred solution of 6-bromoquinoline-4-carboxylic acid (0.200 g, 0.79 mmol, 1.0 equiv) in DMF (5 mL) was added 2-(methyl)-4-vinylpyridine (0.141 g, 1.19 mmol, 1.5 equiv) and Triethyl amine (0.34 ml, 2.38 mmol, 3.0 equiv). The resulting reaction mixture was purged with N₂ gas for 5 min followed by addition of Pd(dppf)Cl₂ (0.058 g, 0.079 mmol, 0.1 equiv). The reaction mixture was heated at 100° C. for overnight. Product formation was confirmed by LCMS. Reaction mixture was cool to RT, diluted with water (40 mL) washed with ethyl acetate (40 mL×2), Aqueous layer was separated and freeze dried over lyophilyzer to obtain (E)-6-(2-(6-methylpyridin-3-yl)vinyl)quinoline-4-carboxylic acid (0.200 g, 86% Yield) as a yellow solid.

LCMS 291.1 [M+H]⁺

Step 3: Synthesis of (S,E)-N-(2-(2-cyano-4,4-difluoropyrrolidin-1-yl)-2-oxoethyl)-6-(2-(6-methylpyridin-3-yl)vinyl)quinoline-4-carboxamide. To a stirred solution of (E)-6-(2-(6-methylpyridin-3-yl)vinyl)quinoline-4-carboxylic acid (0.200 g, 0.689 mmol, 1.0 equiv) in DMF (4 mL), was added (S)-4,4-difluoro-1-glycylpyrrolidine-2-carbonitrile hydrochloride (0.232 g, 1.034 mmol, 1.5 equiv), HOBt (0.139 g, 1.034 mmol, 1.5 equiv) & EDCI.HCl (0.197 g, 1.034 mmol, 1.5 equiv). The mixture was allowed to stir at RT for 10 min. triethyl amine (0.19 mL) was added and the mixture was allowed to stir at RT for overnight. Product formation was confirmed by LCMS and TLC. After completion of reaction, the mixture was diluted with water (50 mL) and extracted with ethyl acetate (60 mL×3). Combined organic extracts were washed with water (50 mL×3) and brine (50 mL), dried over anhydrous Na₂SO₄ and concentrated. The crude product obtained was purified by flash chromatography (5% MeOH in DCM as an eluent) followed by reversed phase purification to obtained (S,E)-N-(2-(2-cyano-4,4-difluoropyrrolidin-1-yl)-2-oxoethyl)-6-(2-(6-methylpyridin-3-yl)vinyl)quinoline-4-carboxamide (0.090 g, 28% Yield) as an off white solid.

LCMS 462.4 [M+H]⁺

¹H NMR (400 MHz, DMSO-d₆) δ 9.20 (t, J=6.14 Hz, 1H) 8.96 (d, J=3.95 Hz, 1H) 8.69 (s, 1H) 8.44 (d, J=4.82 Hz, 1H) 8.12 (q, J=8.62 Hz, 2H) 7.71 (d, J=16.22 Hz, 1H) 7.58 (d, J=4.39 Hz, 1H) 7.51-7.56 (m, 2H) 7.46 (d, J=4.82 Hz, 1H) 5.25 (d, J=6.58 Hz, 1H) 4.12-4.41 (m, 4H) 2.81-3.03 (m, 2H).

### Example S44

### Synthesis of (S,E)-N-(2-(2-cyano-4,4-difluoropyrrolidin-1-yl)-2-oxoethyl)-6-(2-(2-methoxypyridin-4-yl)vinyl)quinoline-4-carboxamide

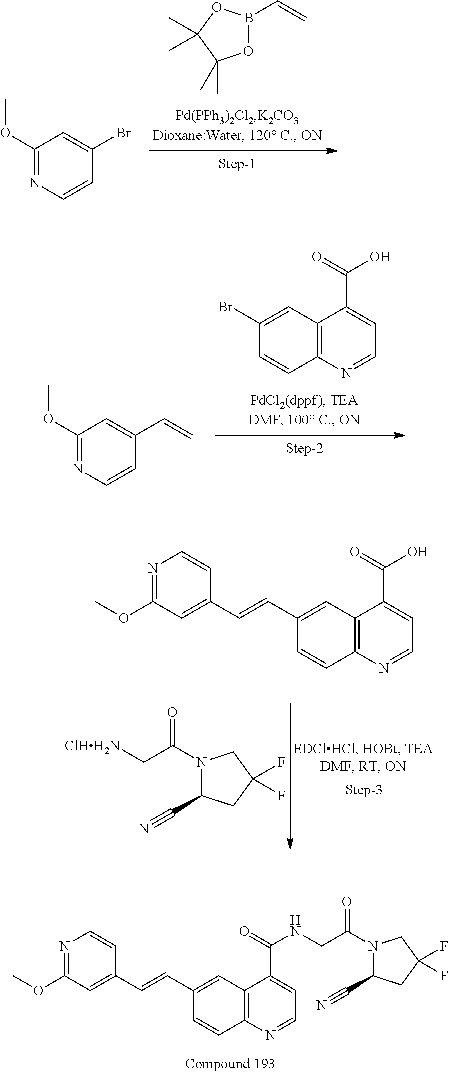

Step 1: Synthesis of 4-ethenyl-2-methoxy-pyridine. To a stirred solution of 4-bromo-2-methoxy-pyridine (0.500 g, 2.66 mmol, 1.0 equiv) in Dioxane (8 mL) was added 2-Vinyl-4,4,5,5-tetramethyl-1,3,2-dioxaoborolane (0.614 g, 3.99 mmol, 1.5 equiv) and K₂CO₃ (0.734 g, 5.32 mmol, 2.0 equiv) in water (4 mL), and resulting reaction mixture purged with N₂ gas for 10 minute, followed by the addition of Pd(PPh₃)Cl₂ (0.093 g, 0.132 mmol. 0.05 equiv). The resulting reaction mixture was heated at 120° C. for overnight. Product formation was confirmed by TLC. Reaction mixture was cooled to RT, diluted with water (50 mL) extracted with ethyl acetate (50 mL×3).

Combined organic extracts were washed with water (50 mL×2) and brine (50 mL), dried over anhydrous Na₂SO₄ and concentrated. The crude product obtained was purified by flash chromatography (0-10% ethyl acetate in hexane as an eluent) to obtain 4-ethenyl-2-methoxy-pyridine (0.180 g, 50% Yield) as an oil.

¹H NMR (400 MHz, CHLOROFORM-d) δ 8.11 (d, J=5.26 Hz, 1H) 6.91 (dd, J=5.26, 1.32 Hz, 1H) 6.69 (s, 1H) 6.62 (dd, J=17.54, 10.96 Hz, 1H) 5.91 (d, J=17.54 Hz, 1H) 5.44 (d, J=10.52 Hz, 1H) 3.94 (s, 3H).

Step 2: Synthesis of (E)-6-(2-(6-methoxypyridin-3-yl)vinyl)quinoline-4-carboxylic acid. To a stirred solution of 6-bromoquinoline-4-carboxylic acid (0.200 g, 0.793 mmol, 1.0 equiv) in DMF (5 mL) was added 4-ethenyl-2-methoxy-pyridine (0.160 g, 1.190 mmol, 1.5 equiv) and triethyl amine (0.34 ml, 2.380 mmol, 3.0 equiv). The resulting reaction mixture was purged with N₂ gas for 5 min followed by addition of Pd(dppf)Cl₂ (0.058 g, 0.079 mmol, 0.1 equiv). The reaction mixture was heated at 100° C. for overnight. Product formation was confirmed by LCMS. Reaction mixture was cooled to RT, diluted with water (40 mL) washed with ethyl acetate (40 mL×3), Aqueous layer was separated and freeze dried over lyophilyzer to obtained (E)-6-(2-(6-methoxypyridin-3-yl)vinyl)quinoline-4-carboxylic acid (0.200 g, 82% Yield) as a yellow solid.

LCMS 307.1 [M+H]⁺

Step 3: Synthesis of (S,E)-N-(2-(2-cyano-4,4-difluoropyrrolidin-1-yl)-2-oxoethyl)-6-(2-(6-methoxypyridin-3-yl)vinyl)quinoline-4-carboxamide. To a stirred solution of (E)-6-(2-(6-methoxypyridin-3-yl)vinyl)quinoline-4-carboxylic acid (0.200 g, 0.653 mmol, 1.0 equiv) in DMF (5 mL), was added (S)-4,4-difluoro-1-glycylpyrrolidine-2-carbonitrile hydrochloride (0.220 g, 0.980 mmol, 1.5 equiv), HOBt (0.132 g, 0.980 mmol, 1.5 equiv) & EDCI.HCl (0.187 g, 0.980 mmol, 1.5 equiv). The mixture was allowed to stir at RT for 10 min. triethyl amine (0.18 mL) was added and the mixture was allowed to stir at RT for overnight. Product formation was confirmed by LCMS and TLC. After completion of reaction, the mixture was diluted with water (50 mL) and extracted with ethyl acetate (100 mL×3). Combined organic extracts were washed with water (100 mL×3) and brine (100 mL), dried over anhydrous Na₂SO₄ and concentrated. The crude product obtained was purified by flash chromatography (5% MeOH in DCM as an eluent) to obtain S,E)-N-(2-(2-cyano-4,4-difluoropyrrolidin-1-yl)-2-oxoethyl)-6-(2-(6-methoxypyridin-3-yl)vinyl)quinoline-4-carboxamide (0.110 g, 35% Yield) as a yellow solid.

LCMS 478.4 [M+H]⁺

¹H NMR (400 MHz, DMSO-d₆) δ 9.20 (br. s., 1H) 8.97 (d, J=4.39 Hz, 1H) 8.60 (s, 1H) 8.16 (d, J=5.70 Hz, 2H) 8.07-8.13 (m, 1H) 7.70 (d, J=16.22 Hz, 1H) 7.59 (d, J=4.39 Hz, 1H) 7.49 (d, J=16.66 Hz, 1H) 7.31 (d, J=5.70 Hz, 1H) 7.04 (s, 1H) 5.22 (d, J=6.58 Hz, 1H) 4.24-4.41 (m, 2H) 4.10-4.24 (m, 1H) 3.87 (s, 3H) 2.96 (br. s., 1H) 2.88 (d, J=12.28 Hz, 1H).

### Example S45

### Synthesis of (S,E)-N-(2-(2-cyano-4,4-difluoropyrrolidin-1-yl)-2-oxoethyl)-6-(2-(2-(trifluoromethyl)pyridin-4-yl)vinyl)quinoline-4-carboxamide

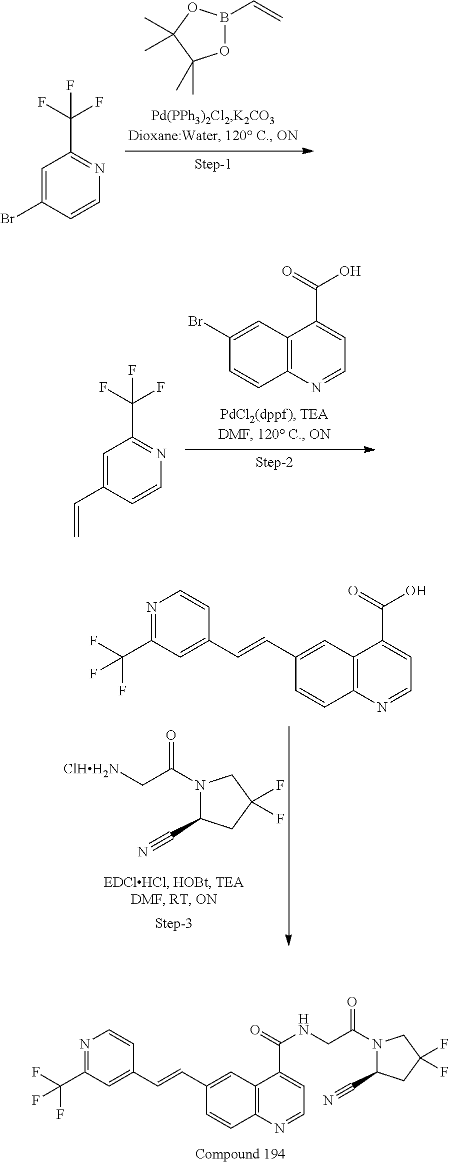

Step 1: Synthesis of 2-(trifluoromethyl)-4-vinylpyridine. To a stirred solution of 4-Bromo-2-(trifluoromethyl)pyridine (0.500 g, 2.21 mmol, 1.0 equiv) in dioxane (8 mL) was added 2-Vinyl-4,4,5,5-tetramethyl-1,3,2-dioxaoborolane (0.511 g, 3.31 mmol, 1.5 equiv) and K₂CO₃ (0.610 g, 4.42 mmol, 2.0 equiv) in water (4 mL). The resulting reaction mixture purged with N₂ gas for 10 minute, followed by the addition of Pd(PPh₃)Cl₂ (0.077 g, 0.110 mmol. 0.05 equiv). The resulting reaction mixture was heated at 120° C. for overnight. Product formation was confirmed by TLC. Reaction mixture was cooled to RT, diluted with water (100 mL) extracted with ethyl acetate (100 mL×3). Combined organic extracts were washed with water (100 mL×2) and brine (100 mL), dried over anhydrous Na₂SO₄ and concentrated. The crude product obtained was purified by flash chromatography (0-10% ethyl acetate in hexane as an eluent) to obtain 2-(trifluoromethyl)-4-vinylpyridine (0.210 g, 55% Yield) as a yellow semi solid.

¹H NMR (400 MHz, CHLOROFORM-d) 68.68 (d, J=5.26 Hz, 1H) 7.66 (s, 1H) 7.45 (d, J=3.95 Hz, 1H) 6.73 (dd, J=17.76, 10.74 Hz, 1H) 6.07 (d, J=17.54 Hz, 1H) 5.62 (d, J=10.96 Hz, 1H).

Step 2: Synthesis of (E)-6-(2-(6-(trifluoromethyl)pyridin-3-yl)vinyl)quinoline-4-carboxylic acid. To a stirred solution of 6-bromoquinoline-4-carboxylic acid (0.200 g, 0.793 mmol, 1.0 equiv) in dioxane (4 mL) was added 2-(trifluoromethyl)-4-vinylpyridine (0.205 g, 1.190 mmol, 1.5 equiv) and triethyl amine (0.34 ml, 2.380 mmol, 3.0 equiv). The resulting reaction mixture was purged with N₂ gas for 5 min followed by addition of Pd(dppf)Cl₂ (0.058 g, 0.0793 mmol, 0.1 equiv). The reaction mixture was heated at 120° C. for overnight. Product formation was confirmed by LCMS. Reaction mixture was cooled to RT, diluted with water (50 mL) washed with ethyl acetate (50 mL×2), Aqueous layer was separated and freeze dried over lyophilizer to obtain (E)-6-(2-(6-(trifluoromethyl)pyridin-3-yl)vinyl)quinoline-4-carboxylic acid (0.200 g, 73% Yield) as a yellow solid.

LCMS 345.1 [M+H]⁺

Step 3: Synthesis of (S,E)-N-(2-(2-cyano-4,4-difluoropyrrolidin-1-yl)-2-oxoethyl)-6-(2-(6-(trifluoromethyl)pyridin-3-yl)vinyl)quinoline-4-carboxamide. To a stirred solution of (E)-6-(2-(6-(trifluoromethyl)pyridin-3-yl)vinyl)quinoline-4-carboxylic acid (0.200 g, 0.581 mmol, 1.0 equiv) in DMF (4 mL), was added (S)-4,4-difluoro-1-glycylpyrrolidine-2-carbonitrile hydrochloride (0.196 g, 0.872 mmol, 1.5 equiv), HOBt (0.117 g, 0.872 mmol, 1.5 equiv) & EDCI.HCl (0.166 g, 0.872 mmol, 1.5 equiv). The mixture was allowed to stir at RT for 10 min. triethylamine (0.2 mL) was added and the mixture was allowed to stir at RT for overnight. Product formation was confirmed by LCMS and TLC. After completion of reaction, the mixture was diluted with water (40 mL) and extracted with ethyl acetate (50 mL×2). Combined organic extracts were washed with water (50 mL×2) and brine (50 mL), dried over anhydrous Na₂SO₄ and concentrated. The crude product obtained was purified by flash chromatography (5% MeOH in DCM as an eluent) followed by reversed phase purification to obtain (S,E)-N-(2-(2-cyano-4,4-difluoropyrrolidin-1-yl)-2-oxoethyl)-6-(2-(6-(trifluoromethyl)pyridin-3-yl)vinyl)quinoline-4-carboxamide (0.09 g, 3% Yield) as an off white solid.

LCMS 516.4 [M+H]⁺

¹H NMR (400 MHz, DMSO-d₆) δ 9.22 (br. s., 1H) 8.99 (d, J=4.38 Hz, 1H) 8.75 (d, J=4.38 Hz, 1H) 8.68 (s, 1H) 8.10-8.24 (m, 2H) 7.90-8.02 (m, 2H) 7.69 (s, 1H) 7.59-7.65 (m, 1H) 5.23 (d, J=7.45 Hz, 1H) 4.36 (br. s., 1H) 4.30 (br. s., 3H) 2.88 (d, J=12.28 Hz, 2H).

### Example S46

### Synthesis of (S)—N-(2-(2-cyano-4,4-difluoropyrrolidin-1-yl)-2-oxoethyl)-6-(4-(2-hydroxypropan-2-yl)phenyl)quinoline-4-carboxamide

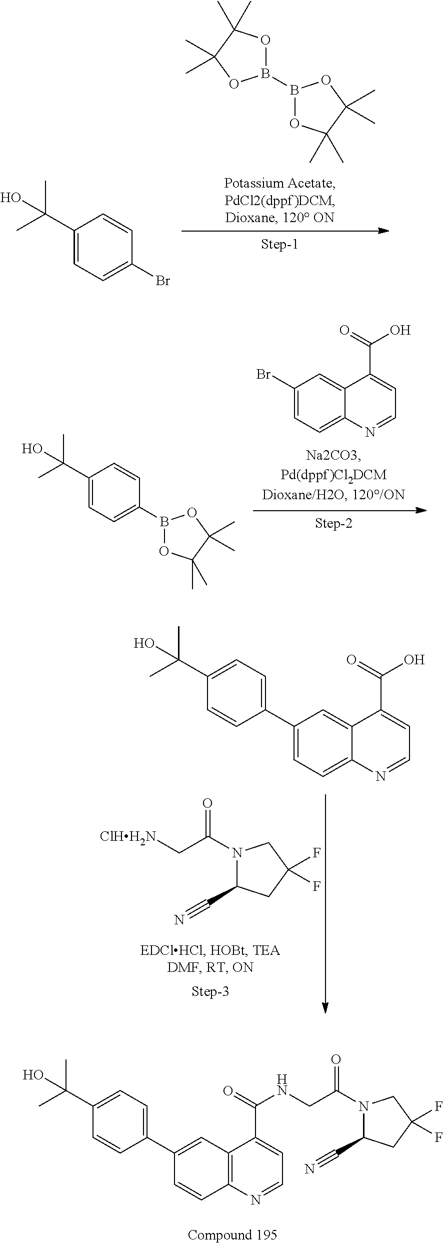

Step 1: Synthesis of 2-(4-(4,4,5,5-tetramethyl-1,3,2-dioxaborolan-2-yl)phenyl)propan-2-ol. To a solution of 2-(4-bromophenyl)propan-2-ol (0.3 g, 1.401 mmol, 1.0 equiv) in Dioxane (08 mL) was added 4,4,4′,4′,5,5,5′,5′-octamethyl-2,2′-bi(1,3,2-dioxaborolane) (0.42 g, 1.681 mmol, 1.0 equiv), potassium acetate (0.41 g, 4.203 mmol, 3.0 equiv). The resulting mixture was purged with N₂ gas for 10 minute, followed by the addition of Pd(dppf)Cl₂DCM (0.057 g, 0.070 mmol. 0.05 equiv). The resulting reaction mixture was heated at 120° C. for overnight. Product formation was confirmed by TLC. After the completion of reaction, the mixture was diluted with water solution (20 mL×2) and extracted with ethyl acetate (40 mL×2). Combined organic extracts were washed with water (10 mL×2), dried over anhydrous Na₂SO₄ and concentrated. The crude product obtained was purified by flash chromatography to obtain 2-(4-(4,4,5,5-tetramethyl-1,3,2-dioxaborolan-2-yl)phenyl)propan-2-ol (0.230 g, 68% yield) as an off-white solid.

¹H NMR (400 MHz, DMSO-d₆) δ 7.61 (m, J=8.33 Hz, 2H) 7.47 (m, J=8.33 Hz, 2H) 5.04 (s, 1H) 1.40 (s, 6H) 1.28 (s, 12H).

Step 2: Synthesis of 6-(4-(2-hydroxypropan-2-yl)phenyl)quinoline-4-carboxylic acid. To a solution of 2-(4-(4,4,5,5-tetramethyl-1,3,2-dioxaborolan-2-yl)phenyl)propan-2-ol (0.2 g, 0.763 mmol, 1.0 equiv) in Dioxane (5 mL) was added 6-bromoquinoline-4-carboxylic acid (0.192 g, 0.763 mmol, 1.0 equiv), Na₂CO₃(0.161 g, 1.526 mmol, 1.0 equiv) in H₂O (2 ml). The resulting reaction mixture was purged with N₂ gas for 10 minute, followed by the addition of Pd(dppf)Cl₂ DCM (0.031 g, 0.038 mmol. 0.05 equiv) and heated at 120° C. for overnight. Product formation was confirmed by LCMS. After the completion of reaction, the mixture was diluted with water solution (20 mL) and washed with ethyl acetate (20 mL×2). The aqueous layer was freeze dried over liophalizer to obtain 6-(4-(2-hydroxypropan-2-yl)phenyl)quinoline-4-carboxylic acid (0.150 g, 64% Yield) as an off white solid.

LCMS 308.2 [M+H]⁺

¹H NMR (400 MHz, DMSO-d₆) δ 9.00 (s, 1H) 8.75 (d, J=4.38 Hz, 1H) 7.97 (d, J=3.51 Hz, 2H) 7.66 (m, J=8.33 Hz, 2H) 7.58 (m, J=8.33 Hz, 2H) 7.48 (d, J=4.38 Hz, 1H) 5.08 (br. s., 1H) 1.46 (s, 6H).

Step 3: Synthesis of (S)—N-(2-(2-cyano-4,4-difluoropyrrolidin-1-yl)-2-oxoethyl)-6-(4-(2-hydroxypropan-2-yl)phenyl)quinoline-4-carboxamide. To a stirred solution of 6-(4-(2-hydroxypropan-2-yl)phenyl)quinoline-4-carboxylic acid (0.150 g, 0.488 mmol, 1.0 equiv) in DMF (4 mL), was added (S)-4,4-difluoro-1-glycylpyrrolidine-2-carbonitrile hydrochloride (0.109 g, 0.488 mmol, 1.0 equiv), EDCI.HCl (0.111 g, 0.585 mmol, 1.2 equiv) & HOBt (0.078 g, 0.585 mmol, 1.2 equiv). The mixture was allowed to stir at RT for 10 min. TEA (0.1 mL) was added and the mixture was allowed to stir at RT for overnight. Product formation was confirmed by LCMS and TLC. After completion of reaction, the mixture was diluted with water (20 mL) and extracted with ethyl acetate (40 mL×2). Combined organic extracts were washed with water (20 mL×4), dried over anhydrous Na₂SO₄ and concentrated. The crude product obtained was purified by reversed phase HPLC to obtain (S)—N-(2-(2-cyano-4,4-difluoropyrrolidin-1-yl)-2-oxoethyl)-6-(4-(2-hydroxypropan-2-yl)phenyl)quinoline-4-carboxamide (0.015 g, 6% Yield) as an off white solid.

LCMS 479.5 [M+H]⁺

¹H NMR (400 MHz, DMSO-d₆) δ 9.13-9.24 (m, 1H) 8.97 (d, J=4.39 Hz, 1H) 8.72 (s, 1H) 8.06-8.25 (m, 2H) 7.83 (d, J=8.33 Hz, 2H) 7.52-7.68 (m, 3H) 5.13-5.22 (m, 1H) 5.08 (br. s., 1H) 4.32-4.40 (m, 1H) 4.06-4.32 (m, 3H) 2.79-3.06 (m, 2H) 1.47 (br. s., 6H).

### Example S47

### Synthesis of (S,E)-N-(2-(2-cyano-4,4-difluoropyrrolidin-1-yl)-2-oxoethyl)-3-(2-(1-oxo-1,2,3,4-tetrahydroisoquinolin-6-yl)vinyl)isonicotinamide

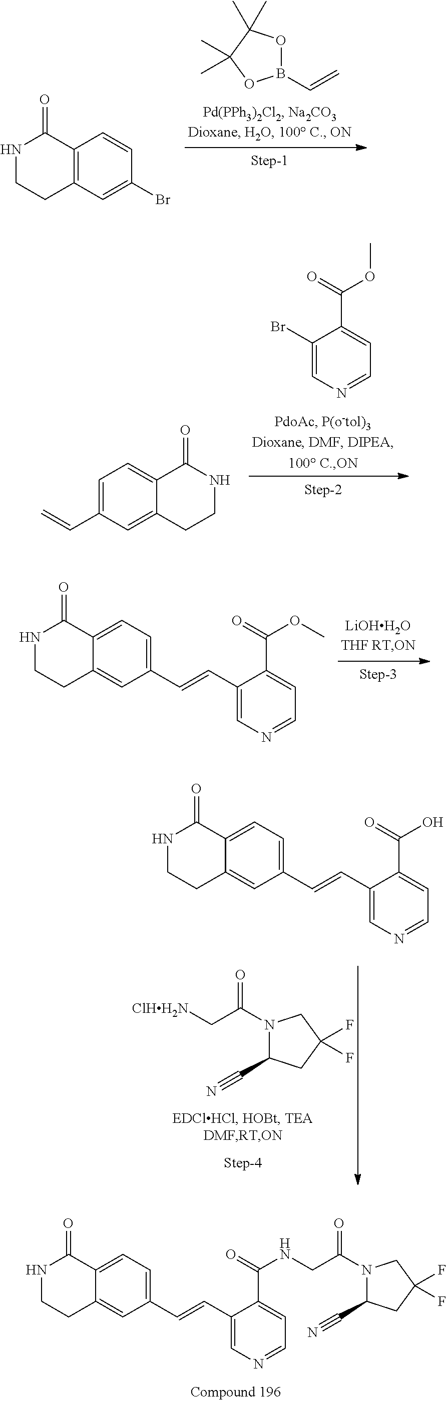

Step 1: Synthesis of 6-vinyl-3,4-dihydroisoquinolin-1(2H)-one. To a solution of 4,4,5,5-tetramethyl-2-vinyl-1,3,2-dioxaborolane (0.500 g, 3.24 mmol, 1.0 equiv) and 6-bromo-3,4-dihydroisoquinolin-1(2H)-one (0.587 g, 2.59 mmol, 0.8 equiv) and, in dioxane (10 mL) and water (2 mL) was added Na₂CO₃ (0.686 g, 6.48 mmol, 2.0 equiv). The resulting reaction mixture purged with N₂ gas for 10 min. followed by the addition of Pd(PPh₃)Cl₂ (0.114 g, 0.162 mmol, 0.05 equiv). The resulting reaction mixture was heated at 80° C. for overnight. Product formation was confirmed by LCMS and TLC. After the completion of reaction, the mixture was diluted with water (50 mL) and extracted with ethyl acetate (100 mL×2). Combined organic layer was washed with water (50 mL×2), dried over anhydrous Na₂SO₄ and concentrated. The crude product was purified by flash chromatography (0-30% ethyl acetate in hexane as an eluent) to obtain 6-vinyl-3,4-dihydroisoquinolin-1(2H)-one (0.250 g, 64% Yield) as a yellow semi solid.

LCMS 174.1 [M+H]⁺

Step 2: Synthesis of methyl (E)-3-(2-(1-oxo-1,2,3,4-tetrahydroisoquinolin-6-yl)vinyl)isonicotinate). To a solution of 6-vinyl-3,4-dihydroisoquinolin-1(2H)-one (0.150 g, 0.867 mmol, 1.0 equiv) in dioxane (1 mL) and DMF (2 mL) was added methyl 3-bromoisonicotinate (0.280 g, 1.30 mmol, 1.5 equiv) was added in P(O-tol)₃(0.052 g, 0.173 mmol, 0.2 equiv) and DIPEA (0.335 g, 2.601 mmol, 3.0 equiv). The resulting reaction mixture purged with N₂ gas for 10 min. followed by the addition of Pd(OAc) (0.019 g, 0.086 mmol, 0.1 equiv). The resulting reaction mixture was heated at 100° C. for overnight. Product formation was confirmed by LCMS. After the completion of reaction, the mixture was diluted with water (50 mL) extracted with ethyl acetate (100 mL). Combined organic extracts were washed with water (10 mL×4), dried over anhydrous Na₂SO₄ and concentrated. The crude product obtained was purified by flash chromatography (0-5% MeOH in DCM as an eluent) to obtain methyl (E)-3-(2-(1-oxo-1,2,3,4-tetrahydroisoquinolin-6-yl)vinyl)isonicotinate (0.1 g, 37% Yield) as an yellow solid.

LCMS 309.1 [M+H]⁺

Step 3: Synthesis of (E)-3-(2-(1-oxo-1,2,3,4-tetrahydroisoquinolin-6-yl)vinyl)isonicotinic acid. To a stirred solution of methyl (E)-3-(2-(1-oxo-1,2,3,4-tetrahydroisoquinolin-6-yl)vinyl)isonicotinate (0.1 g, 0.323 mmol, 1.0 equiv) in THF (3 mL) and water (4 mL), was added LiOH.H₂O (0.016 g, 0.388 mmol, 1.2 equiv). The mixture was allowed to stir at RT for overnight. Product formation was confirmed by LCMS. The reaction mixture was concentrated under reduced pressure and diluted with water (20 mL). The aqueous layer was washed with ethyl acetate (10 mL×2) and freeze dried over lyophilizer to obtain E)-3-(2-(1-oxo-1,2,3,4-tetrahydroisoquinolin-6-yl)vinyl)isonicotinic acid quantitatively yield (0.094 g, Quant. Yield) as a yellow solid.

LCMS 295.0 [M+H]⁺

Step 4: Synthesis of (S,E)-N-(2-(2-cyano-4,4-difluoropyrrolidin-1-yl)-2-oxoethyl)-3-(2-(1-oxo-1,2,3,4-tetrahydroisoquinolin-6-yl)vinyl)isonicotinamide. To a stirred solution of (E)-3-(2-(1-oxo-1,2,3,4-tetrahydroisoquinolin-6-yl)vinyl)isonicotinic acid (0.1 g, 0.340 mmol, 1.0 equiv) in DMF (5 mL), was added (S)-4,4-difluoro-1-glycylpyrrolidine-2-carbonitrile hydrochloride (0.114 g, 0.510 mmol, 1.5 equiv), HOBt (0.068 g, 0.510 mmol, 1.5 equiv) & EDCI.HCl (0.097 g, 0.510 mmol, 1.5 equiv). The mixture was allowed to stir at RT for 10 min. triethylamine (0.103 g, 1.02 mmol, 3.0 equiv) was added and the mixture was allowed to stir at RT for overnight. Product formation was confirmed by LCMS and TLC. After completion of reaction, the mixture was diluted with water (20 mL) and extracted with ethyl acetate (20 mL×2). Combined organic extracts were washed with water (20 mL×4), dried over anhydrous Na₂SO₄ and concentrated. The crude product was purified by flash chromatography (0-5% MeOH in DCM as an eluent) which was further purified by reversed phase HPLC to obtain (S,E)-N-(2-(2-cyano-4,4-difluoropyrrolidin-1-yl)-2-oxoethyl)-3-(2-(1-oxo-1,2,3,4-tetrahydroisoquinolin-6-yl)vinyl)isonicotinamide (0.007 g, 4% Yield) as an off white solid.

LCMS 466.5 [M+H]⁺

¹H NMR (400 MHz, DMSO-d₆) δ 9.17 (s, 1H) 9.04 (br. s., 1H) 8.55 (d, J=4.82 Hz, 1H) 7.90 (br. s., 1H) 7.86 (d, J=7.89 Hz, 2H) 7.60-7.72 (m, 2H) 7.52 (d, J=16.66 Hz, 2H) 7.37 (d, J=5.26 Hz, 1H) 5.19 (d, J=9.21 Hz, 1H) 4.33 (br. s., 1H) 4.20 (d, J=6.14 Hz, 1H) 4.12 (d, J=11.40 Hz, 2H) 3.38 (t, 2H) 2.96 (t, 2H) 2.83 (m, 2H).

### Biological Examples

### Example B1

### Inhibition of FAPα by Test Compounds was Assessed by In Vitro Enzymatic Activity Assays

FAPα enzymatic exopeptidase (dipeptidase) activity assay. To assay baseline FAPα enzymatic exopeptidase activity, 40 ng of recombinant human FAPα (rhFAPα, R&S system, #3715-SE) was incubated with 100 μM of Z-Gly-Pro-AMC peptide (BACHEM, # L-1145) in a FAPα assay buffer (50 mM Tris pH 7.4, 100 mM NaCl, 0.1 mg/ml bovine serum albumin) for 1 h at 37° C. protected from light in 96-well black plates (Nunc, #237108). To assay FAPα enzymatic exopeptidase activity inhibition by test compounds, all test compounds were pre-incubated with the enzyme for 15 min at 37° C. before starting the reaction by substrate addition in 96-well black plates (Nunc, #237108). 7-Amino-4-Methylcoumarin (AMC) release was detected by measuring fluorescence at Ex/Em 380/460 nm using a Multifunction Microplate Reader (Synergy 4, Biotek). All measurements were carried out in duplicate. Val-boroPro, a non-specific prolyl peptidase inhibitor, was used as a positive control. Percent inhibition of rmFAPα or rhFAPα enzymatic exopeptidase activity at 1 μM was determined for certain compounds, as shown in Table 2. For the calculations, the average measurements from reactions containing only vehicle and substrate, without enzyme, were used as a blank and were subtracted from the rest of the measurements. Percent inhibition was calculated using the average measurements from reactions containing vehicle, enzyme, and substrate as the maximum of enzymatic activity. Additionally, IC₅₀ for the rmFAPα or rhFAPα enzymatic exopeptidase activity of certain compounds are also shown in Table 2. Measurements were performed as a single point.

FAPα enzymatic endopeptidase (collagenase) activity assay. To assay baseline FAPα enzymatic exopeptidase activity, 50 ng of recombinant human FAPα (rhFAPα) (R&S system, #3715-SE) diluted in FAPα assay buffer (50 mM Tris pH 7.4, 100 mM NaCl, 0.1 mg/ml bovine serum albumin) was incubated with 5 μg of substrate DQ collagen solution (Molecular Probes # D-12060) with for 5 h at 37° C. and protected from light in 384-well optiplates (Perkin Elmer, #384-F). To assay FAPα enzymatic endopeptidase activity inhibition by test compounds, all test compounds were pre-incubated with the enzyme for 30 min at 37° C. before starting the reaction by substrate addition in 384-well OptiPlates (Perkin Elmer, #384-F). Collagen hydrolysis was determined by measuring fluorescence at Ex/Em 495/515 nm using a multifunction Microplate Reader (Synergy 4, Biotek). All measurements were performed as a single point. Val-boroPro, a non-specific prolyl peptidase inhibitor, was used as a positive control. IC₅₀ for the rhFAPα enzymatic endopeptidase activity (as determined by the collagenase assay) of certain compounds are also shown in Table 2.

TABLE 2
Exopeptidase or Endopeptidase inhibition of rmFAPα
or rhFAPα by Test Compounds

| | rhFAPα | | |
|---|---|---|---|
| | (% exo inh @ | rhFAPα (exo | rhFAPα (endo |
| Compound No. | 1 μM) | IC₅₀, μM) | IC₅₀, μM) |
| Val-boroPro | − | ++ | ++ |
| Ref. Comp. | +++ | +++ | +++ |
| 1 | +++ | +++ | − |
| 2 | +++ | +++ | − |
| 3 | +++ | +++ | +++ |
| 4 | +++ | +++ | − |
| 5 | +++ | +++ | − |
| 6 | +++ | +++ | − |
| 7 | +++ | +++ | − |
| 8 | +++ | +++ | − |
| 9 | +++ | +++ | +++ |
| 10 | +++ | +++ | − |
| 11 | +++ | +++ | − |
| 12 | +++ | +++ | − |
| 13 | +++ | +++ | − |
| 14 | +++ | +++ | − |

Ref. Comp.: Compound 60 as described in Jansen, K., et al., J Med Chem, 2014. 57(7): p. 3053-74; for % of inhibition: +++ refers to >50% inhibition at 1 μM test compound; ++ refers to 25% < % inhibition < 50% a 1 μM test compound; + refers to <25% inhibition at 1 μM; for IC₅₀: +++ refers to IC₅₀ < 1 μM; ++ refers to 1 μM < IC₅₀ < 10 μM; + refers to IC₅₀ > 10 μM; − represents compound not tested;; rhFAPα: recombinant human fibroblast activation protein alpha; endo: endopeptidase; exo: exopeptidase; inh: inhibition.

### Example B2

### Selectivity of the Inhibition of FAPα by Test Compounds was Assessed Compared to Other Prolyl Oligopeptidase Family S9 Members: DPPIV, PREP, and DPP9

### DPPIV Enzymatic Activity Assay

To assay baseline dipeptidyl peptidase-4 (DPPIV) activity, 40 ng of recombinant human DPPIV (rhDPPIV) (R&S system, #1180-SE) was incubated with 400 μM of H-Gly-Pro-pNA substrate (BACHEM, # L-1880) in a DPPIV assay buffer (25 mM Tris, pH 8.3) for 30 min at 37° C. protected from the light in 96-well black plates (Nunc, #237108). To assay DPPIV inhibition by test compounds, test compounds were pre-incubated with the enzyme for 15 min at 37° C. before starting the reaction by substrate addition in 96-well black plates (Nunc, #237108). Para-nitroaniline (pNA) release was detected by measuring absorbance at 405 nm using a Multifunction Microplate Reader (Synergy 4, Biotek). All measurements were carried out in triplicate. Val-boroPro, a non-specific prolyl peptidase inhibitor, was used as a positive control.

### PREP Enzymatic Activity Assay

To assay baseline prolyl endopeptidase (PREP) activity, 20 ng of recombinant human PREP (rhPREP) (R&S system, #4308-SE) was incubated with 100 μM of Z-Gly-Pro-AMC peptide (BACHEM, # L-1145) in a PREP assay buffer (25 mM Tris, 250 mM NaCl, 10 mM DTT, pH 7.5) for 30 min at 37° C. protected from light in 96-well black plates (Nunc, #237108). To assay PREP activity inhibition by test compounds, test compounds were pre-incubated with the enzyme for 15 min at 37° C. before starting the reaction by substrate addition in 96-well black plates (Nunc, #237108). 7-Amino-4-Methylcoumarin (AMC) release was detected by measuring fluorescence at Ex/Em 380/460 nm using a Multifunction Microplate Reader (Synergy 4, Biotek). All measurements were carried out in triplicate. Val-boroPro, a non-specific prolyl peptidase inhibitor, was used as a positive control.

### DPP9 Enzymatic Activity Assay

To assay baseline dipeptidyl peptidase 9 (DPP9) activity, 40 ng of recombinant human DPP9 (rhDPP9) (R&S system, #5419-SE) was incubated with 100 μM of H-Gly-Pro-AMC peptide (BACHEM, # L-1215) in a DDP9 assay buffer (50 mM HEPES, pH 8) for 30 min at 37° C. in 96-well black plates (Nunc, #237108). To assay rhDPP9 activity inhibition by test compounds, test compounds were pre-incubated with the enzyme for 15 min at 37° C. before starting the reaction by substrate addition in 96-well black plates (Nunc, #237108). 7-Amino-4-Methylcoumarin (AMC) release was detected by measuring fluorescence at Ex/Em 380/460 nm using a Multifunction Microplate Reader (Synergy 4, Biotek). All measurements were carried out in triplicate. Val-boroPro, a non-specific prolyl peptidase inhibitor, was used as a positive control.

To determine if new FAPα inhibitors were selective or if they also inhibited other prolyl peptidases, the IC₅₀ of certain test compounds, a reference compound (compound 60 as described in Jansen, K., et al., J Med Chem, 2014. 57(7): p. 3053-74), and Val-boroPro were determined, as shown in Table 3A.

TABLE 3A
Selectivity of FAPα Inhibition by Test Compounds

| Compound | rhFAPα | rhDPPIV | rhPREP | rhDPP9 |
|---|---|---|---|---|
| No. | (exo IC₅₀, μM) | (IC₅₀, μM) | (IC₅₀, μM) | (IC₅₀, μM) |
| Val-boroPro | ++ | +++ | ++ | +++ |
| Ref. Comp. | +++ | + | ++ | ++ |
| 1 | +++ | + | + | + |
| 2 | +++ | + | + | + |
| 3 | +++ | ++ | + | ++ |
| 4 | +++ | ++ | + | + |
| 5 | +++ | ++ | + | ++ |
| 6 | +++ | − | − | ++ |
| 7 | +++ | + | ++ | ++ |
| 8 | +++ | + | + | ++ |
| 9 | +++ | + | + | +++ |
| 10 | +++ | ++ | ++ | ++ |
| 11 | +++ | + | ++ | ++ |
| 12 | +++ | ++ | + | ++ |
| 13 | +++ | + | + | ++ |
| 14 | +++ | + | − | ++ |

Ref. Comp.: Compound 60 as described in Jansen, K., et al., J Med Chem, 2014. 57(7): p. 3053-74; for IC₅₀: +++ refers to IC₅₀ < 1 μM; ++ refers to 1 μM < IC₅₀ < 10 μM; + refers to IC₅₀ > 10 μM; − represents compound not tested; rhFAPα: recombinant human fibroblast activation protein alpha; rhDPPIV: recombinant human dipeptidyl peptidase-4; rhDPP9: recombinant human dipeptidyl peptidase 9; exo: exopeptidase.

Furthermore, to assay the inhibition of dipeptidyl peptidase 9 (DPP9) activity, aliquots of L of diluted exemplary compounds, reference compounds or vehicle were mixed in a 96-well black plate with 40 μL of DPP9 assay buffer (25 mM Tris-HCl pH 8.0 and 0.01% BSA) containing 10 ng of recombinant human DPP9 (rhDPP9) (Cat. No. #5419-SE, R&D systems). Compounds were allowed to interact with the enzyme for 15 min at 37° C. before the start of the reaction by adding 50 μL of the synthetic dipeptide substrate, 200 μM H-Gly-ProAMC (Cat. No. # L-1215, Bachem) in DPPIV assay buffer. Reactions were carried out for 30 min at 37° C. protected from light. AMC release was detected by measuring fluorescence at Ex/Em 380/460 nm using a Multifunction Microplate Reader. Results for the inhibition of other prolyl peptidases from S9 family for exemplary compounds are shown in Table 3B.

TABLE 3B
Inhibition of other prolyl peptidases from
S9 family members by exemplary compounds

| | rhFAPα | | | | |
|---|---|---|---|---|---|
| Compound | % exo inh | rhFAP | rhDPP9 | rhDPPIV | rhPREP |
| No. | @ 1 μM | IC₅₀, μM | IC₅₀, μM | IC₅₀, μM | IC₅₀, μM |
| Val- | ++ | ++ | +++ | +++ | ++ |
| boroPro | | | | | |
| Ref. | +++ | +++ | +++ | + | +++ |
| Comp. | | | | | |
| 1 | +++ | +++ | ++ | + | + |
| 2 | +++ | +++ | + | + | + |
| 3 | +++ | +++ | ++ | ++ | + |
| 4 | +++ | +++ | ++ | ++ | + |
| 5 | +++ | +++ | ++ | ++ | + |
| 6 | +++ | +++ | ++ | − | − |
| 7 | +++ | +++ | ++ | + | ++ |
| 8 | +++ | +++ | ++ | + | + |
| 9 | +++ | +++ | +++ | + | + |
| 10 | +++ | +++ | ++ | ++ | ++ |
| 11 | +++ | +++ | ++ | + | ++ |
| 12 | +++ | +++ | ++ | ++ | + |
| 13 | +++ | +++ | ++ | + | + |
| 14 | +++ | +++ | ++ | + | ++ |
| 15 | +++ | +++ | +++ | + | + |
| 19 | +++ | +++ | ++ | + | + |
| 20 | +++ | +++ | ++ | + | + |
| 24 | +++ | +++ | ++ | ++ | + |
| 26 | +++ | +++ | +++ | + | + |
| 29 | +++ | +++ | ++ | + | + |
| 33 | +++ | +++ | +++ | + | + |
| 34 | +++ | +++ | +++ | + | + |
| 35 | +++ | +++ | +++ | + | + |
| 36 | +++ | +++ | + | + | + |
| 37 | +++ | +++ | + | + | + |
| 38 | +++ | +++ | +++ | + | + |
| 39 | +++ | +++ | +++ | + | + |
| 40 | +++ | +++ | +++ | ++ | + |
| 41 | +++ | +++ | +++ | + | + |
| 42 | +++ | +++ | +++ | + | + |
| 43 | +++ | +++ | +++ | + | + |
| 44 | +++ | +++ | +++ | + | + |
| 45 | +++ | +++ | +++ | + | + |
| 46 | +++ | +++ | +++ | + | + |
| 47 | +++ | +++ | +++ | + | + |
| 48 | +++ | +++ | +++ | + | + |
| 49 | +++ | +++ | +++ | − | − |
| 50 | +++ | +++ | ++ | − | − |
| 51 | +++ | +++ | +++ | − | − |
| 52 | +++ | +++ | ++ | − | − |
| 139 | +++ | +++ | +++ | + | + |
| 190 | +++ | +++ | − | + | − |
| 191 | +++ | +++ | − | + | ++ |
| 192 | +++ | +++ | +++ | ++ | + |
| 193 | +++ | +++ | +++ | + | + |
| 194 | +++ | +++ | +++ | ++ | + |
| 195 | +++ | +++ | − | + | + |

Ref. Comp.: Compound 60 as described in Jansen, K., et al., J Med Chem, 2014. 57(7): p. 3053-74; for % of inhibition: +++ refers to >50% inhibition at 1 μM test compound; ++ refers to 25% < % inhibition < 50% at 1 μM test compound; + refers to <25% inhibition at 1 μM; for IC₅₀: +++ refers to IC₅₀ < 1 μM; ++ refers to 1 μM < IC₅₀ < 10 μM; + refers to IC₅₀ > 10 μM; rhFAPα: recombinant human fibroblast activation protein alpha; exo: exopeptidase; inh: inhibition; rhFAP: recombinant human fibroblast activation protein; rhDPPIV: recombinant human dipeptidyl peptidase-4; rhDPP9: recombinant human dipeptidyl peptidase 9; rhPREP: recombinant human prolyl endopeptidase.

### Example B3

### Validation of Selective PRXS-AMC Substrate for FAPα Activity Measurements

FAPα activity can be measured by a general fluorescence intensity assay for dipeptidyl-peptidases using a peptide substrate attached to a chemically quenched dye, such as Ala-Pro-7-amino-4-trifluoromethyl-coumarin (AFC) or a substrate containing the consensus Gly-Pro dipeptide such as Z-Gly-Pro-AMC (Levy, M. T., et al., Hepatology, 1999, 29(6): 1768-78; Santos, A. M., et al., J Clin Invest, 2009, 119(12): 3613-25; Park, J. E., et al., J Biol Chem, 1999, 274(51): 36505-12; Niedermeyer, J., et al., Mol Cell Biol, 2000, 20(3): 1089-94; Narra, K., et al., Cancer Biol Ther, 2007, 6(11): 1691-9; Lee, K. N., et al., J Thromb Haemost, 2011, 9(5): 987-96; Li, J., et al., Bioconjug Chem, 2012, 23(8): 1704-11). These substrates are likely targeted also by other circulating proline-specific endopeptidases such as PREP that could be present in the reaction. By contrast, a proprietary substrate reagent, named PRXS-AMC, can specifically monitor FAPα activity.

To validate the high selectivity of this proprietary substrate, enzymatic activity assays for FAP, DPPIV, PREP and DPP9 were carried out using Z-Gly-Pro-AMC or PRXS-AMC as described in Examples B1 and B2.

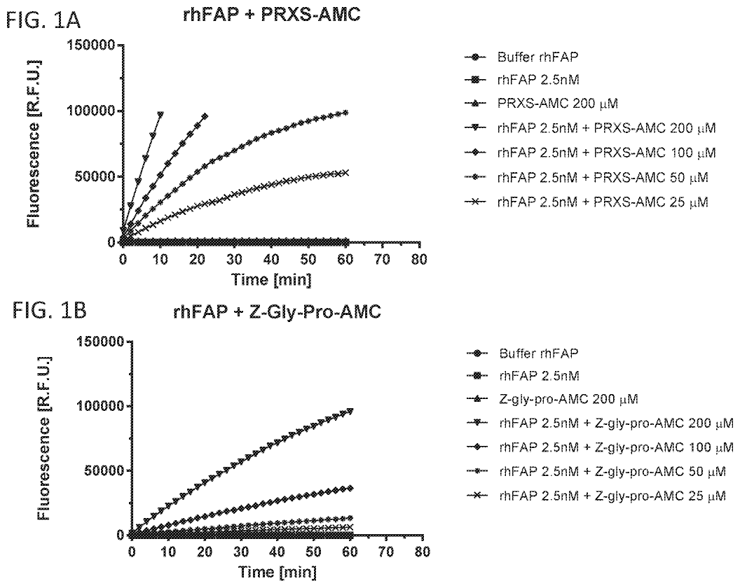

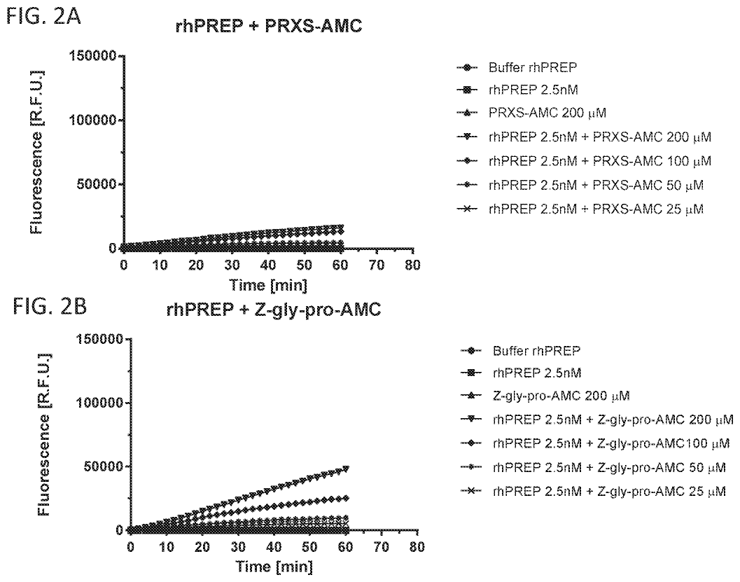

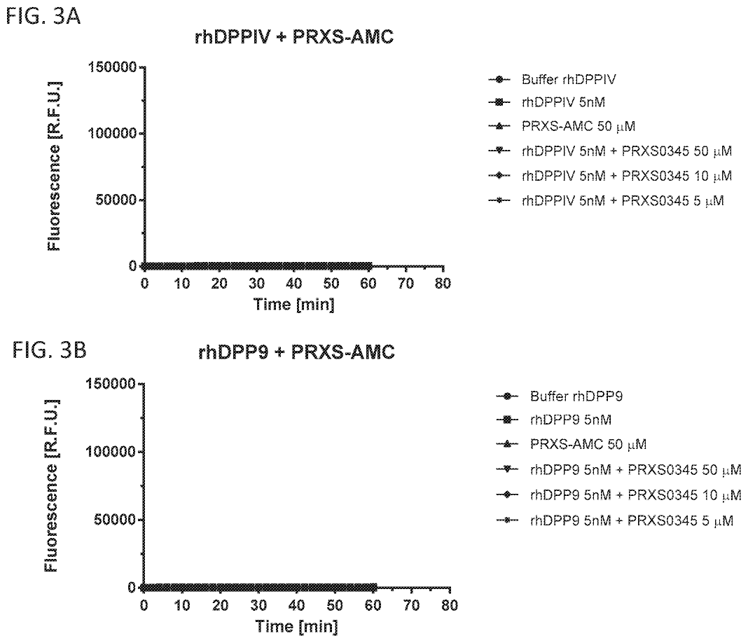

To assay FAPα, DPPIV, DPP9 and PREP enzymatic activities, human recombinant enzymes were used at 5, 2.5, 2.5 and 5 nM final concentrations, respectively. Z-Gly-Pro-AMC or PRXS-AMC were used at 25, 50, 100 and 200 μM final concentrations. Reactions were carried out for 60 min at 37° C. and were protected from light. AMC release was detected by measuring fluorescence at Ex/Em 380/460 nm using a Multifunction Microplate Reader in kinetic mode. Measurements were performed as a single point. Resulting fluorescence over time for PRXS-AMC and Z-gly-pro-AMC in the presence of rhFAPα is shown in FIG. 1A and FIG. 1B, respectively; resulting fluorescence over time for PRXS-AMC and Z-gly-pro-AMC in the presence of rhPREP is shown in FIG. 2A and FIG. 2B, respectively; and resulting fluorescence over time for PRXS-AMC in the presence of rhDPPIV or rhDPP9 is shown in FIG. 3A and FIG. 3B, respectively.

PRXS-AMC is processed to a lesser extent than Z-Gly-Pro-AMC by the closely related prolyl oligopeptidase PREP at similar concentrations (see FIGS. 2A-2B). PRXS-AMC is not processed by DPPIV or DPP9 (FIGS. 3A-3B). In addition, PRXS-AMC showed an improved solubility in aqueous buffers.

### Example B4

### Enzymatic Activity in Plasma

### FAPα Enzymatic Activity in Mouse Plasma

Approximately 500 μL of whole blood from one C57BL/6 mouse was harvested into BD Microtainer® tubes (K2) EDTA (#365974, Becton Dickinson and Co.) via terminal cardiac puncture. The blood sample was immediately centrifuged at approximately 9000 g at 4° C. for 5 minutes. Plasma was separated and stored at −80° C. in aliquots of 300 μL. To assay baseline FAPα enzymatic exopeptidase activity, 5 μL of thawed, plasma was diluted (1:5) with cFAP buffer (100 mM Tris-HCl, 400 mM NaCl, 50 mM salicylic acid, 1 mM EDTA, pH 7.5) and mixed with 35 μL of the same buffer before being pre-incubated with different concentrations of 10 μL of test compounds or DMSO vehicle for 15 minutes at 37° C. in 96-well black plates (Nunc, #237108). After pre-incubation, 50 μL of 200 μM PRXS-AMC were added to the mixture. The assay was performed for 1 hour at 37° C. protected from light. 7-Amino-4-Methylcoumarin (AMC) release was detected measuring fluorescence at an excitation wavelength of 380 nm and an emission wavelength of 460 nm using a Multifunction Microplate Reader (Synergy 4, Biotek). All measurements were carried out at least as a single point. Results are shown in Table 4.

### DPPIV Enzymatic Activity in Mouse Plasma

Approximately 500 μL of whole blood from one C57BL/6 mouse was harvested into BD Microtainer® tubes (K2) EDTA (#365974, Becton Dickinson and Co.) via terminal cardiac puncture. The blood sample was immediately centrifuged at approximately 9000 g at 4° C. for 5 minutes. Plasma was separated and stored at −80° C. in aliquots of 300 μL. To assay baseline DPPIV enzymatic exopeptidase activity, 5 μL of thawed, mouse plasma was diluted (1:5) in buffer (100 mM Tris-HCl, 400 mM NaCl, 50 mM salicylic acid, 1 mM EDTA, pH 7.5) and mixed with 35 μL of the same buffer before being pre-incubated with different concentrations of 10 μL of test compounds or DMSO vehicle for 15 minutes at 37° C. in 96-well black plates (Nunc, #237108). After pre-incubation, 50 μL of 200 μM dipeptide substrate H-Gly-Pro-AMC (Bachem, # L-1225) was added to the mixture. The assay was performed for 1 hour at 37° C. 7-Amino-4-Methylcoumarin (AMC) release was detected measuring fluorescence at an excitation wavelength of 360 nm and an emission wavelength of 460 nm using a Multifunction Microplate Reader (Synergy 4, Biotek). All measurements were carried out at least in duplicate. Results are shown in Table 4.

TABLE 4
Inhibition and Specificity of Test
Compounds in Biological Samples

| | mouse plasma | |
|---|---|---|
| | FAPα | mouse plasma |
| Compound | (PRXS-AMC) | DPPIV |
| Number | (exo IC₅₀, μM) | (IC₅₀, μM) |
| Ref. Comp. | − | + |
| 2 | +++ | + |
| 3 | +++ | ++ |
| 5 | +++ | +++ |
| 7 | +++ | + |
| 8 | +++ | + |
| 9 | +++ | + |

Ref. Comp.: Compound 60 as described in Jansen, K., et al., J Med Chem, 2014. 57(7): p. 3053-74; IC₅₀: +++ refers to IC₅₀ < 1 μM; ++ refers to 1 μM < IC₅₀ < 10 μM; + refers to IC₅₀ < 10 μM; exo: exopeptidase.

### Example B5

### Ex Vivo Inhibition of Circulating FAPα Activity from Plasma of Different Species

Human Plasma

Human blood was obtained from healthy young volunteers. Blood samples were collected in tubes coated with EDTA-K2 by venipuncture method, mixed gently, then kept on ice and centrifuged at 2,500×g for 15 minutes at 4° C. After plasma separation, samples were stored at −80° C. in aliquots of 300 μL.

To determinate the inhibitory potency of exemplary test compounds over circulating FAPα activity from human plasma, 20 μL of thawed plasma were mixed with 20 μL of cFAP buffer (100 mM Tris-HCl, 400 mM NaCl, 50 mM salicylic acid, 1 mM EDTA, pH 7.5) and 10 μL different concentrations of exemplary test compounds or vehicle (DMSO).

Exemplary compounds were allowed to interact with the enzyme for 15 minutes at 37° C. After pre-incubation, 50 μl of 200 μM PRXS-AMC substrate were added to the all mixtures. All reactions were carried out for 1 h at 37° C. protected from light. AMC release was detected measuring fluorescence at an excitation/emission wavelength of 380/460 nm using a Multifunction Microplate Reader. All measurements were carried out as single point.

Results of IC₅₀ of exemplary test compounds over circulating FAPα from human are shown in Table 5.

Hamster Plasma

Male Golden Syrian hamsters were provided by National Laboratory Animal Center (NLAC) in Taiwan. The animals were maintained in a hygienic environment under controlled temperature (20-24° C.) and humidity (50%-80%) with 12 hours light/dark cycles. Free access to standard lab diet [MFG (Oriental Yeast Co., Ltd. Japan)] and autoclaved tap water were granted. All aspects of this work including housing, experimentation and disposal of animals were performed in general accordance with the “Guide for the Care and Use of Laboratory Animals: Eighth Edition” (National Academies Press, Washington, D.C., 2011). In addition, the animal care and use protocol was reviewed and approved by the IACUC at Pharmacology Discovery Services Taiwan, Ltd.

Immediately after the sacrifice of hamsters, blood samples were collected via terminal cardiac puncture in tubes coated with EDTA-K2, mixed gently, then kept on ice and centrifuged at 2,500×g for 15 minutes at 4° C. After plasma separation, samples were stored at −80° C. in aliquots of 300 μL.

To assay exemplary compounds in hamster plasma, a similar protocol as described for human plasma was performed diluting thawed plasma 1:2 in cFAP buffer. In a 96-well black plate, 5 μl of diluted hamster plasma were mixed with 35 μl of the same buffer and 10 μl of exemplary test compounds at different concentrations or vehicle (DMSO).

Exemplary test compounds were allowed to interact with the enzyme for 15 minutes at 37° C. After pre-incubation, 50 μl of 200 μM PRXS-AMC substrate were added to the all mixtures. All reactions were carried out for 1 h at 37° C. protected from light. AMC release was detected measuring fluorescence at an excitation/emission wavelength of 380/460 nm using a Multifunction Microplate Reader. All measurements were carried out as single point.

Results of IC₅₀ of exemplary test compounds over circulating FAPα from hamster plasma are shown in Table 5.

TABLE 5
Inhibition ex-vivo by exemplary compounds of circulating
FAPα activity from human and hamster plasma.

| | FAPα activity in | FAPα activity in |
|---|---|---|
| | human plasma | hamster plasma |
| Compound | (PRXS-AMC) | (PRXS-AMC) |
| No. | IC₅₀, μM | IC₅₀, μM |
| 3 | +++ | +++ |
| 9 | +++ | +++ |

For IC₅₀: +++ refers to IC₅₀ < 1 μM; ++ refers to 1 μM < IC₅₀ < 10 μM; + refers to IC₅₀ > 10 μM.

### Example B6

### Cytoxicity Assays in Human Leukemia Cell Lines

Human acute myeloid leukemia (AML) and non-AML cell lines are purchased from ATCC and cultured following their indications. At the day of the experiment, AML and non-AML cell lines are seeded in white 96-well plate in 100 μL growing medium containing either vehicle (DMSO) or an exemplary compound of the invention at different concentrations. After 48 hours post-treatment, luminescent-based cell viability is determined using Cell-Titer Glo (CTG) assay according to the manufacturer's instructions (Cat. No.: G7573, Promega). Percent of cell viability is calculated by normalizing luminescence signal to the average value from vehicle-treated wells, assumed as the maximum of viability (100%). In every experiment, all treatments are performed in triplicate and reported as % inhibition of viability±standard deviation (SD).

Val-boroPro, a non-specific prolyl peptidase inhibitor, was used as a positive control as described Johnson D C et al, Nature Medicine, 2018. IC₅₀ of certain compounds in the cytotoxicity assays using the human AML cell line MV4-11 are shown in Table 6.

TABLE 6
Inhibition of viability of the human AML
cell line MV4-11 by certain compounds.

| Compound | MV4-11 viability |
|---|---|
| No. | IC50, μM |
| Val-boroPro | +++ |
| 9 | + |
| 15 | + |
| 34 | +++ |
| 40 | ++ |
| 41 | + |
| 44 | +++ |
| 45 | ++ |
| 46 | +++ |
| 47 | ++ |
| 139 | + |
| 192 | +++ |
| 193 | +++ |
| 194 | +++ |

For IC₅₀: +++ refers to IC₅₀ < 3 μM; ++ refers to 3 μM < IC₅₀ < 10 μM; + refers to IC₅₀ > 10 μM.

### EMBODIMENTS

### Embodiment 1

A compound of formula (I):

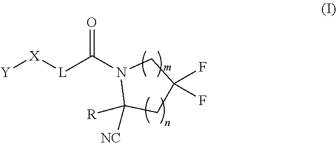

or a pharmaceutically acceptable salt thereof, wherein:
R is hydrogen, C₁-C₆ alkyl, C₃-C₈ cycloalkyl, 3- to 12-membered heterocyclyl, 5- to 10-membered heteroaryl, or C₆-C₁₄ aryl, wherein the C₁-C₆ alkyl, C₃-C₈ cycloalkyl, 3- to 12-membered heterocyclyl, 5- to 10-membered heteroaryl, and C₆-C₁₄ aryl of R are independently optionally substituted by R^{d};
m is 0, 1, 2, 3, or 4;
n is 0, 1, 2, 3, or 4,

wherein m+n is 1, 2, 3, or 4;

### X is —C(═O)—, —O—, —CH(OH)—, —S—, —S(═O)—, or —S(═O)₂—;

### L is

(a)

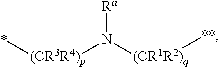

wherein
* represents the point of attachment to the Y—X— moiety,
** represents the point of attachment to the remainder of the molecule,
R^{a} is hydrogen, C₁-C₆ alkyl, C₃-C₈ cycloalkyl, 3- to 12-membered heterocyclyl, 5- to 10-membered heteroaryl, or C₆-C₁₄ aryl, wherein the C₁-C₆ alkyl, C₃-C₈ cycloalkyl, 3- to 12-membered heterocyclyl, 5- to 10-membered heteroaryl, and C₆-C₁₄ aryl of R^{a} are independently optionally substituted by R^{e},
R¹ and R², independently of each other and independently at each occurrence, are hydrogen, C₁-C₂ alkyl, C₃-C₈ cycloalkyl, 3- to 12-membered heterocyclyl, 5- to 10-membered heteroaryl, or C₆-C₁₄ aryl, wherein the C₃-C₈ cycloalkyl, 3- to 12-membered heterocyclyl, 5- to 10-membered heteroaryl, and C₆-C₁₄ aryl of R¹ and R² are independently optionally substituted by R^{f},
   or R¹ and R² are taken together with the carbon atom or atoms to which they are attached to form a 3- to 8-membered cycloalkylene optionally substituted by R^{f}, q is 1, 2, or 3,
R³ and R⁴, independently of each other and independently at each occurrence, are hydrogen, C₃-C₈ cycloalkyl, 3- to 12-membered heterocyclyl, 5- to 10-membered heteroaryl, or C₆-C₁₄ aryl, wherein the C₃-C₈ cycloalkyl, 3- to 12-membered heterocyclyl, 5- to 10-membered heteroaryl, and C₆-C₁₄ aryl of R³ and R⁴ are independently optionally substituted by R^{g},
   or R³ and R⁴ are taken together with the carbon atom to which they are attached to form a 3- to 8-membered cycloalkylene optionally substituted by R^{g}, and
p is 0, 1, or 2;

(b)

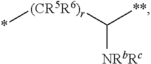

wherein
* represents the point of attachment to the Y—X— moiety,
** represents the point of attachment to the remainder of the molecule,
R⁵ and R⁶, independently of each other and independently at each occurrence, are H, C₁-C₆ alkyl, C₃-C₈ cycloalkyl, 3- to 12-membered heterocyclyl, 5- to 10-membered heteroaryl, or C₆-C₁₄ aryl, wherein the C₁-C₆ alkyl, C₃-C₈ cycloalkyl, 3- to 12-membered heterocyclyl, 5- to 10-membered heteroaryl, and C₆-C₁₄ aryl of R⁵ and R⁶ are independently optionally substituted by R^{h},
R^{b} and R^{c} are independently H, C₁-C₆ alkyl, C₂-C₆ alkenyl, C₂-C₆ alkynyl, C₃-C₈ cycloalkyl, 3- to 12-membered heterocyclyl, 5- to 10-membered heteroaryl, C₆-C₁₄ aryl, or —C(═O)OR¹⁷, wherein the C₁-C₆ alkyl, C₃-C₈ cycloalkyl, 3- to 12-membered heterocyclyl, 5- to 10-membered heteroaryl, and C₆-C₁₄ aryl of R^{b} and R^{c} are independently optionally substituted by Rⁱ, and
r is 1, 2, or 3; or

(c)

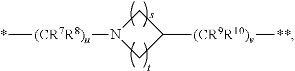

wherein
* represents the point of attachment to the Y—X— moiety,
** represents the point of attachment to the remainder of the molecule,
R⁷ and R⁸, independently of each other and independently at each occurrence, are hydrogen, C₃-C₈ cycloalkyl, 3- to 12-membered heterocyclyl, 5- to 10-membered heteroaryl, or C₆-C₁₄ aryl, wherein the C₃-C₈ cycloalkyl, 3- to 12-membered heterocyclyl, 5- to 10-membered heteroaryl, and C₆-C₁₄ aryl of R⁷ and R⁸ are independently optionally substituted by R^{j},
   or R⁷ and R⁸ are taken together with the carbon atom to which they are attached to form a 3- to 8-membered cycloalkylene optionally substituted by R^{j},
R⁹ and R¹⁰, independently of each other and independently at each occurrence, are H, C₁-C₆ alkyl, C₃-C₈ cycloalkyl, 3- to 12-membered heterocyclyl, 5- to 10-membered heteroaryl, or C₆-C₁₄ aryl, wherein the C₁-C₆ alkyl, C₃-C₈ cycloalkyl, 3- to 12-membered heterocyclyl, 5- to 10-membered heteroaryl, and C₆-C₁₄ aryl of R⁹ and R¹⁰ are independently optionally substituted by R^{k},
s is 1, 2, or 3,
t is 1, 2, or 3,
wherein s+t is 2, 3, or 4,
u is 0 or 1, and
v is 0 or 1;
   Y is C₆-C₉ aryl substituted by R¹¹, 6- to 10-membered heteroaryl substituted by R¹², or 3- to 12-membered heterocyclyl substituted by R¹³, wherein

each R¹, R¹², and R¹³, are independently C₁-C₆ alkyl, C₂-C₆ alkenyl, C₂-C₆ alkynyl, C₃-C₈ cycloalkyl, C₄-C₈ cycloalkenyl, 3- to 12-membered heterocyclyl, 5- to 10-membered heteroaryl, C₆-C₁₄ aryl, —OR¹⁴, —NR¹⁵R¹⁶, —SR¹⁴, —NO₂, —C═NH(OR¹⁴), —C(O)R¹⁴, —OC(O)R¹⁴, —C(O)OR¹⁴, —C(O)NR¹⁵R¹⁶, —NR¹⁴C(O)R¹⁵, —NR¹⁴C(O)OR¹⁵, —NR¹⁴C(O)NR¹⁵R¹⁶, —S(O)R¹⁴, —S(O)₂R¹⁴, —NR¹⁴S(O)R¹⁵, —NR¹⁴S(O)₂R¹⁵, —S(O)NR¹⁵R¹⁶, —S(O)₂NR¹⁵R¹⁶, or —P(O)(OR¹⁵)(OR¹⁵)(OR¹⁶), wherein the C₁-C₆ alkyl, C₂-C₆ alkenyl, C₂-C₆ alkynyl, C₃-C₈ cycloalkyl, C₄-C₈ cycloalkenyl, 3- to 12-membered heterocyclyl, 5- to 10-membered heteroaryl, and C₆-C₁₄ aryl of R¹¹, R¹², and R¹³ are substituted by R^{L};

R¹⁴, R¹⁵ and R¹⁶, independently of each other and independently at each occurrence, are hydrogen, C₁-C₆ alkyl, C₂-C₆ alkenyl, C₂-C₆ alkynyl, C₃-C₈ cycloalkyl, C₆-C₁₄ aryl, 5- to 10-membered heteroaryl, or 3- to 12-membered heterocyclyl, wherein the C₁-C₆ alkyl, C₂-C₆ alkenyl, C₂-C₆ alkynyl, C₃-C₈ cycloalkyl, C₆-C₁₄ aryl, 5- to 10-membered heteroaryl, and 3- to 12-membered heterocyclyl of R¹⁴, R¹⁵ and R¹⁶ are independently substituted by C₁-C₆ perhaloalkyl, C₁-C₆ alkoxy, C₁-C₆ perhaloalkoxy, C₆-C₁₄ aryl or C₆-C₁₄ aryloxy wherein the C₆-C₁₄ aryl or C₆-C₁₄ aryloxy is further optionally substituted by halogen, —OH, cyano, C₁-C₆ alkyl, C₁-C₆ perhaloalkyl, C₁-C₆ alkoxy, or C₁-C₆ perhaloalkoxy; and, wherein at least one of R¹⁴, R¹⁵ and R¹⁶, when present, is not hydrogen;

R^{L} is C₁-C₆ alkyl, C₂-C₆ alkenyl, C₂-C₆ alkynyl, C₃-C₈ cycloalkyl, C₆-C₁₄ aryl, 5- to 10-membered heteroaryl, or 3- to 12-membered heterocyclyl, wherein the C₁-C₆ alkyl, C₂-C₆ alkenyl, C₂-C₆ alkynyl, C₃-C₈ cycloalkyl, C₆-C₁₄ aryl, 5- to 10-membered heteroaryl, or 3- to 12-membered heterocyclyl of R^{L} is substituted by halogen, —OH, cyano, C₁-C₆ alkyl, C₁-C₆ perhaloalkyl, C₁-C₆ alkoxy, C₁-C₆ perhaloalkoxy or C₆-C₁₄ aryl, wherein the C₆-C₁₄ aryl is further optionally substituted by halogen, —OH, cyano, C₁-C₆ alkyl, C₁-C₆ perhaloalkyl, C₁-C₆ alkoxy, or C₁-C₆ perhaloalkoxy; and

R^{d}, R^{e}, R^{f}, R^{g}, R^{h}, Rⁱ, R^{j}, and R^{k}, independently of each other and independently at each occurrence, are halogen, C₁-C₆ alkyl, C₂-C₆ alkenyl, C₂-C₆ alkynyl, C₃-C₈ cycloalkyl, C₆-C₁₄ aryl, 5- to 10-membered heteroaryl, 3- to 12-membered heterocyclyl, —OR¹⁴, —NR¹⁵R¹⁶, cyano, or nitro.

### Embodiment 2

The compound of embodiment 1, or a salt thereof, wherein X is —C(═O)—.

### Embodiment 3

The compound of embodiment 1, or a salt thereof, wherein X is —O—.

### Embodiment 4

The compound of embodiment 1, or a salt thereof, wherein X is —CH(OH)—.

### Embodiment 5

The compound of any one of embodiments 1 to 4, or a salt thereof, wherein L is —NH—CR¹R²—.

### Embodiment 6

The compound of embodiment 5, or a salt thereof, wherein L is —NH—CH₂—.

### Embodiment 7

The compound of embodiment 5, or a salt thereof, wherein L is —NH—CH(CH₃)—.

### Embodiment 8

The compound of embodiment 5, or a salt thereof, wherein L is —NH—CR¹R²—, wherein R¹ and R² are taken together with the carbon atom to which they are attached to form a 3- to 8-membered cycloalkylene.

### Embodiment 9

The compound of embodiment 8, or a salt thereof, wherein R¹ and R² are taken together with the carbon atom to which they are attached to form a cyclopropylene.

### Embodiment 10

The compound of any one of embodiments 1 to 4, or a salt thereof, wherein L is

—CR⁵R⁶—CH(NR^{b}R^{c})—.

### Embodiment 11

The compound of embodiment 10, or a salt thereof, wherein L is —CR⁵R⁶—CH(NR^{b}R^{c})—, wherein R⁶, R^{b}, and R^{c} are H, and R⁵ is H or C₁-C₆ alkyl.

### Embodiment 12

The compound of any one of embodiments 1 to 4, or a salt thereof, wherein L is

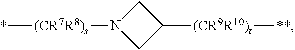

wherein * represents the point of attachment to the Y—X— moiety, ** represents the point of attachment to the remainder of the molecule.

### Embodiment 13

The compound of embodiment 12, or a salt thereof, wherein L is

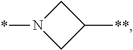

wherein * represents the point of attachment to the Y—X— moiety, and ** represents the point of attachment to the remainder of the molecule.

### Embodiment 14

The compound of embodiment 1, or a salt thereof, wherein the —X-L- moiety is selected from the group consisting of

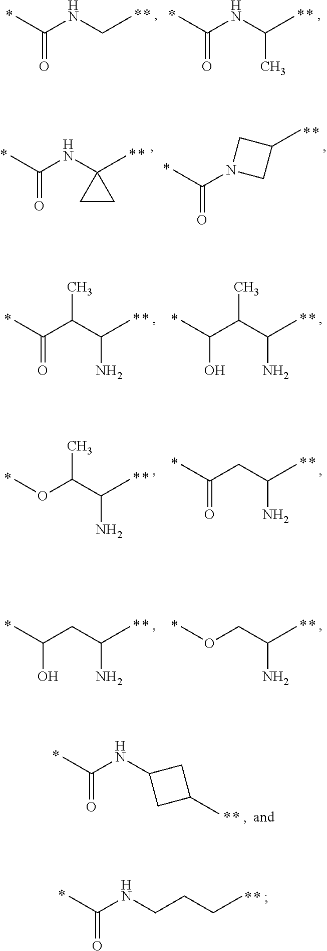

wherein * represents the point of attachment to the Y moiety, and ** represents the point of attachment to the remainder of the molecule.

### Embodiment 15

The compound of any one of embodiments 1 to 14, or a salt thereof, wherein Y is C₆-C₉ aryl substituted by R¹.

### Embodiment 16

The compound of any one of embodiments 1 to 14, or a salt thereof, wherein Y is 6- to 10-membered heteroaryl substituted by R¹².

### Embodiment 17

The compound of embodiment 16, or a salt thereof, wherein Y is pyridin-4-yl substituted by R¹² in the 3-position.

### Embodiment 18

The compound of embodiment 16 or 17, or a salt thereof, wherein R¹² is C₁-C₆ alkyl substituted by R^{L}.

### Embodiment 19

The compound of embodiment 16 or 17, or a salt thereof, wherein R¹² is C₂-C₆ alkenyl substituted by R^{L}.

### Embodiment 20

The compound of embodiment 16 or 17, or a salt thereof, wherein R¹² is 3- to 12-membered heterocyclyl substituted by R^{L}.

### Embodiment 21

The compound of any one of embodiments 18 to 20, or a salt thereof, wherein R^{L} is C₆-C₁₄ aryl substituted by halogen, —OH, cyano, C₁-C₆ alkyl, C₁-C₆ perhaloalkyl, C₁-C₆ alkoxy, C₁-C₆ perhaloalkoxy or C₆-C₁₄ aryl.

### Embodiment 22

The compound of embodiment 16 or 17, or a salt thereof, wherein R¹² is —NR¹⁴C(O)R¹⁵

### Embodiment 23

The compound of embodiment 22, or a salt thereof, wherein at least one of R¹⁴ and R¹⁵ is C₁-C₆ alkyl, or C₆-C₁₄ aryl, wherein the C₁-C₆ alkyl, or C₆-C₁₄ aryl of R¹⁴ and R¹⁵ are independently substituted by C₁-C₆ perhaloalkyl, C₁-C₆ alkoxy, C₁-C₆ perhaloalkoxy, C₆-C₁₄ aryl or C₆-C₁₄ aryloxy, wherein the C₆-C₁₄ aryl or C₆-C₁₄ aryloxy is further optionally substituted by halogen, —OH, cyano, C₁-C₆ alkyl, C₁-C₆ perhaloalkyl, C₁-C₆ alkoxy, or C₁-C₆ perhaloalkoxy.

### Embodiment 24

The compound of any one of embodiments 1 to 14, or a salt thereof, wherein Y is 3- to 12-membered heterocyclyl substituted by R¹³.

### Embodiment 25

The compound of any one of embodiments 1 to 24, or a salt thereof, wherein m=n=1.

### Embodiment 26

The compound of any one of embodiments 1 to 25, or a salt thereof, wherein R is hydrogen.

### Embodiment 27

A compound of Table 1, or a salt thereof.

### Embodiment 28

A pharmaceutical composition comprising a compound of any one of embodiments 1-27, or a pharmaceutically acceptable salt thereof, and a pharmaceutically acceptable carrier.

### Embodiment 29

A method of treating a disease or disorder mediated by fibroblast activation protein (FAP) in an individual in need thereof comprising administering to the individual a therapeutically effective amount of a compound of any one of embodiments 1-27, or a pharmaceutically acceptable salt thereof, or a pharmaceutical composition of embodiment 28.

### Embodiment 30

A method of treating a disease or disorder characterized by proliferation, tissue remodeling, chronic inflammation, obesity, glucose intolerance, or insulin insensitivity in an individual in need thereof, comprising administering to the individual a therapeutically effective amount of a compound of any one of embodiments 1-27, or a pharmaceutically acceptable salt thereof, or a pharmaceutical composition of embodiment 28.

### Embodiment 31

The method of embodiments 29 or 30, wherein the disease or disorder is breast cancer, colorectal cancer, ovarian cancer, prostate cancer, pancreatic cancer, kidney cancer, lung cancer, melanoma, fibrosarcoma, bone sarcoma, connective tissue sarcoma, renal cell carcinoma, giant cell carcinoma, squamous cell carcinoma, leukemia, skin cancer, soft tissue cancer, liver cancer, gastrointestinal carcinoma, or adenocarcinoma.

### Embodiment 32

The method of embodiment 31, wherein the disease or disorder is metastatic kidney cancer, chronic lymphocytary leukemia, pancreatic adenocarcinoma, or non-small cell lung cancer.

### Embodiment 33

The method of embodiment 29 or 30, wherein the disease or disorder is fibrotic disease, wound healing, keloid formation, osteoarthritis, rheumatoid arthritis and related disorders involving cartilage degradation, atherosclerotic disease, Crohn's disease, or Type II diabetes

### Embodiment 34

A method of reducing tumor growth, tumor proliferation, or tumorigenicity in an individual in need thereof, comprising administering to the individual a compound of any one of embodiments 1-27, or a pharmaceutically acceptable salt thereof, or a pharmaceutical composition of embodiment 28.

### Embodiment 35

A method of inhibiting FAP in an individual comprising administering to the individual a compound of any one of embodiments 1-27, or a pharmaceutically acceptable salt thereof, or a pharmaceutical composition of embodiment 28.

### Embodiment 36

A method of inhibiting FAP in a cell comprising administering or delivering to the cell a compound of any one of embodiments 1-27, or a pharmaceutically acceptable salt thereof, or a pharmaceutical composition of embodiment 28, or a metabolite of the foregoing.

### Embodiment 37

The method of embodiment 36, wherein the cell is a fibroblast.

### Embodiment 38

The method of embodiment 36 or 37, wherein the cell is a cancer associated fibroblast (CAF) or a reactive stromal fibroblast.

### Embodiment 39

A method of inhibiting FAP in a tumor comprising administering or delivering to the tumor a compound of any one of embodiments 1-27, or a pharmaceutically acceptable salt thereof, or a pharmaceutical composition of embodiment 28, or a metabolite of the foregoing.

### Embodiment 40

A method of inhibiting FAP in plasma comprising administering or delivering to the plasma a compound of any one of embodiments 1-27, or a pharmaceutically acceptable salt thereof, or a pharmaceutical composition of embodiment 28, or a metabolite of the foregoing.

### Embodiment 41

The method of any one of embodiments 35-40, wherein inhibiting FAP comprises inhibiting an endopeptidase activity of FAP.

### Embodiment 42

The method of any one of embodiments 35-40, wherein inhibiting FAP comprises inhibiting an exopeptidase activity of FAP.

### Embodiment 43

A method of enhancing an immune response in an individual comprising administering (a) an immune checkpoint inhibitor and (b) a compound of any one of embodiments 1-27, or a pharmaceutically acceptable salt thereof, or a pharmaceutical composition of embodiment 28.

### Embodiment 44

A method of increasing the level of FGF21 expression in an individual comprising administering to the individual a compound of any one of embodiments 1-27, or a pharmaceutically acceptable salt thereof, or a pharmaceutical composition of embodiment 28.

### Embodiment 45

The method of embodiment 44, further comprising administering an inducer of FGF21 expression

### Embodiment 46

The method of embodiment 45, wherein the inducer of FGF21 expression is PPARα agonist.

### Embodiment 47

The method of embodiment 46, wherein the PPARα agonist is fibrate or fenofibrate.

### Embodiment 48

The composition of embodiment 28 for use as a human or veterinary medicament.

### Embodiment 49

Use of a compound of any one of embodiments 1-27, or a pharmaceutically acceptable salt thereof, or a pharmaceutical composition of embodiment 28, in the manufacture of a medicament for the prevention and/or treatment of a disorder or disease mediated by FAP.

All references throughout, such as publications, patents, patent applications and published patent applications, are incorporated herein by reference in their entireties.

What is claimed is:

1. A compound of formula (I): 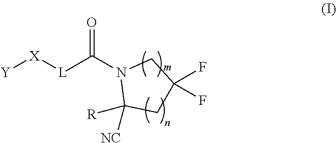
a pharmaceutically acceptable salt, stereoisomer or tautomer thereof, wherein:
R is hydrogen, C₁-C₆ alkyl, C₃-C₈ cycloalkyl, 3- to 12-membered heterocyclyl, 5- to 10-membered heteroaryl, or C₆-C₁₄ aryl, wherein the C₁-C₆ alkyl, C₃-C₈ cycloalkyl, 3- to 12-membered heterocyclyl, 5- to 10-membered heteroaryl, and C₆-C₁₄ aryl of R are independently optionally substituted by R^{d};
m is 0, 1, 2, 3, or 4;
n is 0, 1, 2, 3, or 4,
wherein m+n is 1, 2, 3, or 4;
X is —C(═O)—, —O—, —CH(OH)—, —S—, —S(═O)—, or —S(═O)₂—;
L is
(a) 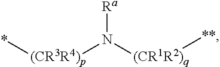
wherein
* represents the point of attachment to the Y—X— moiety,
** represents the point of attachment to the remainder of the molecule,
R^{a} is hydrogen, C₁-C₆ alkyl, C₃-C₈ cycloalkyl, 3- to 12-membered heterocyclyl, 5- to 10-membered heteroaryl, or C₆-C₁₄ aryl, wherein the C₁-C₆ alkyl, C₃-C₈ cycloalkyl, 3- to 12-membered heterocyclyl, 5- to 10-membered heteroaryl, and C₆-C₁₄ aryl of R^{a} are independently optionally substituted by R^{e},
R¹ and R², independently of each other and independently at each occurrence, are hydrogen, C₁-C₂ alkyl, C₃-C₈ cycloalkyl, 3- to 12-membered heterocyclyl, 5- to 10-membered heteroaryl, or C₆-C₁₄ aryl, wherein the C₃-C₈ cycloalkyl, 3- to 12-membered heterocyclyl, 5- to 10-membered heteroaryl, and C₆-C₁₄ aryl of R¹ and R² are independently optionally substituted by R^{f},
or R¹ and R² are taken together with the carbon atom or atoms to which they are attached to form a 3- to 8-membered cycloalkylene optionally substituted by R^{f},
q is 1, 2, or 3,
R³ and R⁴, independently of each other and independently at each occurrence, are hydrogen, C₃-C₈ cycloalkyl, 3- to 12-membered heterocyclyl, 5- to 10-membered heteroaryl, or C₆-C₁₄ aryl, wherein the C₃-C₈ cycloalkyl, 3- to 12-membered heterocyclyl, 5- to 10-membered heteroaryl, and C₆-C₁₄ aryl of R³ and R⁴ are independently optionally substituted by R^{g},
or R³ and R⁴ are taken together with the carbon atom to which they are attached to form a 3- to 8-membered cycloalkylene optionally substituted by R^{g}, and
p is 0, 1, or 2;
(b) 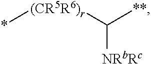
wherein
* represents the point of attachment to the Y—X— moiety,
** represents the point of attachment to the remainder of the molecule,
R⁵ and R⁶, independently of each other and independently at each occurrence, are H, C₁-C₆ alkyl, C₃-C₈ cycloalkyl, 3- to 12-membered heterocyclyl, 5- to 10-membered heteroaryl, or C₆-C₁₄ aryl, wherein the C₁-C₆ alkyl, C₃-C₈ cycloalkyl, 3- to 12-membered heterocyclyl, 5- to 10-membered heteroaryl, and C₆-C₁₄ aryl of R⁵ and R⁶ are independently optionally substituted by R^{h},
R^{b} and R^{c} are independently H, C₁-C₆ alkyl, C₂-C₆ alkenyl, C₂-C₆ alkynyl, C₃-C₈ cycloalkyl, 3- to 12-membered heterocyclyl, 5- to 10-membered heteroaryl, C₆-C₁₄ aryl, or —C(═O)OR¹⁷ wherein the C₁-C₆ alkyl, C₃-C₈ cycloalkyl, 3- to 12-membered heterocyclyl, 5- to 10-membered heteroaryl, and C₆-C₁₄ aryl of R^{b} and R^{c} are independently optionally substituted by R¹, and
r is 1, 2, or 3; or
(c) 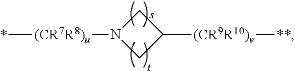
wherein
* represents the point of attachment to the Y—X— moiety,
** represents the point of attachment to the remainder of the molecule,
R⁷ and R⁸, independently of each other and independently at each occurrence, are hydrogen, C₃-C₈ cycloalkyl, 3- to 12-membered heterocyclyl, 5- to 10-membered heteroaryl, or C₆-C₁₄ aryl, wherein the C₃-C₈ cycloalkyl, 3- to 12-membered heterocyclyl, 5- to 10-membered heteroaryl, and C₆-C₁₄ aryl of R⁷ and R⁸ are independently optionally substituted by R^{j},
or R⁷ and R⁸ are taken together with the carbon atom to which they are attached to form a 3- to 8-membered cycloalkylene optionally substituted by R^{j},
R⁹ and R¹⁰, independently of each other and independently at each occurrence, are H, C₁-C₆ alkyl, C₃-C₈ cycloalkyl, 3- to 12-membered heterocyclyl, 5- to 10-membered heteroaryl, or C₆-C₁₄ aryl, wherein the C₁-C₆ alkyl, C₃-C₈ cycloalkyl, 3- to 12-membered heterocyclyl, 5- to 10-membered heteroaryl, and C₆-C₁₄ aryl of R⁹ and R¹⁰ are independently optionally substituted by R^{k},
s is 1, 2, or 3,
t is 1, 2, or 3,
wherein s+t is 2, 3, or 4,
u is 0 or 1, and
v is 0 or 1;
Y is C₆-C₉ aryl substituted by R¹¹, 6- to 10-membered heteroaryl substituted by R¹², or 3- to 12-membered heterocyclyl substituted by R¹³, wherein
each R¹¹, R¹², and R¹³, are independently C₁-C₆ alkyl, C₂-C₆ alkenyl, C₂-C₆ alkynyl, C₃-C₈ cycloalkyl, C₄-C₈ cycloalkenyl, 3- to 12-membered heterocyclyl, 5- to 10-membered heteroaryl, C₆-C₁₄ aryl, —OR¹⁴, —NR¹⁵R¹⁶, —SR¹⁴, —NO₂, —C═NH(OR¹⁴), —C(O)R¹⁴, —OC(O)R¹⁴, —C(O)OR¹⁴, —C(O)NR¹⁵R¹⁶, —NR¹⁴C(O)R¹⁵, —NR¹⁴C(O)OR¹⁵, —NR¹⁴C(O)NR¹⁵R¹⁶, —S(O)R¹⁴, —S(O)₂R¹⁴, —NR¹⁴S(O)R¹⁵, —NR¹⁴S(O)₂R¹⁵, —S(O)NR¹⁵R¹⁶, —S(O)₂NR¹⁵R¹⁶, or —P(O)(OR¹⁵)(OR¹⁶), wherein the C₁-C₆ alkyl, C₂-C₆ alkenyl, C₂-C₆ alkynyl, C₃-C₈ cycloalkyl, C₄-C₈ cycloalkenyl, 3- to 12-membered heterocyclyl, 5- to 10-membered heteroaryl, and C₆-C₁₄ aryl of R¹¹, R¹², and R¹³ are substituted by R^{L};
R¹⁴, R¹⁵ and R¹⁶, independently of each other and independently at each occurrence, are hydrogen, C₁-C₆ alkyl, C₂-C₆ alkenyl, C₂-C₆ alkynyl, C₃-C₈ cycloalkyl, C₆-C₁₄ aryl, 5- to 10-membered heteroaryl, or 3- to 12-membered heterocyclyl, wherein the C₁-C₆ alkyl, C₂-C₆ alkenyl, C₂-C₆ alkynyl, C₃-C₈ cycloalkyl, C₆-C₁₄ aryl, 5- to 10-membered heteroaryl, and 3- to 12-membered heterocyclyl of R¹⁴, R¹⁵ and R¹⁶ are independently substituted by C₁-C₆ alkoxy, C₁-C₆ perhaloalkoxy, C₆-C₁₄ aryl or C₆-C₁₄ aryloxy wherein the C₆-C₁₄ aryl or C₆-C₁₄ aryloxy is further optionally substituted by halogen, —OH, cyano, C₁-C₆ alkyl, C₁-C₆ perhaloalkyl, C₁-C₆ alkoxy, or C₁-C₆ perhaloalkoxy; and, wherein at least one of R¹⁴, R¹⁵ and R¹⁶, when present, is not hydrogen;
R^{L} is C₁-C₆ alkyl, C₂-C₆ alkenyl, C₂-C₆ alkynyl, C₃-C₈ cycloalkyl, C₆-C₁₄ aryl, 5- to 10-membered heteroaryl, or 3- to 12-membered heterocyclyl, wherein the C₁-C₆ alkyl, C₂-C₆ alkenyl, C₂-C₆ alkynyl, C₃-C₈ cycloalkyl, C₆-C₁₄ aryl, 5- to 10-membered heteroaryl, or 3- to 12-membered heterocyclyl of R^{L} is substituted by halogen, —OH, cyano, oxo, —NH₂, —NH-(3- to 12-membered heterocyclyl), —O-(3- to 12-membered heterocyclyl), C₁-C₆ alkyl, C₁-C₆ perhaloalkyl, C₁-C₆ alkoxy, C₁-C₆ perhaloalkoxy or C₆-C₁₄ aryl, wherein
the C₁-C₆ alkyl is further optionally substituted by 3- to 12-membered heterocyclyl, wherein the 3- to 12-membered heterocyclyl is further optionally substituted by C₁-C₆ alkyl,
the 3- to 12-membered heterocyclyl of the —NH-(3- to 12-membered heterocyclyl) and the —O-(3- to 12-membered heterocyclyl) is further optionally substituted by C₁-C₆ alkyl, and
the C₆-C₁₄ aryl is further optionally substituted by halogen, —OH, cyano, C₁-C₆ alkyl, C₁-C₆ perhaloalkyl, C₁-C₆ alkoxy, or C₁-C₆ perhaloalkoxy; and
R^{d}, R^{e}, R^{f}, R^{g}, R^{h}, Rⁱ, R^{j}, and R^{k}, independently of each other and independently at each occurrence, are halogen, C₁-C₆ alkyl, C₂-C₆ alkenyl, C₂-C₆ alkynyl, C₃-C₈ cycloalkyl, C₆-C₁₄ aryl, 5- to 10-membered heteroaryl, 3- to 12-membered heterocyclyl, —OR¹⁴, —NR¹⁵R¹⁶, cyano, or nitro.

2. The compound of claim 1, a pharmaceutically acceptable salt, stereoisomer or tautomer thereof, wherein X is —C(═O)—.

3. The compound of claim 1, a pharmaceutically acceptable salt, stereoisomer or tautomer thereof, wherein X is —O—.

4. The compound of claim 1, a pharmaceutically acceptable salt, stereoisomer or tautomer thereof, wherein X is —CH(OH)—.

5. The compound of claim 1, a pharmaceutically acceptable salt, stereoisomer or tautomer thereof, wherein L is —NH—CR¹R²—.

6. The compound of claim 5, a pharmaceutically acceptable salt, stereoisomer or tautomer thereof, wherein L is —NH—CH₂—.

7. The compound of claim 5, a pharmaceutically acceptable salt, stereoisomer or tautomer thereof, wherein L is —NH—CH(CH₃)—.

8. The compound of claim 5, a pharmaceutically acceptable salt, stereoisomer or tautomer thereof, wherein L is —NH—CR¹R²—, wherein R¹ and R² are taken together with the carbon atom to which they are attached to form a 3- to 8-membered cycloalkylene.

9. The compound of claim 8, a pharmaceutically acceptable salt, stereoisomer or tautomer thereof, wherein R¹ and R² are taken together with the carbon atom to which they are attached to form a cyclopropylene.

10. The compound of claim 1, a pharmaceutically acceptable salt, stereoisomer or tautomer thereof, wherein L is —CR⁵R⁶—CH(NR^{b}R^{c})—.

11. The compound of claim 10, a pharmaceutically acceptable salt, stereoisomer or tautomer thereof, wherein L is —CR⁵R⁶—CH(NR^{b}R^{c})—, wherein R⁶, R^{b}, and R^{c} are H, and R⁵ is H or C₁-C₆ alkyl.

12. The compound of claim 1, a pharmaceutically acceptable salt, stereoisomer or tautomer thereof, wherein L is 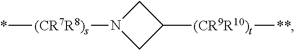
wherein * represents the point of attachment to the Y—X— moiety, ** represents the point of attachment to the remainder of the molecule.

13. The compound of claim 12, a pharmaceutically acceptable salt, stereoisomer or tautomer thereof, wherein L is 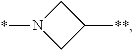
wherein * represents the point of attachment to the Y—X— moiety, and ** represents the point of attachment to the remainder of the molecule.

14. The compound of claim 1, a pharmaceutically acceptable salt, stereoisomer or tautomer thereof, wherein the —X-L- moiety is selected from the group consisting of 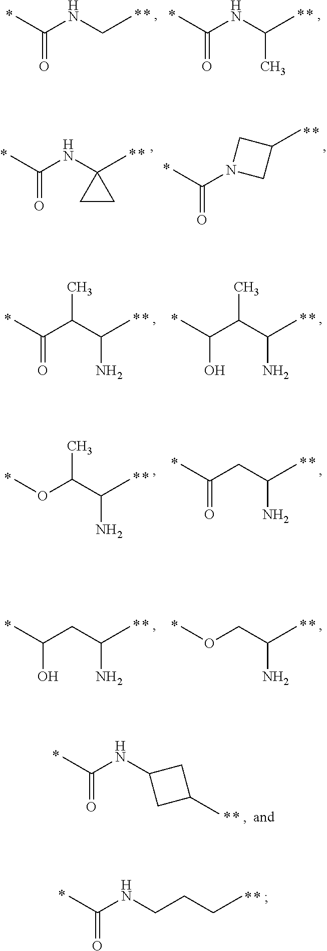
wherein * represents the point of attachment to the Y moiety, and ** represents the point of attachment to the remainder of the molecule.

15. The compound of claim 1, a pharmaceutically acceptable salt, stereoisomer or tautomer thereof, wherein Y is C₆-C₉ aryl substituted by R¹¹.

16. The compound of claim 1, a pharmaceutically acceptable salt, stereoisomer or tautomer thereof, wherein Y is 6- to 10-membered heteroaryl substituted by R¹².

17. The compound of claim 16, a pharmaceutically acceptable salt, stereoisomer or tautomer thereof, wherein Y is pyridin-4-yl substituted by R¹² in the 3-position.

18. The compound of claim 16, a pharmaceutically acceptable salt, stereoisomer or tautomer thereof, wherein R¹² is C₁-C₆ alkyl substituted by R^{L}.

19. The compound of claim 16, a pharmaceutically acceptable salt, stereoisomer or tautomer thereof, wherein R¹² is C₂-C₆ alkenyl substituted by R^{L}.

20. The compound of claim 16, a pharmaceutically acceptable salt, stereoisomer or tautomer thereof, wherein R¹² is 3- to 12-membered heterocyclyl substituted by R^{L}.

21. The compound of claim 18, a pharmaceutically acceptable salt, stereoisomer or tautomer thereof, wherein R^{L} is C₆-C₁₄ aryl substituted by halogen, —OH, cyano, C₁-C₆ alkyl, C₁-C₆ perhaloalkyl, C₁-C₆ alkoxy, C₁-C₆ perhaloalkoxy or C₆-C₁₄ aryl.

22. The compound of claim 16, a pharmaceutically acceptable salt, stereoisomer or tautomer thereof, wherein R¹² is —NR¹⁴C(O)R¹⁵.

23. The compound of claim 22, a pharmaceutically acceptable salt, stereoisomer or tautomer thereof, wherein at least one of R¹⁴ and R¹⁵ is C₁-C₆ alkyl, or C₆-C₁₄ aryl, wherein the C₁-C₆ alkyl, or C₆-C₁₄ aryl of R¹⁴ and R¹⁵ are independently substituted by C₁-C₆ perhaloalkyl, C₁-C₆ alkoxy, C₁-C₆ perhaloalkoxy, C₆-C₁₄ aryl or C₆-C₁₄ aryloxy, wherein the C₆-C₁₄ aryl or C₆-C₁₄ aryloxy is further optionally substituted by halogen, —OH, cyano, C₁-C₆ alkyl, C₁-C₆ perhaloalkyl, C₁-C₆ alkoxy, or C₁-C₆ perhaloalkoxy.

24. The compound of claim 1, a pharmaceutically acceptable salt, stereoisomer or tautomer thereof, wherein Y is 3- to 12-membered heterocyclyl substituted by R¹³.

25. The compound of claim 1, a pharmaceutically acceptable salt, stereoisomer or tautomer thereof, wherein m=n=1.

26. The compound of claim 1, a pharmaceutically acceptable salt, stereoisomer or tautomer thereof, wherein R is hydrogen.

27. A compound of Table 1, a pharmaceutically acceptable salt, stereoisomer or tautomer thereof.

28. A pharmaceutical composition comprising a compound of claim 1, or a pharmaceutically acceptable salt thereof, and a pharmaceutically acceptable carrier.

29. A method of treating a disease or disorder mediated by fibroblast activation protein (FAP) in an individual in need thereof comprising administering to the individual a therapeutically effective amount of a compound of claim 1, or a pharmaceutically acceptable salt thereof.

30. A method of treating a disease or disorder characterized by proliferation, tissue remodeling, chronic inflammation, obesity, glucose intolerance, or insulin insensitivity in an individual in need thereof, comprising administering to the individual a therapeutically effective amount of a compound claim 1, or a pharmaceutically acceptable salt thereof.

31.-33. (canceled)

34. A method of reducing tumor growth, tumor proliferation, or tumorigenicity in an individual in need thereof, comprising administering to the individual a compound of claim 1, or a pharmaceutically acceptable salt thereof.

35. A method of inhibiting FAP in an individual comprising administering to the individual a compound of claim 1, or a pharmaceutically acceptable salt thereof.

36. A method of inhibiting FAP in a cell comprising administering or delivering to the cell a compound of claim 1, or a pharmaceutically acceptable salt thereof.

37. (canceled)

38. (canceled)

39. A method of inhibiting FAP in a tumor comprising administering or delivering to the tumor a compound of claim 1, or a pharmaceutically acceptable salt thereof.

40. A method of inhibiting FAP in plasma comprising administering or delivering to the plasma a compound of claim 1, or a pharmaceutically acceptable salt thereof.

41. (canceled)

42. (canceled)

43. A method of enhancing an immune response in an individual comprising administering (a) an immune checkpoint inhibitor and (b) a compound of claim 1, or a pharmaceutically acceptable salt thereof.

44. A method of increasing the level of FGF21 expression in an individual comprising administering to the individual a compound of claim 1, or a pharmaceutically acceptable salt thereof.

45.-49. (canceled)
